(19) United States
(12) Patent Application Publication
(10) Pub. No.: US 2004/0072191 A1
(43) Pub. Date: Apr. 15, 2004

(54) Antisense RNA standardizing control

(76) Inventor: Alex Chenchik, Emerald Hills, CA (US)

Correspondence Address: BECTON, DICKINSON AND COMPANY, 1 BECTON DRIVE, FRANKLIN LAKES, NJ, 07417-1880, US

(21) Appl. No.: 10/384,245

(22) Filed: Mar. 7, 2003

Related U.S. Application Data
(60) Provisional application No. 60/362,823, filed on Mar. 7, 2002.

(51) Int. Cl. C12Q 1/68; C07H 21/02

(52) U.S. Cl. 435/6; 536/23.1

(57) ABSTRACT

Methods for producing a population of distinct aRNA molecules from an initial population of distinct mRNA molecules are provided. In the subject methods, an initial mRNA sample is contacted with a population of distinct tagged antisense molecules to produce a population of hybrid or duplex molecules. The resultant population of hybrid molecules, or template derivatives thereof, is then contacted with a DNA dependent RNA polymerase to produce the population of distinct aRNA molecules. Also provided are kits for practicing these methods. The subject methods find use a variety of different applications in which the preparation of aRNA is desired, e.g., the preparation of nucleic acid targets for use in array based hybridization applications.

### BACKGROUND OF THE INVENTION

The technical field of this invention is molecular biology, and particularly tools for nucleic acid array standardization.

The characterization of cell specific gene expression finds application in a variety of disciplines, such as in the analysis of differential expression between different tissue types, different stages of cellular growth or between normal and diseased states. Fundamental to the characterization of cell specific gene expression is the detection, qualitative or quantitative, of mRNA. However, the detection of mRNA is often complicated by one or more of the following factors: cell heterogeneity, paucity of material, or limits of low abundance mRNA detection.

One method which has been developed to address at least some of the problems associated with mRNA detection is known as “antisense RNA” (aRNA) amplification. In this method, first strand cDNA is prepared from mRNA using an oligo dT primer that comprises an RNA polymerase promoter located at the 5′ end of the oligo dT region. The first strand cDNA is then converted to ds cDNA. Finally, the ds cDNA is contacted with the appropriate RNA polymerase under conditions sufficient to produce aRNA. The method can be adjusted to obtain amplification of the initial mRNA of up to 106 fold. The aRNA can then be used in a variety of applications as hybridization target, for cDNA library construction and the like, where such applications include assays for differential gene expression.

Current methods of antisense RNA amplification as described above that employ RNA intermediates are not entirely satisfactory. For example, methods currently employed require the synthesis of one or more DNA strands, e.g., first and second cDNA, in addition to RNA transcription with the RNA polymerase, and therefore require multiple steps using multiple reagents.

Accordingly, there is interest in the development of improved methods of antisense RNA amplification which do not suffer from one or more of the above deficiencies experienced using current methods.

U.S. patents disclosing methods of antisense RNA synthesis include: U.S. Pat. Nos. 6,312,928; 6,309,384; 6,132,997; 5,932,451; 5,869,249; 5,716,785; 5,593,863; 5,554,516; 5,545,522; 5,514,545; 5,512,462; 5,470,724; 5,437,990; 5,399,491; 5,130,238; 5,021,335; and 4,683,195. Antisense RNA synthesis is also discussed in Phillips & Eberwine, Methods: A Companion to Methods in Enzymology (1996) 10:283-288; Eberwine et al., Proc. Natl. Acad. Sci. USA (1992) 89: 3010-3014; Eberwine, Biotechniques (1996) 20:584-591; and Methods in Enzymology (1992) 216:80-100.

### SUMMARY OF THE INVENTION

Methods for producing a population of distinct aRNA molecules from an initial population of distinct mRNA molecules are provided. In the subject methods, an initial mRNA sample is contacted with a population of distinct tagged antisense nucleic acid molecules to produce a population of hybrid molecules. The resultant hybrid molecules, or derivatives thereof (e.g., template structures produced there from) are then transcribed into aRNA molecules using a DNA dependent RNA polymerase transcription step Also provided are kits for practicing these methods. The subject methods find use a variety of different applications in which the preparation of aRNA is desired, e.g., the preparation of nucleic acid targets for use in array based hybridization applications, such as differential gene expression analysis applications.

The subject invention provides methods for producing at least one aRNA molecule corresponding to an mRNA molecule by the following steps: (a) contacting the mRNA molecule with tagged antisense molecule to produce hybrid structure, where the tagged antisense molecule includes an antisense domain complementary to at least about 20 nt of the mRNA molecule; and (b) transcribing the aRNA from the hybrid structure or a template derivative thereof to produce the at least one aRNA molecule corresponding to the mRNA molecule. In certain embodiments, the hybrid structure produced in step (a) is converted to a template prior to the transcription step (b). In certain embodiments, a plurality of aRNA molecules corresponding to the mRNA molecule is produced. In certain embodiments, the antisense domain is complementary to at least about 25 nt, or 30nt, or 50 nt of the mRNA molecule. In certain embodiments, the tagged antisense molecule includes a tag domain that includes an RNA polymerase promoter. In certain embodiments, the mRNA molecule is present in a complex nucleic acid mixture. In certain embodiments, the method further includes separating the hybrid structure from any single stranded tagged antisense molecules prior to the transcribing step. In certain embodiments, the method is a method of producing at least one aRNA molecule for at least two different mRNA molecules, e.g., a plurality of different mRNA molecules, in a sample.

Also provided are array-based hybridization assays that include the steps of: (a) generating a population of target nucleic acids using a population of tagged antisense molecules according to the provided methods; (b) contacting an array of probe nucleic acids on a surface of a solid support with the population of target nucleic acids; and (c) detecting hybridization complexes on a surface of the array. In certain embodiments, at least a subset of the probe nucleic acids present on the array are represented in the population of tagged antisense molecules, and in certain embodiments, all of the probe nucleic acids present on the array are represented in the population of tagged antisense molecules.

Also provided is a set of tagged antisense molecules each for use in preparation of target nucleic acids for hybridization to an array of probe nucleic acids corresponding to a plurality of different genes, wherein the set includes at least 20 distinct tagged antisense molecules, wherein each of the tagged antisense molecules includes an mRNA complementary domain of at least about 20 nt in length, and at least a subset of said probe nucleic acids on the array is represented in the set of tagged antisense molecules. In certain embodiments, each of the tagged antisense molecules of the set is of known sequence and is present in known amount. In certain embodiments, each of the probe nucleic acids on said array is represented in the control set of target nucleic acids. In certain embodiments, the mRNA complementary domain is at least about 25 nt or at least about 30 nt in length.

Also provided is a kit for use in producing at least one aRNA molecule, where the kit includes: (a) at least one RNA polymerase promoter tagged antisense molecule, e.g., at least about 20, 30 or 50 nt in length; and (b) a DNA dependent RNA polymerase, e.g., T7 RNA polymerase. In certain embodiments, the kit includes a population of distinct RNA polymerase promoter tagged antisense molecules. In certain embodiments, the kit further includes ribonucleotides. In certain embodiments, the kit further includes an array of probe nucleic acids on a surface of a substrate.

### BRIEF DESCRIPTION OF THE DRAWINGS

FIG. 1 provides schematic representation of a first embodiment of the subject methods in which a hybrid structure is used directly in an RNA polymerase mediated transcription step to produce aRNA.

FIGS. 2a and 2b provide a schematic representation of a second embodiment of the subject methods in which a hybrid structure is first converted to a template structure, which template structure is then employed in a polymerase mediate transcription step to produce aRNA.

FIG. 3. Gene expression analysis in rat liver revealed by hybridization of ³²P-labeled aRNA target with Atlas Rat Plastic 4K Microarray. Set of 1090 of tagged antisense oligonucleotides was hybridized with 10 μg of rat liver total RNA and fraction of hybridized oligonucleotides was amplified, converted to ³²P-labeled aRNA and hybridized with the microarray as described in more details in Example 1.

FIG. 4 provides Table 1, which is a list of the sequences of 1090 tagged antisense oligonucleotides. All oligos are single-stranded oxyribooligonucleotides (80-mers) with the same 8 nucleotide tag sequences on the 5′- and 3′-ends. The tag sequences at the 3′- and 5′-ends are different from each other. Each antisense oligo correspond individual rat gene, the gene names listed in a separate column.

FIGS. 5a and b provides a diagram showing method of mixing antisense pools with different point in printing, and subsequent comparisons.

FIG. 6 provides a pictures of a human glass array hybridized with an antisense oligo pool.

### DETIALED DISCRIPTION OF THE INVENTION

Methods for producing a population of distinct aRNA molecules from an initial population of distinct mRNA molecules are provided. In the subject methods, an initial mRNA sample is contacted with a population of distinct tagged antisense nucleic acid molecules to produce a population of hybrid molecules. The resultant population of hybrid molecules, or derivatives thereof (e.g., template structures produced there from) is then contacted with a DNA dependent RNA polymerase to produce the population of distinct aRNA molecules via RNA polymerase mediated transcription. Also provided are kits for practicing these methods. The subject methods find use a variety of different applications in which the preparation of aRNA is desired, e.g., the preparation of nucleic acid targets for use in array based hybridization applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing those components that are described in the publications which might be used in connection with the presently described invention.

Methods of Producing aRNA

The subject invention provides methods for producing at least one aRNA molecule corresponding to an initial mRNA molecule. The subject invention can be used to produce aRNA from a single mRNA molecule or simultaneously produce aRNA from a population of distinct mRNA molecules. Each of these representative embodiments is described in greater detail below.

Production of aRNA From a Single mRNA Molecule

In certain embodiments, the subject invention provides methods of producing one or more, including amplified amounts of, aRNA molecules from an initial mRNA molecule, i.e., a plurality of aRNA molecules corresponding to the same mRNA molecule. As such, methods of producing amplified amounts of aRNA molecules from an initial mRNA molecule are provided. By amplified amounts is meant that for each initial mRNA molecule of interest, multiple corresponding aRNA molecules, where the term aRNA stands for antisense ribonucleic acid, are produced. In certain embodiments, the number of corresponding aRNA molecules produced for each initial mRNA molecule during the subject linear amplification method will be at least about 10, usually at least about 50 and more usually at least about 100, where the number may be as great as 1000 or greater.

By corresponding is meant that the aRNA molecule and the mRNA molecule have at least a region of the same sequence of ribonucleotides, where the sequence of the entire aRNA molecule produced by the subject methods is typically found in the mRNA molecule to which it corresponds. As such, the aRNA molecule has a region that shares a substantial amount of, and typically complete, sequence identity with the sequence of the initial mRNA molecule to which it corresponds, where substantial amount means at least 95%, usually at least 98% and more usually at least 99%, where sequence identity is determined using the BLAST algorithm, as described in Altschul et al. (1990), J. Mol. Biol. 215:403-10 (using the published default settings, i.e. parameters w=4 and t=17).

In practicing the subject methods, the first step is to contact a tagged nucleic acid molecule, e.g., tagged DNA molecule, tagged RNA molecule, and in many embodiments a tagged DNA molecule, with the mRNA molecule for which the one or more aRNA molecules is to be produced.

Tagged antisense nucleic acid molecules include at least the following two domains: (1) an mRNA antisense domain complementary to at least a substantial portion of the mRNA molecule; and (2) at least one tag domain. The mRNA antisense domain is a region that is sufficiently long to provide for specific hybridization to its corresponding mRNA molecule and to generate an aRNA molecule of the desired length, as described below. As mentioned above, the mRNA complementary antisense DNA domain is complementary to a substantial portion of the mRNA molecule. By “substantial portion” is meant a length of at least about 20 nt, usually at least about 50 nt, often at least about 65 nt, 100 nt or longer, including and up to the full length of the mRNA, e.g., 150 nt, 200 nt, 300nt or longer. In certain embodiments, the tagged antisense molecules are synthetic nucleic acids, e.g., synthesized by phosphoramidite chemistry, and range in length from about 30 to 150 nt, and often from about 65 to 85 nt.

The tagged antisense nucleic acids may be polymers of synthetic nucleotide analogs. Such tagged antisense nucleic acids may be preferred in certain embodiments because of their superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Locked nucleic acids give additional conformational stability of sugar moiety due to additional bonds between 2′-carboxil and 5′-carboxil or 4′-carboxil groups of deoxyribose. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases that find use in the method of the invention are those capable of appropriate base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively. Examples of non-naturally occurring bases that are capable of forming base-pairing relationships include, but are not limited to, aza and deaza pyrimidine analogues, aza and deaza purine analogues, and other heterocyclicbase analogues, wherein one or more of the carbon and nitrogen atoms of the purine and pyrimidine rings have been substituted by heteroatoms, e.g., oxygen, sulfur, selenium, phosphorus, and the like.

In addition to the antisense domain, the tagged antisense DNA molecules further include at least one tag domain. The main function of the tag domains is to provide a hybrid molecule, as described in greater detail below, that be transcribed into aRNA by DNA dependent RNA polymerase mediated transcription or can readily be converted to a derivative template structure that is employed in a such a transcription step. Typically, the tagged antisense DNA molecules include from one to two tags or tag domains, where the tag domains are typically located at one or both of the termini of the molecule, i.e., the 5′ and 3′ termini. Typically, a population of different tagged antisense DNA molecules shares the same tag domain or domains.

The tag domains can be any sequence and typically range in length from about 4 to 50 nt, sometimes from about 6 to 25 nt and often from about 8 to 20 nt. The tag domains are typically not complementary to the mRNA sequence opposing them when the antisense tagged DNAs are hybridized to their corresponding mRNA molecules, i.e., the tag domains do not hybridize (e.g., under stringent conditions) to their opposing domains in the mRNA molecule.

Where the tagged antisense molecules include two different tag domains, i.e., a domain at each termini, the two different tag domains could be the same but in certain embodiments they are generally different, i.e., they have a different sequence, where two given tag domains are considered to have a different sequence if they share less than 80% homology, e.g., as determined by BLAST, supra.

The tag domain(s) can be single or double stranded.

The tag domain or domains can include a variety of different types of sites and, therefore, functionality. Sites of interest include, but are not limited to: restriction sites/regions/subdomains, primer binding sites/regions/subdomains, RNA polymerase promoter sites/regions/subdomains, etc. Restriction site/region/subdomains of interest can include any sequence of nucleotide residues that are recognized by a restriction endonuclease, e.g., Rsa I, EcoRI, etc. Primer binding sites/regions/subdomains of interest are typically single stranded and include a primer complementary sequence of at least 6 nt in length, typically at least 8 nt in length and more typically at least 9 nt in length. The primer binding sequences are chosen to hybridize with primers, typically the 3′ end of primers, where the primers typically range in length from about 10 to 75 nt, usually from about 15 to 35 nt and more usually from about 18 to 25 nt. In certain embodiments, the primers can include a sequence of an RNA polymerase promoter. The primers can find use in primer extension reactions, PCR or other amplification steps, e.g., in order to amplify tagged antisense DNA molecules, to convert tagged antisense DNA molecules to template DNA structures that include the RNA polymerase promoter domain, etc. In yet other embodiments, primers can be employed as a template for antisense DNA extension reactions, where the extended product can be labeled by detectable probes. This approach is useful in the generation of labeled hybridization target from non-amplified or amplified antisense DNA.

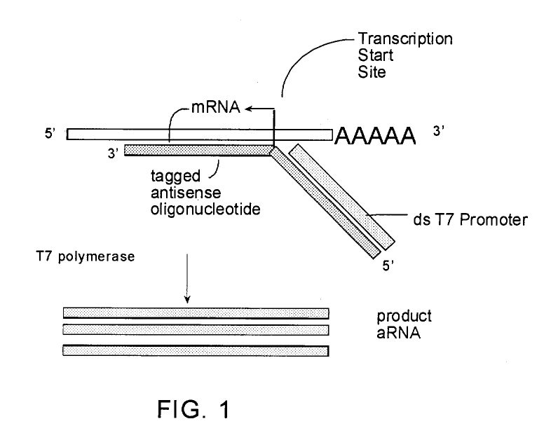

In certain embodiments (such as those illustrated in FIG. 1), the tag domain includes includes an RNA polymerase promoter domain. In these embodiments, the tag domain is a 5′ tag domain that includes the RNA polymerase promoter domain. In these embodiments, the promoter domain is linked in an orientation to permit transcription of the mRNA sense domain strand or nucleic acid corresponding to the sequence of the mRNA domain (template strand). A linker oligonucleotide between the promoter and the DNA may be present and, if present, will typically comprise between about 5 and 20 bases, but may be smaller or larger as desired.

The tagged antisense nucleic acid molecule is contacted with the mRNA molecule of interest under hybridization conditions sufficient to produce a duplex structure, i.e., hybrid, made up of the tagged antisense DNA molecule hybridized to the mRNA molecule. In many embodiments, the two molecules are contacted with each other under stringent hybridization conditions in order to produce the desired duplex or hybrid structure. An example of stringent hybridization conditions is hybridization at 50° C. or higher and 6.0×SSC (900 mM NaCl/90 mM sodium citrate). Another example of stringent hybridization conditions is overnight incubation at 42° C. or higher in a solution: 50% formamide, 6×SSC (900 mM NaCl, 90 mM trisodium citrate), 50 mM sodium phosphate (pH 7.6), 10% dextran sulfate, and 20 μg/ml denatured, sheared salmon sperm DNA. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed.

Following production of the tagged antisense DNA/mRNA structures by the above hybridization step, the resultant hybrid/duplex structures, or derivates thereof (such as the template structures described in greater detail below) aRNA is produced via DNA dependent RNA polymerase mediated transcription. In other words, following production of the tagged nucleic acid/mRNA hybrid or duplex structure, one or more aRNA molecules is produced from the hybrid or duplex structure or a derivative thereof.

In certain embodiments, the above described hybrid or duplex structure is employed directly in aRNA production via RNA polymerase mediated transcription, as depicted in FIG. 1. In these embodiments, the hybrid structure includes a suitable RNA polymerase promoter domain.

With respect to the RNA polymerase promoter domain that is employed in the transcription steps of the subject invention, a number of RNA polymerase promoters may be used. Suitable promoter regions are promoters that are capable of initiating transcription of an operably linked DNA sequence, e.g., the anstisense DNA domain of a hybrid structure, in the presence of ribonucleotides and an RNA polymerase under suitable conditions.

The promoter region usually includes between about 15 and 150 nucleotides, preferably between about 15 and 25 nucleotides, from a naturally occurring RNA polymerase promoter or a consensus promoter region, as described in Alberts et al., in Molecular Biology of the Cell, 2d Ed., Garland, N.Y. (1989). In general, prokaryotic promoters are preferred over eukaryotic promoters, and phage or virus promoters most preferred. As used herein, the term “operably linked” refers to a functional linkage between the affecting sequence (typically a promoter) and the controlled sequence. The promoter regions that find use are regions where RNA polymerase binds tightly to the DNA and contain the start site and signal for RNA synthesis to begin. In E. coli, typically the RNA polymerase molecule covers about 60 nucleotides when it binds to the DNA. Native strong promoters typically contain two highly conserved DNA sequences, each about six nucleotides long, which are located upstream from the start site and separated from each other by about 17 nucleotides of unrecognized DNA. A wide variety of promoters are known. Representative promoter regions of interest that find use include SP6, T3 and T7 as described in Chamberlin and Ryan, The Enzymes (ed P. Boyer, Academic Press, New York) (1982) pp 87-108. See also, Enzymology Primer for Recombinant DNA Technology (Academic Press, 1996).

In certain embodiments, following hybrid structure production via hybridization, as described above, and prior to transcription, the derivative of the hybrid structure is produced from the hybrid structure. More specifically, the hybrid structure is converted to a template structure, which template structure is then employed in the transcription step. This conversion to template structure can include one or more different steps, depending on the nature of the initial hybrid structure from which the template structure is to be derived. In certain embodiments where an RNA polymerase promoter sequence is not present in the tag domain, it is necessary to incorporate an RNA polymerase promoter domain into the hybrid structure, as described above. Incorporation of the RNA polymerase promoter domain can be accomplished using any convenient protocol, where representative protocols include, but are not limited to: primer extension, fusion PCR, ligation, site-specific recombination, etc.

In certain embodiments, the mRNA strand of the hybrid structure is converted to DNA, where any convenient protocol for converting mRNA to DNA may be employed. For example, the mRNA strand can be converted to DNA using second strand cDNA synthesis protocols, such as using a combination of RNase H and an enzyme with DNA polymerase activity, e.g., DNA polymerase 1. Alternatively, a primer extension reaction using a primer complementary to a 3′ tag domain can be employed to generate a second strand DNA and therefore a template structure. In yet an alternative embodiment, a PCR protocol can be used to generate template structure from the initial hybrid structure, e.g., by employing PCR primers to both the 5′ and 3′ tags and amplifying a template structure from the initial hybrid structure.

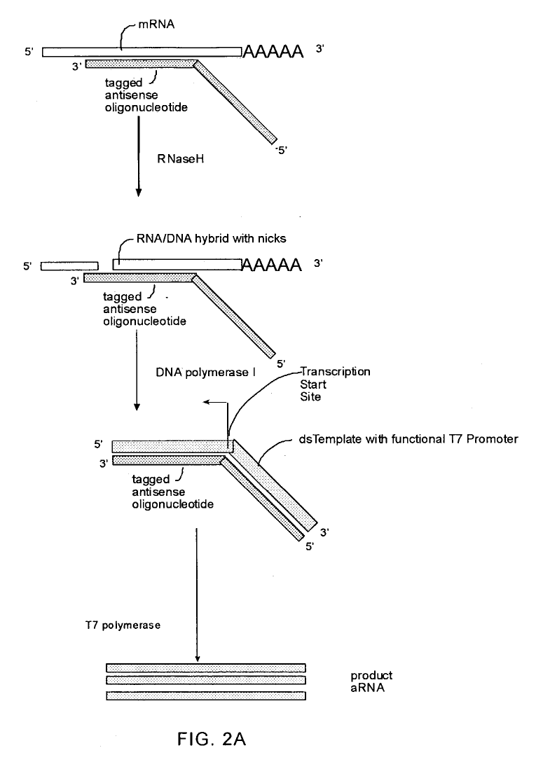

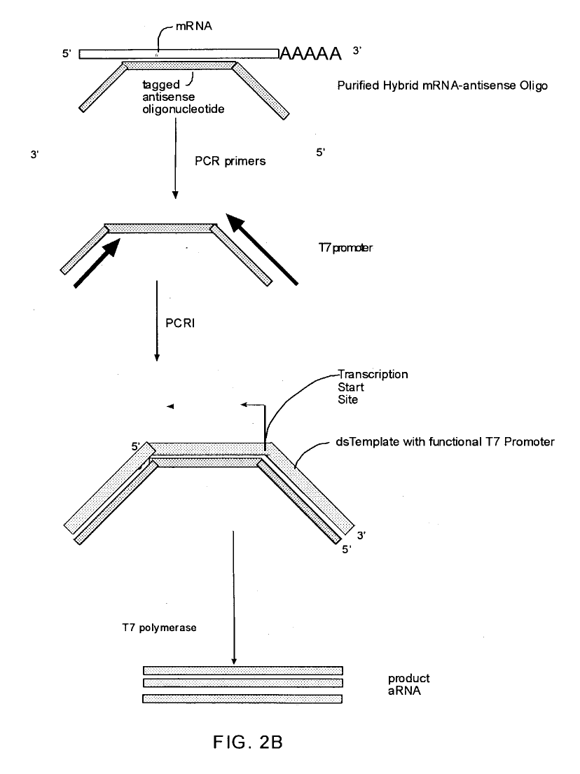

FIGS. 2A and B provide a flow diagram of a protocol that includes a step in which the hybrid structure is converted to a template structure prior to transcription, as described above.

As summarized above, following hybrid production and, optionally template structure derivatization of the hybrid, the hybrid or template structure derivative thereof is employed in DNA dependent RNA polymerase mediated transcription. In many embodiments, prior to the transcription step, the hybrid structures and/or derivatives thereof, are separated from unhybridized tagged antisense nucleic acids. This separation step may be accomplished using any convenient protocol, e.g., via physical (e.g. size separation) and/or chemical/enzymatic (e.g., nuclease) protocols.

For the transcription step, the presence of the RNA polymerase promoter region of the hybrid structure or template derivative thereof is exploited for the production of aRNA. To synthesize the aRNA, the hybrid structure or template derivative thereof is contacted with the appropriate RNA polymerase in the presence of the four ribonucleotides, e.g. rGTP, rCTP, rATP and rUTP, under conditions sufficient for RNA transcription to occur, where the particular polymerase employed will be chosen based on the promoter region present in the ds DNA, e.g. T7 RNA polymerase, T3 or SP6 RNA polymerases, E. coli RNA polymerases, and the like. Suitable conditions for RNA transcription using RNA polymerases are known in the art, see e.g. the references described in the Relevant Literature section, supra. In certain embodiments, the ribonucleic acid strand of the duplex is separated from the DNA strand prior to transcription, e.g., by enzymatic digestion, chemical modification, dissociation and separation, etc.

The above two steps result in the production of at least one aRNA molecule that corresponds to the initial mRNA molecule as described above.

Methods of Producing aRNA From a Plurality of Distinct mRNA Molecules

As indicated above, in many embodiments, the subject methods are employed to simultaneously produce one or more, e.g., a plurality or population of, aRNA molecules from a plurality of initial distinct mRNA molecules, e.g., as is found in an initial mRNA population isolated from a cellular source. In other words, the subject methods of this embodiment are methods of producing a population of distinct aRNA molecules that correspond to a population of distinct mRNA molecules. In other words, one or more aRNA molecules are produced simultaneously from a plurality of distinct initial mRNA molecules (where distinct means that the molecules have a different sequence). More specifically, in certain embodiments, the subject methods simultaneously produce one or more aRNA molecules, e.g., amplified amounts of aRNA molecules, from a plurality of different mRNA molecules. For example, the subject methods can take an initial population of 10 different mRNA molecules and simultaneously produce 10 or more aRNA molecules for each of the distinct mRNA molecules in the initial population.

The resultant population of aRNA molecules reflects or is a representation of the initial mRNA population. In other words, the plurality of distinct aRNA molecules generated from the initial mRNA population in similar in terms of copy number to the plurality of distinct mRNA molecules in the initial mRNA population. More specifically, in certain embodiments, the subject methods simultaneously produce amplified amounts of aRNA, where at least 90% of the distinct molecules have less than 10 fold, often less than 5 fold and sometimes less than 3-fold differences in copy number from the corresponding copy number of their corresponding mRNAs in the initial mRNA sample. The initial mRNA population that is employed in these embodiments may be present in a variety of different samples, where the sample will typically be derived from a physiological source. The physiological source may be derived from a variety of eukaryotic or prokaryotic sources, with physiological sources of interest including sources derived from single cell organisms such as yeast or bacteria and multicellular organisms, including plants and animals, particularly mammals, where the physiological sources from multicellular organisms may be derived from particular organs, biological fluids (e.g., blood) or tissues of the multicellular organism, or from isolated cells derived there from.

In obtaining the sample of RNAs to be analyzed from the physiological source from which it is derived, the physiological source may be subjected to a number of different processing steps, where such processing steps might include tissue/cell homogenation, cell isolation, cell fractionation and cytoplasmic extraction, nucleic acid extraction and the like, where such processing steps are known to the those of skill in the art. Methods of isolating total or polyA+ RNA from cells, tissues, organs, biological fluids or whole organisms are known to those of skill in the art and are described in Maniatis et al., Molecular Cloning: A Laboratory Manual (Cold Spring Harbor Press) (1989). Alternatively, at least some of the initial steps of the subject method may be performed in situ, as described in U.S. Pat. No. 5,514,545, the disclosure of which is herein incorporated by reference. In certain embodiments, total RNA comprising mRNA is used for hybridization with tagged antisense nucleic acids molecule in hybrid structure production.

Following provision of the initial mRNA population, e.g., sample containing the initial mRNA population, the initial population is contacted with a population or set of distinct tagged antisense nucleic acid molecules, where the population or set includes a tagged antisense molecule for each different mRNA molecule of interest that is present in the initial population.

The tagged antisense DNA molecules of the subject sets are deoxyribonucleic acids, preferably single-stranded full-length or fragments of sequences that hybridize to the corresponding mRNAs. In certain embodiments, the tagged antisense molecules are synthetic single stranded deoxyribonucleotides complimentary to gene specific portions of the mRNAs of interest. In certain embodiments, all of the tagged antisense nucleic acids have the same size (e.g., the are all 80 mers) and have the same or at least similar sized mRNA complementary region or domain, which typically ranges from about 25 to 70 nt and often from about 45 to 60 nt. Furthermore, in certain embodiments the tagged antisense nucleic acids have similar GC content, which often ranges from about 40 to 80%, usually from about 45 to 60%, and similar melting temperatures (where there is typically not more than a 10° C. variation in this parameter and often not more than a 5° C. variation in this temperature). Among the population of tagged antisense molecules, all of the tag domains are constant or same in many embodiments, with the only variation occurring in the mRNA complementary regions of the tagged antisense nucleic acids.

A feature of the sets of target nucleic acids is that they include at least one tagged antisense molecule that is complementary to each mRNA of a predetermined collection or set of mRNAs of interest, e.g., a collection or set of mRNAs that is represented by probe nucleic acids on a nucleic acid microarray. In other words, each of the distinct mRNAs of a given predetermined set or collection of distinct mRNAs are represented in the set of tagged antisense nucleic acid molecules. For example, where a given predetermined set of mRNAs of interest includes 500 distinct mRNAs which are distinct from each other based on sequence, a set of tagged antisense nucleic acid molecules includes at least 500 different tagged antisense nucleic acid molecules—one for each probe nucleic acid on the array.

The number of unique tagged antisense molecules (where any two sequences are unique if they differ from each other in terms of sequence, where the difference may be a minimal as a 1 to 10 base difference) in the set or pool of tagged mRNA antisense molecules will, in most embodiments, be at least about 10, 20, 50, 100, 200 or more where the number may be as high as about 1,000; 20,000 or higher, but in many embodiments will not exceed about 10,000; 5,000, or 1,000.

In certain embodiments, the sets include a tagged antisense nucleic acid for each mRNA that may be present in the sample. In other words, the set includes a tagged antisense nucleic acid for each different mRNA molecule that may be present in the sample.

In yet other embodiments, the sets are sets that include a representative or representational number of tagged antisense molecules. As the subject sets include a representational number of tagged antisense molecules, the total number of different tagged mRNA antisense molecules in any given set will be only a fraction of the total number of different or distinct mRNAs in the sample that is employed to generate the aRNA, where the total number of tagged antisense molecules in the set will generally not exceed 80%, usually will not exceed 60-50% and more usually will not 40-20% of the total number of distinct mRNAs in the original sample, e.g., the total number of distinct messenger RNAs (mRNAs) in the original sample. Any two given RNAs in a sample will be considered distinct or different if they comprise a stretch of at least 100 nucleotides in length in which the sequence similarity is less then 95% or lower, as determined using the FASTA program (default settings). As the sets of tagged antisense molecules comprise only a representational number of target nucleic acids compared to the initial mRNA population of the sample from which the aRNA population is to be produced, with sources comprising from 5,000 to 50,000 distinct mRNAs, the number of different tagged antisense molecules in the set typically ranges from about 20 to 40,000 or 20 to 10,000, usually from about 50 to 2,000 or 50 to 30,000 and more usually from about 100 to 20,000 and sometimes from about 75 to 1500.

In some embodiments, a feature of the sets of tagged antisense molecules is that the concentration of each tagged antisense molecule present in the set is known. In other words, the amount of each individual RNA polymerase promoter tagged antisense DNA molecule in the control set is known. For example, an equal weight amount of each distinct tagged antisense molecules may present in the mixture. In certain embodiments, an equal molar amount or equimolar amount of each tagged antisense molecule may be present. In yet other embodiments, different known amounts or ratios of the various constituent tagged antisense molecules may be present. However, in any set of tagged antisense molecules employed according to the subject invention, the amount of each constituent member present in the set is known, either in absolute terms or in terms relative to each other.

The sets of tagged antisense molecules are further characterized in that at least two different gene functional classes are typically represented in a given set, where the number of different functional classes of genes represented in the a given set will generally be at least 3, and will usually be at least 5. In other words, the sets of tagged antisense molecules comprise nucleotide sequences complementary to RNA transcripts of at least 2 gene functional classes, usually at least 3 gene functional classes, and more usually at least 5 gene functional classes. Gene functional classes of interest include oncogenes; genes encoding tumor suppressors; genes encoding cell cycle regulators; stress response genes; genes encoding ion channel proteins; genes encoding transport proteins; genes encoding intracellular signal transduction modulator and effector factors; apoptosis related genes; DNA synthesis/recombination/repair genes; genes encoding transcription factors; genes encoding DNA-binding proteins; genes encoding receptors, including receptors for growth factors, chemokines, interleukins, interferons, hormones, neurotransmitters, cell surface antigens, cell adhesion molecules etc.; genes encoding cell-cell communication proteins, such as growth factors, cytokines, chemokines, interleukins, interferons, hormones etc.; and the like.

Of particular interest are sets of tagged antisense molecules in which each of the genes collectively listed in the tables of the following applications are represented in the control set: U.S. patent application Ser. No. 08/859,998; U.S. patent application Ser. No. 08/974,298; U.S. patent application Ser. No. 09/225,998; U.S. application Ser. No. 09/221,480; U.S. application Ser. No. 09/222,432; U.S. application Ser. No. 09/222,436; U.S. application Ser. No. 09/222,437; U.S. application Ser. No. 09/222,251; U.S. application Ser. No. 09/221,481; U.S. application Ser. No. 09/222,256; U.S. application Ser. No. 09/222,248; and U.S. application Ser. No. 09/222,253; the disclosures of which are incorporated herein by reference.

Another feature of the sets of tagged antisense molecules is that they are synthetic nucleic acids and not isolated from a biological source. The sets of tagged antisense molecules may be generated using any convenient protocol.

As described above, the initial mRNA population of distinct mRNAs, which includes the mRNAs of interest if present, is contacted with the population or set of tagged antisense molecules under conditions sufficient to produce hybrid or duplex structures between complementary nucleic acids, e.g., hybridization conditions, typically stringent hybridization conditions. In this step duplex or hybrid structures are produced between any tagged antisense molecules present in the employed set that have a complementary mRNA strand in the sample. In many embodiments, the hybridization conditions employed are those that maintain the integrity of the mRNA and tagged antisense molecules, like RNase-free conditions. A representative example of such conditions is: 50° C. in (5× SSPE, pH 7.5, 6M urea, 1×SUPERase·in™ RNase inhibitor (Ambion, Inc.) overnight.

Following production of the hybrid or duplex structures, in many embodiments any remaining single stranded or unbound tagged antisense molecules that are present in the mixture, i.e., those tagged antisense molecules that do not have a complementary mRNA molecule present in the initial mRNA sample, are separated from the resultant duplex structures. Separation may be accomplished using any convenient protocol (where a number of different protocols are known in the art), including enzymatic or chemical modification and physical separation protocols, etc. In enzymatic protocols, a nuclease that selectively degrades or modifies single stranded and not double stranded nucleic acids, and at least single stranded DNAs, may be employed, where representative single strand specific nucleases include, but are not limited to: S1 nuclease, mung bean nuclease, ribonuclease A, T1, and the like. Physical separation protocols of interest include, but are not limited to: gel electrophoresis, chromatography, precipitation, centrifugation, filtration, binding to immobilized ligands specific for the singled stranded tagged antisense molecules or a region thereof, etc. In chemical modification, the non-bound antisense tagged nucleic acid molecules, but not the hybrid structures, are selective modified (inactivated) by a chemical reactant, such as glyoxal, dimehtylsulfoxide, etc.

Following production of the hybrid structures, the hybrid structures are employed directly in aRNA transcription or, are converted to template derivatives which are then employed in aRNA transcription. Where the hybrid structures are converted to template structures prior to aRNA transcription, any of the protocols discussed above for production of template derivatives from hybrid structures may be employed.

Next, aRNA is transcribed from each of the hybrids or template derivatives thereof as described above. For this transcription step, the RNA polymerase promoter region of the hybrid or template structures is exploited for the production of aRNA. To synthesize the aRNA, the hybrids or templates are contacted with the appropriate RNA polymerase in the presence of the four ribonucleotides, e.g. rGTP, rCTP, rATP and rUTP, under conditions sufficient for RNA transcription to occur, where the particular polymerase employed will be chosen based on the promoter region present in the ds DNA, e.g. T7 RNA polymerase, T3 or SP6 RNA polymerases, E. coli RNA polymerases, and the like. Modified nucleotides, e.g., fluoro rNTP (such as Cy3-rUTP) biotin-rUTP, allylamine rNTP, etc., could be used in order to provide higher stability, hybridization efficiency, post synthesis labeling, etc., of the synthesized aRNA. Suitable conditions for RNA transcription using RNA polymerases are known in the art, see e.g. the references described in the Relevant Literature section, supra.

The resultant aRNA produced by the subject methods finds use in a variety of applications. For example, the resultant aRNA can be used for: (1) cDNA library construction; as target or for use in generation of target nucleic acids for use with microarrays, e.g., in expression profiling analysis; construction of “driver” for subtractive hybridization assays; and the like.

For example, the aRNA produced by the subject invention finds use in studies of gene expression in mammalian cell or other cell populations. The cells may be individual cells or tissue derived cells, e.g., tissue derived from a solid organs, such as brain, spleen, bone, heart, vascular, lung, kidney, liver, pituitary, endocrine glands, lymph node, dispersed primary cells, tumor cells, or the like. In these representative methods, one typically identifies nucleic acid sequences that vary in abundance among different populations, such as in comparing mRNA expression among different tissues or within the same tissue according to physiologic state known as subtractive hybridization assays.

Depending on the particular intended use of the subject aRNA, the aRNA may be labeled. A variety of different labels may be employed, where such labels include fluorescent labels, isotopic labels, enzymatic labels, particulate labels, etc. For example, suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), cyanine dyes, e.g. Cy5, Cy3, BODIPY dyes, e.g. BODIPY 630/650, Alexa542, etc. Suitable isotopic labels include radioactive labels, e.g. ³²P, ³³P, ³⁵S, ³H. Other suitable labels include size particles that possess light scattering, fluorescent properties or contain entrapped multiple fluorophores. The label may be a two stage system, where the target DNA is conjugated to biotin, haptens, etc. having a high affinity binding partner, e.g. avidin, specific antibodies, etc. The binding partner is conjugated to a detectable label, e.g. an enzymatic label capable of converting a substrate to a chromogenic product, a fluorescent label, and isotopic label, etc.

Differential Gene Expression Assays

As indicated above, one application in which the aRNA molecules produced by the subject methods find use is as target nucleic acids in differential gene expression analysis. Using the subject methods, one can produce labeled aRNA molecules, or labeled derivatives of such molecules, e.g., first or second strand cDNA molecules, generated from the aRNA molecules, etc., which are then used as target nucleic acids in differential gene expression analysis. In certain embodiments, the labeled target nucleic acids produced according to the subject invention represent the entire or whole mRNA profile of the sample being assayed. In other embodiments, the labeled target nucleic acids produced according to the subject invention provide a representation of the total RNA profile of the particular source from which the labeled nucleic acids are generated, since not all of the mRNAs present in the initial sample are represented in the set of tagged antisense molecules employed to generate the mRNA. Accordingly, the labeled nucleic acids find use in comparing the characteristic RNA profiles of different physiological sources and identifying differences in the RNA profiles between different physiological source. Comparison of the RNA profiles of two or more physiological sources finds particular use in methods of identifying differential gene expression in two physiological samples, such as cells or tissues derived from the same or different individual organisms, where the tissues may represent different diseased or normal states, different organ or tissue types, etc.

The labeled nucleic acids of the plurality of physiological sources may be compared in a number of different ways. Thus, one may compare the labeled nucleic acids from each source by separately resolving the labeled nucleic acids from each source under substantially identical electrophoretic conditions to yield an electrophoretic pattern of resolved bands for each of the different populations of labeled nucleic acids. The resultant electrophoretic patterns can then be resolved to identify differences between the labeled nucleic acid populations, which differences can then be attributed to differences in the RNA profiles of the each of the physiological sources, where such differences can, in turn, be attributed to difference in gene expression. See Liang & Pardee, Science (1992) 257: 967. Conveniently, electrophoretic separation under identical electrophoretic conditions can be achieved by running the labeled nucleic acids derived from each physiological source of interest in separate, side by side lanes on a slab gel. Automated electrophoretic machines as described in U.S. Pat. Nos. 5,410,412; 5,275,710; 5,217,591; and 5,104,512, the disclosures of which are herein incorporated by references, may be employed to resolve the labeled nucleic acids. In a modification of the above, where each set of labeled nucleic acids or targets of each physiological source has been labeled with a distinct and distinguishable label, the opportunity arises to resolve the nucleic acids in the same electrophoretic medium, e.g. the same column or in the same lane of a slab, thereby ensuring that the nucleic acids are resolved under identical electrophoretic conditions.

Alternatively, one may hybridize the labeled nucleic acids to predefined arrays of probe polymeric molecules stably associated with the surface of a substrate, where the probe polymeric molecules are capable of sequence specific base pair hybridization with complementary labeled target nucleic acids. A variety of different arrays which may be used are known in the art. The polymeric or probe molecules of the arrays may be nucleic acids, e.g., oligonucleotides/polynucleotides, or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g. hexose phosphodiester; peptide nucleic acids; and the like. In many embodiments, the length of the probes ranges from 10 to 1000 nts, where oligonucleotide probes usually range from 15 to 150 nts and more usually from 25 to 100 nts in length, and polynucleotide probes usually range in length from 150 to 1000 nts, where the polynucleotide probes may be single or double stranded, usually single stranded, and may be PCR fragments amplified from cDNA.

The probe molecules on the surface of the substrates in certain embodiments correspond to the set of tagged antisense molecules employed to generate the set of aRNA used as, or to generate, the labeled target nucleic acids. The term “correspond” is used in this instance to mean that all of the distinct tagged antisense molecules present in the set used to generate the aRNA have a corresponding probe on the array, where correspond means that the probe and the tagged antisense molecule has the same (at least 95% homology) sequence for the mRNA binding domain, they hybridize to the same mRNA molecule under stringent conditions.

The substrates with which the probe molecules are stably associated may be fabricated from a variety of materials, including plastics, ceramics, metals, gels, membranes, glasses, and the like.

A variety of different methodologies have been developed for producing arrays of probes stably associated to the surface of a substrate. Representative methodologies include spotting methods, in which probes are immobilized or spotted on the surface of substrates as described in WO 95/35505 the disclosure of which is herein incorporated by reference, and methods in which the probes are synthesized or grown on the surface of the substrates, such as EP 0 373 203 B1 and U.S. Pat. No. 5,445,934, the disclosures of which are herein incorporated by reference. Arrays of probes spotted onto nylon membranes are described in Lennon & Lerach, Trends in Genetics (1991) 7:314-317; Gress et al., Mammalian Genome (1992) 3:609-619; Meier-Ewert et al., Nature (1993) 361:375-376; Nguyen et al., Genomics (1995) 29:207-216; Zhao et al., Gene (1995) 156:207-213; Takahashi et al., Gene (1995) 164:219-217; Milosavlijevic et al., Genome Research (1996) 6:132-141; Pietu et al., Genome Research (1996) 6:492-503; and Drmanac, Science (1993) 260:1649-1652. Arrays of probes spotted onto the surface of modified microscope glass slides are described in Shena et al., Science (1995)270: 467470 and Shalon et al., Genome Research (1996) 6: 639-645. Arrays in which the probes have been grown on the surface of a substrate are described in Lockhart et al., Nature Biotechnology (1996) 14:1675.

Of particular interest for use in the analysis of differential gene expression in various human, mouse and rat physiological sources are the arrays sold under the trademark Atlas™ by Clontech Laboratories, Inc., which product line includes nylon, glass and plastic arrays of probe nucleic acids.

In these hybridization assays, the target nucleic acid is contacted with the array under hybridization conditions, where such conditions can be adjusted, as desired, to provide for an optimum level of specificity in view of the particular assay being performed. Suitable hybridization conditions are well known to those of skill in the art and reviewed in Maniatis et al, supra and WO 95/21944. Of particular interest in many embodiments is the use of stringent conditions during hybridization, i.e. conditions that are optimal in terms of rate, yield and stability for specific probe-target hybridization and provide for a minimum of non-specific probe/target interaction. Stringent conditions are known to those of skill in the art. In the present invention, stringent conditions are typically characterized by temperatures ranging from 15° C. to 35° C., usually 20° C. to 30° C. less than the melting temperature of the probe target duplexes, which melting temperature is dependent on a number of parameters, e.g. temperature, buffer compositions, size of probes and targets, concentration of probes and targets, etc. As such, the temperature of hybridization typically ranges from about 55° C. to 70° C., usually from about 60° C. to 68° C. In the presence of denaturing agents, the temperature may range from about 35° C. to 45° C., usually from about 37° C. to 42° C. The stringent hybridization conditions are further typically characterized by the presence of a hybridization buffer, where the buffer is characterized by one or more of the following characteristics: (a) having a high salt concentration, e.g. 3 to 6×SSC (or other salts with similar concentrations); (b) the presence of detergents, like SDS (from 0.1 to 20%), triton X100 (from 0.01 to 1%), Monidet NP40 (from 0.1 to 5%) etc.; (c) other additives, like EDTA (typically from 0.1 to 5 mM), tetramethylammonium chloride; (d) accelerating agents, e.g. PEG, dextran sulfate (5 to 10%), CTAB, SDS and the like; (e) denaturing agents, e.g. formamide, urea etc.; and the like.

In analyzing the differences in the population of labeled target nucleic acids generated from two or more physiological sources using the arrays described above, in certain embodiments each population of labeled target nucleic acids are separately contacted to identical probe arrays or together to the same array under conditions of hybridization, preferably under stringent hybridization conditions, such that labeled target nucleic acids hybridize to complementary probes on the substrate surface. In yet other embodiments, labeled target nucleic acids are combined with a set of distinguishably labeled standard or control target nucleic acids followed by hybridization of the combined populations to the array surface, as described in application Ser. No. 09/298,361; the disclosure of which is herein incorporated by reference.

In certain preferred embodiments, the labeled target nucleic acids generated according to the present methods is used in conjunction with control nucleic acids as described in application Ser. Nos. 09/298,361 and 09/750,452, the disclosures of which are herein incorporated by reference, as well as in international application serial no. PCT/US00/10894 published as WO 00/65095.

In these embodiments, the tagged antisense nucleic acids are structurally as similar as possible to the control target nucleic acids that are employed in the assay, e.g., both sets of tagged antisense nucleic acids and control nucleic acids are oligonucleotides with the same or similar sequences. In other words, the structure of the control target nucleic acids should be similar to that of the aRNA in order to maximally imitate the hybridization of the aRNA with which they are to be employed.

Where all of the target sequences comprise the same label, different arrays will be employed for each physiological source (where different could include using the same array at different times). Alternatively, where the labels of the targets are different and distinguishable for each of the different physiological sources being assayed, the opportunity arises to use the same array at the same time for each of the different target populations. Examples of distinguishable labels are well known in the art and include: two or more different emission wavelength fluorescent dyes, like Cy3 and Cy5, two or more isotopes with different energy of emission, like ³²P and ³³P, gold or silver particles with different scattering spectra, quantum dot particles, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Following hybridization, non-hybridized labeled nucleic acid is removed from the support surface, conveniently by washing, generating a pattern of hybridized nucleic acid on the substrate surface. A variety of wash solutions are known to those of skill in the art and may be used.

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, calorimetric measurement, light emission measurement, light scattering, and the like.

Following detection or visualization, the hybridization patterns may be compared to identify differences between the patterns. Where arrays in which each of the different probes corresponds to a known gene are employed, any discrepancies can be related to a differential expression of a particular gene in the physiological sources being compared.

The provision of appropriate controls on the arrays permits a more detailed analysis that controls for variations in hybridization conditions, cross-hybridization, non-specific binding and the like. Thus, for example, in a preferred embodiment, the hybridization array is provided with normalization controls. These normalization controls are probes complementary to control target sequences added in a known concentration to the sample. Where the overall hybridization conditions are poor, the normalization controls will show a smaller signal reflecting reduced hybridization. Conversely, where hybridization conditions are good, the normalization controls will provide a higher signal reflecting the improved hybridization. Normalization of the signal derived from other probes in the array to the normalization controls thus provides a control for variations in hybridization conditions. Normalization control is also useful to adjust (e.g. correct) for differences which arise from the array quality, the mRNA sample quality, efficiency of first-strand synthesis, etc. Typically, normalization is accomplished by dividing the measured signal from the other probes in the array by the average signal produced by the normalization controls. Normalization may also include correction for variations due to sample preparation and amplification. Such normalization may be accomplished by dividing the measured signal by the average signal from the sample preparation/amplification control probes. The resulting values may be multiplied by a constant value to scale the results.

In certain embodiments, normalization controls are often unnecessary for useful quantification of a hybridization signal. Thus, where optimal probes have been identified, the average hybridization signal produced by the selected optimal probes provides a good quantified measure of the concentration of hybridized nucleic acid. However, normalization controls may still be employed in such methods for other purposes, e.g. to account for array quality, mRNA sample quality, etc.

One may use the subject methods in the differential expression analysis of: (a) diseased and normal tissue, e.g. neoplastic and normal tissue, (b) different tissue or tissue types; (c) developmental stage; (d) response to external or internal stimulus; (e) response to treatment; and the like. The subject arrays therefore find use in broad scale expression screening for drug discovery, diagnostics and research, as well as studying the effect of a particular active agent on the expression pattern of genes in a particular cell, where such information can be used to reveal drug toxicity, carcinogenicity, etc., environmental monitoring, disease research and the like.

Kits

Also provided are kits for use in the subject invention, where such kits may include one or more containers, each with one or more of the various reagents employed in the subject methods, where the reagents may be present in concentrated form. Thus, the kits may include one or more reagents that are employed to generate aRNA from mRNA according to the present invention, where the kits typically include at least solution with one type of tagged antisense molecule, where in many embodiments the kits will include a set of a plurality of distinct tagged antisense molecules. The kits may also include additional reagents used in the subject methods, including: buffers, the appropriate nucleotide triphosphates (e.g., rATP, rCTP, rGTP and UTP, including labeled or modified versions thereof, e.g., for generating labeled target nucleic acids), RNA polymerase, e.g., T7 RNA polymerase, labeled initiator oligonucleotides used by RNA polymerase to prime aRNA synthesis, RNase inhibitors, etc. Also included may be purification or isolation reagents, e.g., for isolating or purifying the tagged antisense/mRNA hybrids, where such reagents included chromatography columns, binding/washing solutions, modification nucleases, glyoxal, and the like. Other reagents of interest include PCR primers, PCR reagents, etc. In certain embodiments, e.g., where the kits are specifically designed for use in differential gene expression analysis, the kits may further include one or more arrays for use in the gene expression analysis application.

In addition to above mentioned components, the subject kits typically further include instructions for using the components of the kit to practice the subject methods with the subject devices, e.g., a user manual. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Commercial kits based on the inventive concept have been shown to be effective in actual practice, see Dudley, A. M. et al. (2002) Proc. Natl. Acad. Sci. USA 99:7554-7559, incorporated here by reference.

The following examples are offered by way of illustration and not by way of limitation.

### EXPERIMENTAL

### Example 1

A. Isolation and Preparation of Test Target Ribonucleic Acids From Rat Tissue Sample.

As described in greater detail below, the Atlas Plastic Rat microarray (BD Clontech, Palo Alto, Calif., Cat. 7909-1) was used to screen a rat liver tissue sample to determine gene expression of each of the 1000 genes in a rat sample. The tissue sample was tested for expression by isolating total RNA from the sample, hybridizing the total RNA with mixture of tagged antisense oligonucleotides, converting the hybridized oligonucleotides to template and subsequently transcribing the template oligonucleotides into labeled aRNA. This labeled pool of aRNA was then used as test target nucleic acids for hybridization against the probe nucleic acids on the array.

Total RNA was isolated from homogenized rat liver tissue using an ATLASPURE RNA isolation (BD CLONTECH, Palo Alto Calif.) according to the manufacturer's protocols. The total RNA of the tissue sample was used to generate target aRNA for hybridization to the probe array.

B. Hybridization of Tagged Antisense Oligonucleotides with Total RNA.

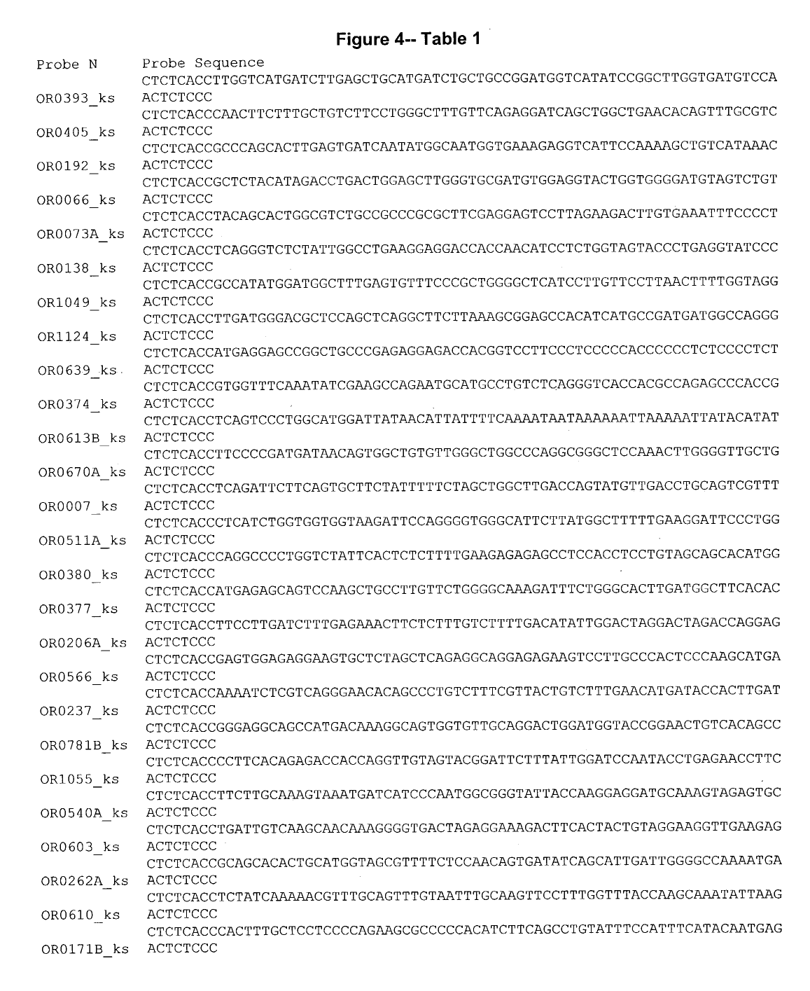

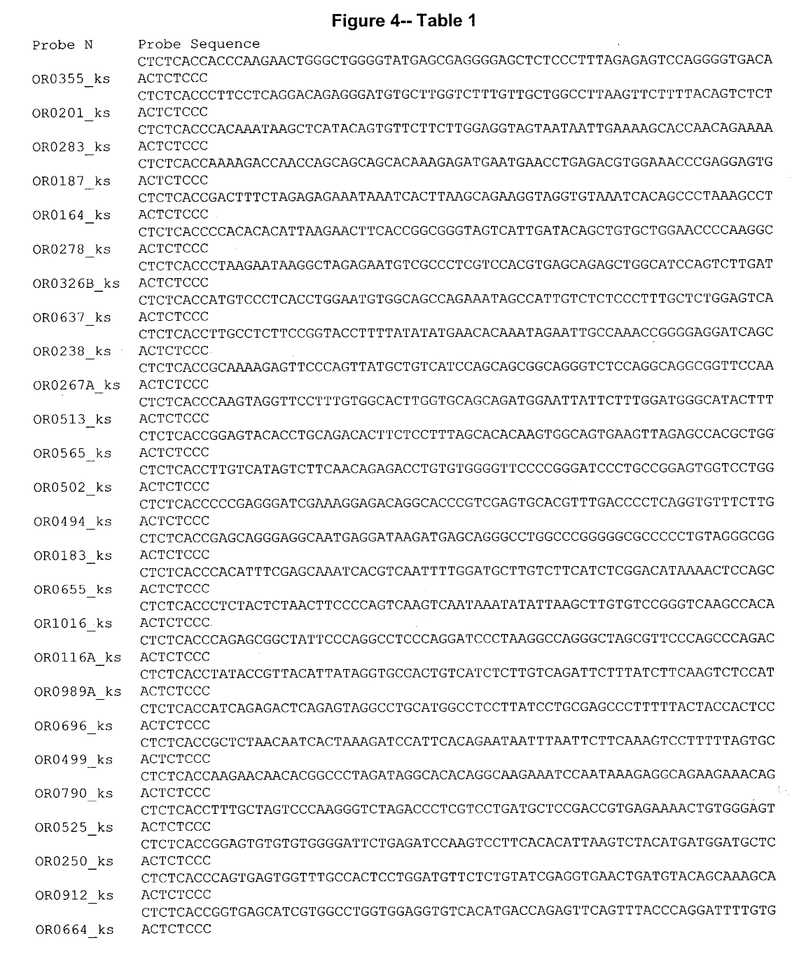

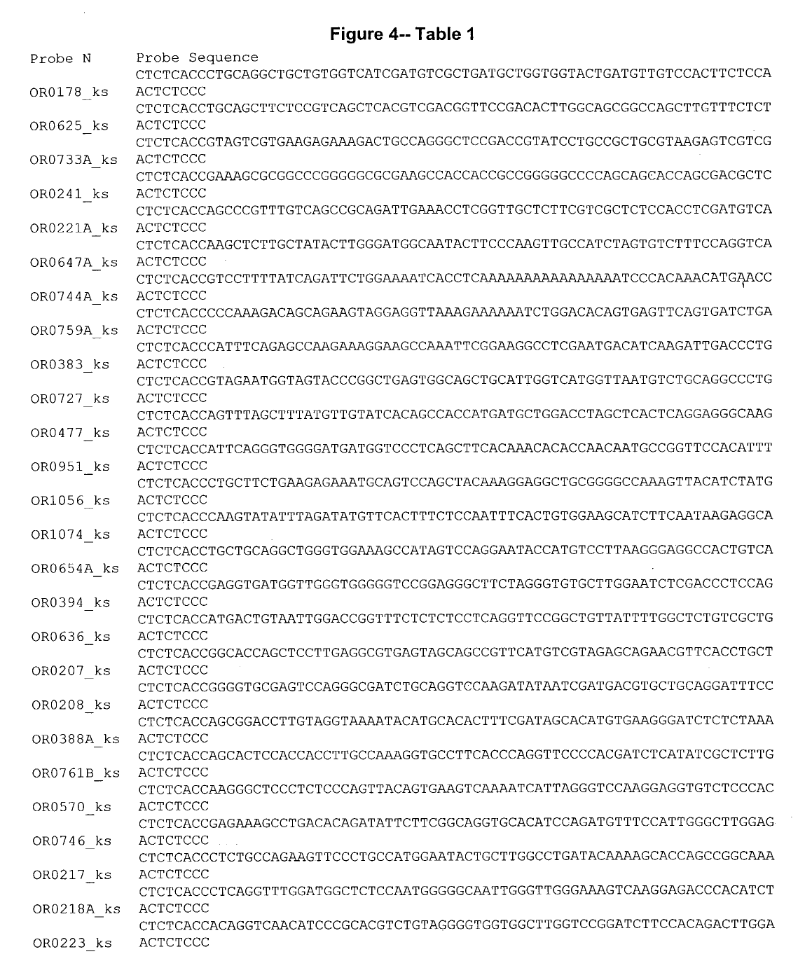

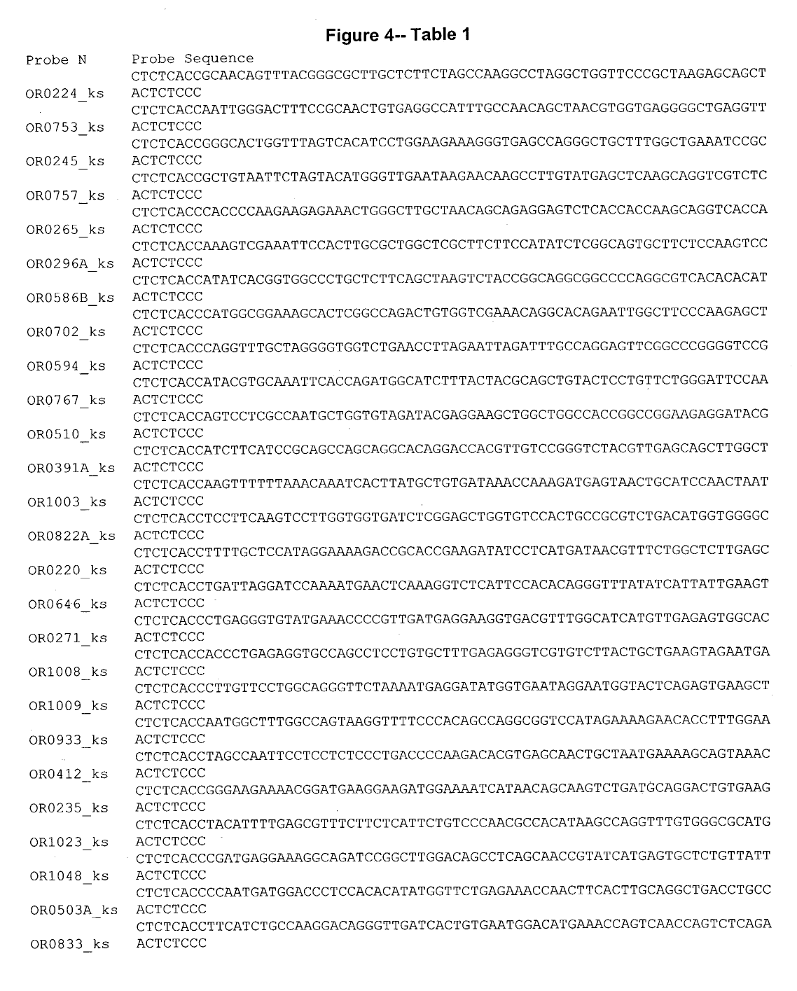

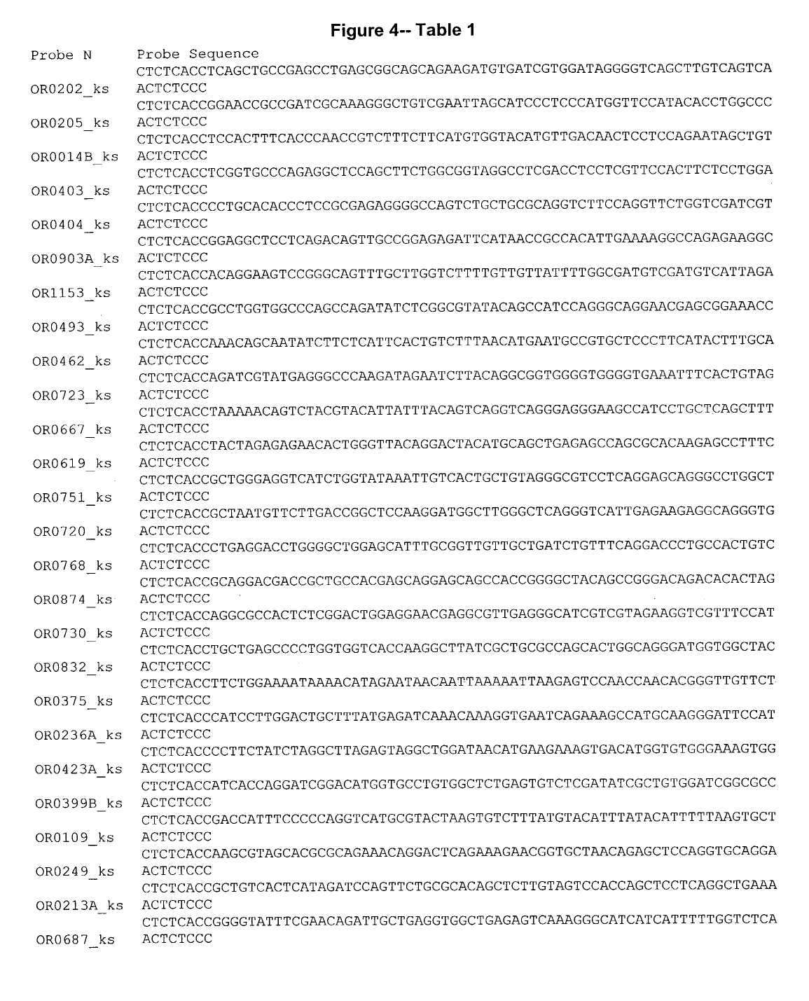

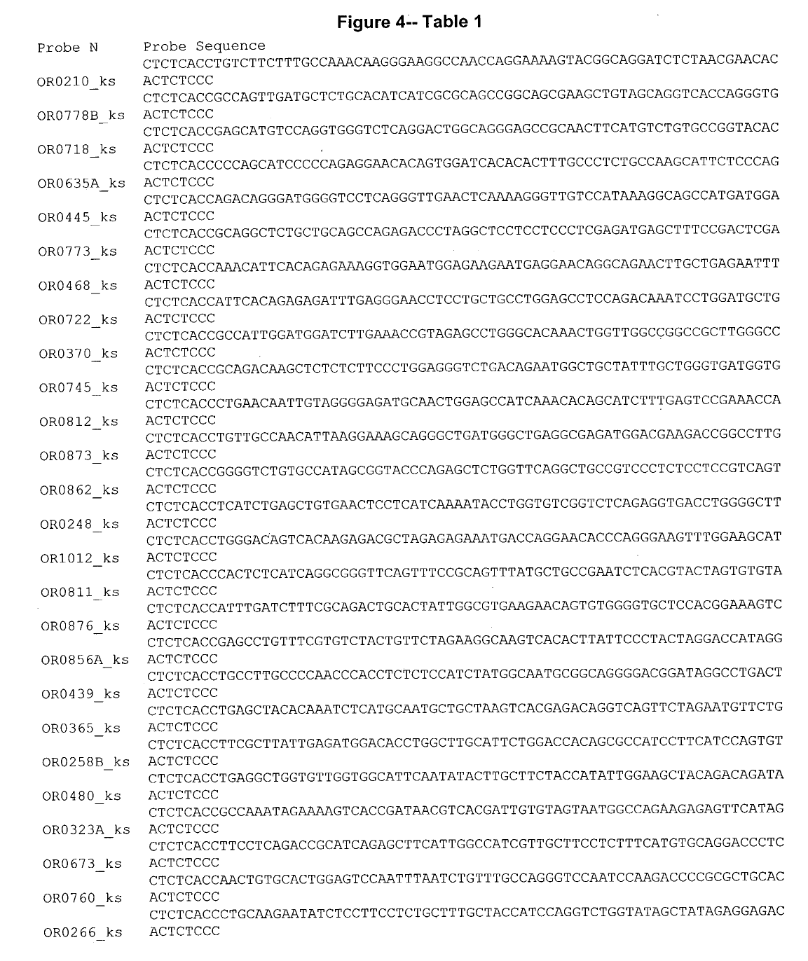

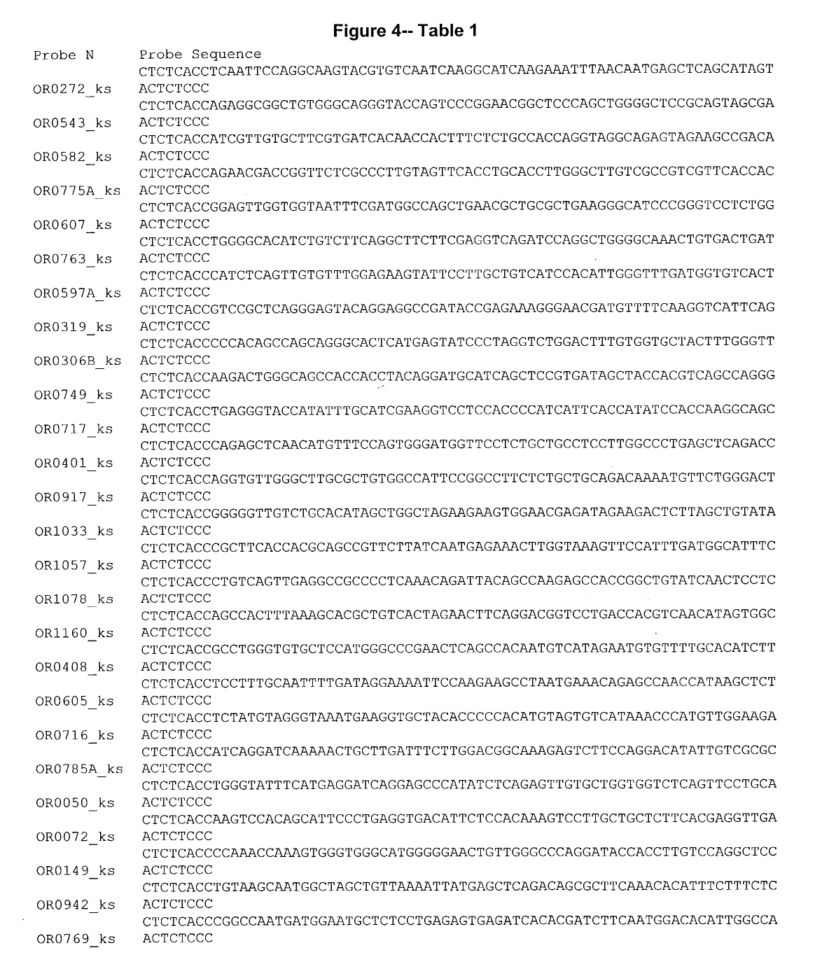

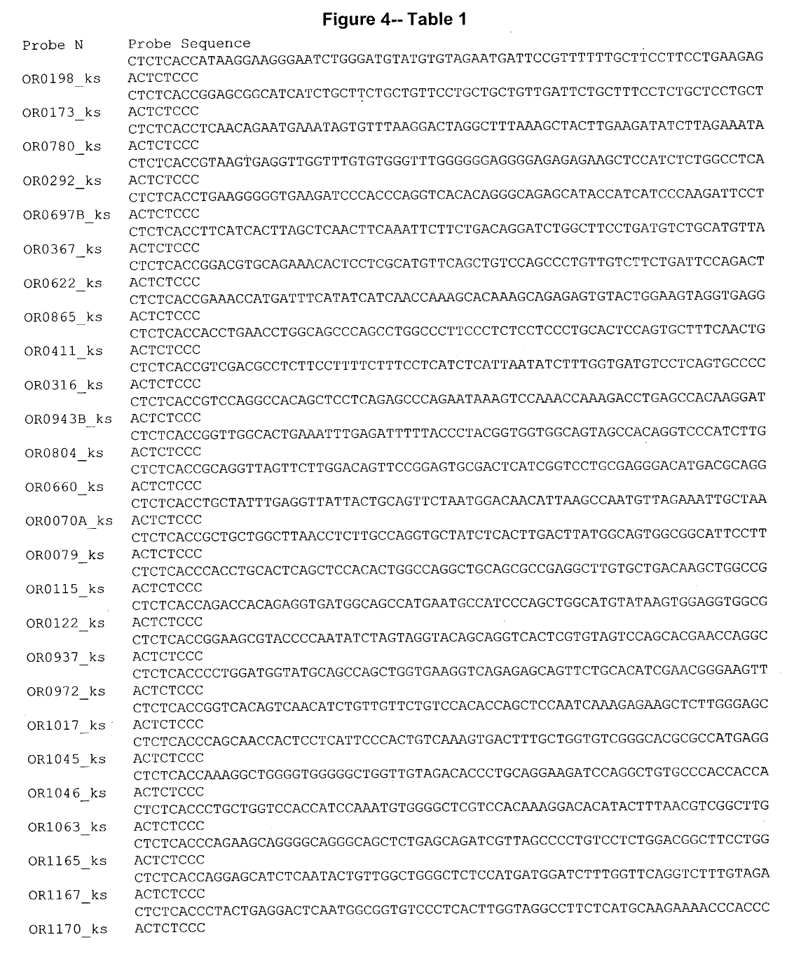

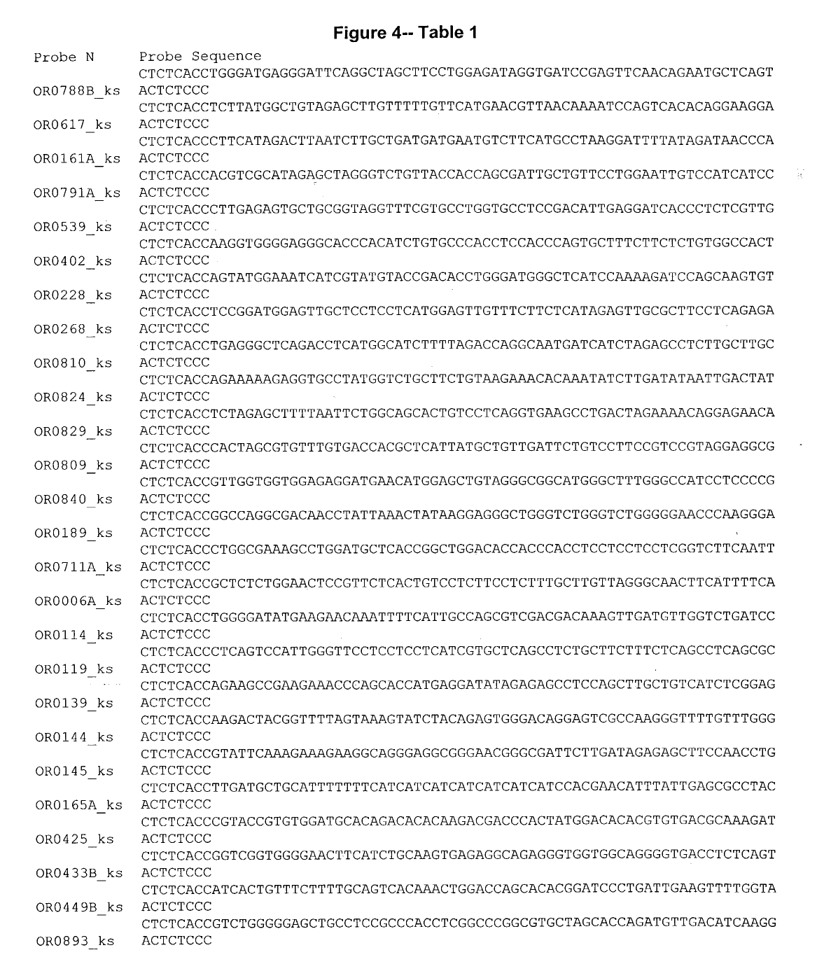

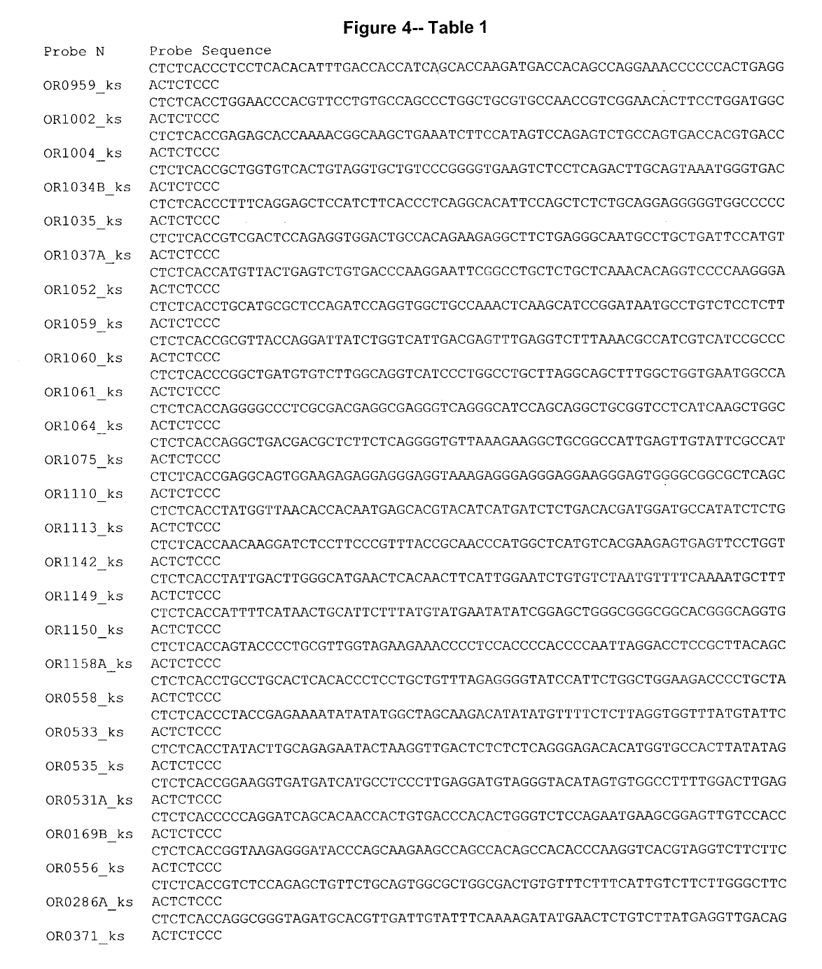

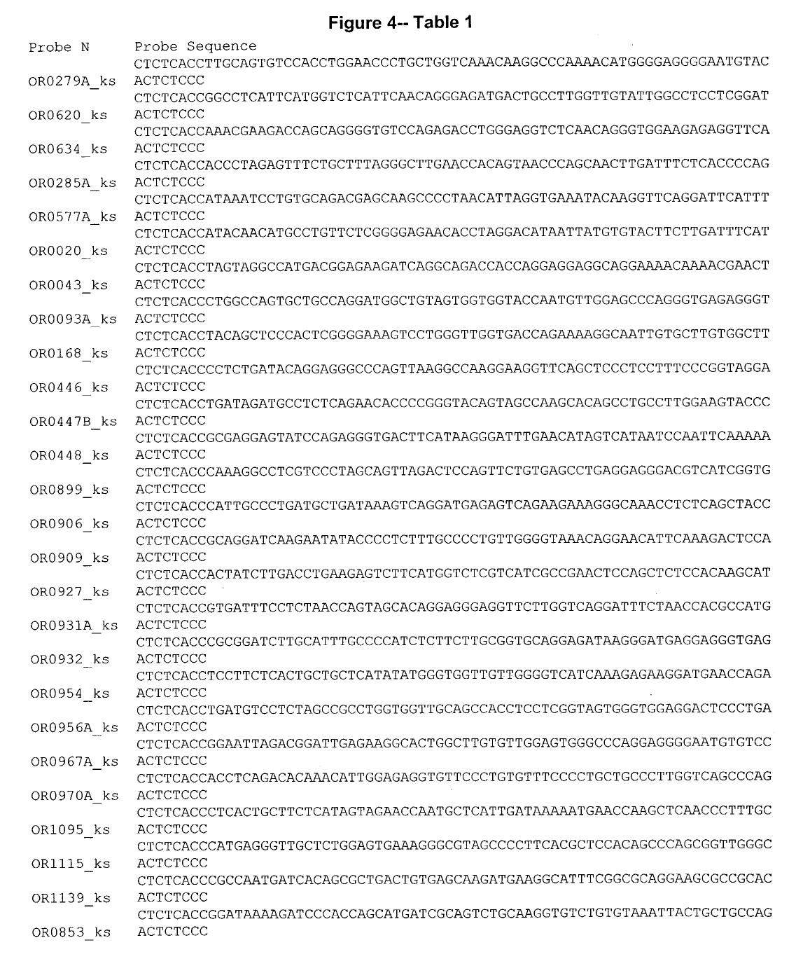

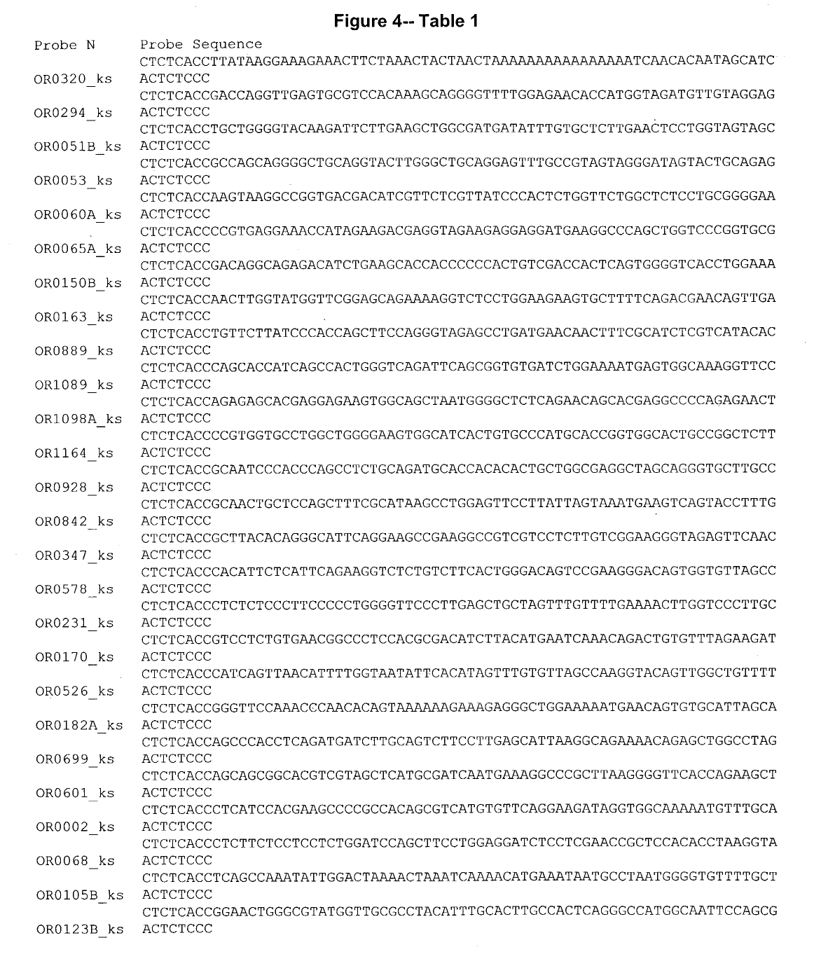

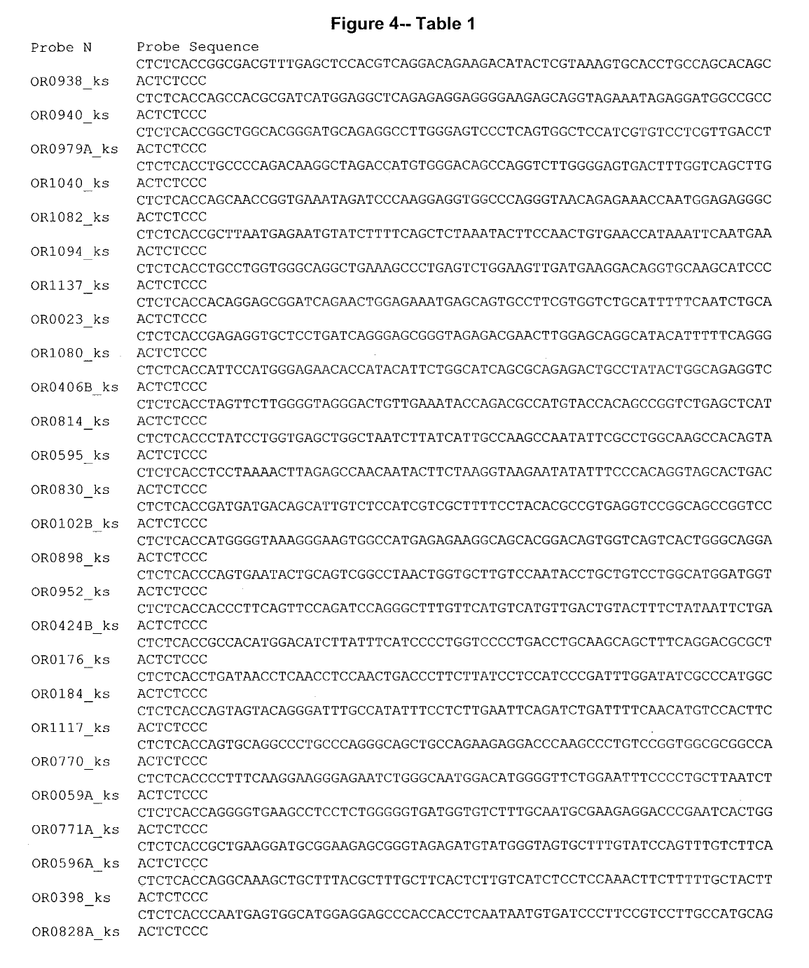

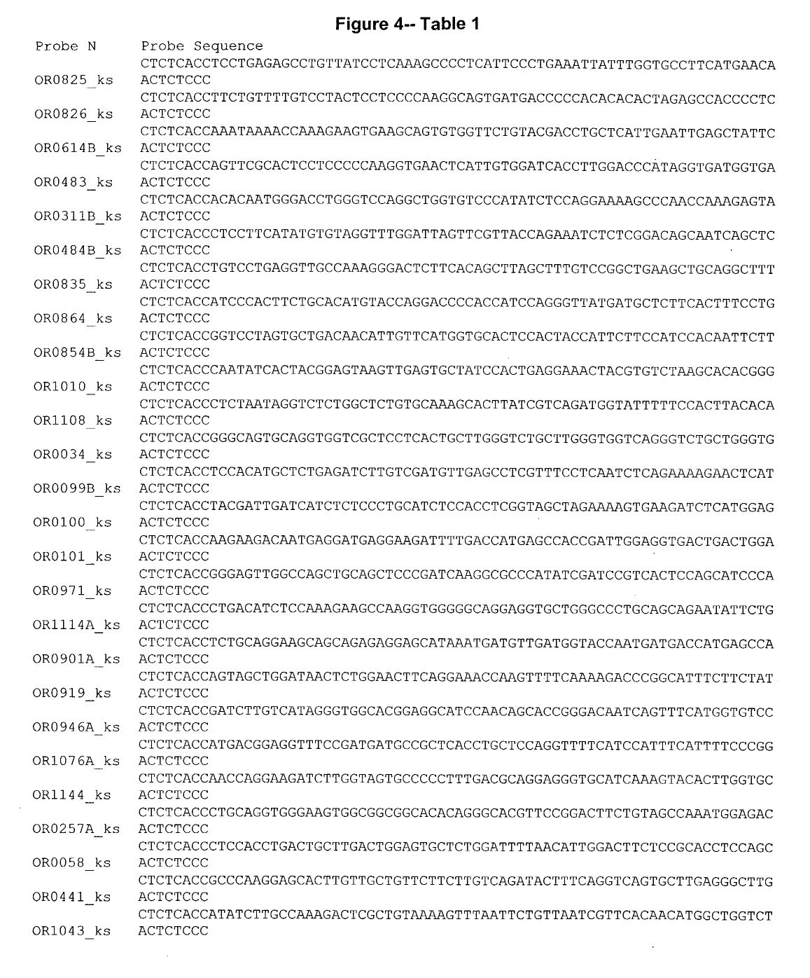

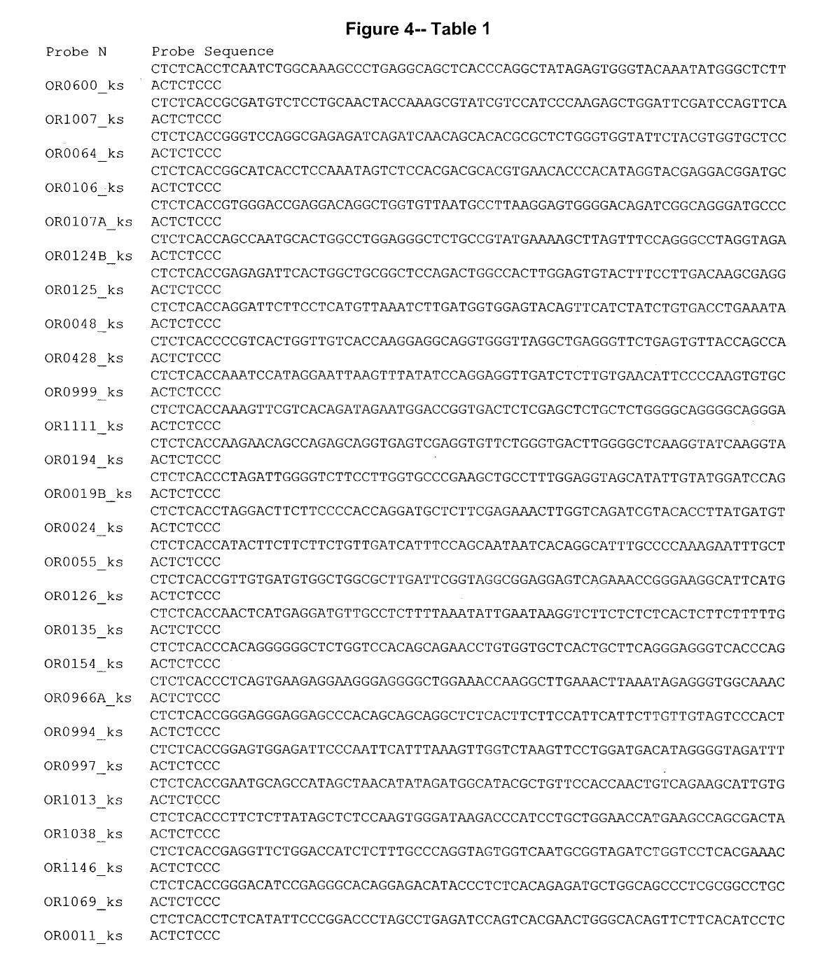

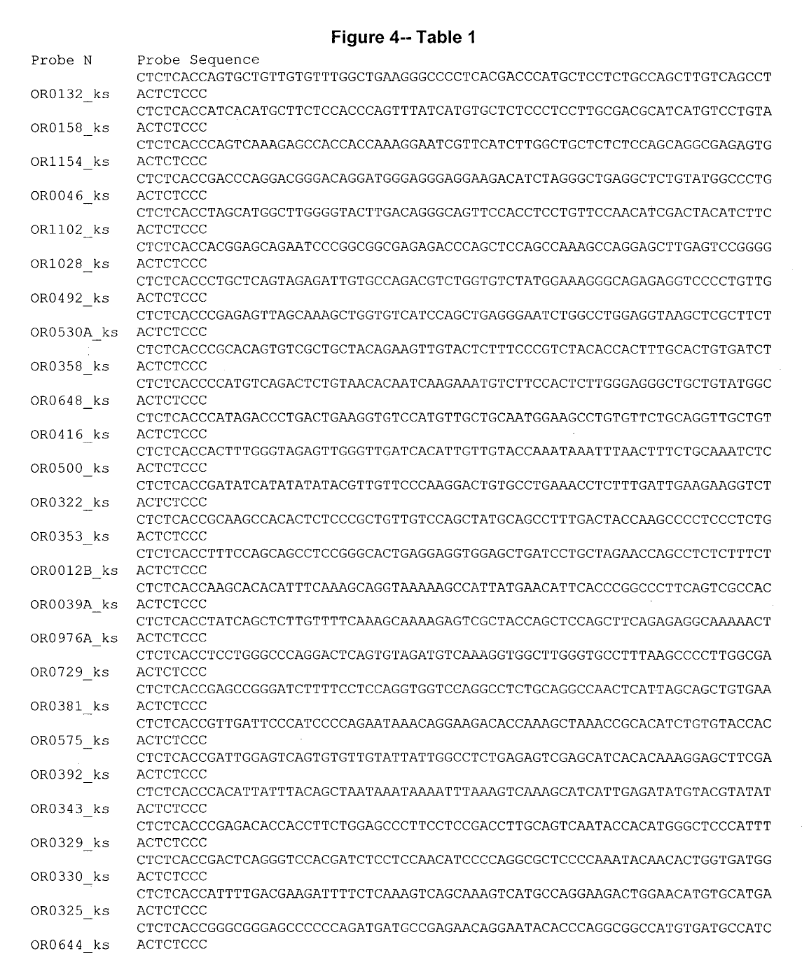

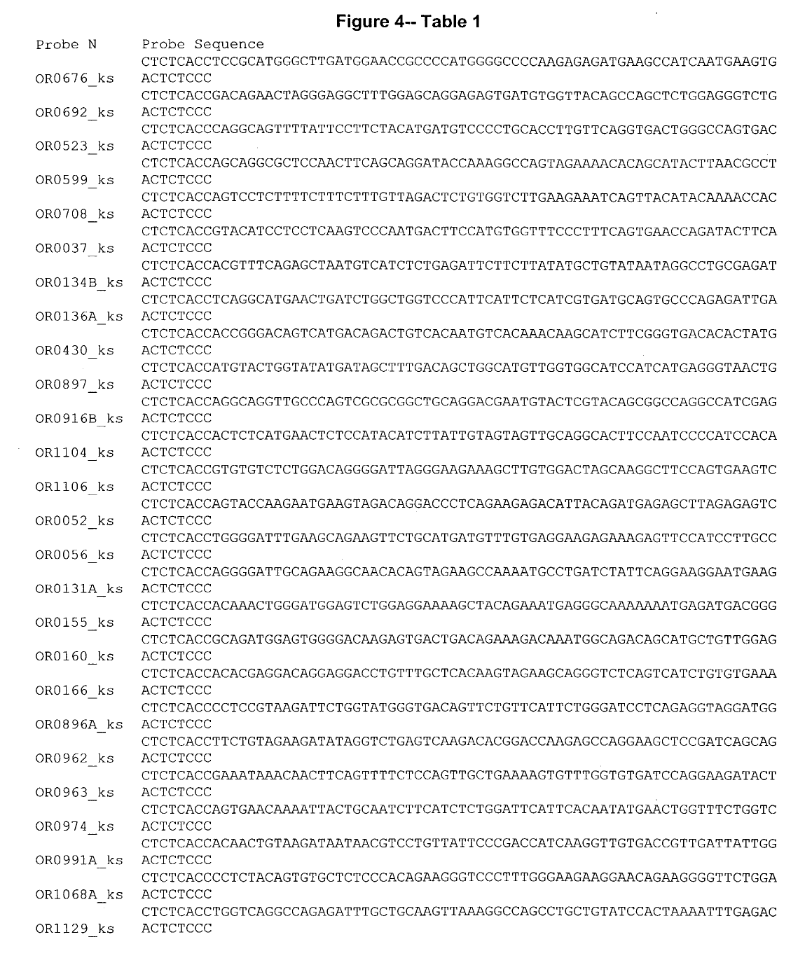

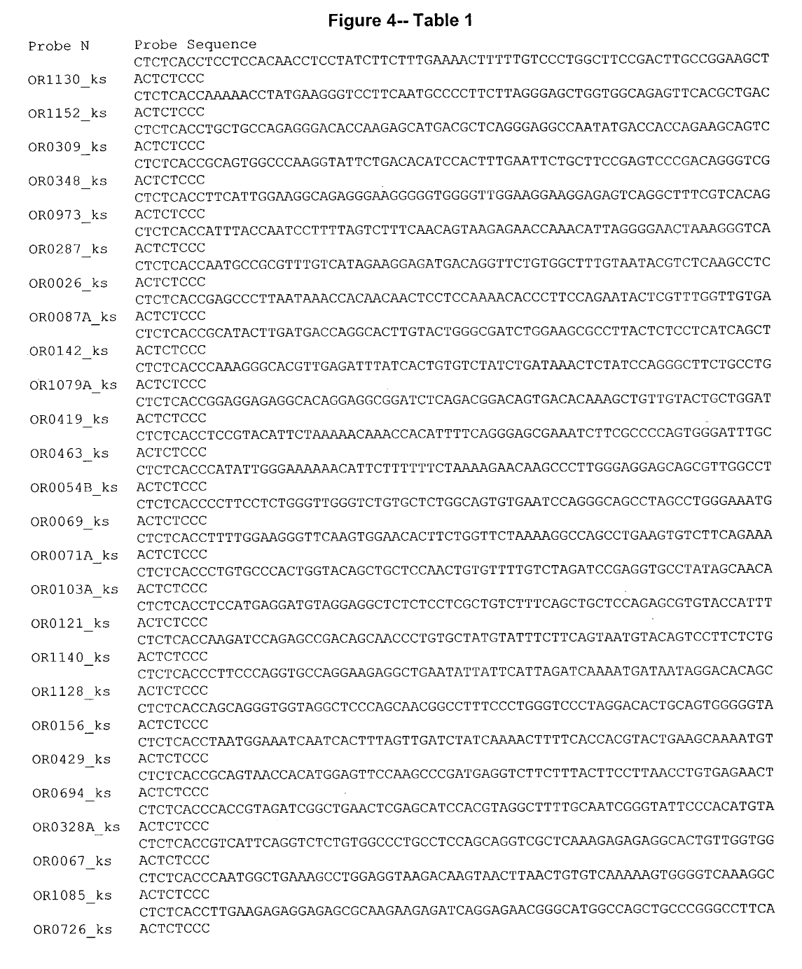

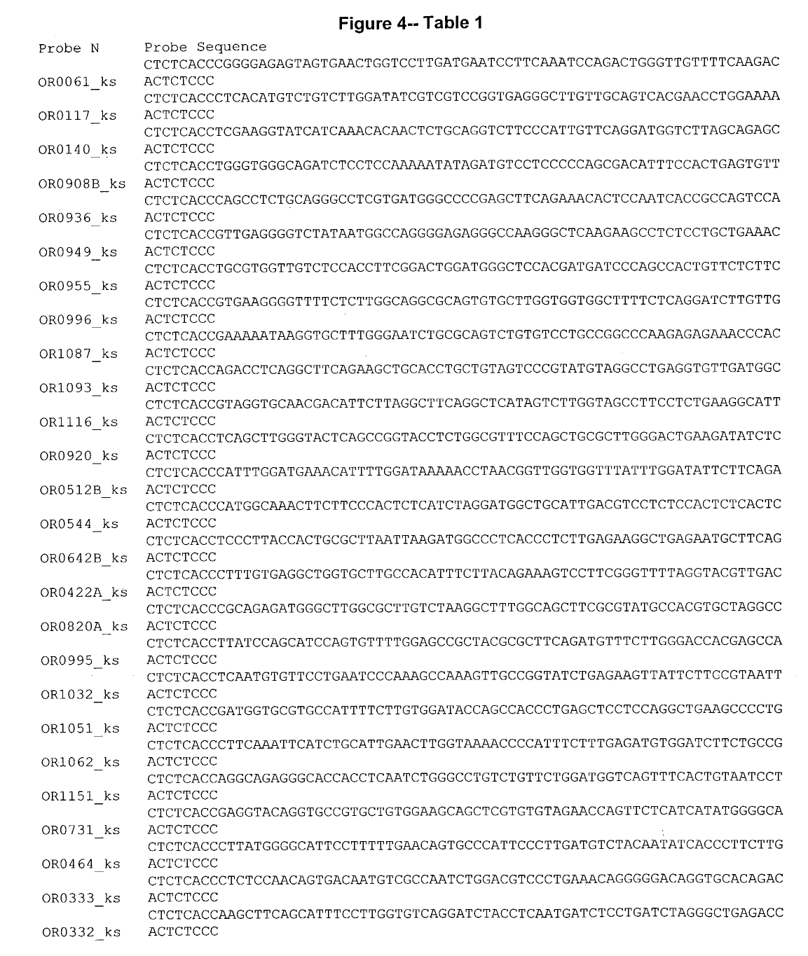

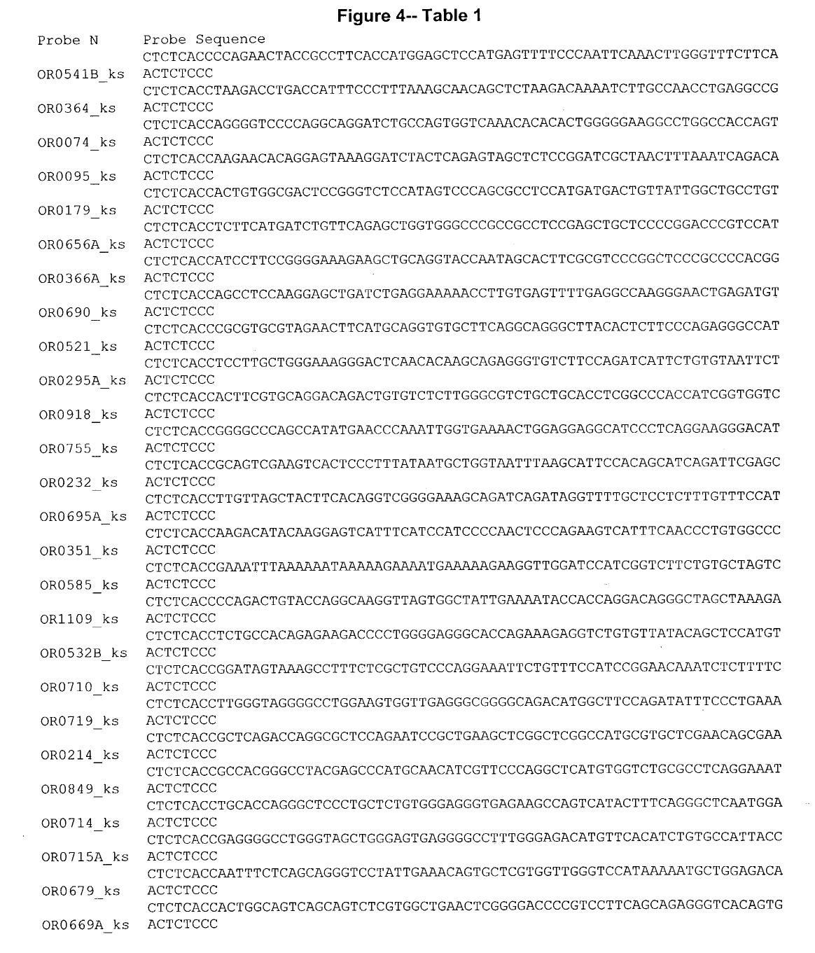

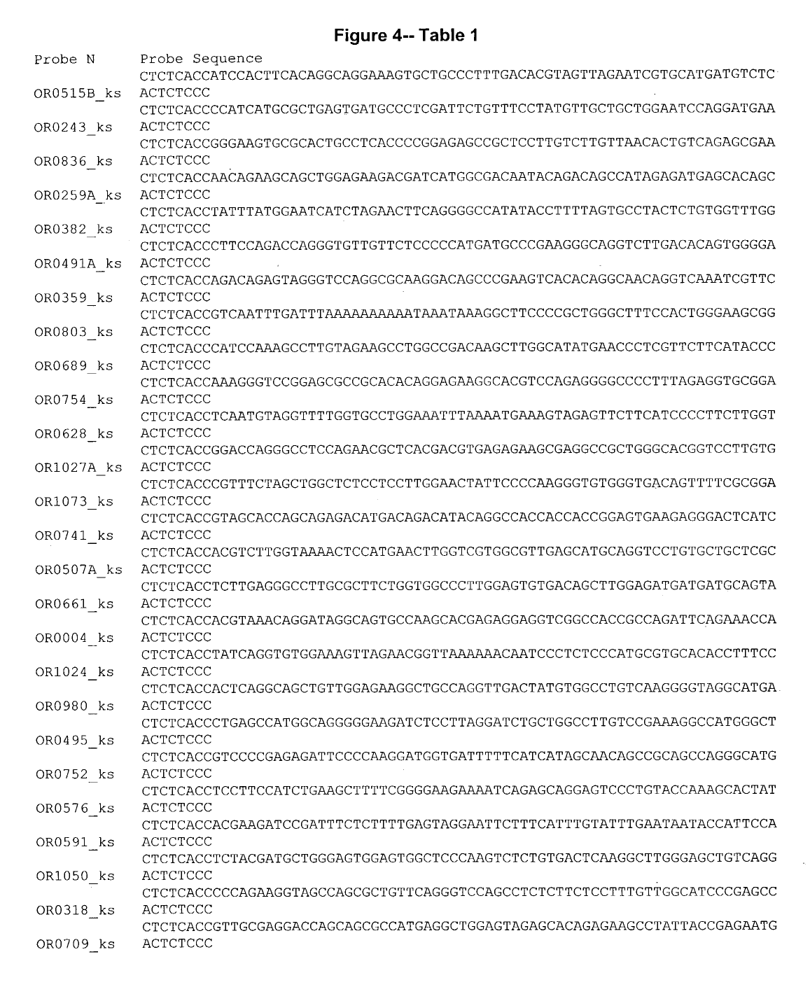

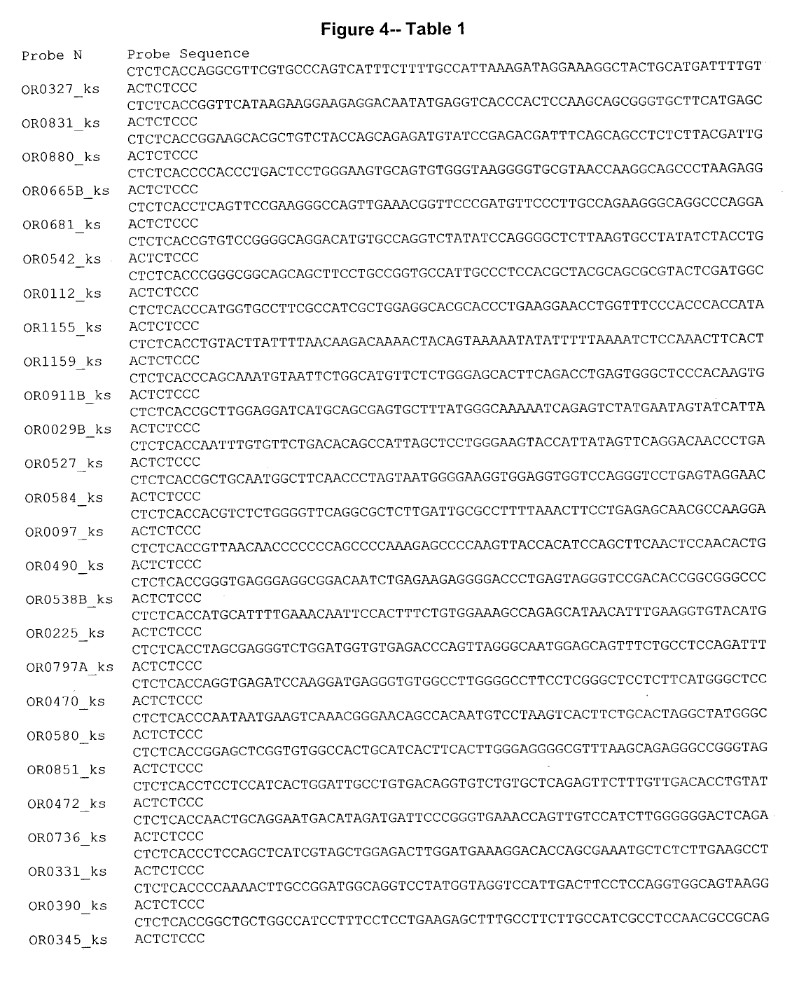

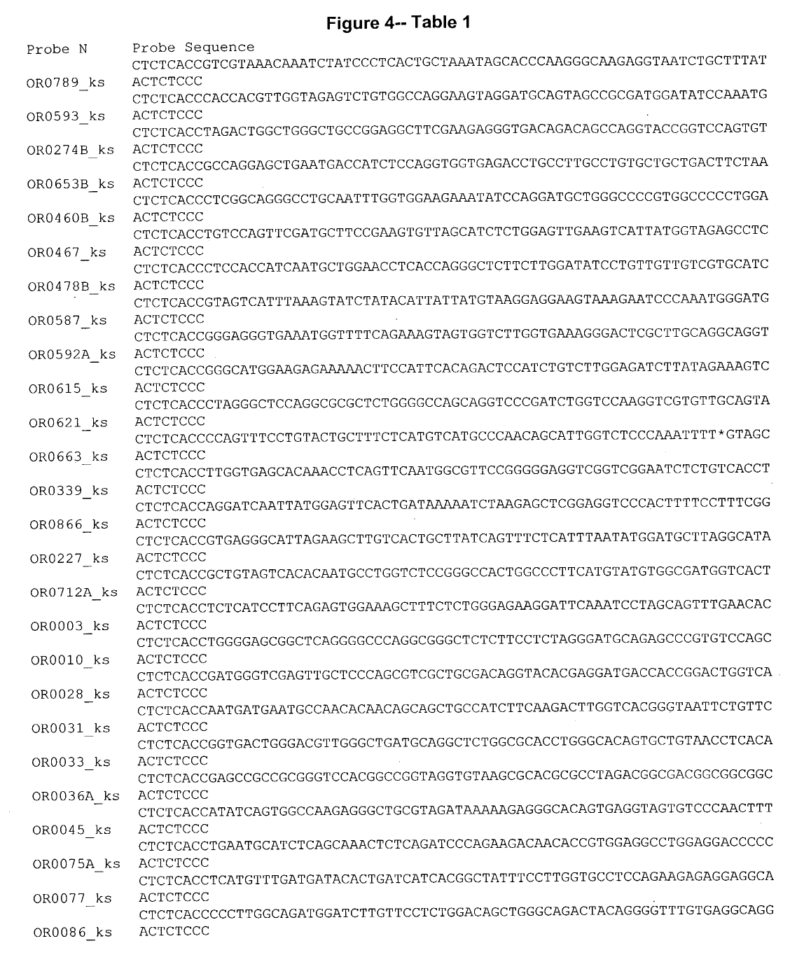

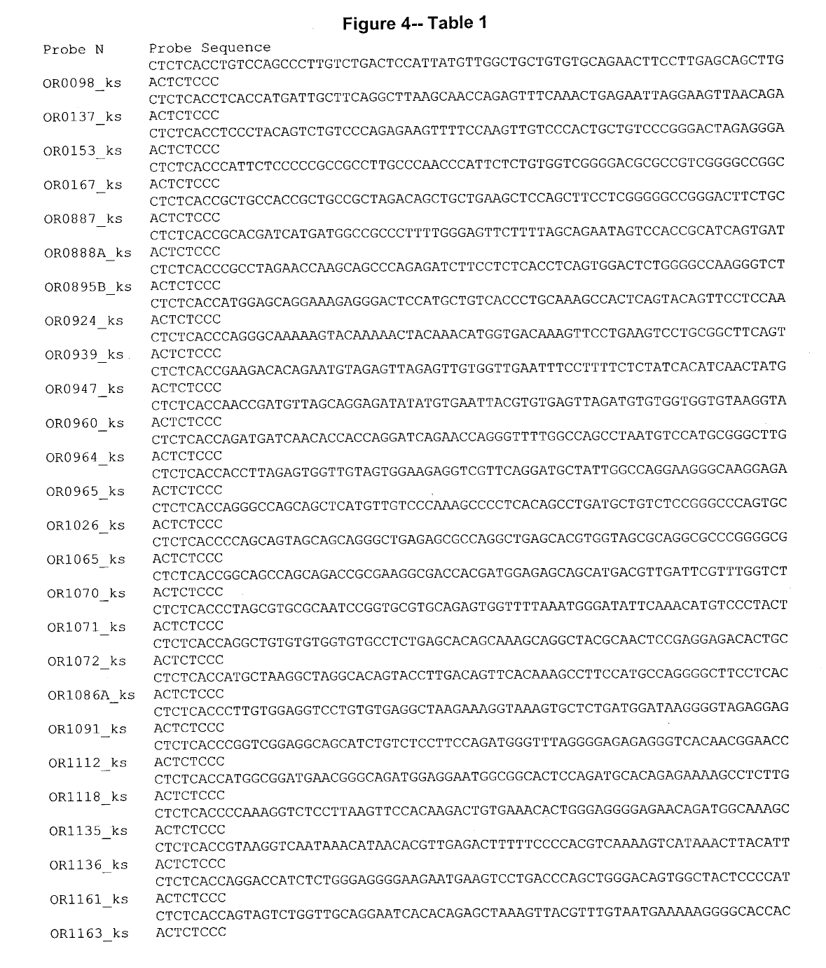

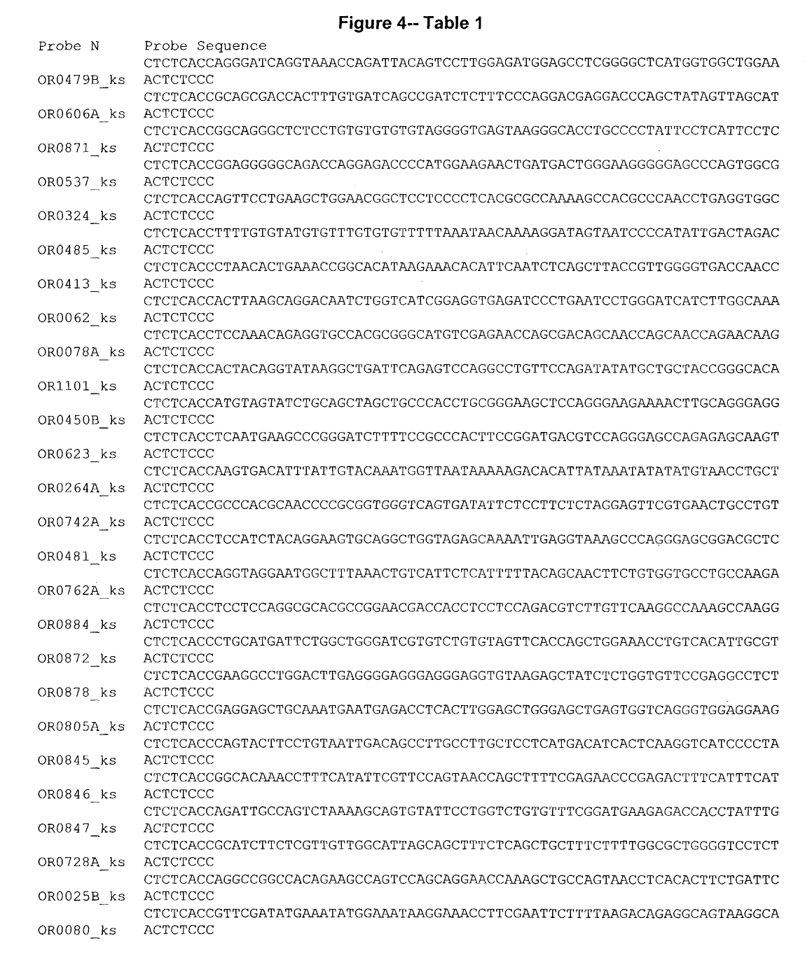

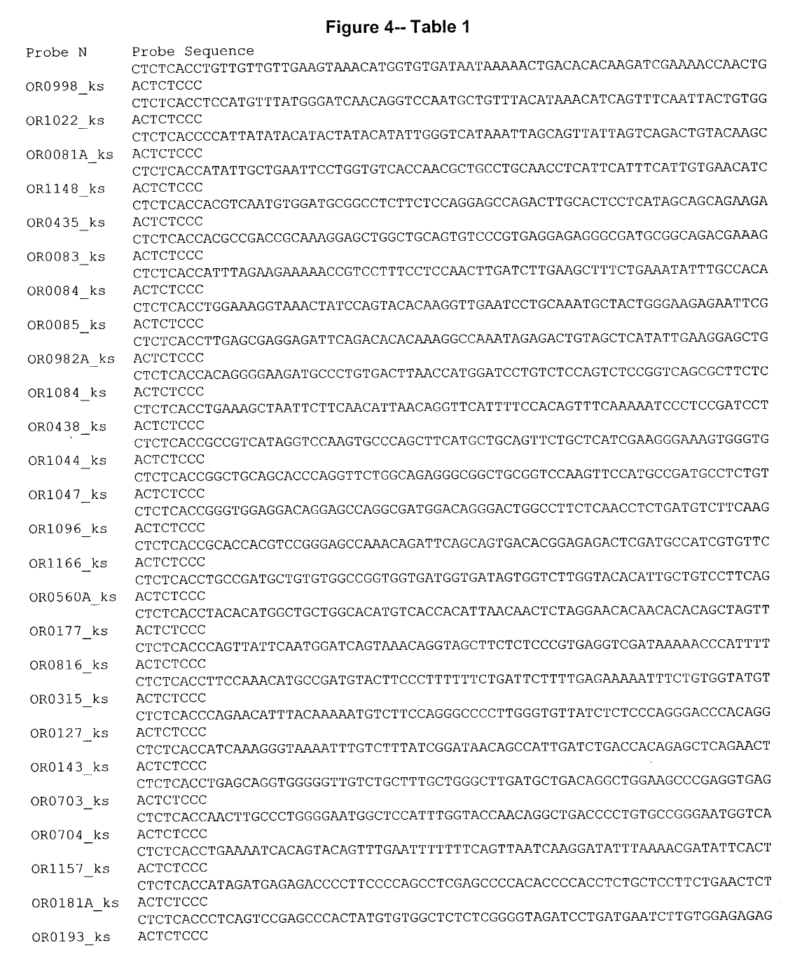

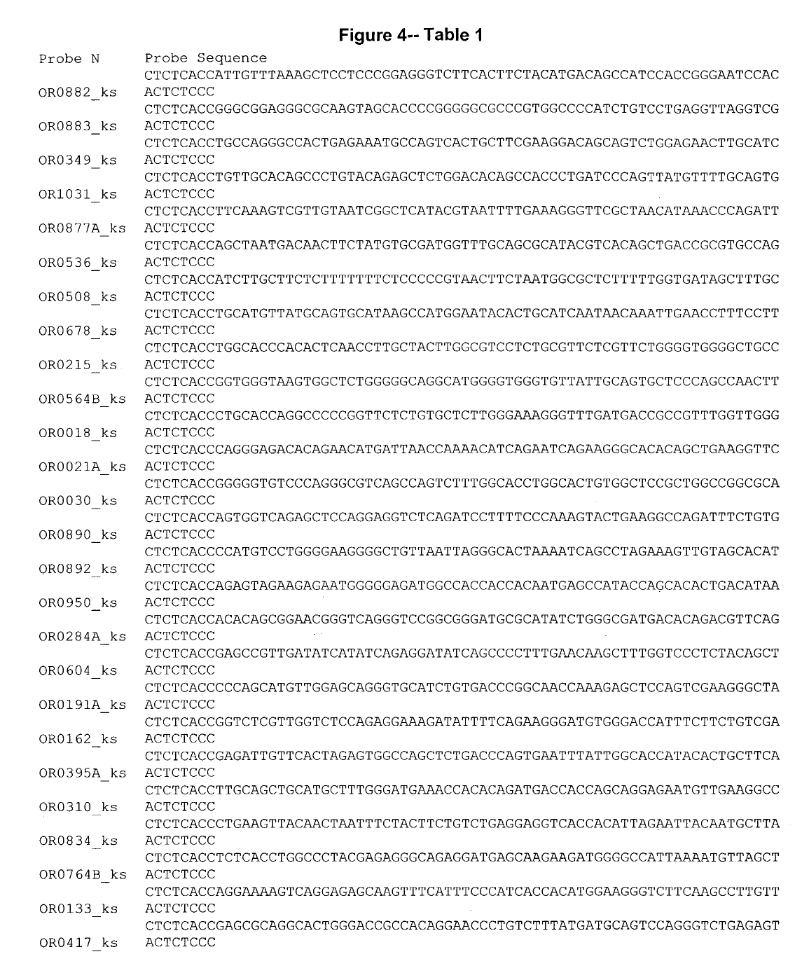

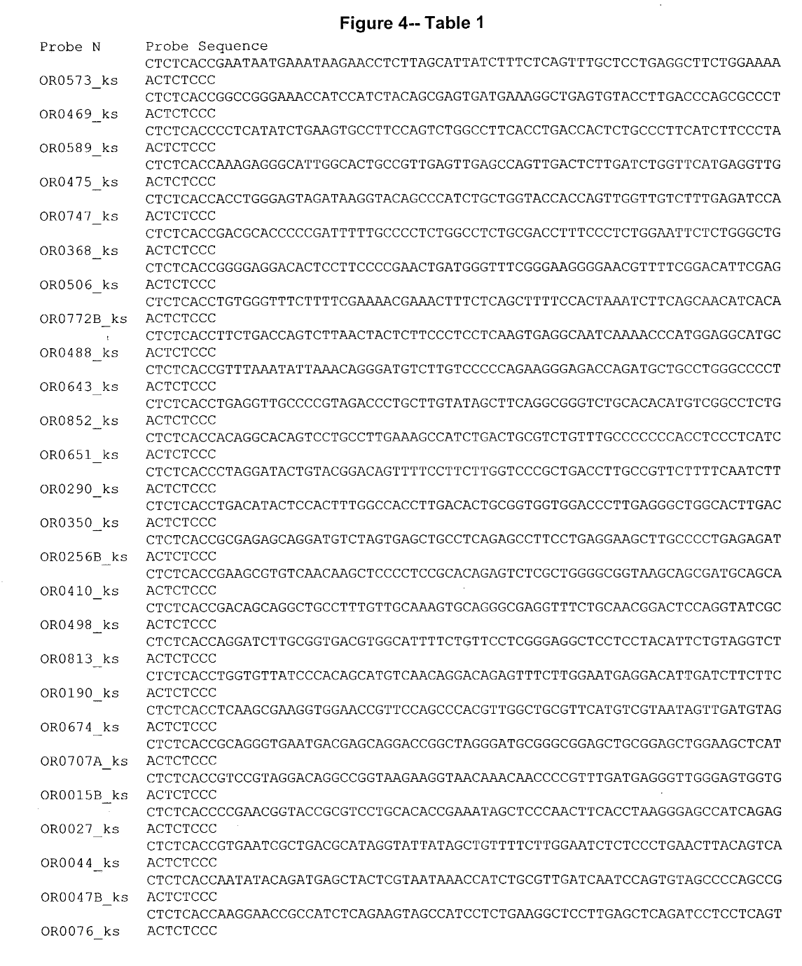

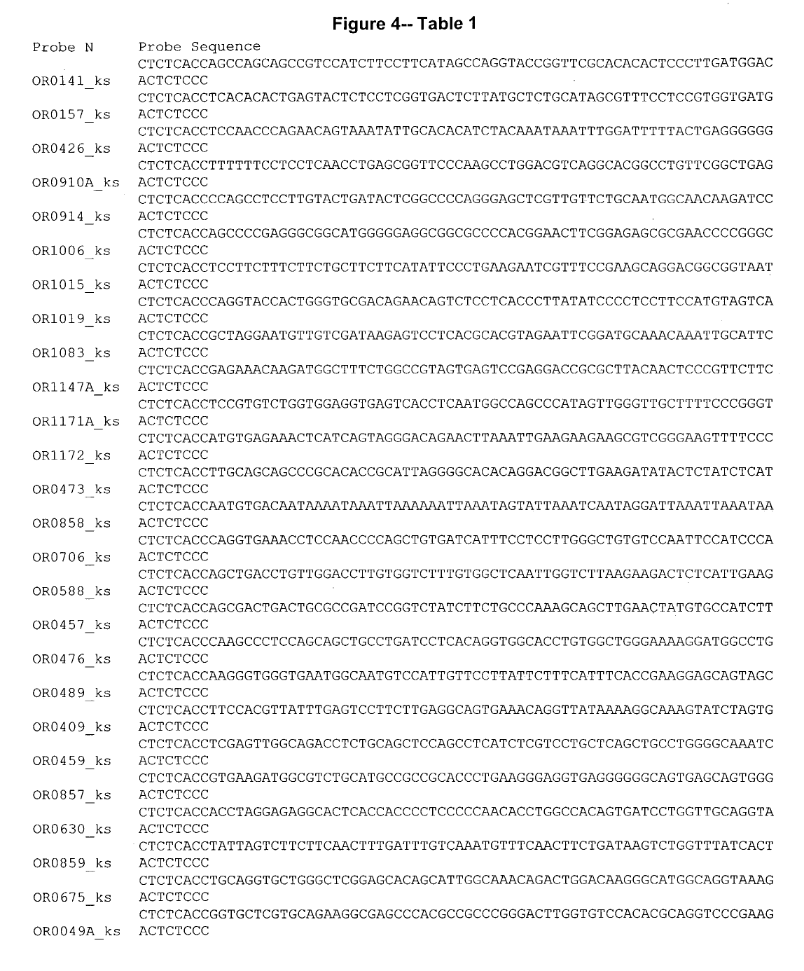

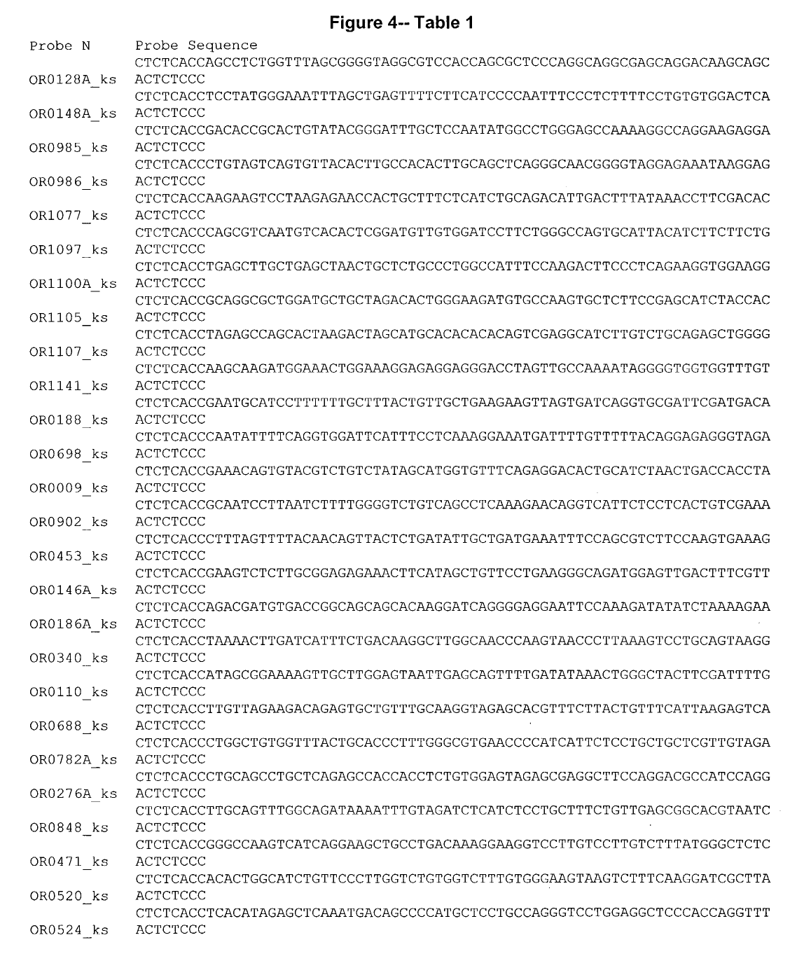

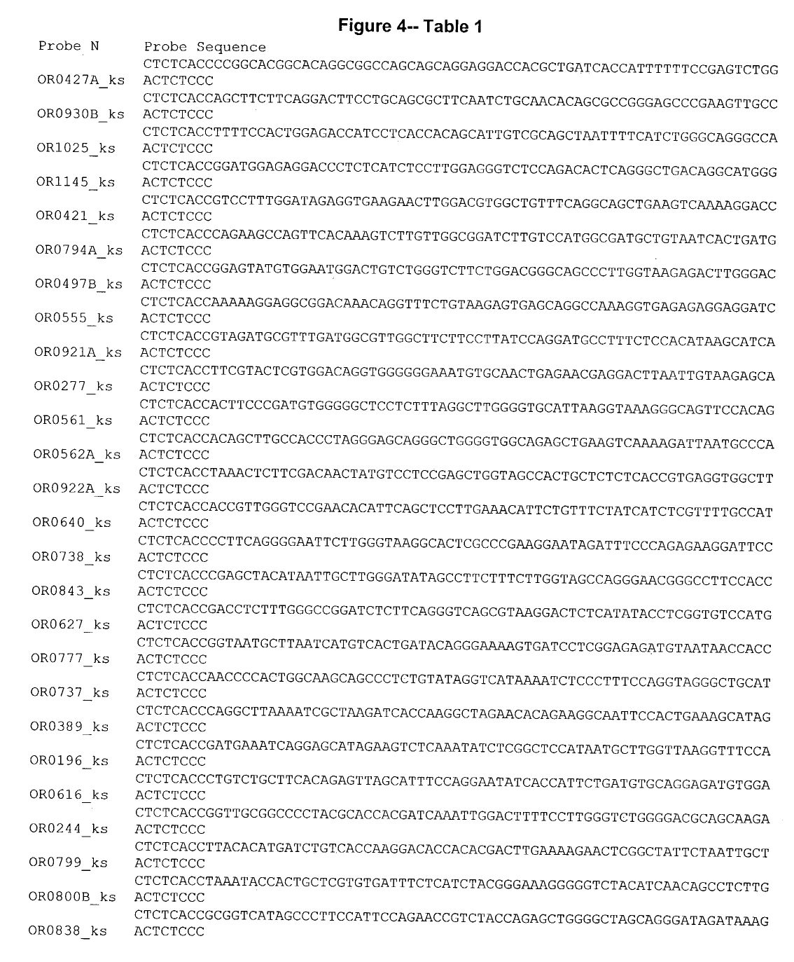

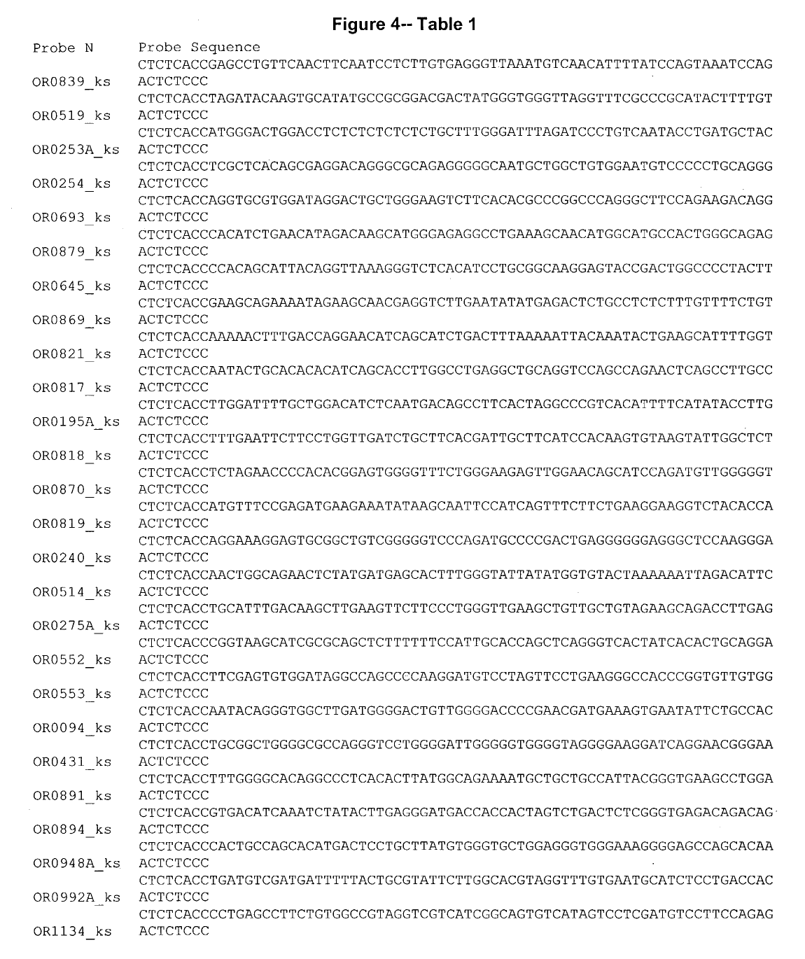

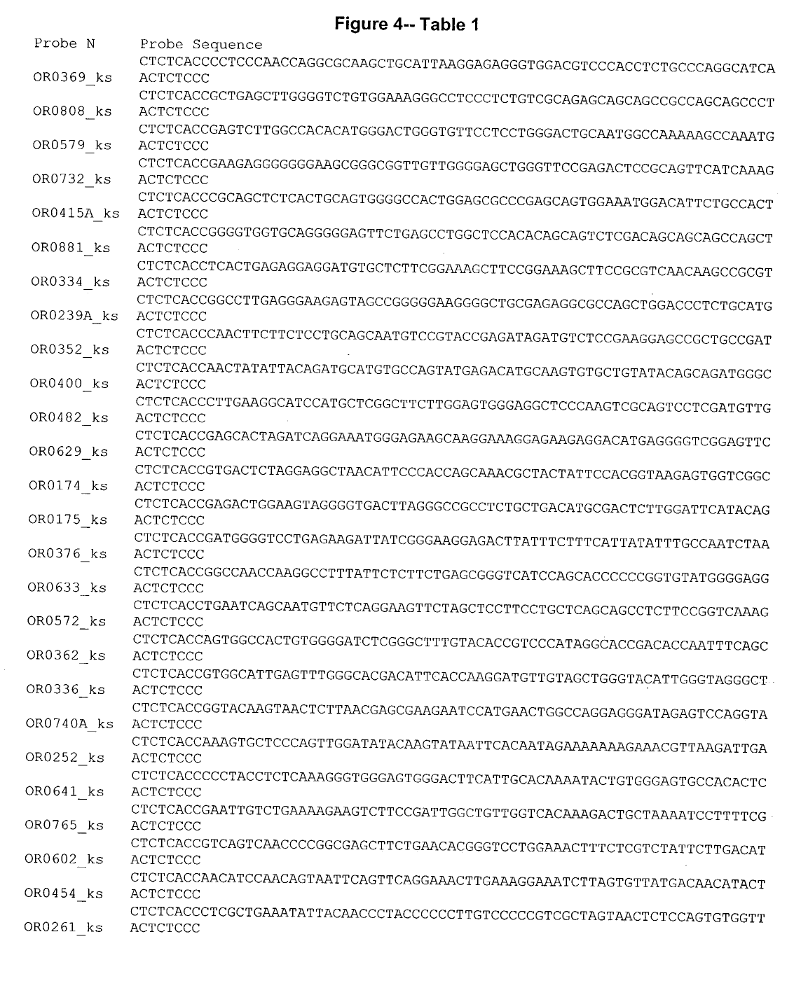

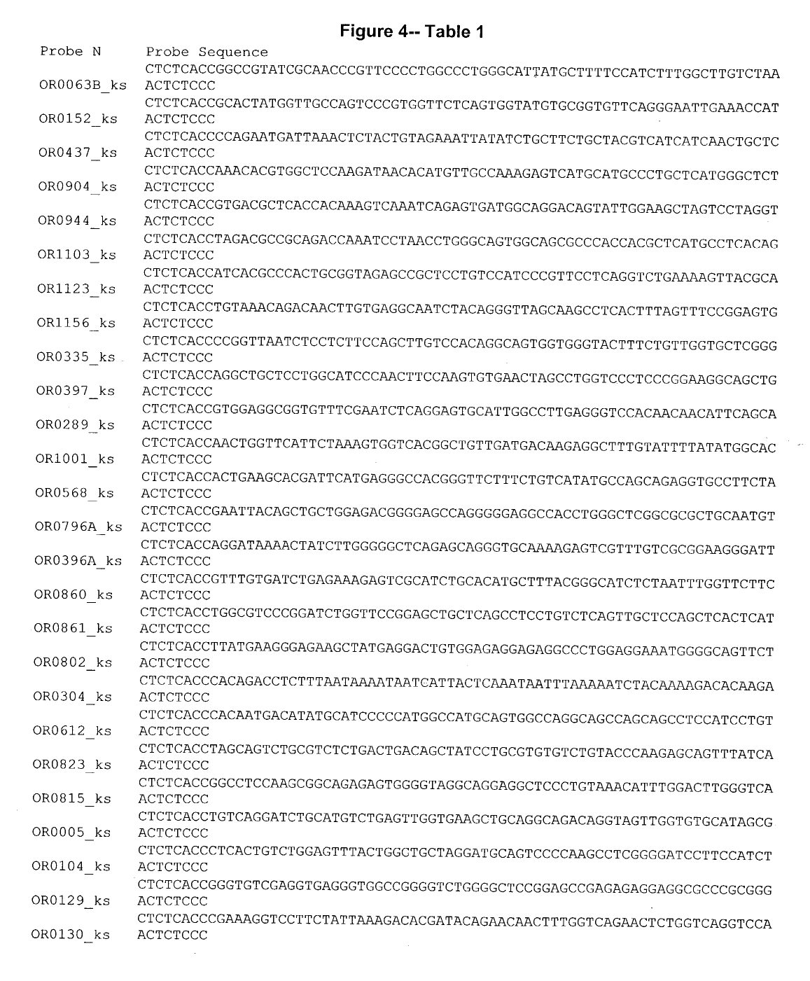

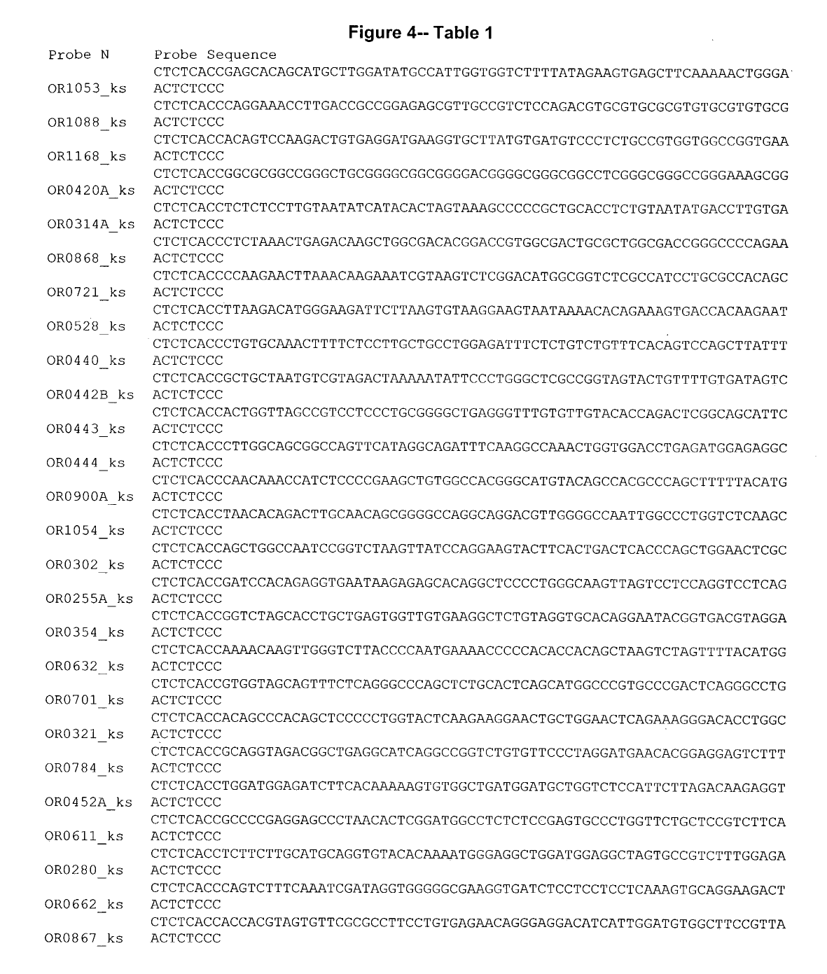

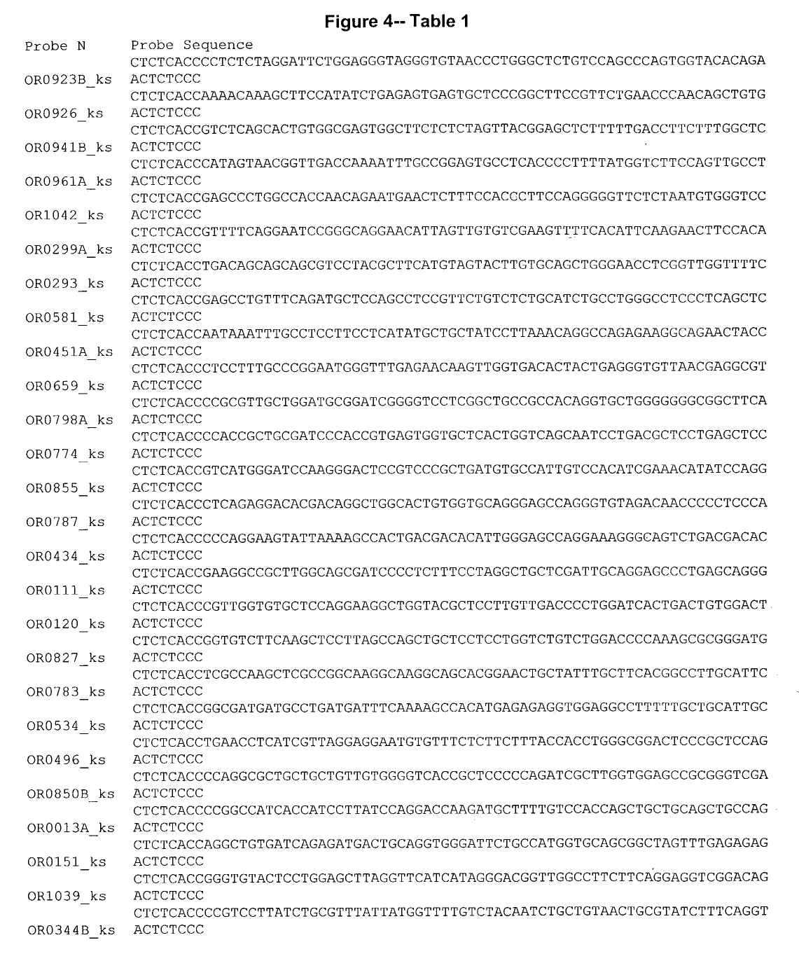

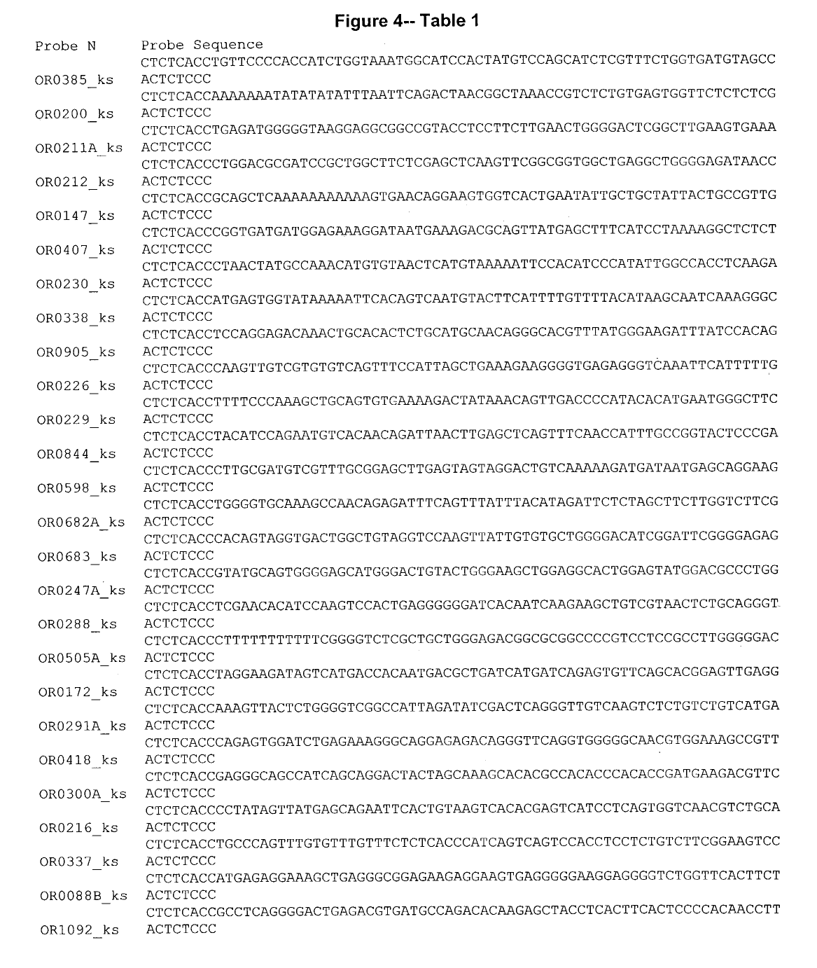

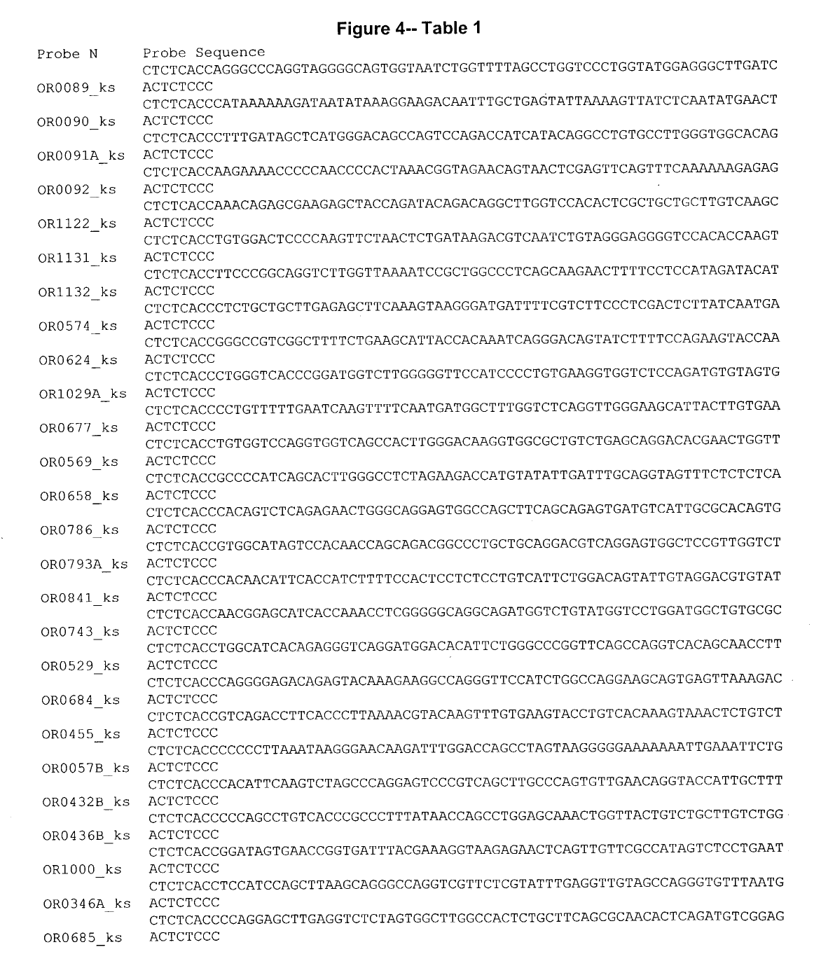

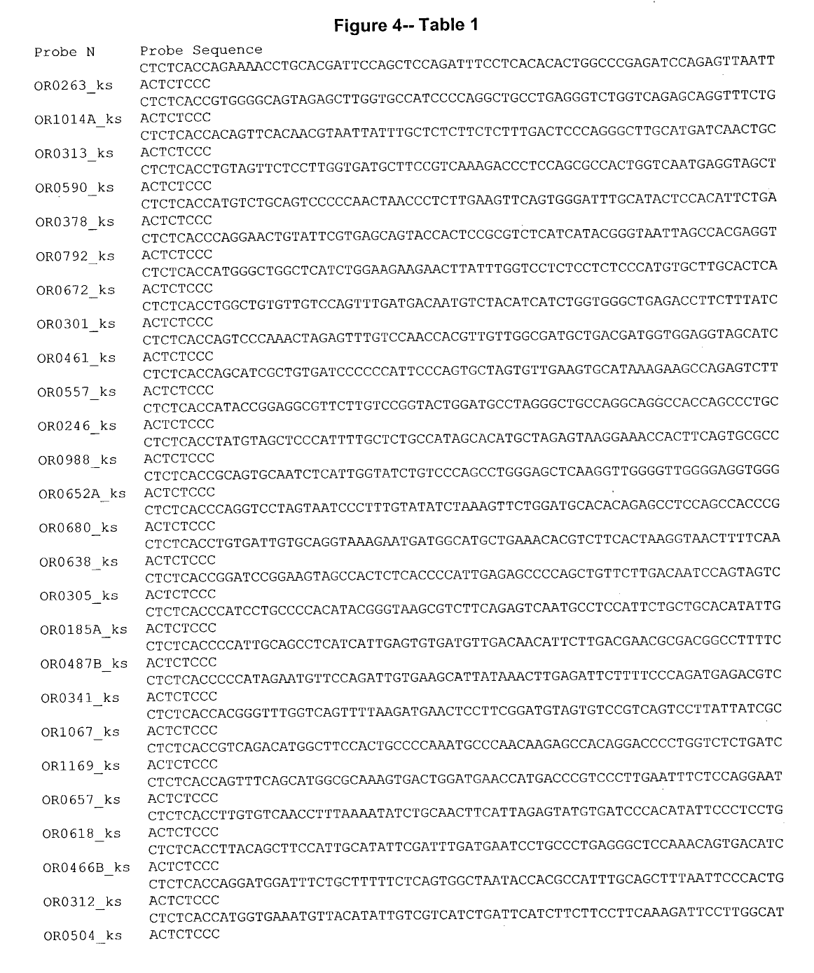

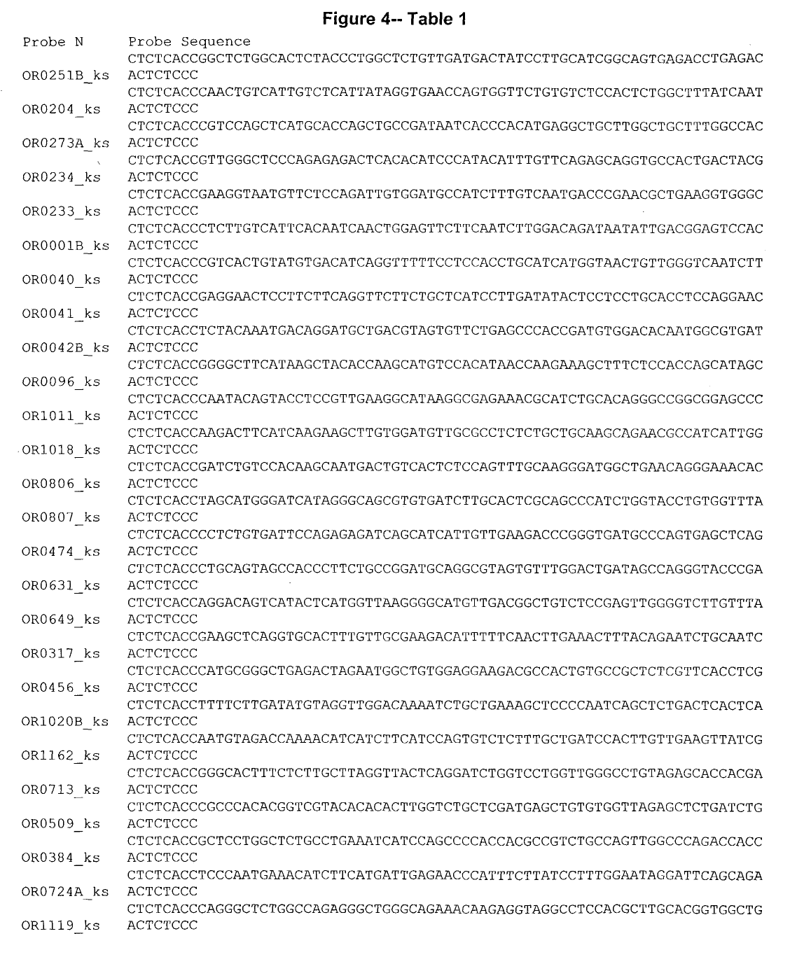

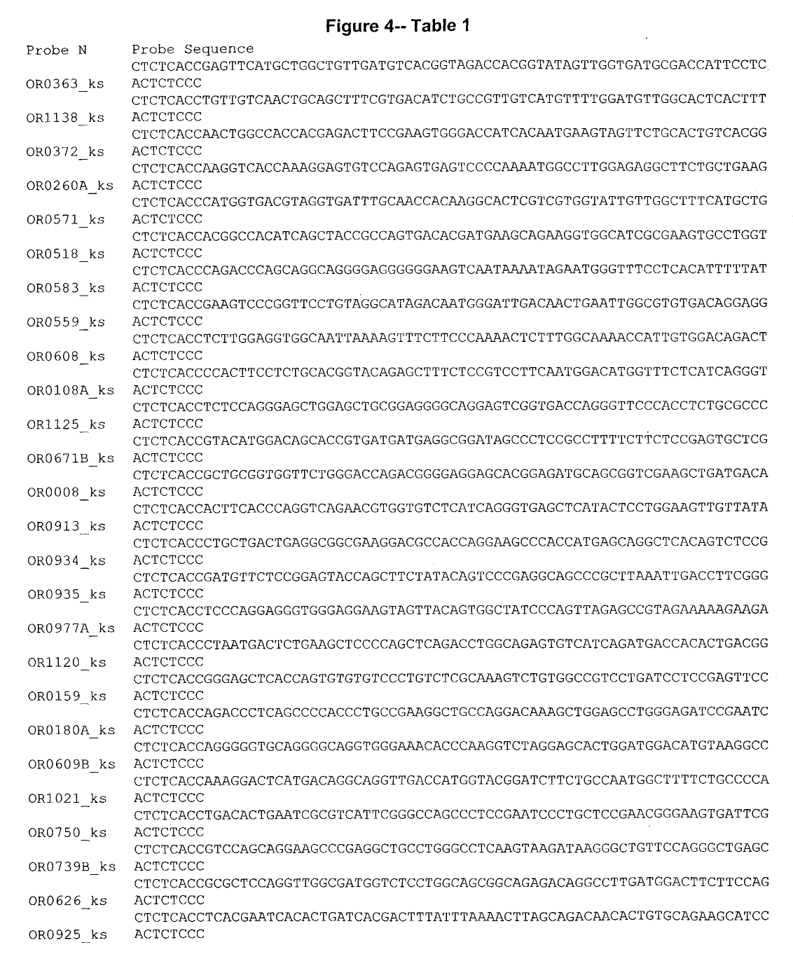

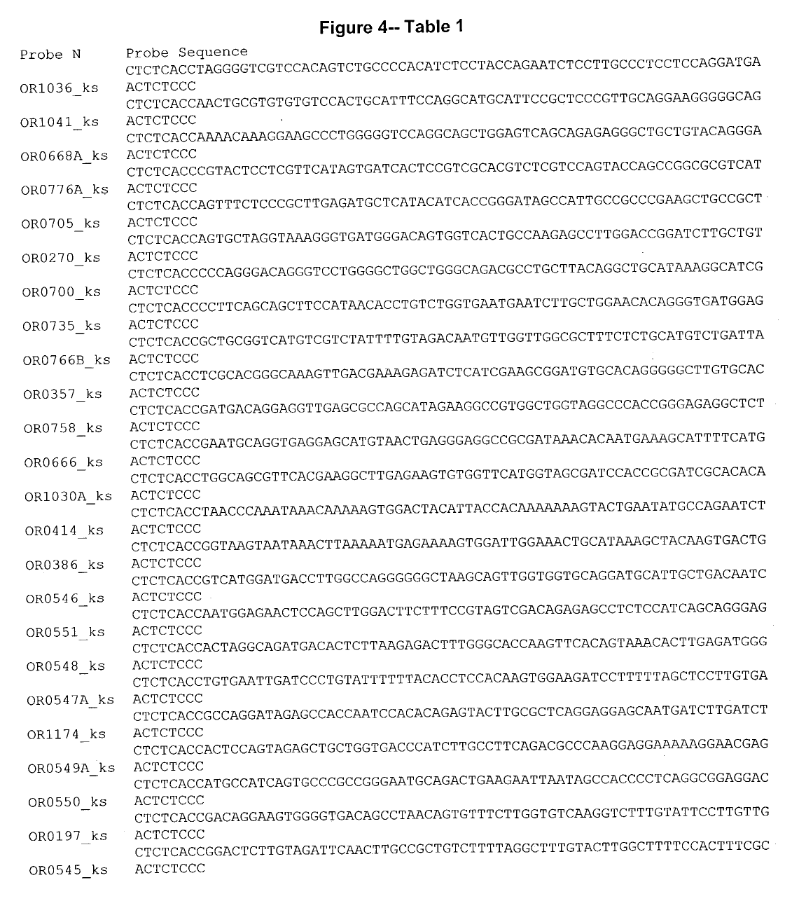

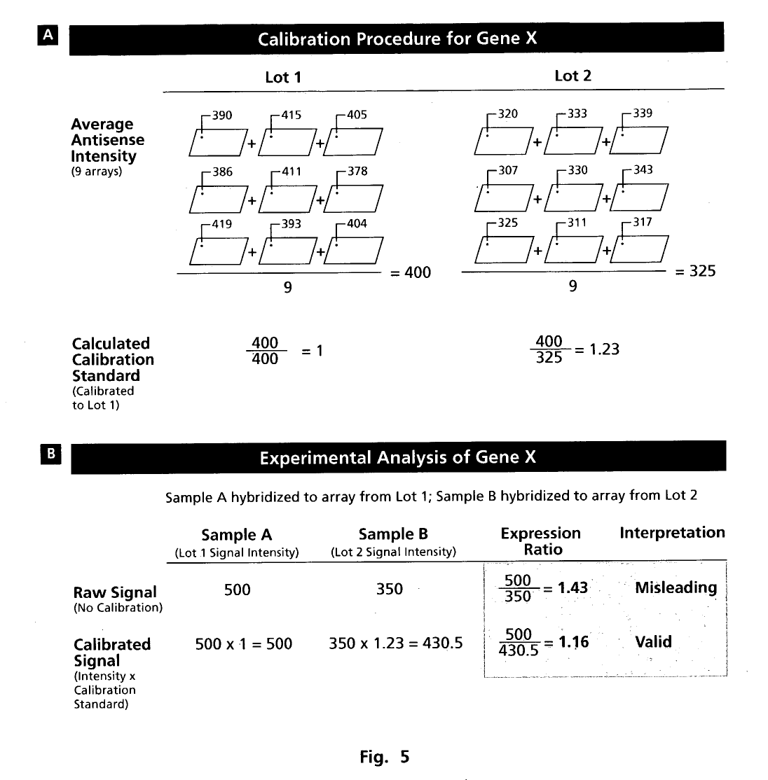

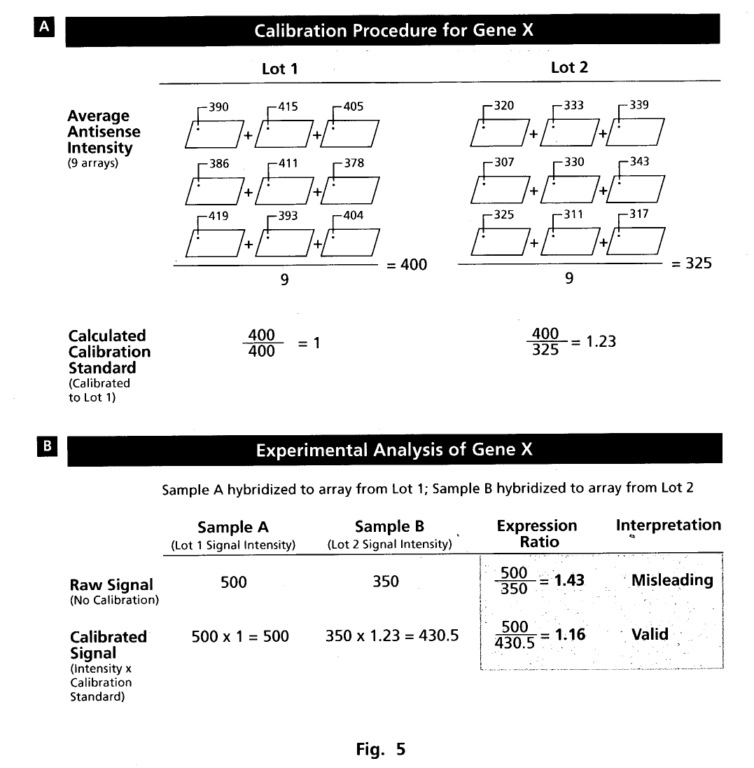

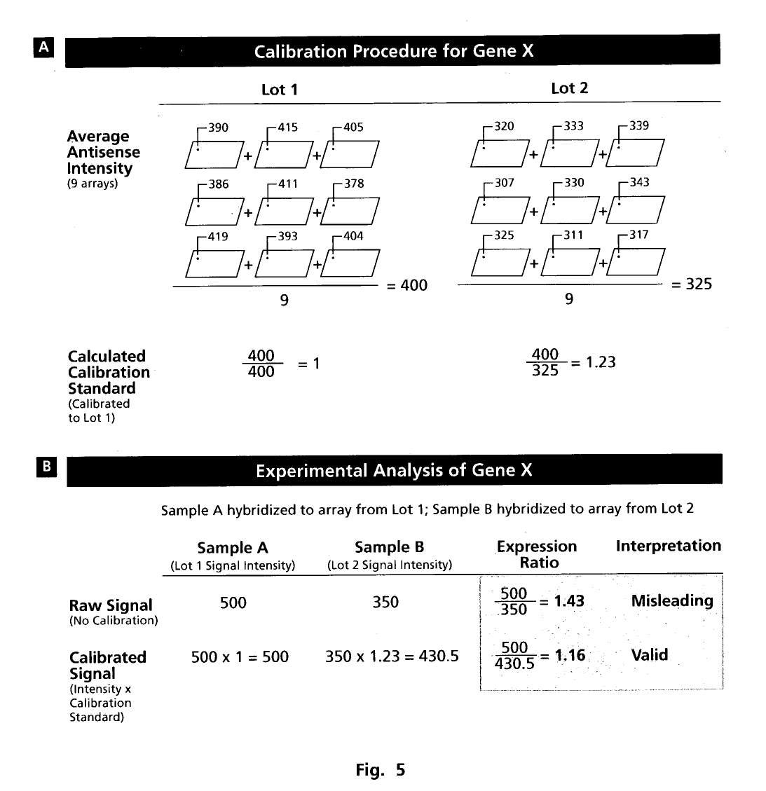

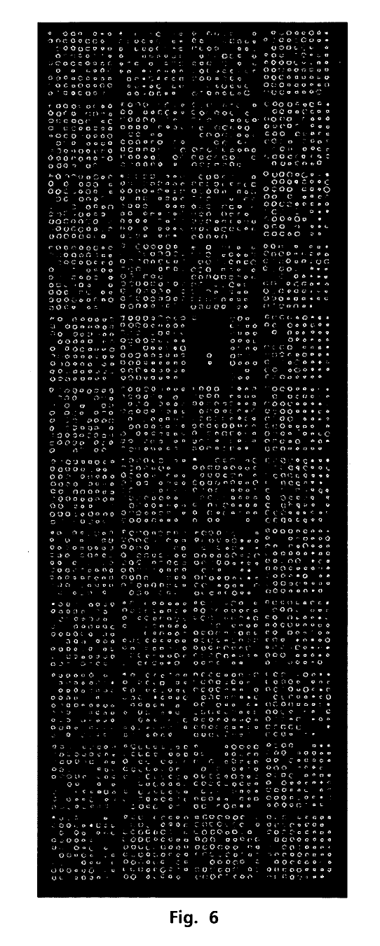

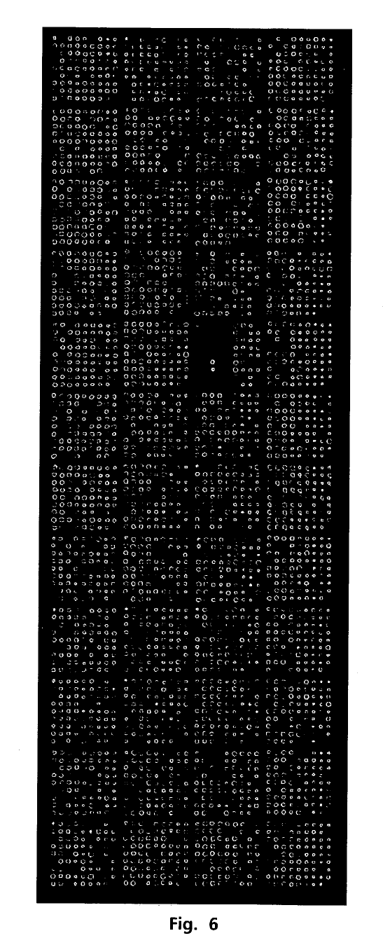

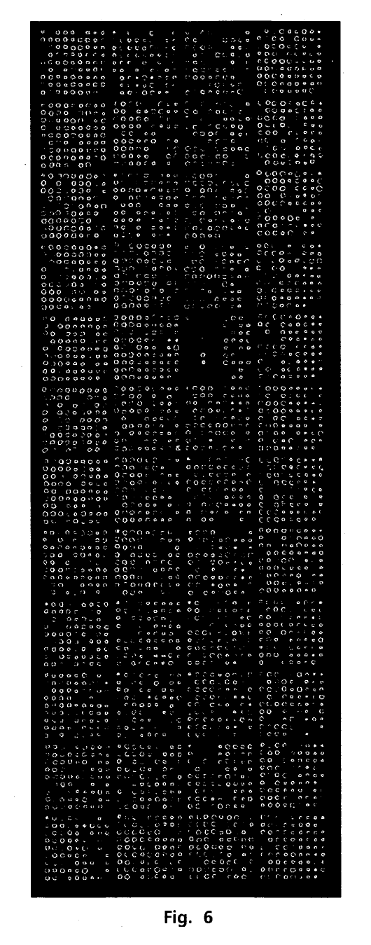

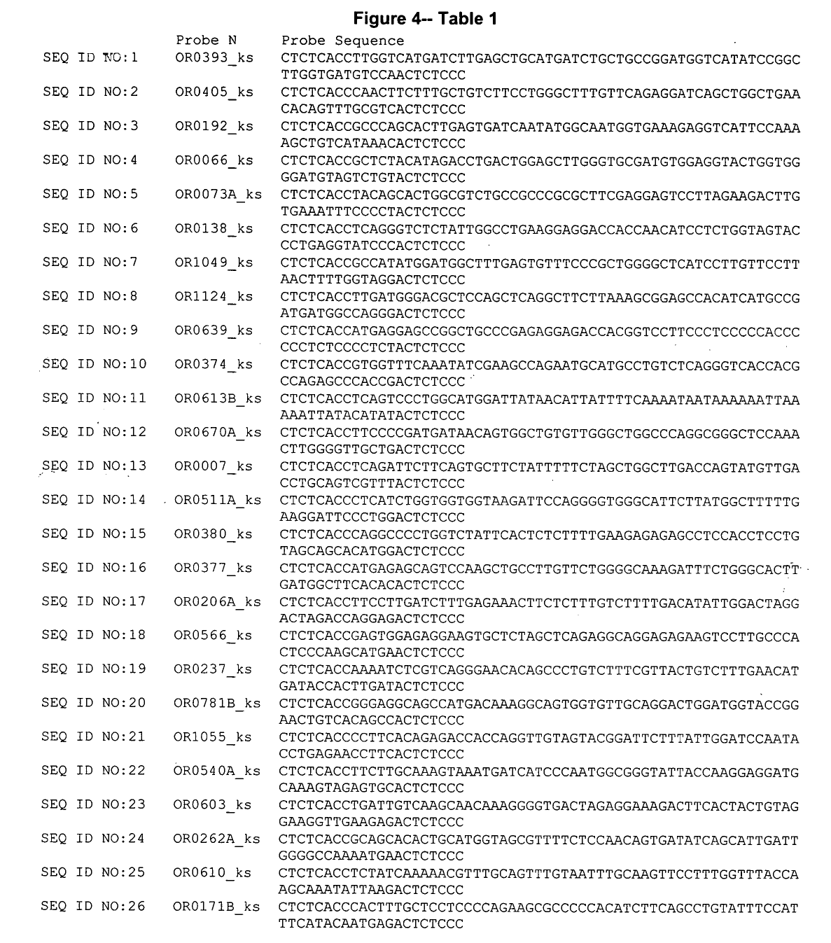

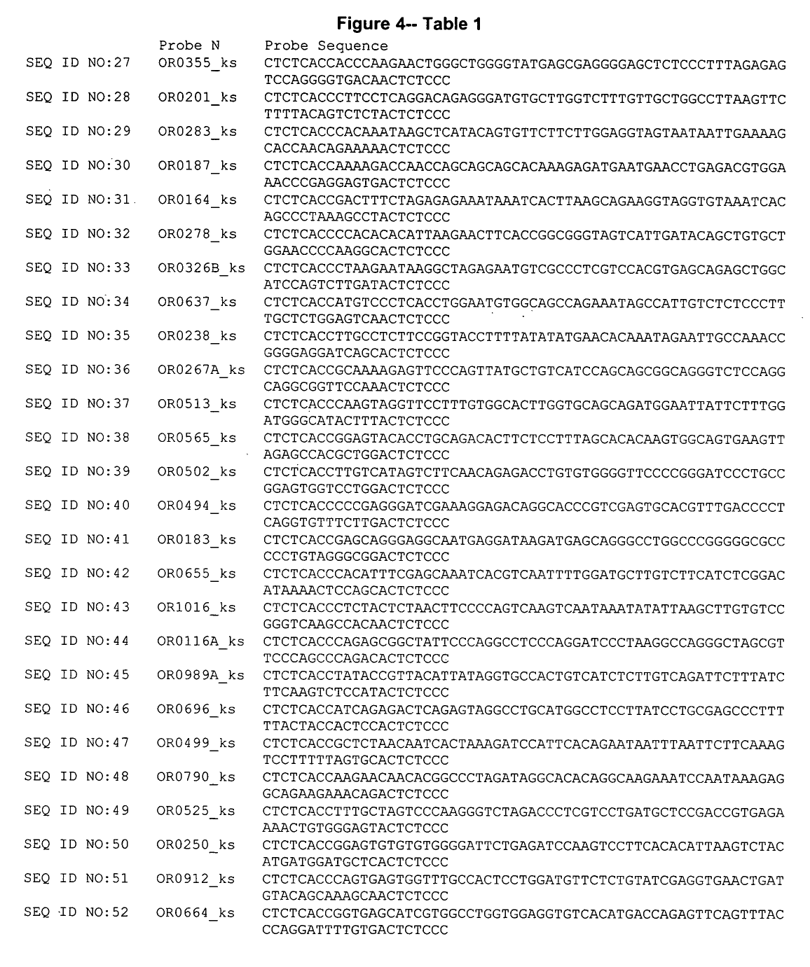

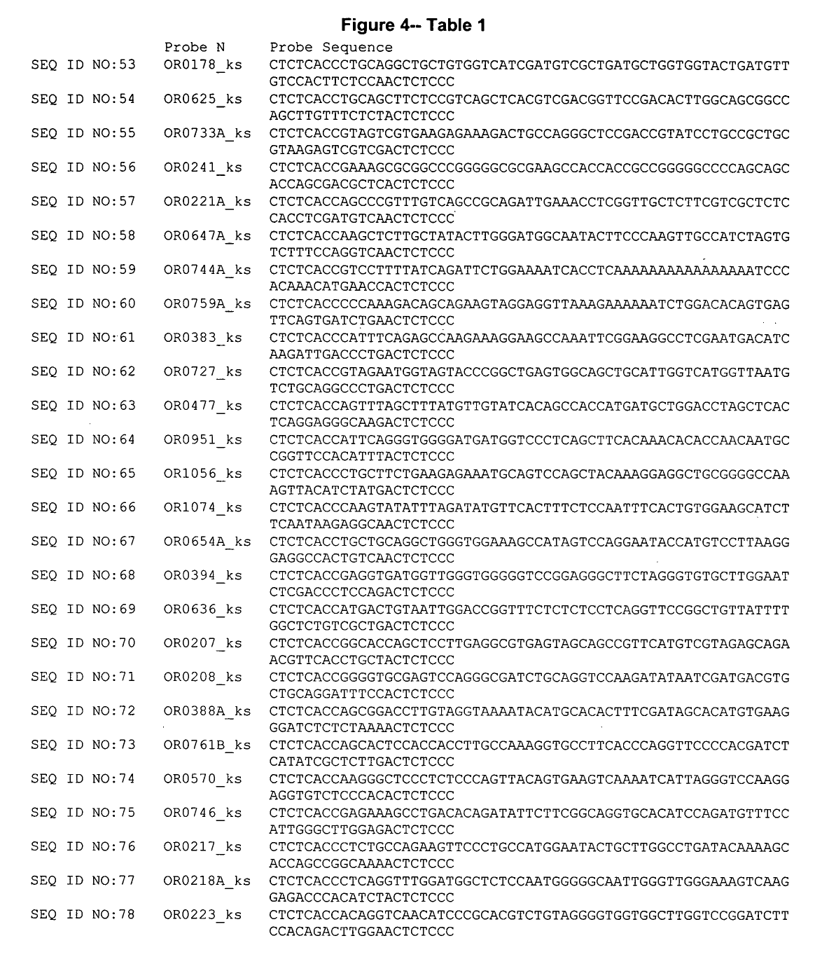

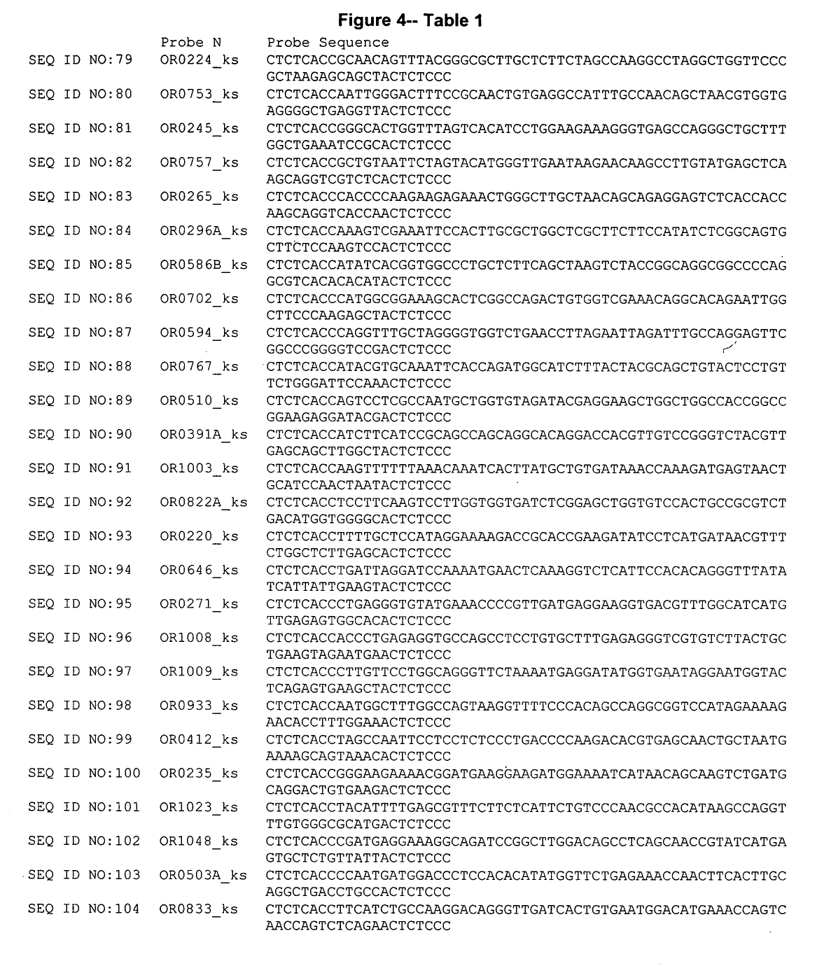

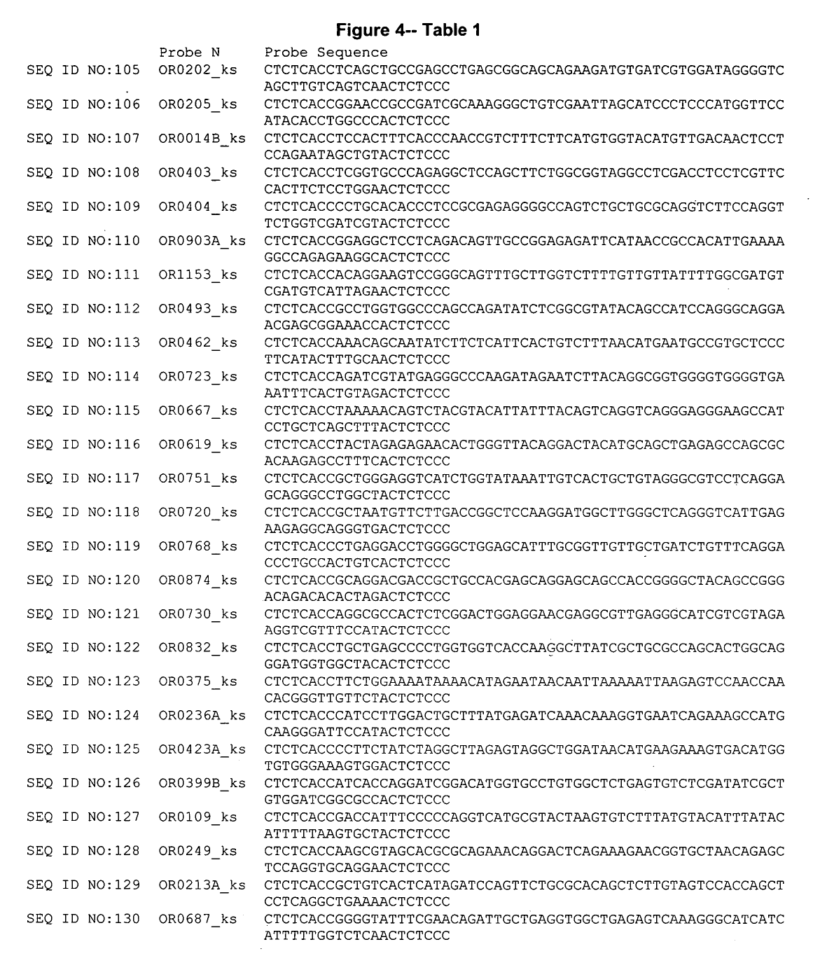

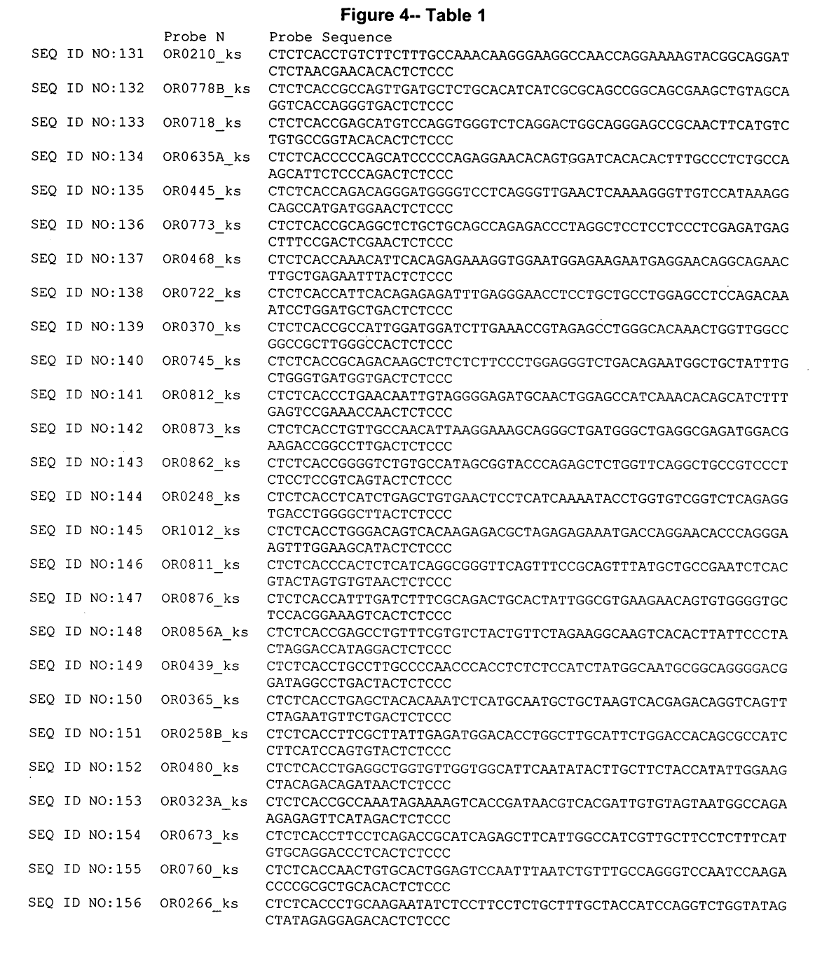

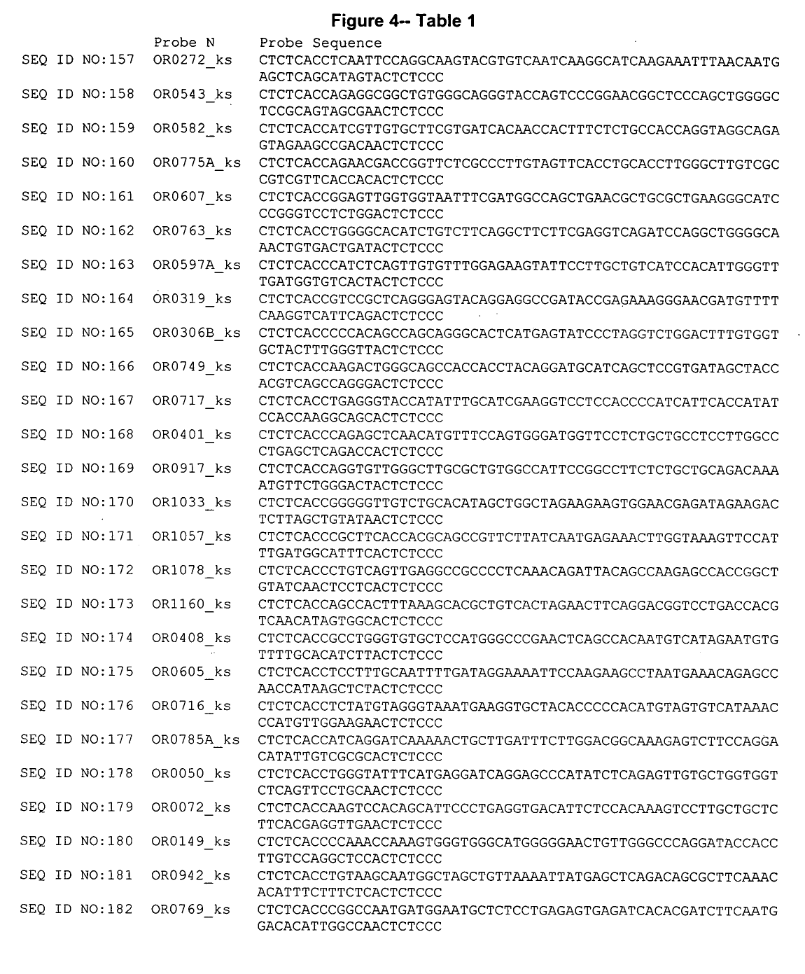

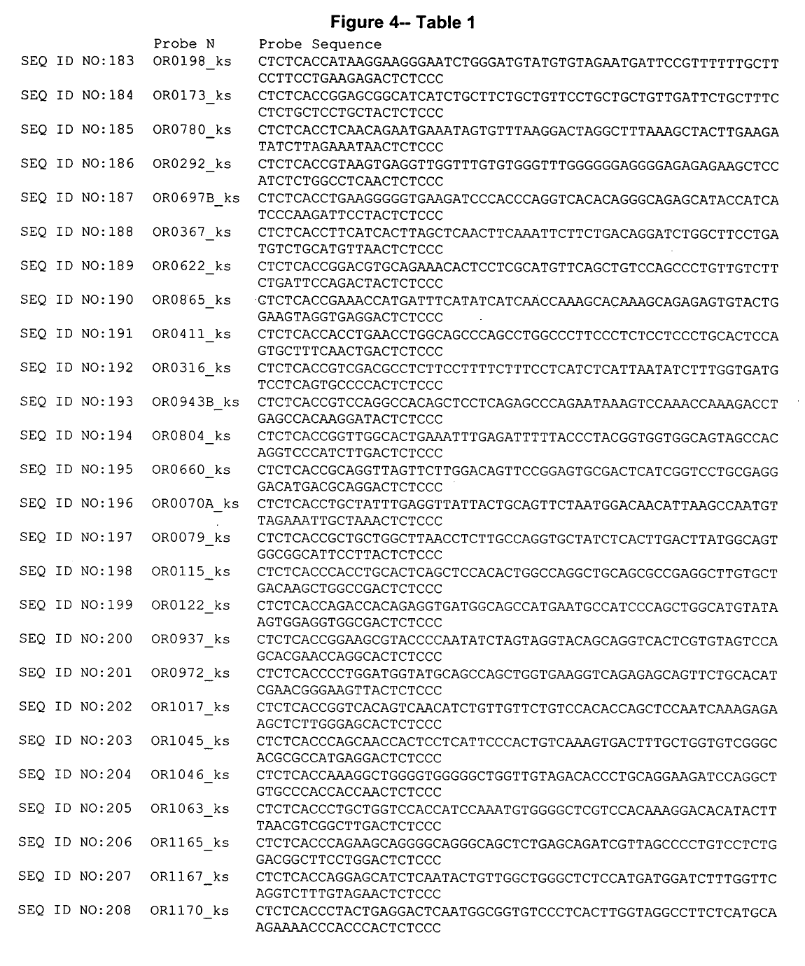

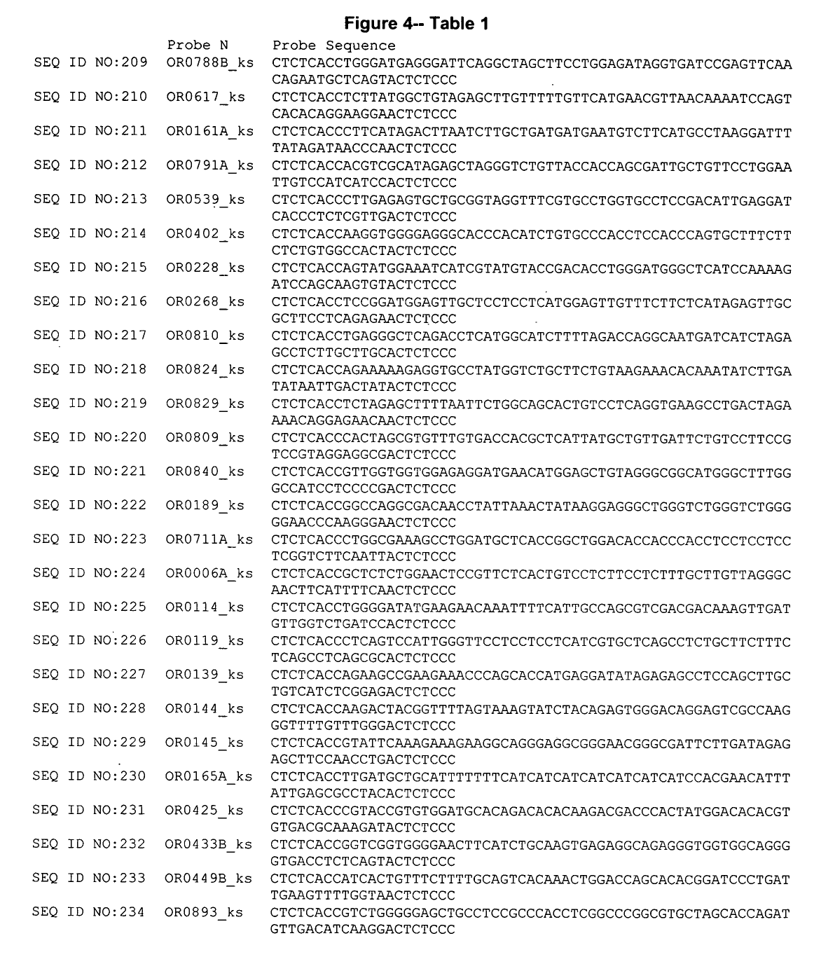

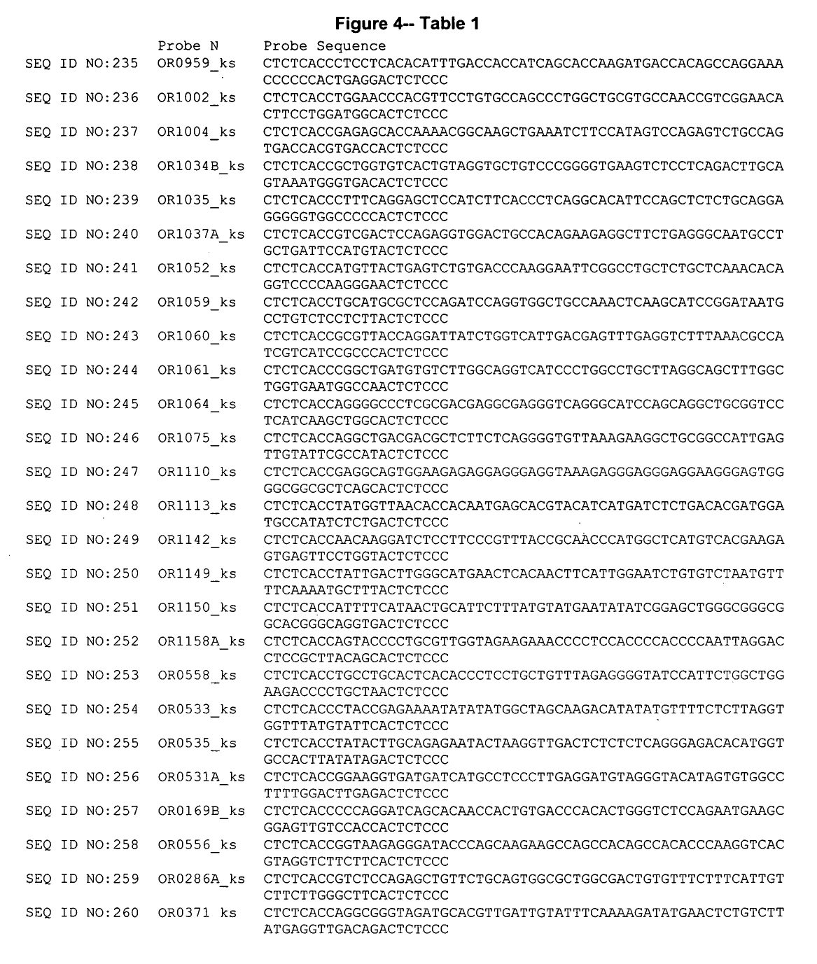

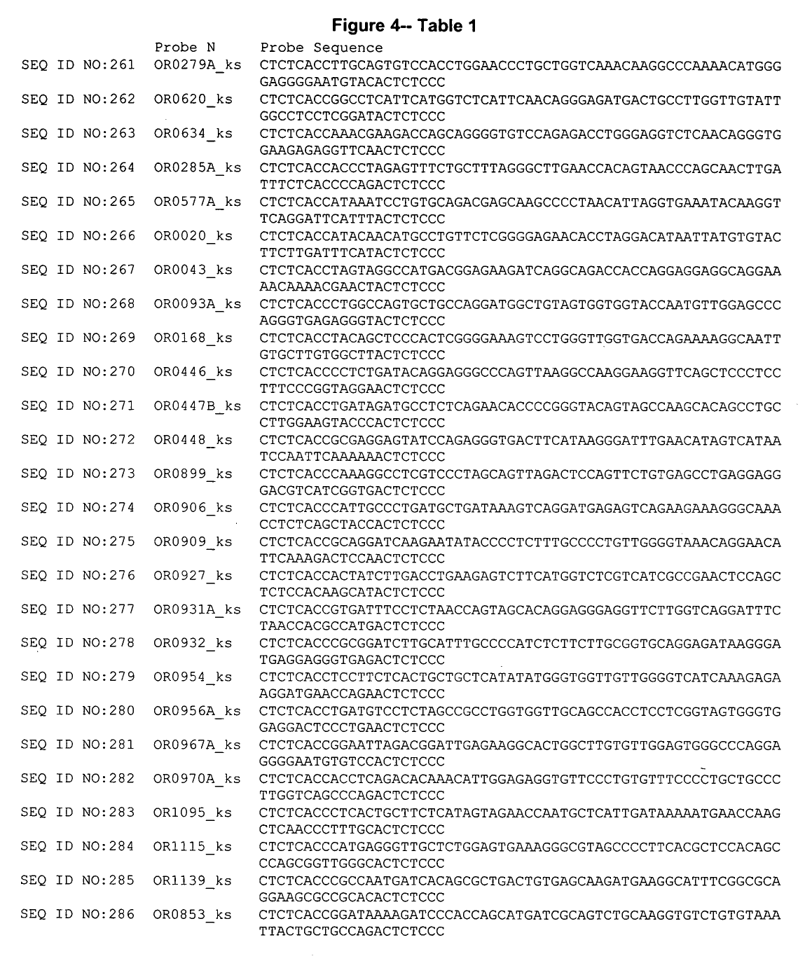

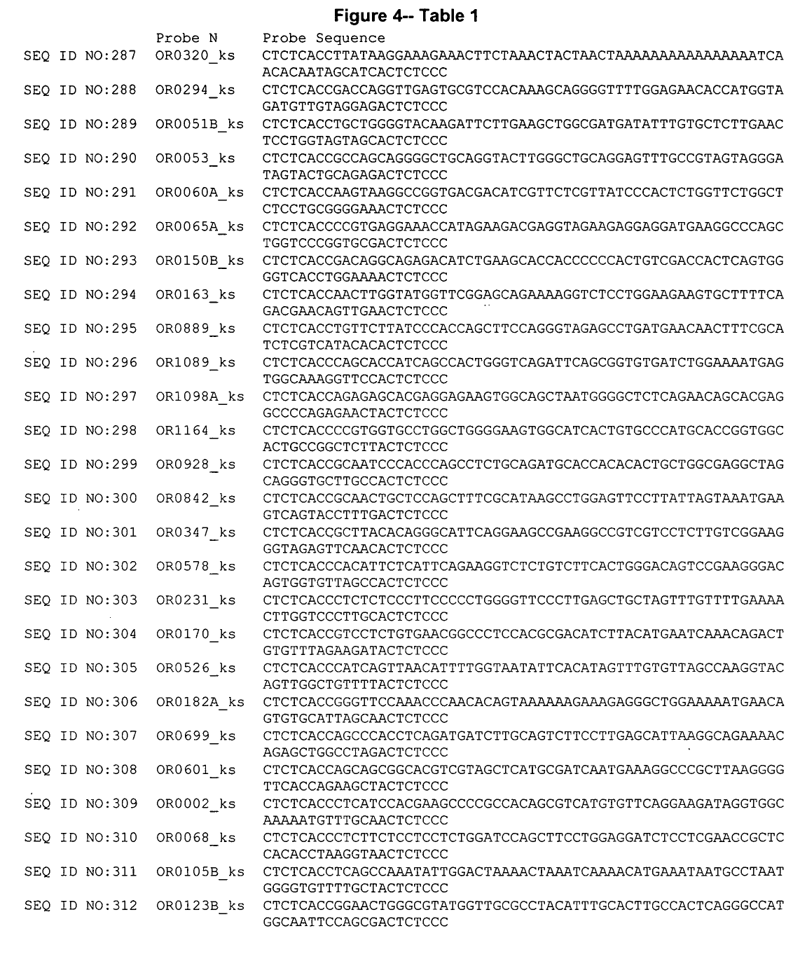

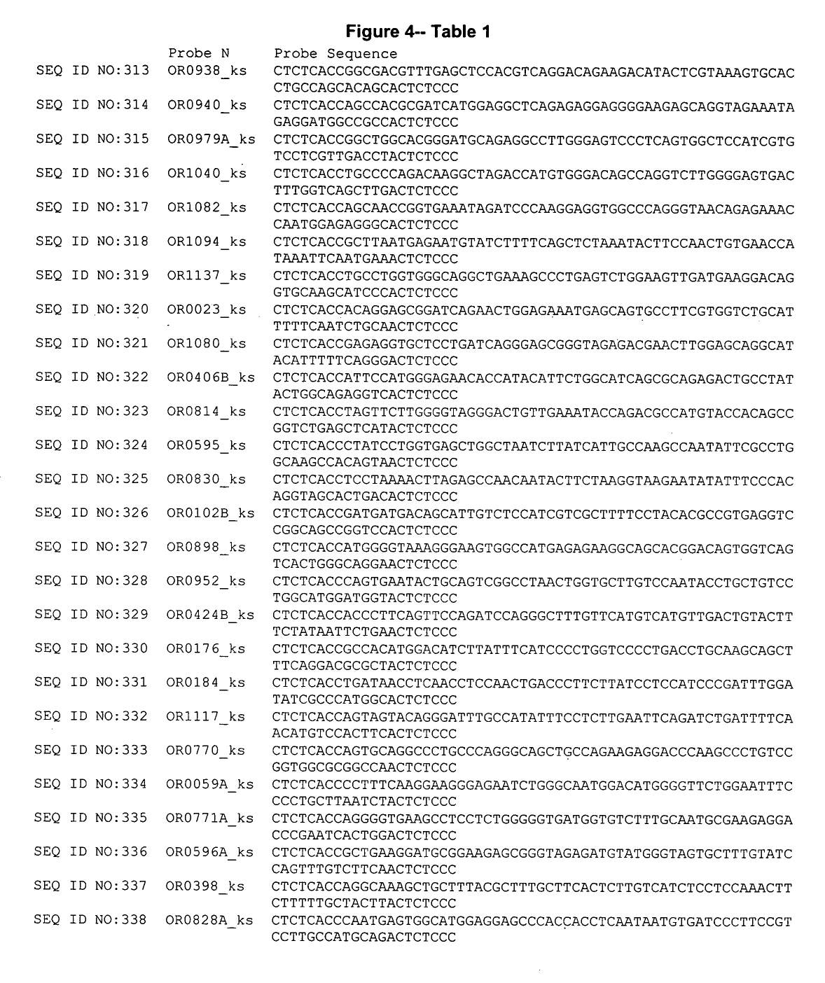

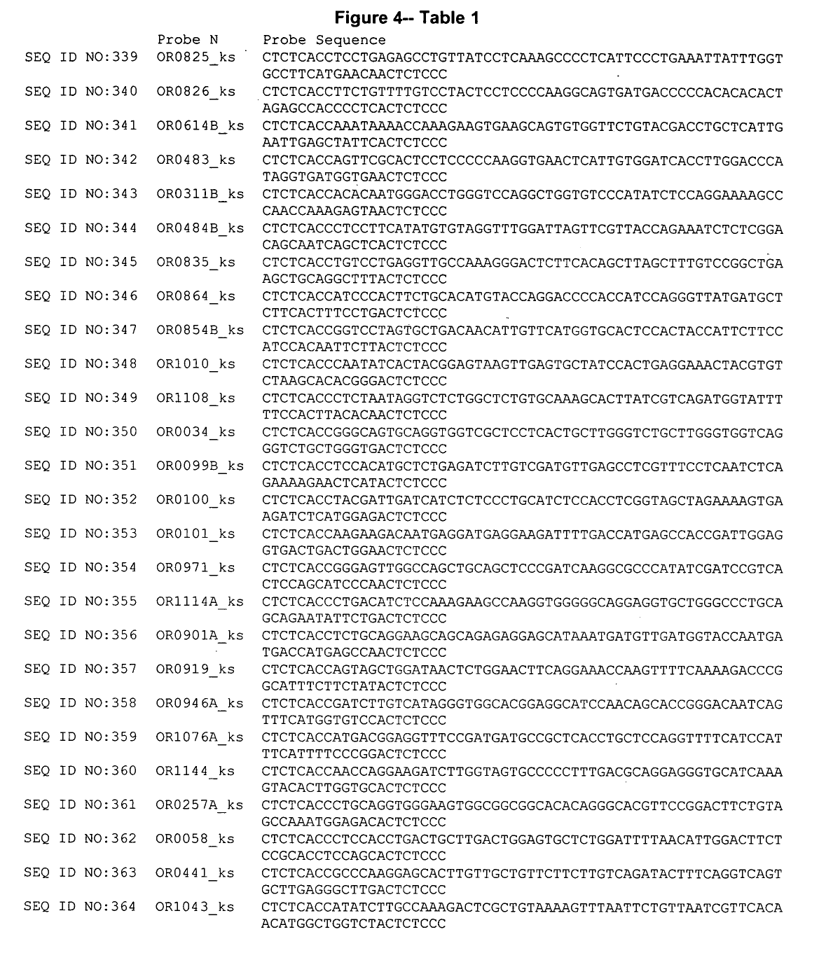

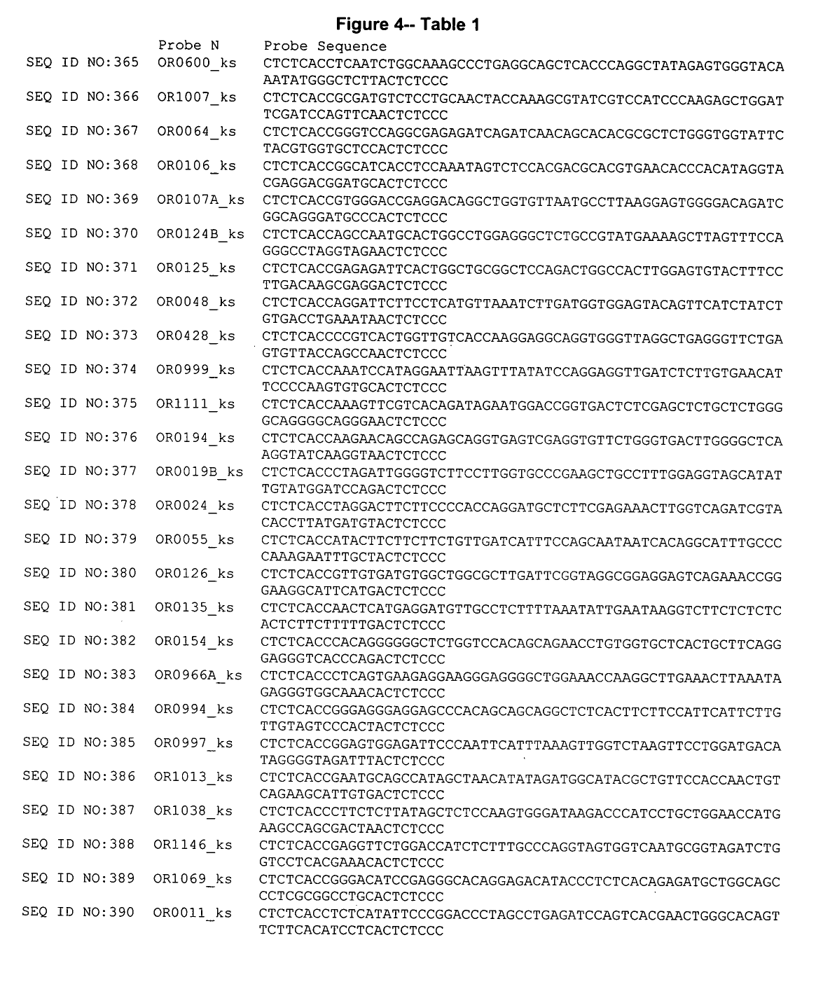

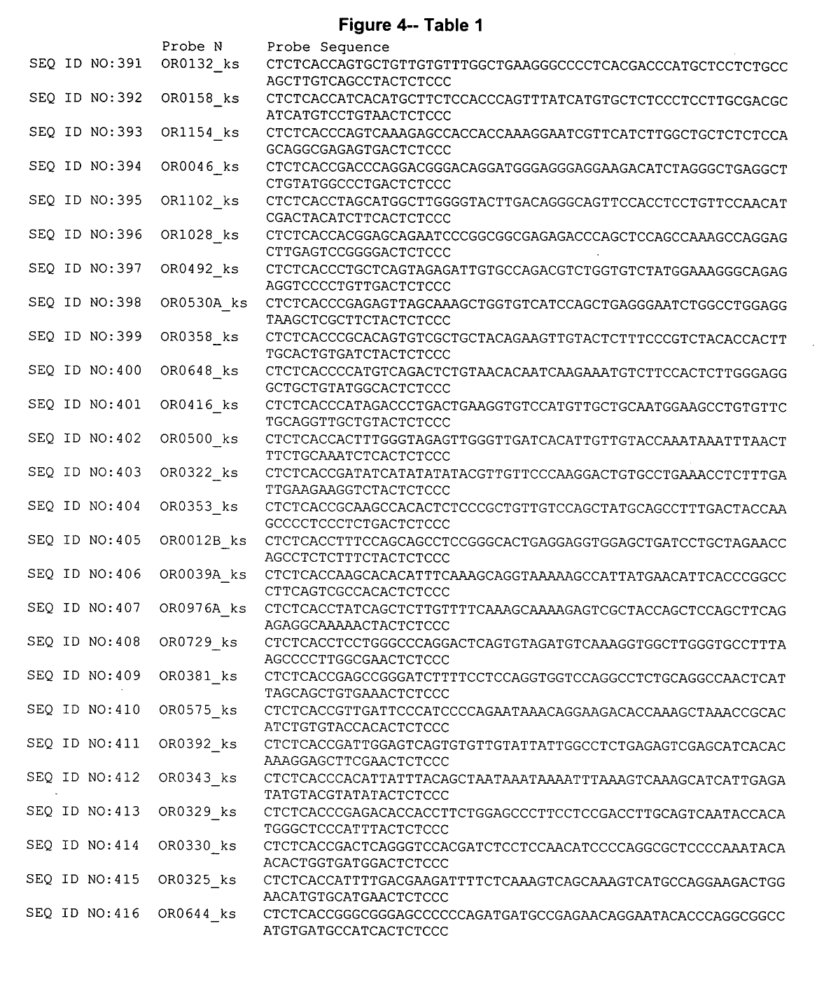

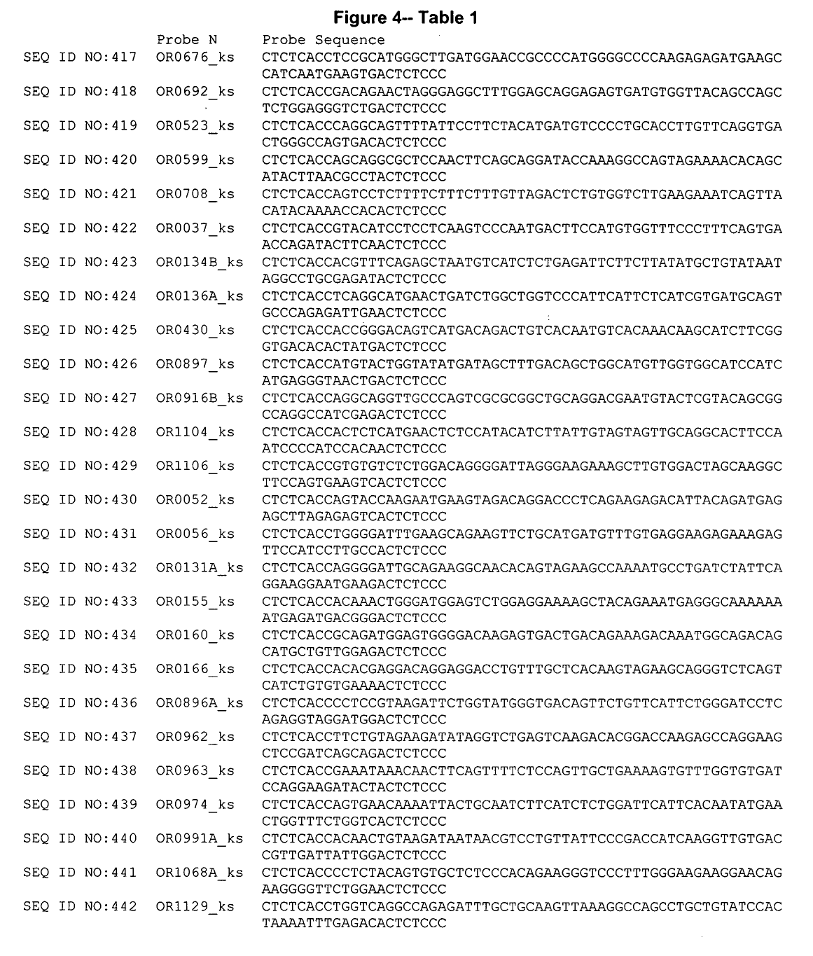

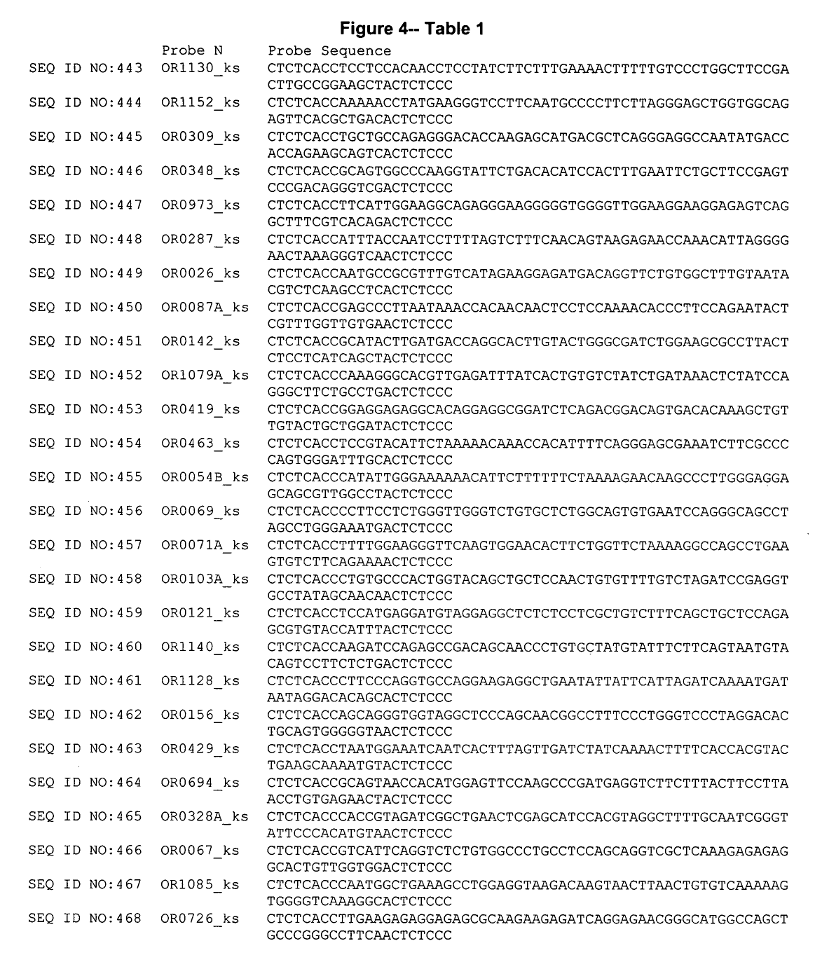

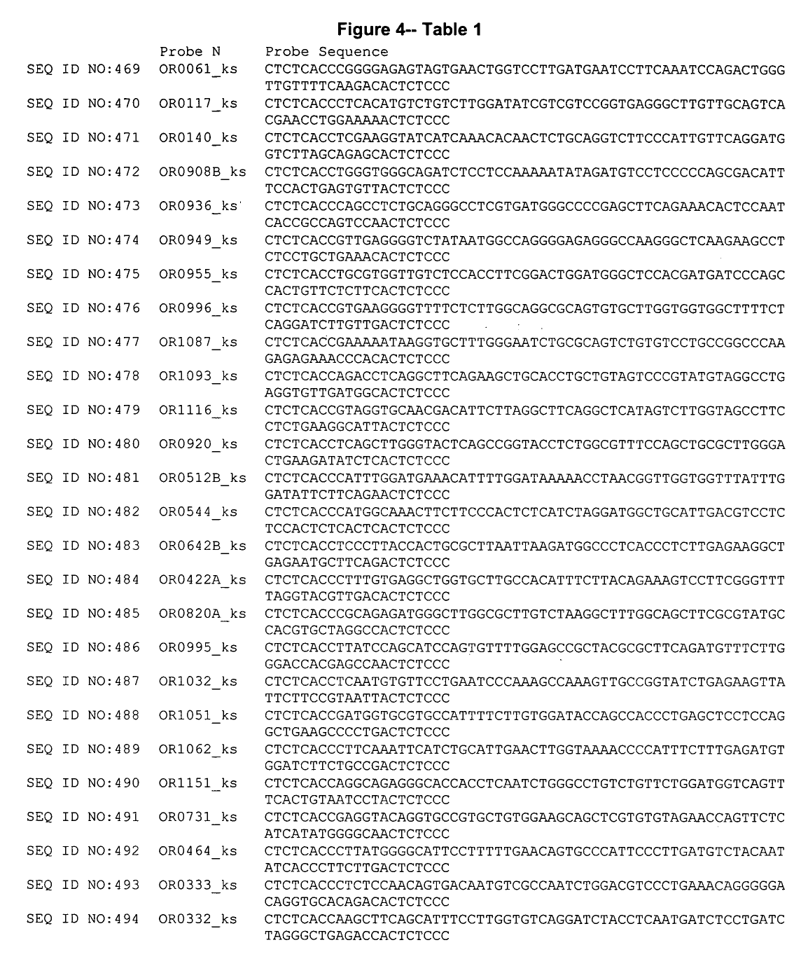

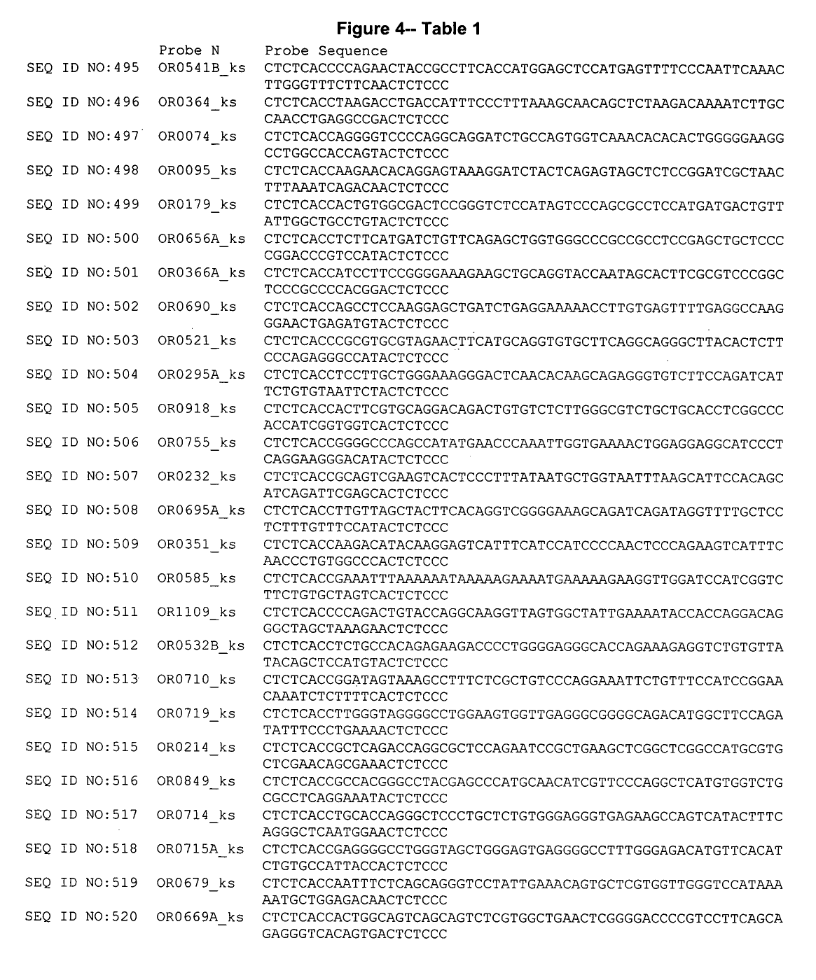

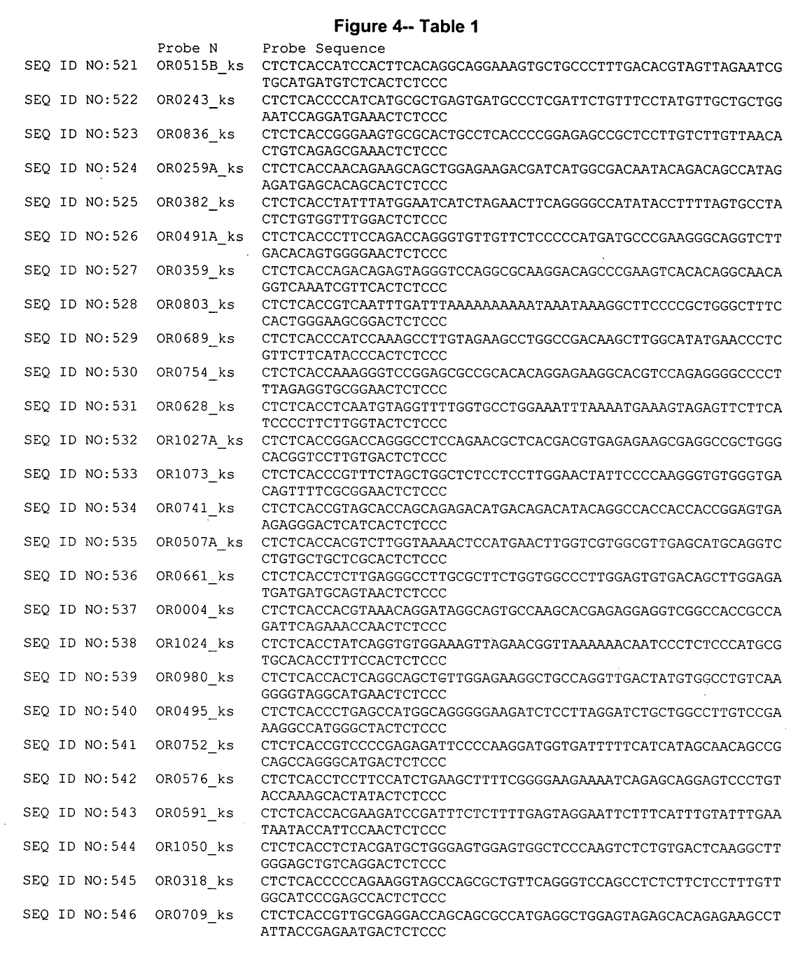

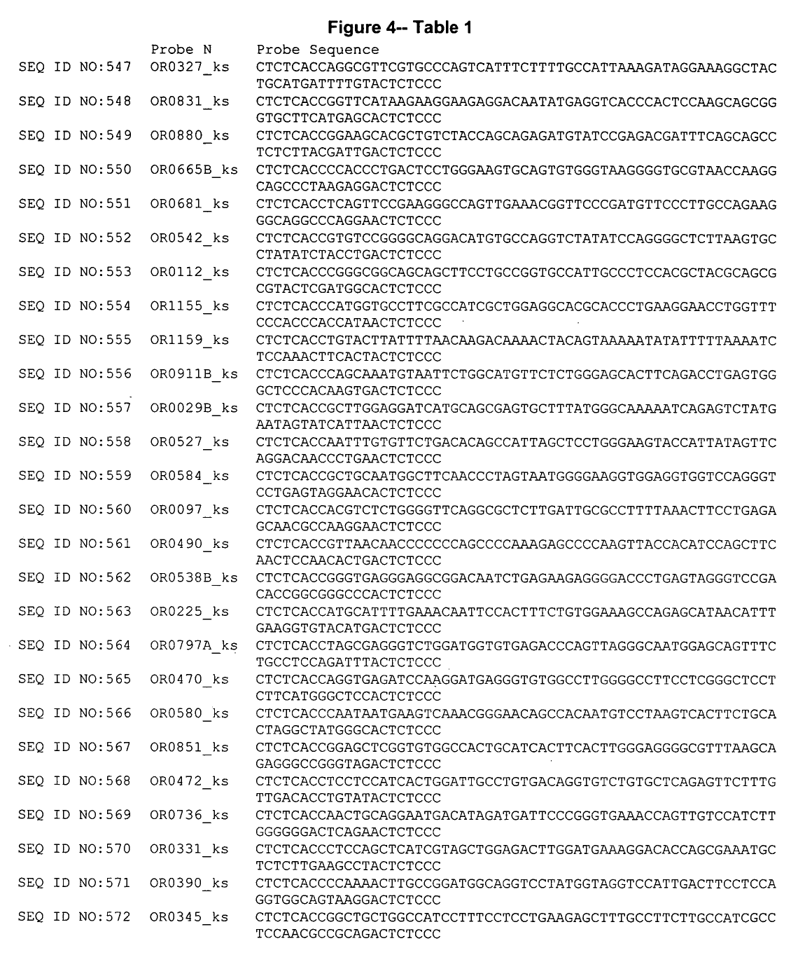

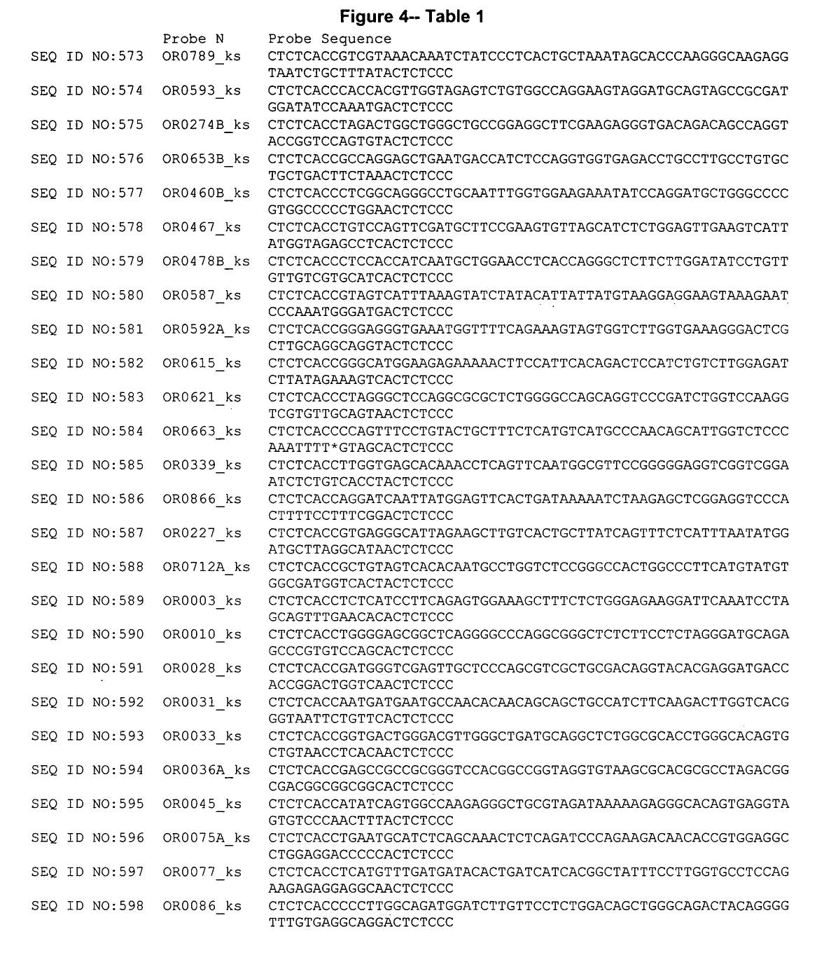

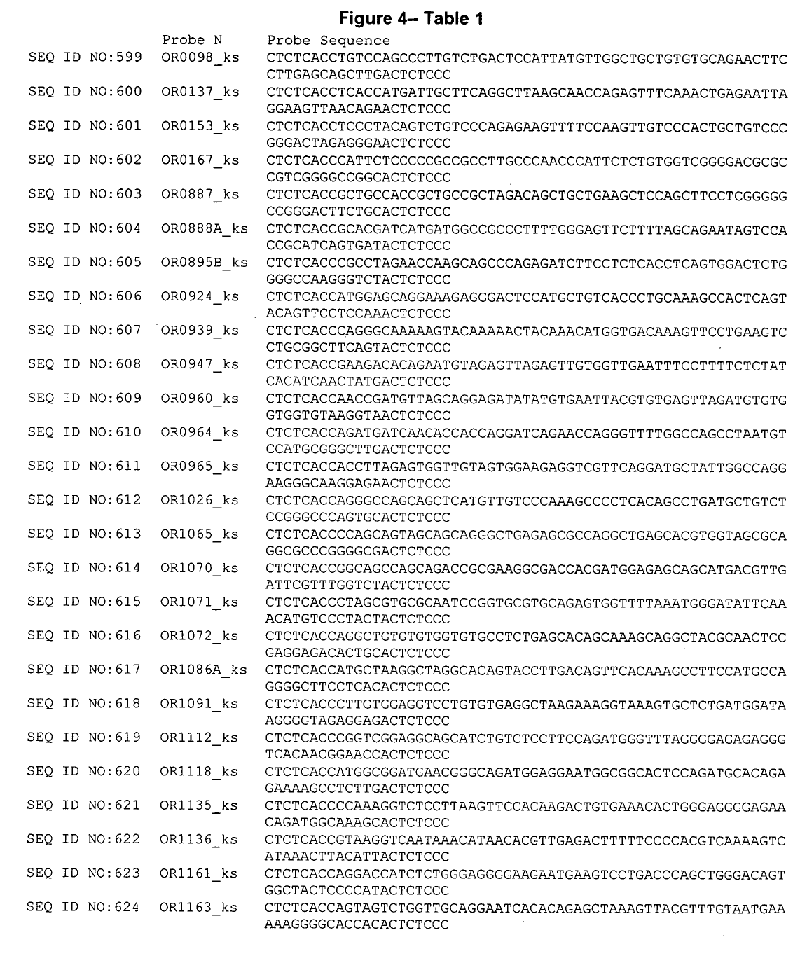

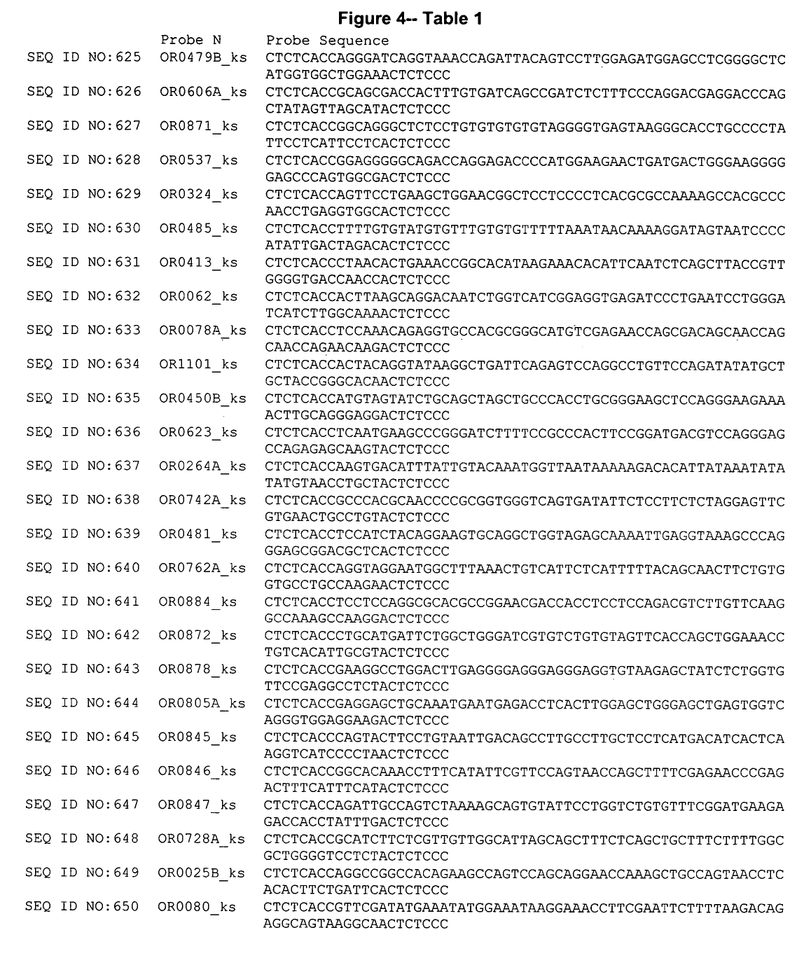

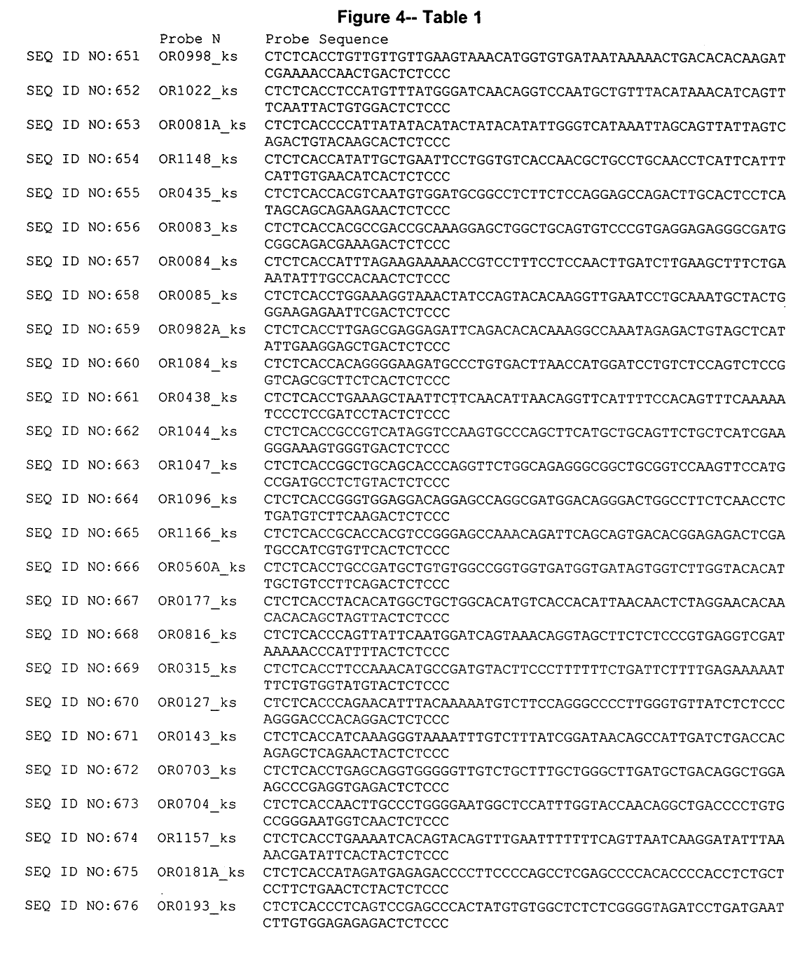

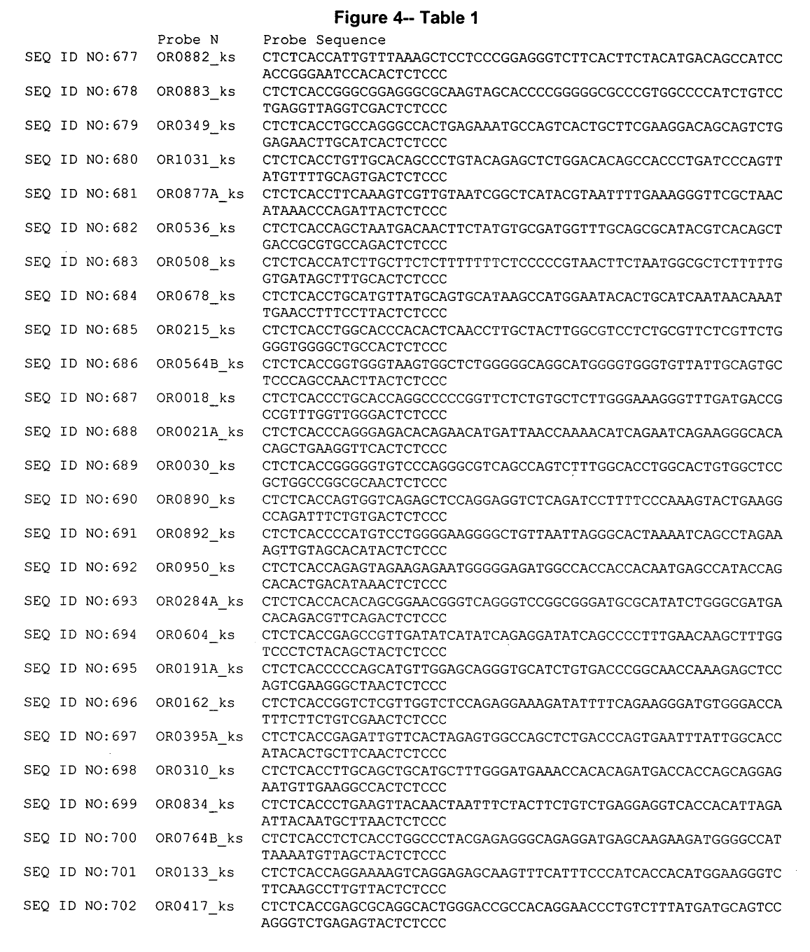

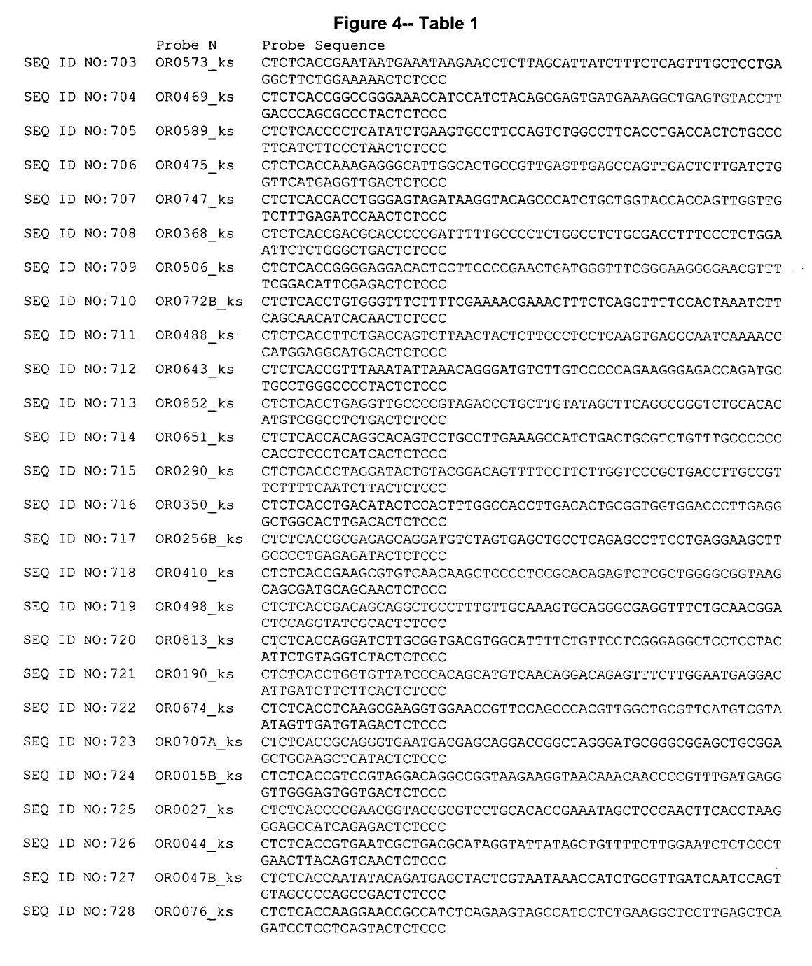

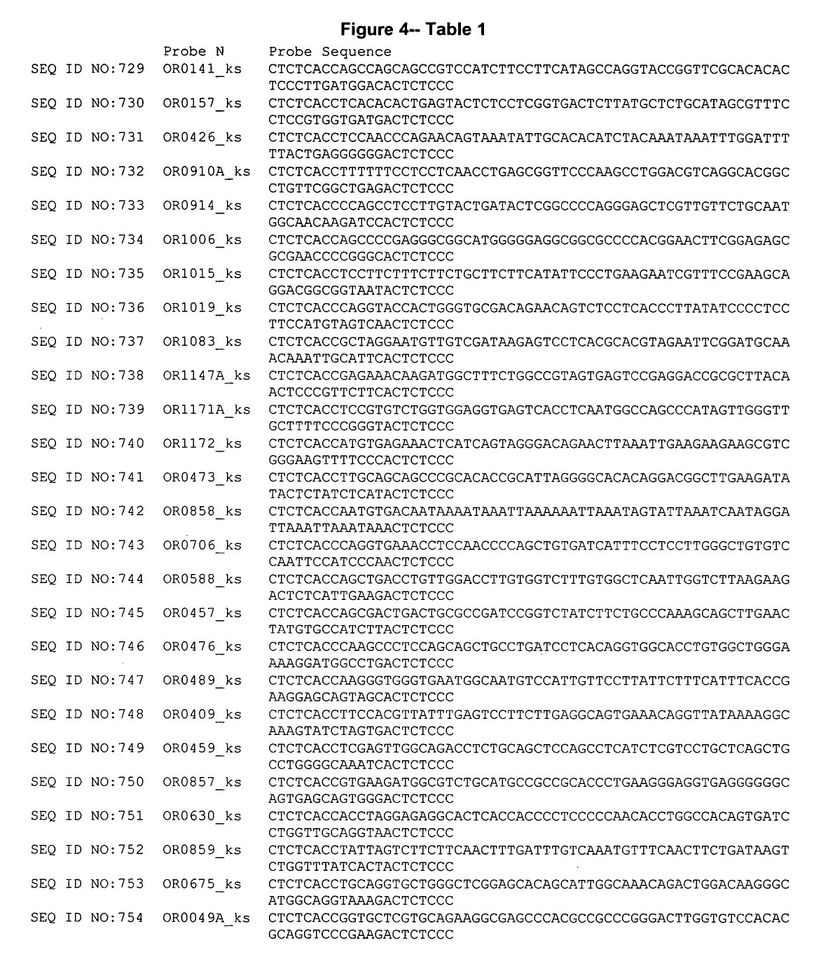

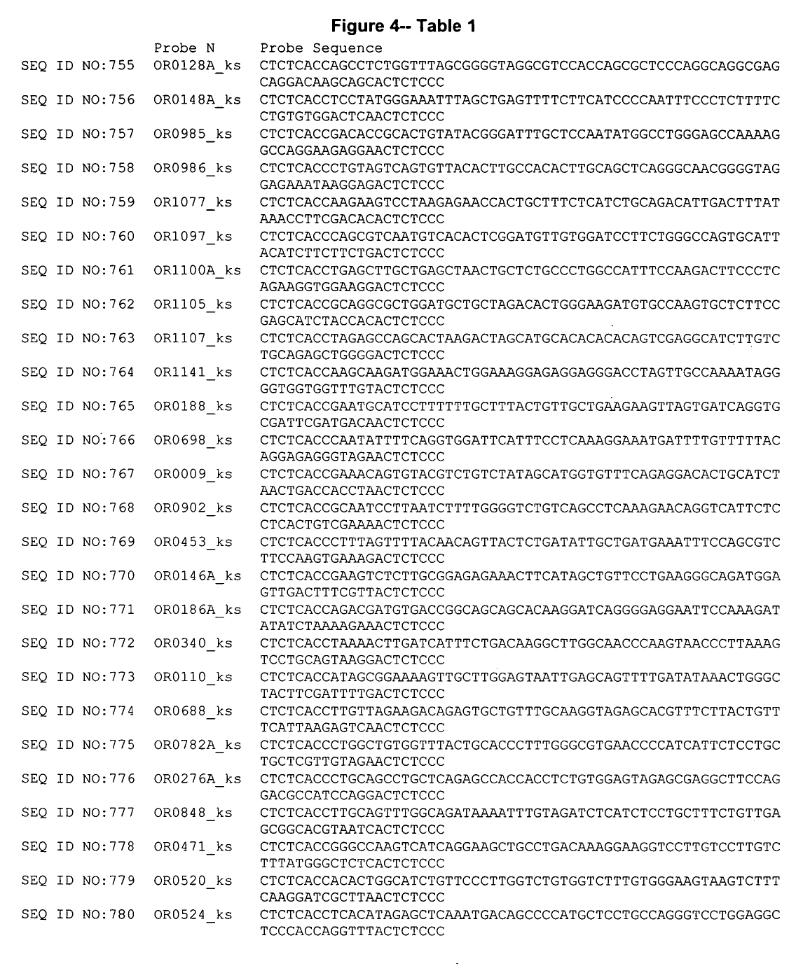

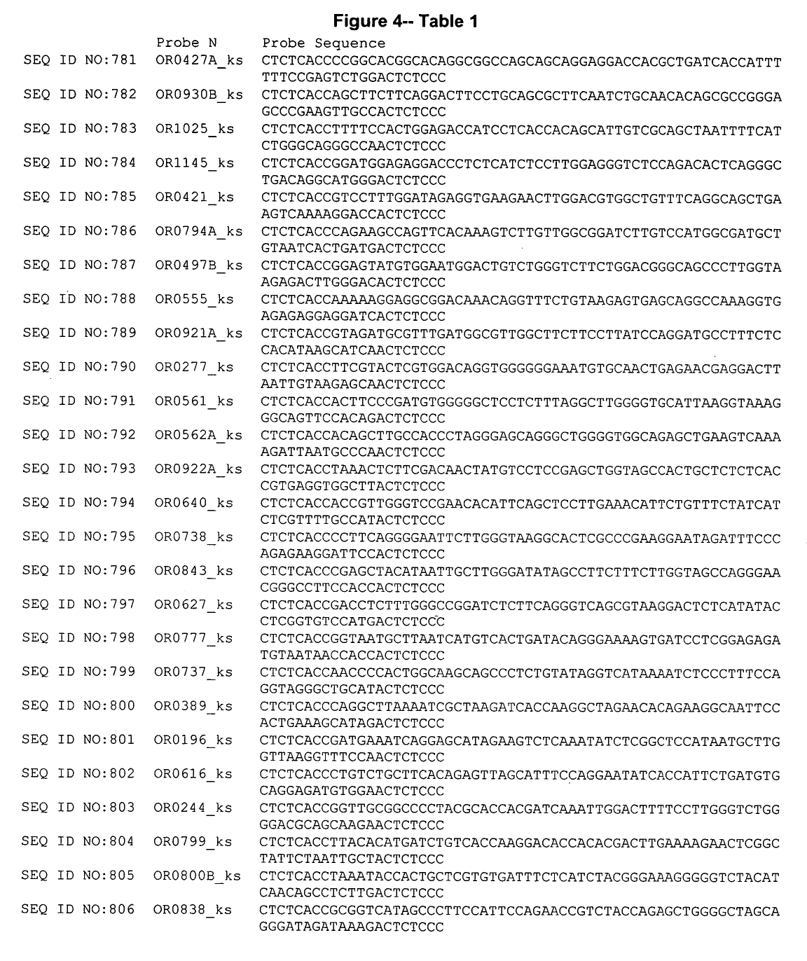

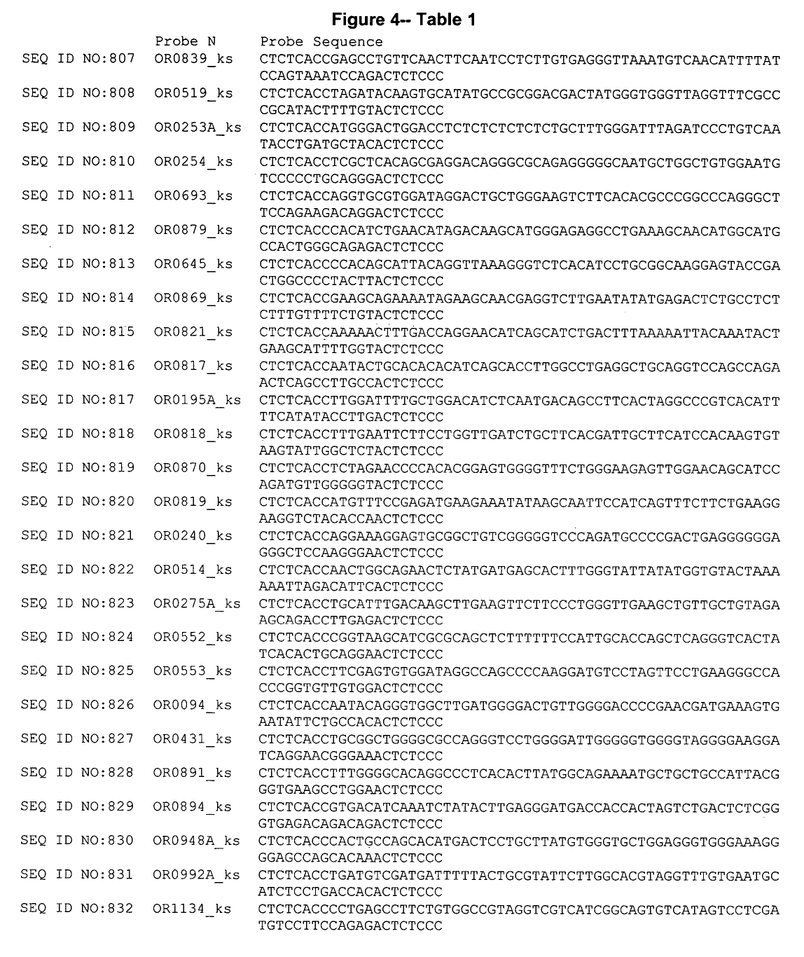

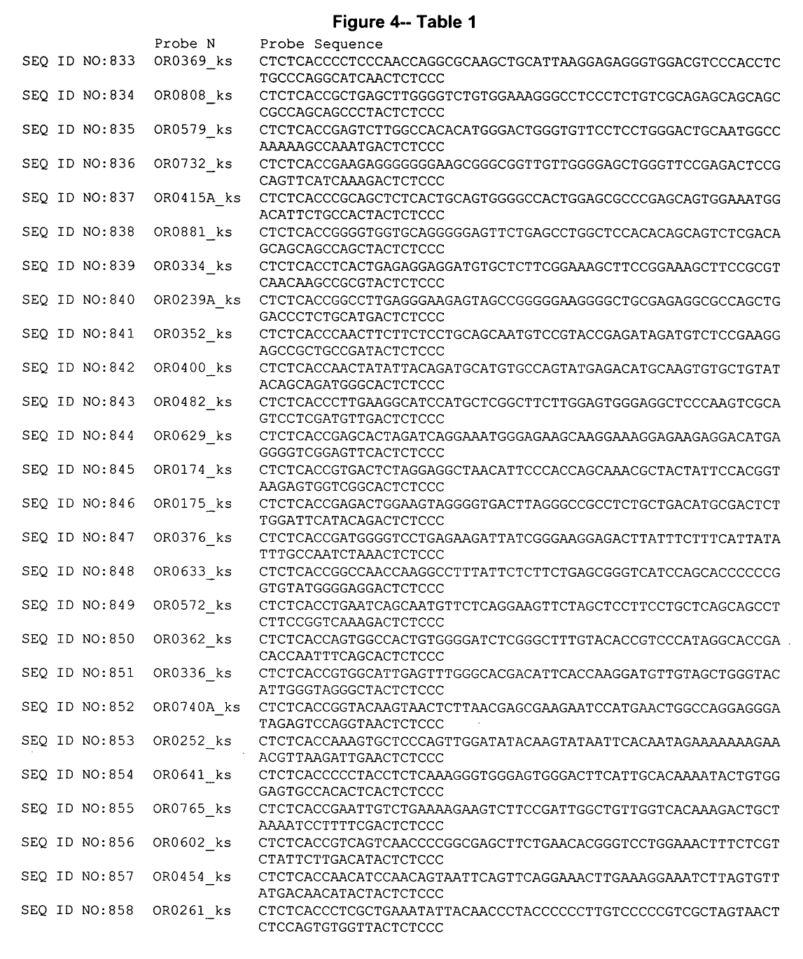

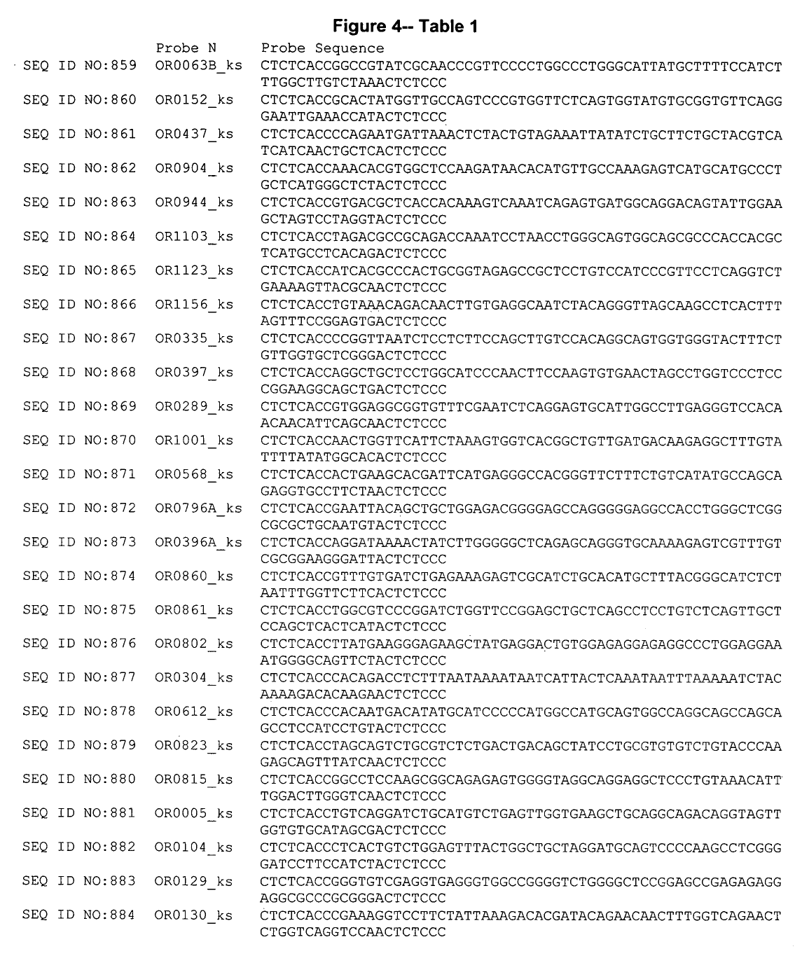

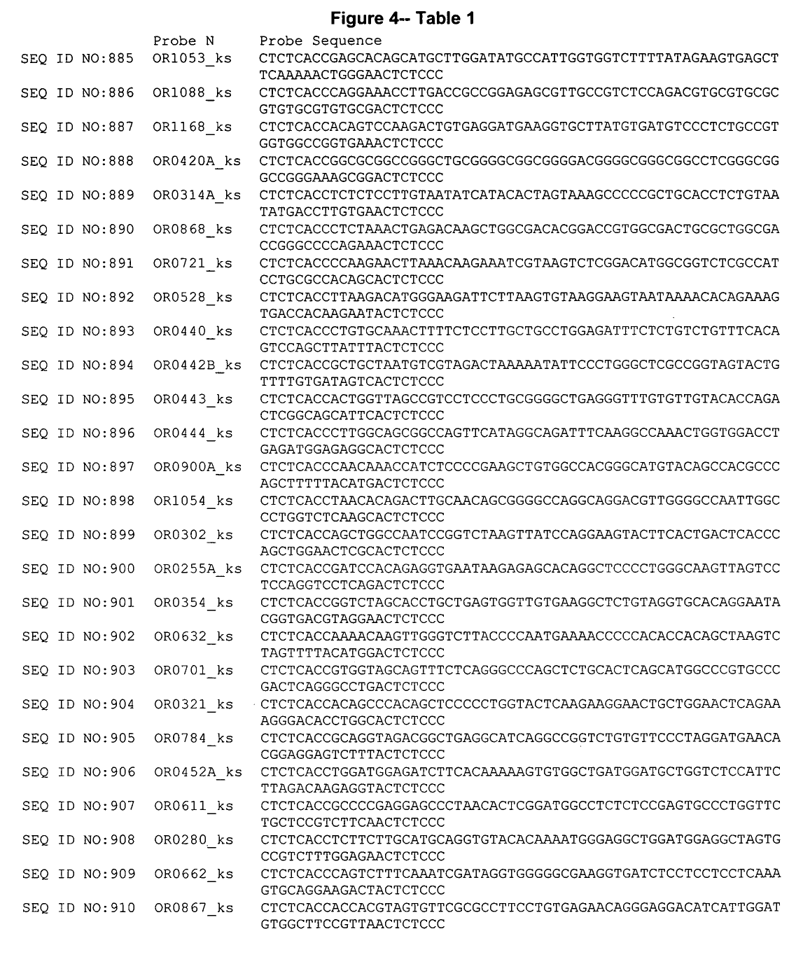

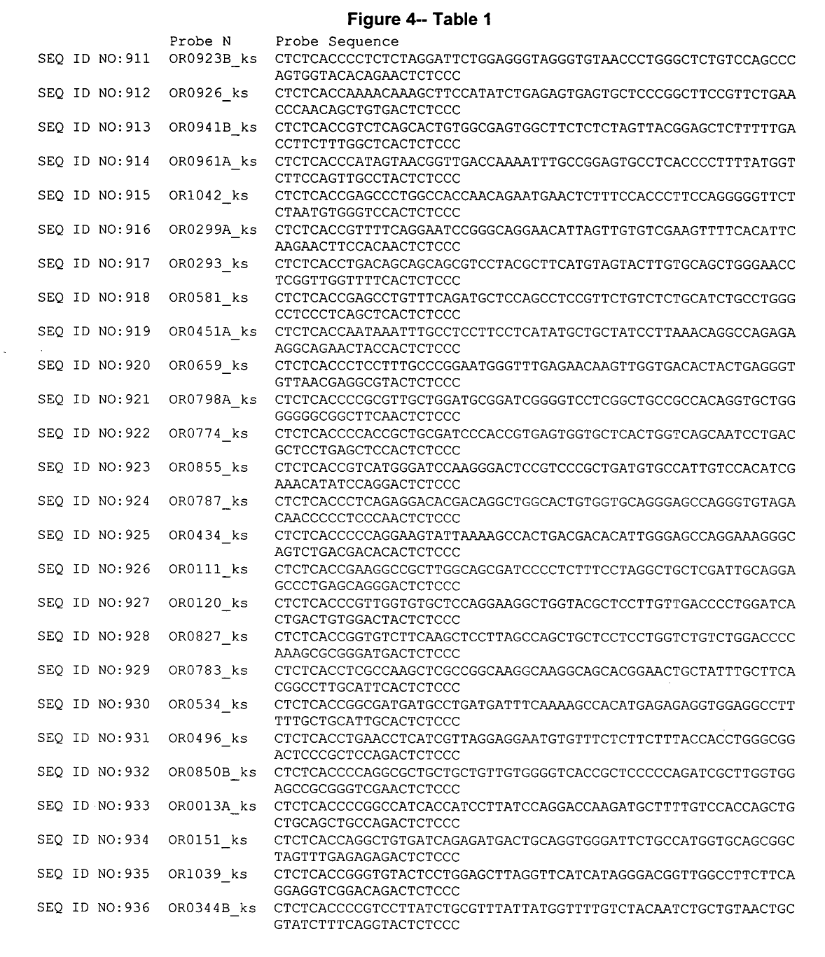

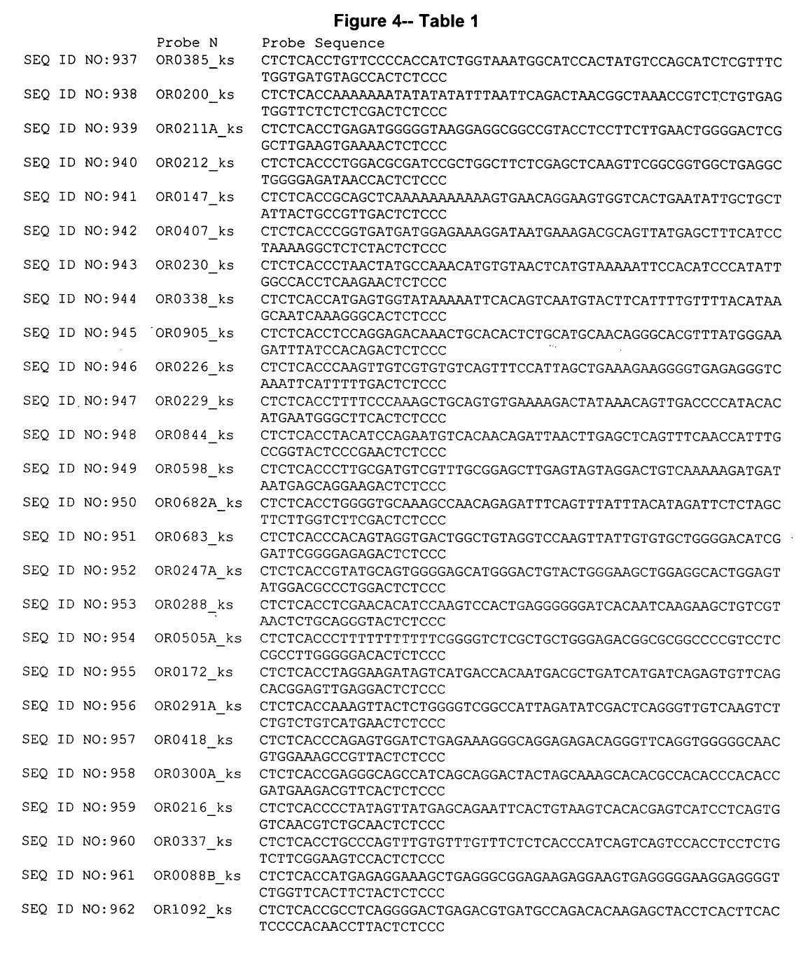

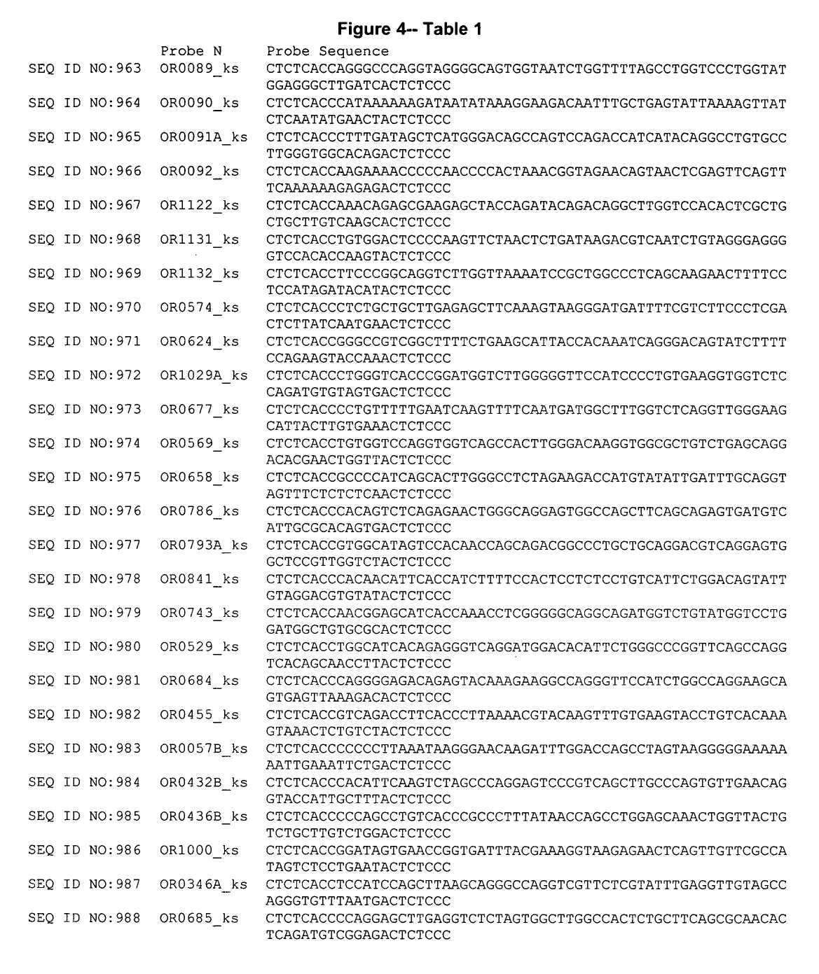

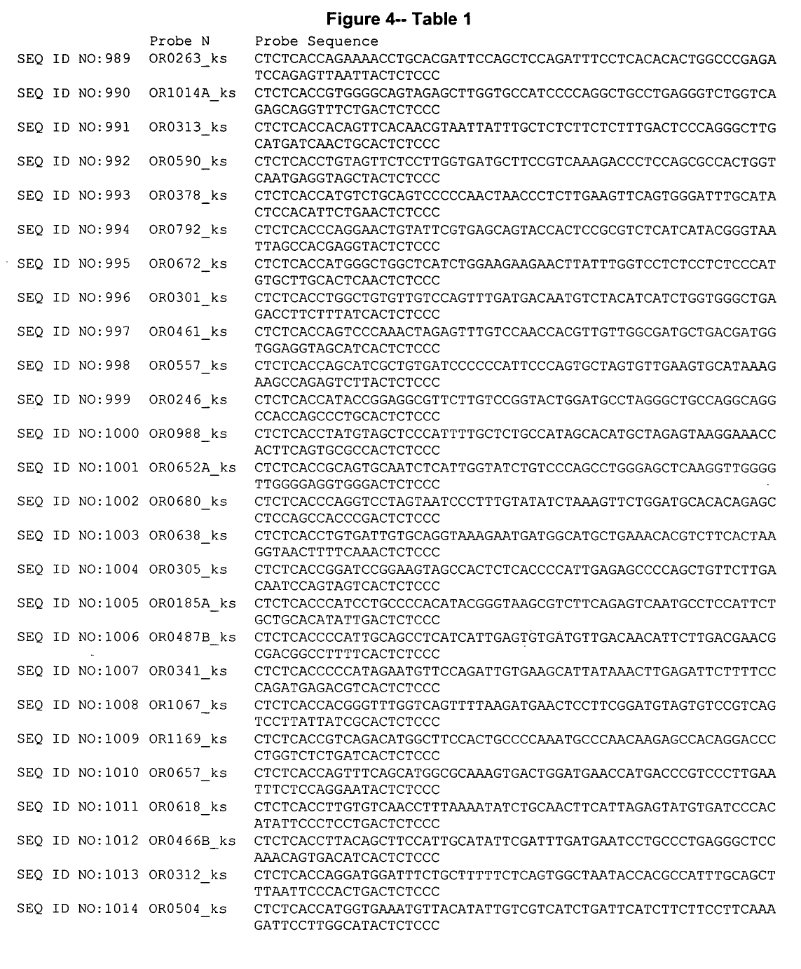

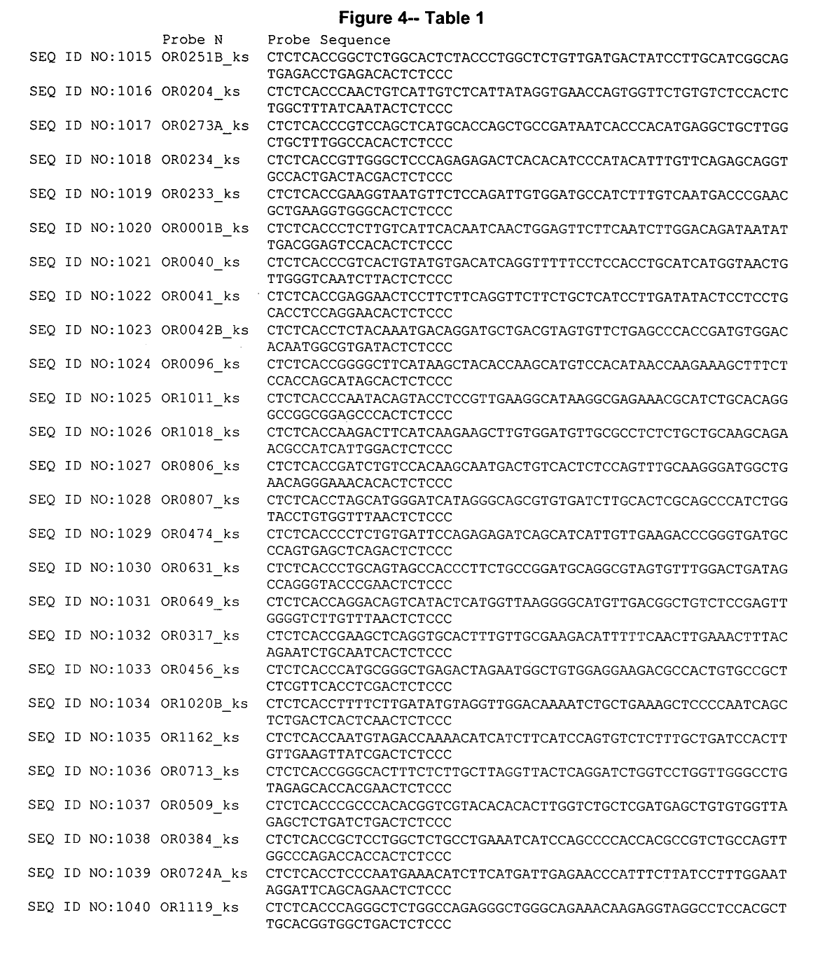

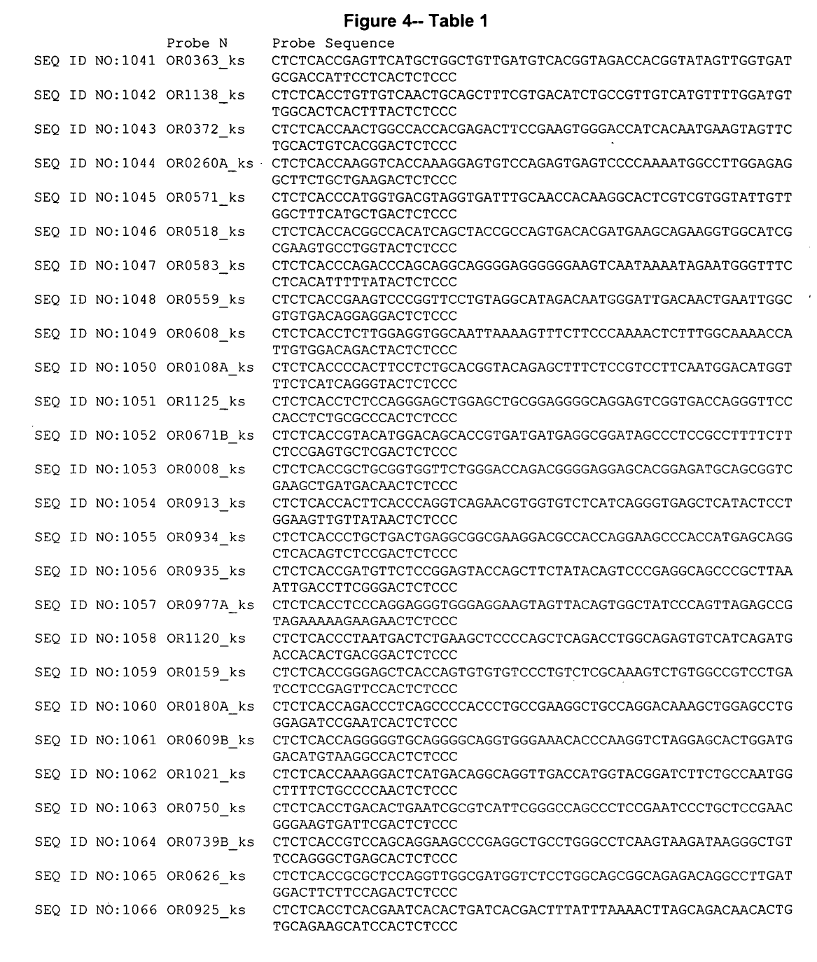

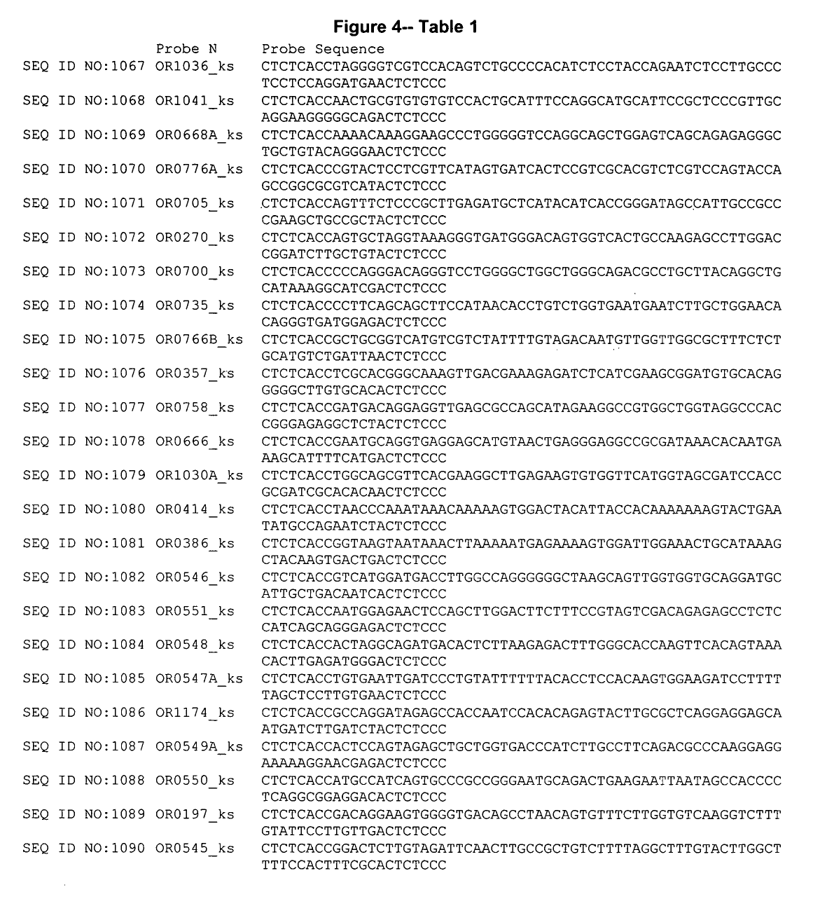

At the first step, a mixture of 1,090 rat tagged antisense oligos was hybridized with rat total liver RNA under the following conditions. The sequences of the individual rat oligos are provided in Table 1 appearing in FIG. 4. 10 μg of total liver RNA was mixed with 2 μl of 20 uM solution of rat 1,000 tagged antisense oligo set, 2 μl of 4 uM solution of Bio-r(U)30-Bio (oligo r(U)30 biotinilated at the 5′ and 3′-ends) and water to 14 μl of total volume, incubate in thermocycler at 75° C. for 3 min then cooled the mix to 50° C. Then 12.5 μl of 2×HWBU buffer (2M NaCl, 100 mM HEPES-NaOH, pH 7.5, 10 mM EDTA, 8M urea) and 1 μl of SUPERASE-IN (Ambion, Austin, Tex.) were added to the mix and continue hybridization at 50° C. overnight.

At the second step the hybridized complexes of RNA and tagged antisense oligos were purified on a streptavidin magnetic beads (Dynal, Oslo, Norway). The hybridization mix was mixed with 20 μl of streptavidin magnetic beads (Dynal, Oslo, Norway) prewashed in 1×HWB buffer and resuspended in 75 μl of 1×HWB buffer (1M NaCl, 50 mM Hepes-NaOH, pH 7.5, 5 mM EDTA). Then we continued binding reaction for 30 min in the ThermoMixer at 1200 rpm at 50° C., collected particles in MPC-S magnetic stand at the room temperature, wash the beads twice in 500 μl of 1×HWB buffer at room temperature, then ones in 500 μl of 2×SSPE and ones in 100 μl of 1×RNase H buffer (50 mM HEPES-NaOH, pH 7.5; 150 mM NaCl; 4 mM MgCl₂). The fraction of antisense oligos bound to RNA was eluted by resuspending the particles in 25 μl of RNase H buffer containing 200 units/ml RNase H and incubating at 25° C. for 30 min.

C. Generation of Labeled aRNA Target.

At the first step the fraction of tagged antisense oligos hybridized to RNA was converted to template comprising T7 promoter by PCR. PCR was conducted in 50-μl reaction, containing the following components: 25 μl of antisense oligos from stage B, 1×Advantage buffer (BD Clontech, Palo Alto, Calif.), 200 μM of each dATP, dGTP, dCTP and dTTP, 1×Advantage enzyme mix and 0.2 μM of each PCR primer T7-adc8F (5′-TMTACCACTCACTATAGGGAGACTCTCACC-3′) and Rev2-17 (5′-CGCTCGAGAGGGAGAGT-3′). PCR cycling parameters were as following: 1 cycle at 92° C. for 2 min, followed by 2 cycles at 92° C. for 30 sec, 45° C. for 15 sec and 68° C. for 15 sec, followed by 21 cycles at 92° C. for 30 sec and 68° C. for 30 sec. PCR product was analyzed by agarose gel electrophoresis and purified by Nucleospin PCR purification kit (BD Clontech, Palo Alto, Calif.) and quantitated by A260 nm UV absorbance.

At the second step the aRNA labeled by detectable label was generated using template comprising T7 promoter at the 5′end of antisense oligos. ³²P-labeled aRNA was synthesized in 25 μl reaction containing: 40 ng of template from first step, 1×MegaScript buffer (Ambion, Austin, Tex.), 500 μM of each rATP, rGTP and rCTP, 25 μCi of ³²P-rUTP (400 Ci/mmol) and 2 μl of T7 RNA polymerase (Ambion, Austin, Tex.). The T7 reaction mix was incubated at 37° C. for 30 min and purified through Nucleospin RNA II purification kit (BD Clontech, Palo Alto, Calif.).

D. Hybridization and Detection of Target Sequences on Array

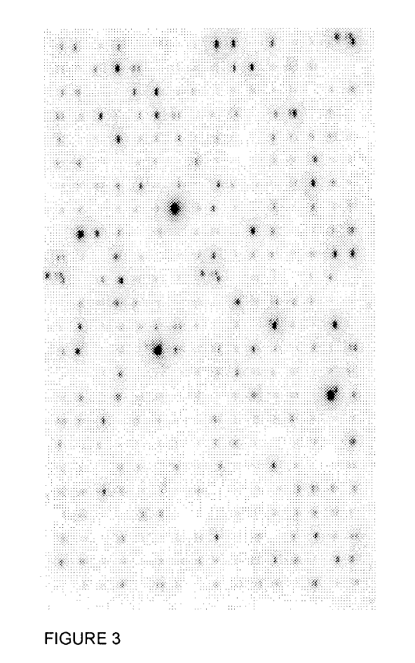

The 100 μl of ³²P-labeled probe generated at stage C was denatured at 75° C. for 3 min, mixed with 10 ml of PlasticHyb solution (BD Clontech, Palo Alto, Calif.) and hybridized overnight with Atlas Rat Plastic 4K microarray (BD Clontech, Palo Alto, Calif.) in a bottles in hybridization incubator at 60° C. and 10 rpm. After hybridization the plastic films were washed three times by 200 ml of prewarmed at 60° C. wash solution (0.1×SSC, 0.1% SDS) for 15 min each followed by rinse in 0.1×SSC. The dried microarray was exposed to phosphorimaging screen overnight and screen was scanned in Phosphorimager Storm at 50 μm resolution (Molecular Dynamics, Mountain View, Calif.). FIG. 3 demonstrates pattern of the signals corresponding expressed genes revealed by labeled aRNA target.

In an alternative to the above protocol, the aRNA could be labeled by any detectable group (for example fluorescent) using any well known in art protocol and signals detected by fluorescence, chemilumenescence, electrochemical, light scattering, surface plasma resonance, etc. approach. For differential gene expression analysis at least two aRNA targets could be labeled by the same label, hybridized with the same array type and differences in pattern of the signals will reflect the differences in the level of expression of the genes. In other case two aRNA targets could be labeled by different labels (for example ³³P and ³²P, Cy3 and Cy5, etc) and hybridized together with the same array. In this approach the ratio in normalized intensities of different labels for every dot on the array will reflect the differences in the level of expression of particular genes. In other preferred approach the direct comparisons of the amounts of expression on each array are carried out by determining the ratio of the intensity of the test aRNA target hybridization with the intensity of the control target (calibration standard) hybridization. Calibration standards (as described in published PCT application no. WO 00/65095; the priority application of which is herein incorporated by reference) could be the set of labeled aRNA corresponding starting population of tagged antisense oligonucleotides. Once determined, these ratios allow for correction of experimental variation between the arrays, and thus allow a direct comparison of the levels of gene expression in the biological samples.

### Example 2

In a specific embodiment of the present invention, the inventors have developed a research tool product, available to the public. These BD Atlas™ Antisense Oligo Mixes provide the ultimate calibration and quality control standard for microarray experiments. Each species-specific mix contains a complete antisense complement to the genes represented on Clontech BD Atlas™ Glass Microarrays at the time of manufacture, providing the most precise and reliable microarray control available. Antisense Oligo Mixes are useful both as a standard to verify lot-to lot consistency in microarray printing and for normalization of experimental data to ensure that changes in hybridization intensity reflect genuine shifts in gene expression. Because the mixes are manufactured on a very large scale, encompassing thousands of kits per batch, the user can reliably compare results from mixes shipped at different times.

High-quality array oligos exhibit two main characteristics: efficient hybridization with the intended target and minimal cross-hybridization with other sequences. When these two parameters are considered, a stronger array signal (or even a high signal to noise ratio) is not necessarily a better signal. In fact, in many cases a strong signal is the result of cross-hybridization and/or bad oligo synthesis.

Based upon many years of ordering and printing oligos, the inventors have determined that about 25% of commercially synthesized oligos are incapable of producing a strong, specific hybridization signal. This failure rate is based on a survey of oligos synthesized from four well-established vendors. Failed oligos often show an increased signal intensity, which is likely due to high non-specific cross hybridization, which may in turn result from inadvertent synthesis of a degenerate oligo.

Because of the wide variability in oligo synthesis quality, BD Biosciences Clontech has chosen to invest in antisense oligo testing. This analysis ensures that each oligo consists of the intended sequence and provides a more stringent test than hybridization with “universal” RNA mixtures. Each long oligo included on our pre-made arrays, custom arrays, or in Ready-to-Print Long Oligo sets has been antisense tested in two ways. First, Klenow enzyme is used to synthesize the antisense sequences for all oligos on a test array. These antisense oligos are labeled in groups of 200-300 and hybridized to a BD Atlas microarray containing several thousand oligos. Printed (sense) oligos that do not effectively hybridize to their antisense are resynthesized along with the corresponding antisense oligo.

Effective hybridization is defined as production of a signal intensity within 10-fold of the strongest array signal in a particular group. In addition, sense oligos printed elsewhere on the array (i.e., outside the group of 200-300 oligos being tested) that exhibit significant cross-hybridization are also resynthesized. This first test confirms that the oligo can yield a strong hybridization signal. The second test is similar to the first, except that antisense oligos are prepared synthetically. This analysis verifies the actual sequence of the oligo. The synthetic test is especially useful for identifying both formatting errors and poor oligo synthesis.

I. Introduction-Use of Antisense Oligo Mixes Because of the rigorous testing that has already been performed on Clontech oligo collection, hybridization with an Antisense Oligo Mix yields highly predictable results. In practice, adequate signal (an intensity within 10-fold of the median array signal) is typically observed for at least 98% of all printed oligos on our premade or custom arrays. As a result, Antisense Mixes provide a highly effective quality or calibration control. Antisense Oligo Mixes are available for human, mouse, and rat arrays and can be used with any BD Atlas Glass Microarray of the corresponding species, regardless of array content. Thus, they are compatible with our premade arrays, custom arrays, or arrays manufactured using BD Atlas Ready-to-Print Long Oligos. Each oligo in an Antisense Mix is tagged on the 5′ end with a primary aliphatic amino group, and can be labeled directly with Cy3, Cy5, or other monofunctional N-hydroxysuccinimide-ester-activated dye.

II. List of Components

Store Oligo Mix and Labeling Buffer at −20° C.

Store DMSO tightly capped at room temperature, in the dark.

Each BD Atlas Antisense Oligo Mix is sufficient for 5 labeling reactions.

Notes:

DMSO may be safely stored at −20° C.

Depending on the complexity of the oligo mix, one labeling reaction may yield sufficient probe for

multiple hybridizations.

50 μl BD Atlas™ Antisense Oligo Mix (50 μM)

100 μl 2× Fluorescent Labeling Buffer

500 μl DMSO

III. Additional Materials Required

Fluorescent dye The fluorescent labeling protocol is optimized for use with the following dyes:

Cy3 Mono-Reactive Dye Pack (Amersham Pharmacia Biotech #PA23001)

Cy5 Mono-Reactive Dye Pack (Amersham Pharmacia Biotech #PA25001)

Each pack of Amersham Pharmacia dye contains five vials. Each vial contains sufficient dye for four Atlas Glass labeling reactions. Please disregard instructions for use and protocol information supplied with these dyes. Complete procedures are supplied in this User Manual. Other mono-functional, N-hydroxysuccinimide-activated fluorescent dyes are also compatible.

MERmaid Spin Kit (Bio 101 #1105-600)

For Purification of Labeled Oligos

0.5 M EDTA (pH 8.0)

3 M Sodium Acetate (pH 5.3)

100% Ethanol (Avoid denatured alcohol; we recommend Spectrum #E1028)

70% Ethanol

Quartz cuvettes

UV/Vis spectrophotometer (Optional: a scanning spectrophotometer will assist you in troubleshooting probe quality.)

IV. Antisense Oligo Labeling

A. General Considerations

The optimal amount of labeled Antisense Oligo Mix to use in your hybridization depends on the complexity of the Oligo Mix and is not affected by the oligo content on the target microarray. The approximate complexity of each Oligo Mix is indicated by the product name. For example, the Human 8K Antisense Oligo Mix contains approximately 8,000 oligos. For each hybridization, you should plan to use approximately 25 pmol of labeled Oligo Mix for every 1,000 oligos in the mix. Thus, for the Human 8K Mix (provided at 50 μM), approximately 4 μl of the initial Oligo Mix are required per hybridization:

25 pmol×8=200 pmol

200 pmol=4 μl

50 μM

The procedure below is designed to label 10 μl of Oligo Mix, and is suitable for 2 hybridizations using the Human 8K Mix. For mixes of higher complexity, you may scale the procedure up, as necessary.

B. Antisense Oligo Re-suspension

Follow the steps below to prepare the Antisense Oligo Mix for labeling.

1. Transfer 10 μl Antisense Oligo Mix to a 0.5-ml tube.

2. Precipitate the Antisense Oligo Mix by adding 1 μl 3M Sodium Acetate (pH 5.3) and 27.5 μl of ice-cold 100% ethanol. Vortex gently.

3. Place tube in a −20° C. freezer for at least 1 hr.

4. Centrifuge tube at 4° C. for 20 min at 14,000 rpm.

5. Carefully pipette off supernatant, and air dry pellet briefly.

6. Wash pellet once with 70 μl of 70% ethanol. Flick tube gently to wash.

7. Centrifuge tube at 4° C. for 5 min at 14,000 rpm.

8. Carefully pipette off supernatant, and air dry pellet.

9. Add 10 μl 2× Fluorescent Labeling Buffer. Do not substitute a different buffer. Proceed directly with Section C.

C. Fluorescent Dye Coupling

Proceed with the steps below to couple fluorescent dye to the antisense oligos. IMPORTANT: Use only the DMSO provided or Atlas Glass Approved DMSO.

1. Prepare a 5 mM stock solution of fluorescent dye by adding the appropriate quantity of DMSO directly to the dye container. If you are using Amersham Pharmacia Cy3 or Cy5 reactive dye, open one pouch of dye. Each pouch contains one tube of dye residue sufficient for four labeling reactions. Add 45 μl DMSO directly to the dye vial. Vortex and briefly spin down.

2. Add 10 μl of the DMSO/dye mixture to your 10 μof oligo mix. Mix well and place the tube at room temperature in the dark or wrapped in aluminum foil. Incubate at room temperature for 30-60 min.

Note: The remaining DMSO/dye solution can be stored tightly capped at −20° C. for at least 1-2 months without noticeable degradation.

3. Add 2 μl 3M Sodium Acetate and 50 μl 100% ethanol; vortex.

4. Place tube in a −20° C. freezer for 2 hr to precipitate the labeled probe.

5. Spin tube at maximum speed in a microcentrifuge for 20 min.

6. Carefully pipette off supernatant, and wash pellet once in 70% ethanol.

7. Dissolve pellet in 100 μl Deionized H₂O.

D. Probe Purification

Follow the steps below to purify your labeled antisense probe using the MERmaid Spin Kit. Each SPIN Filter is suitable for purifying ˜1 nmol of labeled antisense oligos. For larger-scale purifications, perform the following steps in parallel, for each 1-nmol equivalent.

1. Completely resuspend GLASSFOG Bind solution, and transfer 400 μl to a SPIN Filter.

2. Add the entire 100 μl of probe and mix with rotation at room temperature for 5 min.

3. Centrifuge sample at 14,000 rpm for 30 sec.

4. Add 500 μl MERmaid Spin Ethanol Wash, and centrifuge at 14,000 rpm for 30 sec.

5. Repeat Step 4 two times.

6. Empty catch tube and centrifuge at 14,000 rpm for 1 min to dry pellet.

7. Transfer SPIN Filter to an elution catch tube.

8. Add 50 μl MERmaid Elution Solution, and resuspend GLASSFOG by flicking the tube. Centrifuge at 14,000 rpm for 30 sec.

9. Repeat elution Step 8 using 50 μl MERmaid Elution Solution.

10. Add 150 μl of H2O to the catch tube to bring the total volume to 250 μl.

E. Analysis of Probe Quality

To assess the quality of your labeled probe, analyze your entire probe using UV/Vis spectrophotometry. Ensure that your cuvettes and spectrophotometer can accommodate the small volume of labeled probe (−250 μl). The measurements described in this section assume the use of cuvettes having a 10-mm path length (such as Sigma #C1918 or #9917). For the most rigorous analysis, we recommend using a scanning instrument to read the full absorbance spectrum from 200-800 nm for both Cy3 and Cy5 probes, using elution buffer as a blank. The instrument absorbance readings for Cy3 (A550) or Cy5 (A650) probes are typically around 0.06, when starting with 500 pmol of Antisense Oligo Mix. Use the formula below to determine the optimal amount of probe to use in the hybridization (Vopt) based on the absorbance reading and complexity of the probe mixture. This optimal amount of probe assumes use of the BD Atlas Glass Hybridization Chamber, using a hybridization volume of approximately 2 ml. If you are using a different final volume, the amount of probe will need to be adjusted proportionally, so that the final oligo concentration is not altered. Further optimization may be required for other hybridization techniques.

$$Vopt ( µl ) = \frac{M \times 1.25}{A λ}$$

Where: M=Probe Complexity Multiplier

(e.g., M=8 for the Human 8K Mix; M=5 for the Mouse

5K Mix)

Aλ=instrument absorbance reading at 550 nm (Cy3) or 650 nm (Cy5).

Proceed with microarray hybridization.

### Example 3

In this example, a schematic is provided in FIG. 5 showing With each new lot of microarrays printed, several microarrays (some from the beginning, middle, and end of the printing) are hybridized using an antisense oligo calibration mixture. Following quantitation, the resulting lot-specific calibration values are averaged, such as those listed on the Clontech web site. These values can be calculated directly, or can be imported into appropriate software, such as BD AtlasImage™ Software, which will automatically calculate standardized array signals, yielding the most accurate and meaningful array comparisons. This standardization protocol is ideal for database generation, as it allows statistically significant data to be generated from microarrays printed at different times. FIG. 5 describes the importance of array calibration to generate accurate, meaningful results.

Panel A first illustrates the calculation of lot-specific Calibration Standards for a target gene. Then, two different RNA samples are analyzed for target gene expression differences using two arrays—one from each lot (Panel B). Without calibration, the target gene appears up-regulated (Raw Signal, Panel B). Our practice of gene standardization demonstrates how the lot-specific value corrects for typical printing variations across lots (Calibrated Signal, Panel B). In this case, array calibration shows an insignificant difference in gene expression.

FIG. 5 demonstrates a generalized approach to provide more accurate expression data, such as with the use of calibrated BD Atlas™ Plastic Microarrays.

Panel A. After printing each lot of BD Atlas Plastic Microarrays, sample arrays from the beginning, middle, and end of the printing run are hybridized with a mix of synthetic 33P labeled antisense oligonucleotides corresponding to all genes on the array. Then, the intensity of each hybridization signal is quantitated by phosphorimaging and averaged. Average antisense intensities are calculated for each gene, as shown above for hypothetical Gene Calibration Standards are then calculated for each array lot relative to the initial printing run. All genes in the first printed lot (Lot 1, as shown) are assigned a Calibration Standard of “1.0”.

Panel B. After normalizing arrays based on the overall signal intensities from all genes on the array, experimental intensities for Gene X can then be compared using calculations that correct for array printing variations between lots. Without this correction, gene expression comparisons are less accurate and less reliable.

The above results and discussion demonstrate that novel and improved methods of producing aRNA from an initial mRNA are provided. The subject methods provide for an improvement over prior methods of producing aRNA in that the cDNA synthesis step is not required. As such, the subject methods represent a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A standardizing control for RNA samples to be tested on non-control gene sequences on nucleic acid arrays, comprising a pool of unique tagged synthetic antisense mRNA molecules of a known concentration, wherein any two sequences are unique if their sequences differ.

2. The standardizing control of claim 1, wherein said antisense molecules are unique if their sequences differ by about 20%-100% of the nucleic acid sequence from other antisense molecules in the pool.

3. The standardizing control of claim 2 wherein wherein said antisense molecules are unique if their sequences differ by about 25%-80% of the nucleic acid sequence with the other antisense molecules in the pool.

4. The standardizing control of claim 3, wherein said antisense molecules are unique if their sequences differ by about 30%-70% of the nucleic acid sequences with the other antisense molecules in the pool.

5. The standardizing control of claim 1, wherein the number of unique tagged antisense molecules in the set of tagged mRNA antisense molecules is about 50 to 50,000.

6. The standardizing control of claim 5, wherein the number of unique tagged antisense molecules in the set of tagged mRNA antisense molecules is about 100 to 40,000.

7. The standardizing control of claim 6, wherein the number of unique tagged antisense molecules in the pool of tagged mRNA antisense molecules is about 200 to 35,000.

8. The standardizing control of claim 1, wherein the pool includes a tagged antisense nucleic acid for each non-control sequence that may be present in the sample.

9. The standardizing control of claim 1, wherein the pool includes a representative or representational number of tagged antisense molecules.

10. The standardizing control of claim 9, wherein the antisense molecules included in the pool includes a representational number of tagged antisense molecules, the total number of different tagged mRNA antisense molecules in any given set is a fraction of the total number of different or distinct mRNAs in the sample employed to generate an antisense target.

11. The standardizing control of claim 10, wherein the total number of tagged antisense molecules in the pool will not exceed about 80% of the total number of distinct mRNAs in the original sample.

12. The standardizing control of claim 11, wherein the total number of tagged antisense molecules in the pool will not exceed about 60-50% of the total number of distinct mRNAs in the original sample.

13. The standardizing control of claim 11, wherein the total number of tagged antisense molecules in the pool will not exceed about 40-20% of the total number of distinct mRNAs in the original sample.

14. The standardizing control of claim 1, wherein the non-control gene sequences on the nucleic acid array are selected from the group comprising oncogenes; genes encoding tumor suppressors; genes encoding cell cycle regulators; stress response genes; genes encoding ion channel proteins; genes encoding transport proteins; genes encoding intracellular signal transduction modulator and effector factors; apoptosis related genes; DNA synthesis/recombination/repair genes; genes encoding transcription factors; genes encoding DNA-binding proteins; genes encoding receptors, and genes encoding cell-cell communication proteins

15. The standardizing control of claim 14 wherein the genes encoding receptors are selected from the group comprising receptors for growth factors, chemokines, interleukins, interferons, hormones, neurotransmitters, cell surface antigens, and cell adhesion molecules.

16. The standardizing control of claim 14 wherein the genes encoding cell-cell communication proteins are selected from the group comprising growth factors, cytokines, chemokines, interleukins, interferons, and hormones.

17. The standardizing control of claim 1, wherein said non-control gene sequences are mammalian.

18. The standardizing control of claim 17, wherein said mammalian sequences are selected from the group comprising human, rat, mouse, and bovine.

19. The standardizing control of claim 1, wherein the pool of tagged antisense molecules have at least two different gene functional classes represented in a given set.

20. The standardizing control of claim 19, wherein the number of different functional classes of genes represented in a given set is about 2-5.

21. The standardizing control of claim 20, wherein the number of different functional classes of genes represented in a given set is about 24.

22. The standardizing control of claim 21, wherein the number of different functional classes of genes represented in a given set is about 2-3.

23. A test kit for standardizing results from nucleic acid arrays comprising in a least one container the pool of unique tagged mRNA antisense molecules in claim 1.

24. The test kit of claim 23, wherein there are different tagged antisense molecules in the pool.

25. The test kit of claim 24, wherein the number of different tagged antisense molecules in the pool is from about 20 to 40,000.

26. The test kit of claim 25, wherein the number of different tagged antisense molecules in the pool is from about 20 to 10,000.

27. The test kit of claim 26, wherein the number of different tagged antisense molecules in the pool is from about 50 to 2,000.

28. The test kit of claim 27, wherein the number of different tagged antisense molecules in the pool is from about 75 to 1,500.


SEQUENCE LISTING (1090 sequences)

SEQ ID NO: 1 - DNA, 80 bp, Rat
ctctcaccttggtcatgatcttgagctgcatgatctgctgccggatggtcatatccggcttggtgatgtccaactctccc

SEQ ID NO: 2 - DNA, 80 bp, Rat
ctctcacccaacttctttgctgtcttcctgggctttgttcagaggatcagctggctgaacacagtttgcgtcactctccc

SEQ ID NO: 3 - DNA, 80 bp, Rat
ctctcaccgcccagcacttgagtgatcaatatggcaatggtgaaagaggtcattccaaaagctgtcataaacactctccc

SEQ ID NO: 4 - DNA, 80 bp, Rat
ctctcaccgctctacatagacctgactggagcttgggtgcgatgtggaggtactggtggggatgtagtctgtactctccc

SEQ ID NO: 5 - DNA, 80 bp, Rat
ctctcacctacagcactggcgtctgccgcccgcgcttcgaggagtccttagaagacttgtgaaatttcccctactctccc

SEQ ID NO: 6 - DNA, 80 bp, Rat
ctctcacctcagggtctctattggcctgaaggaggaccaccaacatcctctggtagtaccctgaggtatcccactctccc

SEQ ID NO: 7 - DNA, 80 bp, Rat
ctctcaccgccatatggatggctttgagtgtttcccgctggggctcatccttgttccttaacttttggtaggactctccc

SEQ ID NO: 8 - DNA, 80 bp, Rat
ctctcaccttgatgggacgctccagctcaggcttcttaaagcggagccacatcatgccgatgatggccagggactctccc

SEQ ID NO: 9 - DNA, 80 bp, Rat
ctctcaccatgaggagccggctgcccgagaggagaccacggtccttccctcccccacccccctctcccctctactctccc

SEQ ID NO: 10 - DNA, 80 bp, Rat
ctctcaccgtggtttcaaatatcgaagccagaatgcatgcctgtctcagggtcaccacgccagagcccaccgactctccc

SEQ ID NO: 11 - DNA, 80 bp, Rat
ctctcacctcagtccctggcatggattataacattattttcaaaataataaaaaattaaaaattatacatatactctccc

SEQ ID NO: 12 - DNA, 80 bp, Rat
ctctcaccttccccgatgataacagtggctgtgttgggctggcccaggcgggctccaaacttggggttgctgactctccc

SEQ ID NO: 13 - DNA, 80 bp, Rat
ctctcacctcagattcttcagtgcttctatttttctagctggcttgaccagtatgttgacctgcagtcgtttactctccc

SEQ ID NO: 14 - DNA, 80 bp, Rat
ctctcaccctcatctggtggtggtaagattccaggggtgggcattcttatggctttttgaaggattccctggactctccc

SEQ ID NO: 15 - DNA, 80 bp, Rat
ctctcacccaggcccctggtctattcactctcttttgaagagagagcctccacctcctgtagcagcacatggactctccc

SEQ ID NO: 16 - DNA, 80 bp, Rat
ctctcaccatgagagcagtccaagctgccttgttctggggcaaagatttctgggcacttgatggcttcacacactctccc

SEQ ID NO: 17 - DNA, 80 bp, Rat
ctctcaccttccttgatctttgagaaacttctctttgtcttttgacatattggactaggactagaccaggagactctccc

SEQ ID NO: 18 - DNA, 80 bp, Rat
ctctcaccgagtggagaggaagtgctctagctcagaggcaggagagaagtccttgcccactcccaagcatgaactctccc

SEQ ID NO: 19 - DNA, 80 bp, Rat
ctctcaccaaaatctcgtcagggaacacagccctgtctttcgttactgtctttgaacatgataccacttgatactctccc

SEQ ID NO: 20 - DNA, 80 bp, Rat
ctctcaccgggaggcagccatgacaaaggcagtggtgttgcaggactggatggtaccggaactgtcacagccactctccc

SEQ ID NO: 21 - DNA, 80 bp, Rat
ctctcaccccttcacagagaccaccaggttgtagtacggattctttattggatccaatacctgagaaccttcactctccc

SEQ ID NO: 22 - DNA, 80 bp, Rat
ctctcaccttcttgcaaagtaaatgatcatcccaatggcgggtattaccaaggaggatgcaaagtagagtgcactctccc

SEQ ID NO: 23 - DNA, 80 bp, Rat
ctctcacctgattgtcaagcaacaaaggggtgactagaggaaagacttcactactgtaggaaggttgaagagactctccc

SEQ ID NO: 24 - DNA, 80 bp, Rat
ctctcaccgcagcacactgcatggtagcgttttctccaacagtgatatcagcattgattggggccaaaatgaactctccc

SEQ ID NO: 25 - DNA, 80 bp, Rat
ctctcacctctatcaaaaacgtttgcagtttgtaatttgcaagttcctttggtttaccaagcaaatattaagactctccc

SEQ ID NO: 26 - DNA, 80 bp, Rat
ctctcacccactttgctcctccccagaagcgcccccacatcttcagcctgtatttccatttcatacaatgagactctccc

SEQ ID NO: 27 - DNA, 80 bp, Rat
ctctcaccacccaagaactgggctggggtatgagcgaggggagctctccctttagagagtccaggggtgacaactctccc

SEQ ID NO: 28 - DNA, 80 bp, Rat
ctctcacccttcctcaggacagagggatgtgcttggtctttgttgctggccttaagttcttttacagtctctactctccc

SEQ ID NO: 29 - DNA, 80 bp, Rat
ctctcacccacaaataagctcatacagtgttcttcttggaggtagtaataattgaaaagcaccaacagaaaaactctccc

SEQ ID NO: 30 - DNA, 80 bp, Rat
ctctcaccaaaagaccaaccagcagcagcacaaagagatgaatgaacctgagacgtggaaacccgaggagtgactctccc

SEQ ID NO: 31 - DNA, 80 bp, Rat
ctctcaccgactttctagagagaaataaatcacttaagcagaaggtaggtgtaaatcacagccctaaagcctactctccc

SEQ ID NO: 32 - DNA, 80 bp, Rat
ctctcaccccacacacattaagaacttcaccggcgggtagtcattgatacagctgtgctggaaccccaaggcactctccc

SEQ ID NO: 33 - DNA, 80 bp, Rat
ctctcaccctaagaataaggctagagaatgtcgccctcgtccacgtgagcagagctggcatccagtcttgatactctccc

SEQ ID NO: 34 - DNA, 80 bp, Rat
ctctcaccatgtccctcacctggaatgtggcagccagaaatagccattgtctctccctttgctctggagtcaactctccc

SEQ ID NO: 35 - DNA, 80 bp, Rat
ctctcaccttgcctcttccggtaccttttatatatgaacacaaatagaattgccaaaccggggaggatcagcactctccc

SEQ ID NO: 36 - DNA, 80 bp, Rat
ctctcaccgcaaaagagttcccagttatgctgtcatccagcagcggcagggtctccaggcaggcggttccaaactctccc

SEQ ID NO: 37 - DNA, 80 bp, Rat
ctctcacccaagtaggttcctttgtggcacttggtgcagcagatggaattattctttggatgggcatactttactctccc

SEQ ID NO: 38 - DNA, 80 bp, Rat
ctctcaccggagtacacctgcagacacttctcctttagcacacaagtggcagtgaagttagagccacgctggactctccc

SEQ ID NO: 39 - DNA, 80 bp, Rat
ctctcaccttgtcatagtcttcaacagagacctgtgtggggttccccgggatccctgccggagtggtcctggactctccc

SEQ ID NO: 40 - DNA, 80 bp, Rat
ctctcacccccgagggatcgaaaggagacaggcacccgtcgagtgcacgtttgacccctcaggtgtttcttgactctccc

SEQ ID NO: 41 - DNA, 80 bp, Rat
ctctcaccgagcagggaggcaatgaggataagatgagcagggcctggcccgggggcgccccctgtagggcggactctccc

SEQ ID NO: 42 - DNA, 80 bp, Rat
ctctcacccacatttcgagcaaatcacgtcaattttggatgcttgtcttcatctcggacataaaactccagcactctccc

SEQ ID NO: 43 - DNA, 80 bp, Rat
ctctcaccctctactctaacttccccagtcaagtcaataaatatattaagcttgtgtccgggtcaagccacaactctccc

SEQ ID NO: 44 - DNA, 80 bp, Rat
ctctcacccagagcggctattcccaggcctcccaggatccctaaggccagggctagcgttcccagcccagacactctccc

SEQ ID NO: 45 - DNA, 80 bp, Rat
ctctcacctataccgttacattataggtgccactgtcatctcttgtcagattctttatcttcaagtctccatactctccc

SEQ ID NO: 46 - DNA, 80 bp, Rat
ctctcaccatcagagactcagagtaggcctgcatggcctccttatcctgcgagccctttttactaccactccactctccc

SEQ ID NO: 47 - DNA, 80 bp, Rat
ctctcaccgctctaacaatcactaaagatccattcacagaataatttaattcttcaaagtcctttttagtgcactctccc

SEQ ID NO: 48 - DNA, 80 bp, Rat
ctctcaccaagaacaacacggccctagataggcacacaggcaagaaatccaataaagaggcagaagaaacagactctccc

SEQ ID NO: 49 - DNA, 80 bp, Rat
ctctcacctttgctagtcccaagggtctagaccctcgtcctgatgctccgaccgtgagaaaactgtgggagtactctccc

SEQ ID NO: 50 - DNA, 80 bp, Rat
ctctcaccggagtgtgtgtggggattctgagatccaagtccttcacacattaagtctacatgatggatgctcactctccc

SEQ ID NO: 51 - DNA, 80 bp, Rat
ctctcacccagtgagtggtttgccactcctggatgttctctgtatcgaggtgaactgatgtacagcaaagcaactctccc

SEQ ID NO: 52 - DNA, 80 bp, Rat
ctctcaccggtgagcatcgtggcctggtggaggtgtcacatgaccagagttcagtttacccaggattttgtgactctccc

SEQ ID NO: 53 - DNA, 80 bp, Rat
ctctcaccctgcaggctgctgtggtcatcgatgtcgctgatgctggtggtactgatgttgtccacttctccaactctccc

SEQ ID NO: 54 - DNA, 80 bp, Rat
ctctcacctgcagcttctccgtcagctcacgtcgacggttccgacacttggcagcggccagcttgtttctctactctccc

SEQ ID NO: 55 - DNA, 80 bp, Rat
ctctcaccgtagtcgtgaagagaaagactgccagggctccgaccgtatcctgccgctgcgtaagagtcgtcgactctccc

SEQ ID NO: 56 - DNA, 80 bp, Rat
ctctcaccgaaagcgcggcccgggggcgcgaagccaccaccgccgggggccccagcagcaccagcgacgctcactctccc

SEQ ID NO: 57 - DNA, 80 bp, Rat
ctctcaccagcccgtttgtcagccgcagattgaaacctcggttgctcttcgtcgctctccacctcgatgtcaactctccc

SEQ ID NO: 58 - DNA, 80 bp, Rat
ctctcaccaagctcttgctatacttgggatggcaatacttcccaagttgccatctagtgtctttccaggtcaactctccc

SEQ ID NO: 59 - DNA, 80 bp, Rat
ctctcaccgtccttttatcagattctggaaaatcacctcaaaaaaaaaaaaaaaatcccacaaacatgaaccactctccc

SEQ ID NO: 60 - DNA, 80 bp, Rat
ctctcacccccaaagacagcagaagtaggaggttaaagaaaaaatctggacacagtgagttcagtgatctgaactctccc

SEQ ID NO: 61 - DNA, 80 bp, Rat
ctctcacccatttcagagccaagaaaggaagccaaattcggaaggcctcgaatgacatcaagattgaccctgactctccc

SEQ ID NO: 62 - DNA, 80 bp, Rat
ctctcaccgtagaatggtagtacccggctgagtggcagctgcattggtcatggttaatgtctgcaggccctgactctccc

SEQ ID NO: 63 - DNA, 80 bp, Rat
ctctcaccagtttagctttatgttgtatcacagccaccatgatgctggacctagctcactcaggagggcaagactctccc

SEQ ID NO: 64 - DNA, 80 bp, Rat
ctctcaccattcagggtggggatgatggtccctcagcttcacaaacacaccaacaatgccggttccacatttactctccc

SEQ ID NO: 65 - DNA, 80 bp, Rat
ctctcaccctgcttctgaagagaaatgcagtccagctacaaaggaggctgcggggccaaagttacatctatgactctccc

SEQ ID NO: 66 - DNA, 80 bp, Rat
ctctcacccaagtatatttagatatgttcactttctccaatttcactgtggaagcatcttcaataagaggcaactctccc

SEQ ID NO: 67 - DNA, 80 bp, Rat
ctctcacctgctgcaggctgggtggaaagccatagtccaggaataccatgtccttaagggaggccactgtcaactctccc

SEQ ID NO: 68 - DNA, 80 bp, Rat
ctctcaccgaggtgatggttgggtgggggtccggagggcttctagggtgtgcttggaatctcgaccctccagactctccc

SEQ ID NO: 69 - DNA, 80 bp, Rat
ctctcaccatgactgtaattggaccggtttctctctcctcaggttccggctgttattttggctctgtcgctgactctccc

SEQ ID NO: 70 - DNA, 80 bp, Rat
ctctcaccggcaccagctccttgaggcgtgagtagcagccgttcatgtcgtagagcagaacgttcacctgctactctccc

SEQ ID NO: 71 - DNA, 80 bp, Rat
ctctcaccggggtgcgagtccagggcgatctgcaggtccaagatataatcgatgacgtgctgcaggatttccactctccc

SEQ ID NO: 72 - DNA, 80 bp, Rat; identical to SEQ ID NO: 71

SEQ ID NO: 73 - DNA, 80 bp, Rat
ctctcaccagcactccaccaccttgccaaaggtgccttcacccaggttccccacgatctcatatcgctcttgactctccc

SEQ ID NO: 74 - DNA, 80 bp, Rat
ctctcaccaagggctccctctcccagttacagtgaagtcaaaatcattagggtccaaggaggtgtctcccacactctccc

SEQ ID NO: 75 - DNA, 80 bp, Rat
ctctcaccgagaaagcctgacacagatattcttcggcaggtgcacatccagatgtttccattgggcttggagactctccc

SEQ ID NO: 76 - DNA, 80 bp, Rat
ctctcaccctctgccagaagttccctgccatggaatactgcttggcctgatacaaaagcaccagccggcaaaactctccc

SEQ ID NO: 77 - DNA, 80 bp, Rat
ctctcaccctcaggtttggatggctctccaatgggggcaattgggttgggaaagtcaaggagacccacatctactctccc

SEQ ID NO: 78 - DNA, 80 bp, Rat
ctctcaccacaggtcaacatcccgcacgtctgtaggggtggtggcttggtccggatcttccacagacttggaactctccc

SEQ ID NO: 79 - DNA, 80 bp, Rat
ctctcaccgcaacagtttacgggcgcttgctcttctagccaaggcctaggctggttcccgctaagagcagctactctccc

SEQ ID NO: 80 - DNA, 80 bp, Rat
ctctcaccaattgggactttccgcaactgtgaggccatttgccaacagctaacgtggtgaggggctgaggttactctccc

SEQ ID NO: 81 - DNA, 80 bp, Rat
ctctcaccgggcactggtttagtcacatcctggaagaaagggtgagccagggctgctttggctgaaatccgcactctccc

SEQ ID NO: 82 - DNA, 80 bp, Rat
ctctcaccgctgtaattctagtacatgggttgaataagaacaagccttgtatgagctcaagcaggtcgtctcactctccc

SEQ ID NO: 83 - DNA, 80 bp, Rat
ctctcacccaccccaagaagagaaactgggcttgctaacagcagaggagtctcaccaccaagcaggtcaccaactctccc

SEQ ID NO: 84 - DNA, 80 bp, Rat
ctctcaccaaagtcgaaattccacttgcgctggctcgcttcttccatatctcggcagtgcttctccaagtccactctccc

SEQ ID NO: 85 - DNA, 80 bp, Rat
ctctcaccatatcacggtggccctgctcttcagctaagtctaccggcaggcggccccaggcgtcacacacatactctccc

SEQ ID NO: 86 - DNA, 80 bp, Rat
ctctcacccatggcggaaagcactcggccagactgtggtcgaaacaggcacagaattggcttcccaagagctactctccc

SEQ ID NO: 87 - DNA, 80 bp, Rat
ctctcacccaggtttgctaggggtggtctgaaccttagaattagatttgccaggagttcggcccggggtccgactctccc

SEQ ID NO: 88 - DNA, 80 bp, Rat
ctctcaccatacgtgcaaattcaccagatggcatctttactacgcagctgtactcctgttctgggattccaaactctccc

SEQ ID NO: 89 - DNA, 80 bp, Rat
ctctcaccagtcctcgccaatgctggtgtagatacgaggaagctggctggccaccggccggaagaggatacgactctccc

SEQ ID NO: 90 - DNA, 80 bp, Rat
ctctcaccatcttcatccgcagccagcaggcacaggaccacgttgtccgggtctacgttgagcagcttggctactctccc

SEQ ID NO: 91 - DNA, 80 bp, Rat
ctctcaccaagttttttaaacaaatcacttatgctgtgataaaccaaagatgagtaactgcatccaactaatactctccc

SEQ ID NO: 92 - DNA, 80 bp, Rat
ctctcacctccttcaagtccttggtggtgatctcggagctggtgtccactgccgcgtctgacatggtggggcactctccc

SEQ ID NO: 93 - DNA, 80 bp, Rat
ctctcaccttttgctccataggaaaagaccgcaccgaagatatcctcatgataacgtttctggctcttgagcactctccc

SEQ ID NO: 94 - DNA, 80 bp, Rat
ctctcacctgattaggatccaaaatgaactcaaaggtctcattccacacagggtttatatcattattgaagtactctccc

SEQ ID NO: 95 - DNA, 80 bp, Rat
ctctcaccctgagggtgtatgaaaccccgttgatgaggaaggtgacgtttggcatcatgttgagagtggcacactctccc

SEQ ID NO: 96 - DNA, 80 bp, Rat
ctctcaccaccctgagaggtgccagcctcctgtgctttgagagggtcgtgtcttactgctgaagtagaatgaactctccc

SEQ ID NO: 97 - DNA, 80 bp, Rat
ctctcacccttgttcctggcagggttctaaaatgaggatatggtgaataggaatggtactcagagtgaagctactctccc

SEQ ID NO: 98 - DNA, 80 bp, Rat
ctctcaccaatggctttggccagtaaggttttcccacagccaggcggtccatagaaaagaacacctttggaaactctccc

SEQ ID NO: 99 - DNA, 80 bp, Rat
ctctcacctagccaattcctcctctccctgaccccaagacacgtgagcaactgctaatgaaaagcagtaaacactctccc

SEQ ID NO: 100 - DNA, 80 bp, Rat
ctctcaccgggaagaaaacggatgaaggaagatggaaaatcataacagcaagtctgatgcaggactgtgaagactctccc

SEQ ID NO: 101 - DNA, 80 bp, Rat
ctctcacctacattttgagcgtttcttctcattctgtcccaacgccacataagccaggtttgtgggcgcatgactctccc

SEQ ID NO: 102 - DNA, 80 bp, Rat
ctctcacccgatgaggaaaggcagatccggcttggacagcctcagcaaccgtatcatgagtgctctgttattactctccc

SEQ ID NO: 103 - DNA, 80 bp, Rat
ctctcaccccaatgatggaccctccacacatatggttctgagaaaccaacttcacttgcaggctgacctgccactctccc

SEQ ID NO: 104 - DNA, 80 bp, Rat
ctctcaccttcatctgccaaggacagggttgatcactgtgaatggacatgaaaccagtcaaccagtctcagaactctccc

SEQ ID NO: 105 - DNA, 80 bp, Rat
ctctcacctcagctgccgagcctgagcggcagcagaagatgtgatcgtggataggggtcagcttgtcagtcaactctccc

SEQ ID NO: 106 - DNA, 80 bp, Rat
ctctcaccggaaccgccgatcgcaaagggctgtcgaattagcatccctcccatggttccatacacctggcccactctccc

SEQ ID NO: 107 - DNA, 80 bp, Rat
ctctcacctccactttcacccaaccgtctttcttcatgtggtacatgttgacaactcctccagaatagctgtactctccc

SEQ ID NO: 108 - DNA, 80 bp, Rat
ctctcacctcggtgcccagaggctccagcttctggcggtaggcctcgacctcctcgttccacttctcctggaactctccc

SEQ ID NO: 109 - DNA, 80 bp, Rat
ctctcacccctgcacaccctccgcgagaggggccagtctgctgcgcaggtcttccaggttctggtcgatcgtactctccc

SEQ ID NO: 110 - DNA, 80 bp, Rat
ctctcaccggaggctcctcagacagttgccggagagattcataaccgccacattgaaaaggccagagaaggcactctccc

SEQ ID NO: 111 - DNA, 80 bp, Rat
ctctcaccacaggaagtccgggcagtttgcttggtcttttgttgttattttggcgatgtcgatgtcattagaactctccc

SEQ ID NO: 112 - DNA, 80 bp, Rat
ctctcaccgcctggtggcccagccagatatctcggcgtatacagccatccagggcaggaacgagcggaaaccactctccc

SEQ ID NO: 113 - DNA, 80 bp, Rat
ctctcaccaaacagcaatatcttctcattcactgtctttaacatgaatgccgtgctcccttcatactttgcaactctccc

SEQ ID NO: 114 - DNA, 80 bp, Rat
ctctcaccagatcgtatgagggcccaagatagaatcttacaggcggtggggtggggtgaaatttcactgtagactctccc

SEQ ID NO: 115 - DNA, 80 bp, Rat
ctctcacctaaaaacagtctacgtacattatttacagtcaggtcagggagggaagccatcctgctcagctttactctccc

SEQ ID NO: 116 - DNA, 80 bp, Rat
ctctcacctactagagagaacactgggttacaggactacatgcagctgagagccagcgcacaagagcctttcactctccc

SEQ ID NO: 117 - DNA, 80 bp, Rat
ctctcaccgctgggaggtcatctggtataaattgtcactgctgtagggcgtcctcaggagcagggcctggctactctccc

SEQ ID NO: 118 - DNA, 80 bp, Rat
ctctcaccgctaatgttcttgaccggctccaaggatggcttgggctcagggtcattgagaagaggcagggtgactctccc

SEQ ID NO: 119 - DNA, 80 bp, Rat
ctctcaccctgaggacctggggctggagcatttgcggttgttgctgatctgtttcaggaccctgccactgtcactctccc

SEQ ID NO: 120 - DNA, 80 bp, Rat
ctctcaccgcaggacgaccgctgccacgagcaggagcagccaccggggctacagccgggacagacacactagactctccc

SEQ ID NO: 121 - DNA, 80 bp, Rat
ctctcaccaggcgccactctcggactggaggaacgaggcgttgagggcatcgtcgtagaaggtcgtttccatactctccc

SEQ ID NO: 122 - DNA, 80 bp, Rat
ctctcacctgctgagcccctggtggtcaccaaggcttatcgctgcgccagcactggcagggatggtggctacactctccc

SEQ ID NO: 123 - DNA, 80 bp, Rat
ctctcaccttctggaaaataaaacatagaataacaattaaaaattaagagtccaaccaacacgggttgttctactctccc

SEQ ID NO: 124 - DNA, 80 bp, Rat
ctctcacccatccttggactgctttatgagatcaaacaaaggtgaatcagaaagccatgcaagggattccatactctccc

SEQ ID NO: 125 - DNA, 80 bp, Rat
ctctcaccccttctatctaggcttagagtaggctggataacatgaagaaagtgacatggtgtgggaaagtggactctccc

SEQ ID NO: 126 - DNA, 80 bp, Rat
ctctcaccatcaccaggatcggacatggtgcctgtggctctgagtgtctcgatatcgctgtggatcggcgccactctccc

SEQ ID NO: 127 - DNA, 80 bp, Rat
ctctcaccgaccatttcccccaggtcatgcgtactaagtgtctttatgtacatttatacatttttaagtgctactctccc

SEQ ID NO: 128 - DNA, 80 bp, Rat
ctctcaccaagcgtagcacgcgcagaaacaggactcagaaagaacggtgctaacagagctccaggtgcaggaactctccc

SEQ ID NO: 129 - DNA, 80 bp, Rat
ctctcaccgctgtcactcatagatccagttctgcgcacagctcttgtagtccaccagctcctcaggctgaaaactctccc

SEQ ID NO: 130 - DNA, 80 bp, Rat
ctctcaccggggtatttcgaacagattgctgaggtggctgagagtcaaagggcatcatcatttttggtctcaactctccc

SEQ ID NO: 131 - DNA, 80 bp, Rat
ctctcacctgtcttctttgccaaacaagggaaggccaaccaggaaaagtacggcaggatctctaacgaacacactctccc

SEQ ID NO: 132 - DNA, 80 bp, Rat
ctctcaccgccagttgatgctctgcacatcatcgcgcagccggcagcgaagctgtagcaggtcaccagggtgactctccc

SEQ ID NO: 133 - DNA, 80 bp, Rat
ctctcaccgagcatgtccaggtgggtctcaggactggcagggagccgcaacttcatgtctgtgccggtacacactctccc

SEQ ID NO: 134 - DNA, 80 bp, Rat
ctctcacccccagcatcccccagaggaacacagtggatcacacactttgccctctgccaagcattctcccagactctccc

SEQ ID NO: 135 - DNA, 80 bp, Rat
ctctcaccagacagggatggggtcctcagggttgaactcaaaagggttgtccataaaggcagccatgatggaactctccc

SEQ ID NO: 136 - DNA, 80 bp, Rat
ctctcaccgcaggctctgctgcagccagagaccctaggctcctcctccctcgagatgagctttccgactcgaactctccc

SEQ ID NO: 137 - DNA, 80 bp, Rat
ctctcaccaaacattcacagagaaaggtggaatggagaagaatgaggaacaggcagaacttgctgagaatttactctccc

SEQ ID NO: 138 - DNA, 80 bp, Rat
ctctcaccattcacagagagatttgagggaacctcctgctgcctggagcctccagacaaatcctggatgctgactctccc

SEQ ID NO: 139 - DNA, 80 bp, Rat
ctctcaccgccattggatggatcttgaaaccgtagagcctgggcacaaactggttggccggccgcttgggccactctccc

SEQ ID NO: 140 - DNA, 80 bp, Rat
ctctcaccgcagacaagctctctcttccctggagggtctgacagaatggctgctatttgctgggtgatggtgactctccc

SEQ ID NO: 141 - DNA, 80 bp, Rat
ctctcaccctgaacaattgtaggggagatgcaactggagccatcaaacacagcatctttgagtccgaaaccaactctccc

SEQ ID NO: 142 - DNA, 80 bp, Rat
ctctcacctgttgccaacattaaggaaagcagggctgatgggctgaggcgagatggacgaagaccggccttgactctccc

SEQ ID NO: 143 - DNA, 80 bp, Rat
ctctcaccggggtctgtgccatagcggtacccagagctctggttcaggctgccgtccctctcctccgtcagtactctccc

SEQ ID NO: 144 - DNA, 80 bp, Rat
ctctcacctcatctgagctgtgaactcctcatcaaaatacctggtgtcggtctcagaggtgacctggggcttactctccc

SEQ ID NO: 145 - DNA, 80 bp, Rat
ctctcacctgggacagtcacaagagacgctagagagaaatgaccaggaacacccagggaagtttggaagcatactctccc

SEQ ID NO: 146 - DNA, 80 bp, Rat
ctctcacccactctcatcaggcgggttcagtttccgcagtttatgctgccgaatctcacgtactagtgtgtaactctccc

SEQ ID NO: 147 - DNA, 80 bp, Rat
ctctcaccatttgatctttcgcagactgcactattggcgtgaagaacagtgtggggtgctccacggaaagtcactctccc

SEQ ID NO: 148 - DNA, 80 bp, Rat
ctctcaccgagcctgtttcgtgtctactgttctagaaggcaagtcacacttattccctactaggaccataggactctccc

SEQ ID NO: 149 - DNA, 80 bp, Rat
ctctcacctgccttgccccaacccacctctctccatctatggcaatgcggcaggggacggataggcctgactactctccc

SEQ ID NO: 150 - DNA, 80 bp, Rat
ctctcacctgagctacacaaatctcatgcaatgctgctaagtcacgagacaggtcagttctagaatgttctgactctccc

SEQ ID NO: 151 - DNA, 80 bp, Rat
ctctcaccttcgcttattgagatggacacctggcttgcattctggaccacagcgccatccttcatccagtgtactctccc

SEQ ID NO: 152 - DNA, 80 bp, Rat
ctctcacctgaggctggtgttggtggcattcaatatacttgcttctaccatattggaagctacagacagataactctccc

SEQ ID NO: 153 - DNA, 80 bp, Rat
ctctcaccgccaaatagaaaagtcaccgataacgtcacgattgtgtagtaatggccagaagagagttcatagactctccc

SEQ ID NO: 154 - DNA, 80 bp, Rat
ctctcaccttcctcagaccgcatcagagcttcattggccatcgttgcttcctctttcatgtgcaggaccctcactctccc

SEQ ID NO: 155 - DNA, 80 bp, Rat
ctctcaccaactgtgcactggagtccaatttaatctgtttgccagggtccaatccaagaccccgcgctgcacactctccc

SEQ ID NO: 156 - DNA, 80 bp, Rat
ctctcaccctgcaagaatatctccttcctctgctttgctaccatccaggtctggtatagctatagaggagacactctccc

SEQ ID NO: 157 - DNA, 80 bp, Rat
ctctcacctcaattccaggcaagtacgtgtcaatcaaggcatcaagaaatttaacaatgagctcagcatagtactctccc

SEQ ID NO: 158 - DNA, 80 bp, Rat
ctctcaccagaggcggctgtgggcagggtaccagtcccggaacggctcccagctggggctccgcagtagcgaactctccc

SEQ ID NO: 159 - DNA, 80 bp, Rat
ctctcaccatcgttgtgcttcgtgatcacaaccactttctctgccaccaggtaggcagagtagaagccgacaactctccc

SEQ ID NO: 160 - DNA, 80 bp, Rat
ctctcaccagaacgaccggttctcgcccttgtagttcacctgcaccttgggcttgtcgccgtcgttcaccacactctccc

SEQ ID NO: 161 - DNA, 80 bp, Rat
ctctcaccggagttggtggtaatttcgatggccagctgaacgctgcgctgaagggcatcccgggtcctctggactctccc

SEQ ID NO: 162 - DNA, 80 bp, Rat
ctctcacctggggcacatctgtcttcaggcttcttcgaggtcagatccaggctggggcaaactgtgactgatactctccc

SEQ ID NO: 163 - DNA, 80 bp, Rat
ctctcacccatctcagttgtgtttggagaagtattccttgctgtcatccacattgggtttgatggtgtcactactctccc

SEQ ID NO: 164 - DNA, 80 bp, Rat
ctctcaccgtccgctcagggagtacaggaggccgataccgagaaagggaacgatgttttcaaggtcattcagactctccc

SEQ ID NO: 165 - DNA, 80 bp, Rat
ctctcacccccacagccagcagggcactcatgagtatccctaggtctggactttgtggtgctactttgggttactctccc

SEQ ID NO: 166 - DNA, 80 bp, Rat
ctctcaccaagactgggcagccaccacctacaggatgcatcagctccgtgatagctaccacgtcagccagggactctccc

SEQ ID NO: 167 - DNA, 80 bp, Rat
ctctcacctgagggtaccatatttgcatcgaaggtcctccaccccatcattcaccatatccaccaaggcagcactctccc

SEQ ID NO: 168 - DNA, 80 bp, Rat
ctctcacccagagctcaacatgtttccagtgggatggttcctctgctgcctccttggccctgagctcagaccactctccc

SEQ ID NO: 169 - DNA, 80 bp, Rat
ctctcaccaggtgttgggcttgcgctgtggccattccggccttctctgctgcagacaaaatgttctgggactactctccc

SEQ ID NO: 170 - DNA, 80 bp, Rat
ctctcaccgggggttgtctgcacatagctggctagaagaagtggaacgagatagaagactcttagctgtataactctccc

SEQ ID NO: 171 - DNA, 80 bp, Rat
ctctcacccgcttcaccacgcagccgttcttatcaatgagaaacttggtaaagttccatttgatggcatttcactctccc

SEQ ID NO: 172 - DNA, 80 bp, Rat
ctctcaccctgtcagttgaggccgcccctcaaacagattacagccaagagccaccggctgtatcaactcctcactctccc

SEQ ID NO: 173 - DNA, 80 bp, Rat
ctctcaccagccactttaaagcacgctgtcactagaacttcaggacggtcctgaccacgtcaacatagtggcactctccc

SEQ ID NO: 174 - DNA, 80 bp, Rat
ctctcaccgcctgggtgtgctccatgggcccgaactcagccacaatgtcatagaatgtgttttgcacatcttactctccc

SEQ ID NO: 175 - DNA, 80 bp, Rat
ctctcacctcctttgcaattttgataggaaaattccaagaagcctaatgaaacagagccaaccataagctctactctccc

SEQ ID NO: 176 - DNA, 80 bp, Rat
ctctcacctctatgtagggtaaatgaaggtgctacacccccacatgtagtgtcataaacccatgttggaagaactctccc

SEQ ID NO: 177 - DNA, 80 bp, Rat
ctctcaccatcaggatcaaaaactgcttgatttcttggacggcaaagagtcttccaggacatattgtcgcgcactctccc

SEQ ID NO: 178 - DNA, 80 bp, Rat
ctctcacctgggtatttcatgaggatcaggagcccatatctcagagttgtgctggtggtctcagttcctgcaactctccc

SEQ ID NO: 179 - DNA, 80 bp, Rat
ctctcaccaagtccacagcattccctgaggtgacattctccacaaagtccttgctgctcttcacgaggttgaactctccc

SEQ ID NO: 180 - DNA, 80 bp, Rat
ctctcaccccaaaccaaagtgggtgggcatgggggaactgttgggcccaggataccaccttgtccaggctccactctccc

SEQ ID NO: 181 - DNA, 80 bp, Rat
ctctcacctgtaagcaatggctagctgttaaaattatgagctcagacagcgcttcaaacacatttctttctcactctccc

SEQ ID NO: 182 - DNA, 80 bp, Rat
ctctcacccggccaatgatggaatgctctcctgagagtgagatcacacgatcttcaatggacacattggccaactctccc

SEQ ID NO: 183 - DNA, 80 bp, Rat
ctctcaccataaggaagggaatctgggatgtatgtgtagaatgattccgttttttgcttccttcctgaagagactctccc

SEQ ID NO: 184 - DNA, 80 bp, Rat
ctctcaccggagcggcatcatctgcttctgctgttcctgctgctgttgattctgctttcctctgctcctgctactctccc

SEQ ID NO: 185 - DNA, 80 bp, Rat
ctctcacctcaacagaatgaaatagtgtttaaggactaggctttaaagctacttgaagatatcttagaaataactctccc

SEQ ID NO: 186 - DNA, 80 bp, Rat
ctctcaccgtaagtgaggttggtttgtgtgggtttggggggaggggagagagaagctccatctctggcctcaactctccc

SEQ ID NO: 187 - DNA, 80 bp, Rat
ctctcacctgaagggggtgaagatcccacccaggtcacacagggcagagcataccatcatcccaagattcctactctccc

SEQ ID NO: 188 - DNA, 80 bp, Rat
ctctcaccttcatcacttagctcaacttcaaattcttctgacaggatctggcttcctgatgtctgcatgttaactctccc

SEQ ID NO: 189 - DNA, 80 bp, Rat
ctctcaccggacgtgcagaaacactcctcgcatgttcagctgtccagccctgttgtcttctgattccagactactctccc

SEQ ID NO: 190 - DNA, 80 bp, Rat
ctctcaccgaaaccatgatttcatatcatcaaccaaagcacaaagcagagagtgtactggaagtaggtgaggactctccc

SEQ ID NO: 191 - DNA, 80 bp, Rat
ctctcaccacctgaacctggcagcccagcctggcccttccctctcctccctgcactccagtgctttcaactgactctccc

SEQ ID NO: 192 - DNA, 80 bp, Rat
ctctcaccgtcgacgcctcttccttttctttcctcatctcattaatatctttggtgatgtcctcagtgccccactctccc

SEQ ID NO: 193 - DNA, 80 bp, Rat
ctctcaccgtccaggccacagctcctcagagcccagaataaagtccaaaccaaagacctgagccacaaggatactctccc

SEQ ID NO: 194 - DNA, 80 bp, Rat
ctctcaccggttggcactgaaatttgagatttttaccctacggtggtggcagtagccacaggtcccatcttgactctccc

SEQ ID NO: 195 - DNA, 80 bp, Rat
ctctcaccgcaggttagttcttggacagttccggagtgcgactcatcggtcctgcgagggacatgacgcaggactctccc

SEQ ID NO: 196 - DNA, 80 bp, Rat
ctctcacctgctatttgaggttattactgcagttctaatggacaacattaagccaatgttagaaattgctaaactctccc

SEQ ID NO: 197 - DNA, 80 bp, Rat
ctctcaccgctgctggcttaacctcttgccaggtgctatctcacttgacttatggcagtggcggcattccttactctccc

SEQ ID NO: 198 - DNA, 80 bp, Rat
ctctcacccacctgcactcagctccacactggccaggctgcagcgccgaggcttgtgctgacaagctggccgactctccc

SEQ ID NO: 199 - DNA, 80 bp, Rat
ctctcaccagaccacagaggtgatggcagccatgaatgccatcccagctggcatgtataagtggaggtggcgactctccc

SEQ ID NO: 200 - DNA, 80 bp, Rat
ctctcaccggaagcgtaccccaatatctagtaggtacagcaggtcactcgtgtagtccagcacgaaccaggcactctccc

SEQ ID NO: 201 - DNA, 80 bp, Rat
ctctcacccctggatggtatgcagccagctggtgaaggtcagagagcagttctgcacatcgaacgggaagttactctccc

SEQ ID NO: 202 - DNA, 80 bp, Rat
ctctcaccggtcacagtcaacatctgttgttctgtccacaccagctccaatcaaagagaagctcttgggagcactctccc

SEQ ID NO: 203 - DNA, 80 bp, Rat
ctctcacccagcaaccactcctcattcccactgtcaaagtgactttgctggtgtcgggcacgcgccatgaggactctccc

SEQ ID NO: 204 - DNA, 80 bp, Rat
ctctcaccaaaggctggggtgggggctggttgtagacaccctgcaggaagatccaggctgtgcccaccaccaactctccc

SEQ ID NO: 205 - DNA, 80 bp, Rat
ctctcaccctgctggtccaccatccaaatgtggggctcgtccacaaaggacacatactttaacgtcggcttgactctccc

SEQ ID NO: 206 - DNA, 80 bp, Rat
ctctcacccagaagcaggggcagggcagctctgagcagatcgttagcccctgtcctctggacggcttcctggactctccc

SEQ ID NO: 207 - DNA, 80 bp, Rat
ctctcaccaggagcatctcaatactgttggctgggctctccatgatggatctttggttcaggtctttgtagaactctccc

SEQ ID NO: 208 - DNA, 80 bp, Rat
ctctcaccctactgaggactcaatggcggtgtccctcacttggtaggccttctcatgcaagaaaacccacccactctccc

SEQ ID NO: 209 - DNA, 80 bp, Rat
ctctcacctgggatgagggattcaggctagcttcctggagataggtgatccgagttcaacagaatgctcagtactctccc

SEQ ID NO: 210 - DNA, 80 bp, Rat
ctctcacctcttatggctgtagagcttgtttttgttcatgaacgttaacaaaatccagtcacacaggaaggaactctccc

SEQ ID NO: 211 - DNA, 80 bp, Rat
ctctcacccttcatagacttaatcttgctgatgatgaatgtcttcatgcctaaggattttatagataacccaactctccc

SEQ ID NO: 212 - DNA, 80 bp, Rat
ctctcaccacgtcgcatagagctagggtctgttaccaccagcgattgctgttcctggaattgtccatcatccactctccc

SEQ ID NO: 213 - DNA, 80 bp, Rat
ctctcacccttgagagtgctgcggtaggtttcgtgcctggtgcctccgacattgaggatcaccctctcgttgactctccc

SEQ ID NO: 214 - DNA, 80 bp, Rat
ctctcaccaaggtggggagggcacccacatctgtgcccacctccacccagtgctttcttctctgtggccactactctccc

SEQ ID NO: 215 - DNA, 80 bp, Rat
ctctcaccagtatggaaatcatcgtatgtaccgacacctgggatgggctcatccaaaagatccagcaagtgtactctccc

SEQ ID NO: 216 - DNA, 80 bp, Rat
ctctcacctccggatggagttgctcctcctcatggagttgtttcttctcatagagttgcgcttcctcagagaactctccc

SEQ ID NO: 217 - DNA, 80 bp, Rat
ctctcacctgagggctcagacctcatggcatcttttagaccaggcaatgatcatctagagcctcttgcttgcactctccc

SEQ ID NO: 218 - DNA, 80 bp, Rat
ctctcaccagaaaaagaggtgcctatggtctgcttctgtaagaaacacaaatatcttgatataattgactatactctccc

SEQ ID NO: 219 - DNA, 80 bp, Rat
ctctcacctctagagcttttaattctggcagcactgtcctcaggtgaagcctgactagaaaacaggagaacaactctccc

SEQ ID NO: 220 - DNA, 80 bp, Rat
ctctcacccactagcgtgtttgtgaccacgctcattatgctgttgattctgtccttccgtccgtaggaggcgactctccc

SEQ ID NO: 221 - DNA, 80 bp, Rat
ctctcaccgttggtggtggagaggatgaacatggagctgtagggcggcatgggctttgggccatcctccccgactctccc

SEQ ID NO: 222 - DNA, 80 bp, Rat
ctctcaccggccaggcgacaacctattaaactataaggagggctgggtctgggtctgggggaacccaagggaactctccc

SEQ ID NO: 223 - DNA, 80 bp, Rat
ctctcaccctggcgaaagcctggatgctcaccggctggacaccacccacctcctcctcctcggtcttcaattactctccc

SEQ ID NO: 224 - DNA, 80 bp, Rat
ctctcaccgctctctggaactccgttctcactgtcctcttcctctttgcttgttagggcaacttcattttcaactctccc

SEQ ID NO: 225 - DNA, 80 bp, Rat
ctctcacctggggatatgaagaacaaattttcattgccagcgtcgacgacaaagttgatgttggtctgatccactctccc

SEQ ID NO: 226 - DNA, 80 bp, Rat
ctctcaccctcagtccattgggttcctcctcctcatcgtgctcagcctctgcttctttctcagcctcagcgcactctccc

SEQ ID NO: 227 - DNA, 80 bp, Rat
ctctcaccagaagccgaagaaacccagcaccatgaggatatagagagcctccagcttgctgtcatctcggagactctccc

SEQ ID NO: 228 - DNA, 80 bp, Rat
ctctcaccaagactacggttttagtaaagtatctacagagtgggacaggagtcgccaagggttttgtttgggactctccc

SEQ ID NO: 229 - DNA, 80 bp, Rat
ctctcaccgtattcaaagaaagaaggcagggaggcgggaacgggcgattcttgatagagagcttccaacctgactctccc

SEQ ID NO: 230 - DNA, 80 bp, Rat
ctctcaccttgatgctgcatttttttcatcatcatcatcatcatcatccacgaacatttattgagcgcctacactctccc

SEQ ID NO: 231 - DNA, 80 bp, Rat
ctctcacccgtaccgtgtggatgcacagacacacaagacgacccactatggacacacgtgtgacgcaaagatactctccc

SEQ ID NO: 232 - DNA, 80 bp, Rat
ctctcaccggtcggtggggaacttcatctgcaagtgagaggcagagggtggtggcaggggtgacctctcagtactctccc

SEQ ID NO: 233 - DNA, 80 bp, Rat
ctctcaccatcactgtttcttttgcagtcacaaactggaccagcacacggatccctgattgaagttttggtaactctccc

SEQ ID NO: 234 - DNA, 80 bp, Rat
ctctcaccgtctgggggagctgcctccgcccacctcggcccggcgtgctagcaccagatgttgacatcaaggactctccc

SEQ ID NO: 235 - DNA, 80 bp, Rat
ctctcaccctcctcacacatttgaccaccatcagcaccaagatgaccacagccaggaaaccccccactgaggactctccc

SEQ ID NO: 236 - DNA, 80 bp, Rat
ctctcacctggaacccacgttcctgtgccagccctggctgcgtgccaaccgtcggaacacttcctggatggcactctccc

SEQ ID NO: 237 - DNA, 80 bp, Rat
ctctcaccgagagcaccaaaacggcaagctgaaatcttccatagtccagagtctgccagtgaccacgtgaccactctccc

SEQ ID NO: 238 - DNA, 80 bp, Rat
ctctcaccgctggtgtcactgtaggtgctgtcccggggtgaagtctcctcagacttgcagtaaatgggtgacactctccc

SEQ ID NO: 239 - DNA, 80 bp, Rat
ctctcaccctttcaggagctccatcttcaccctcaggcacattccagctctctgcaggagggggtggcccccactctccc

SEQ ID NO: 240 - DNA, 80 bp, Rat
ctctcaccgtcgactccagaggtggactgccacagaagaggcttctgagggcaatgcctgctgattccatgtactctccc

SEQ ID NO: 241 - DNA, 80 bp, Rat
ctctcaccatgttactgagtctgtgacccaaggaattcggcctgctctgctcaaacacaggtccccaagggaactctccc

SEQ ID NO: 242 - DNA, 80 bp, Rat
ctctcacctgcatgcgctccagatccaggtggctgccaaactcaagcatccggataatgcctgtctcctcttactctccc

SEQ ID NO: 243 - DNA, 80 bp, Rat
ctctcaccgcgttaccaggattatctggtcattgacgagtttgaggtctttaaacgccatcgtcatccgcccactctccc

SEQ ID NO: 244 - DNA, 80 bp, Rat
ctctcacccggctgatgtgtcttggcaggtcatccctggcctgcttaggcagctttggctggtgaatggccaactctccc

SEQ ID NO: 245 - DNA, 80 bp, Rat
ctctcaccaggggccctcgcgacgaggcgagggtcagggcatccagcaggctgcggtcctcatcaagctggcactctccc

SEQ ID NO: 246 - DNA, 80 bp, Rat
ctctcaccaggctgacgacgctcttctcaggggtgttaaagaaggctgcggccattgagttgtattcgccatactctccc

SEQ ID NO: 247 - DNA, 80 bp, Rat
ctctcaccgaggcagtggaagagaggagggaggtaaagagggagggaggaagggagtggggcggcgctcagcactctccc

SEQ ID NO: 248 - DNA, 80 bp, Rat
ctctcacctatggttaacaccacaatgagcacgtacatcatgatctctgacacgatggatgccatatctctgactctccc

SEQ ID NO: 249 - DNA, 80 bp, Rat
ctctcaccaacaaggatctccttcccgtttaccgcaacccatggctcatgtcacgaagagtgagttcctggtactctccc

SEQ ID NO: 250 - DNA, 80 bp, Rat
ctctcacctattgacttgggcatgaactcacaacttcattggaatctgtgtctaatgttttcaaaatgctttactctccc

SEQ ID NO: 251 - DNA, 80 bp, Rat
ctctcaccattttcataactgcattctttatgtatgaatatatcggagctgggcgggcggcacgggcaggtgactctccc

SEQ ID NO: 252 - DNA, 80 bp, Rat
ctctcaccagtacccctgcgttggtagaagaaacccctccaccccaccccaattaggacctccgcttacagcactctccc

SEQ ID NO: 253 - DNA, 80 bp, Rat
ctctcacctgcctgcactcacaccctcctgctgtttagaggggtatccattctggctggaagacccctgctaactctccc

SEQ ID NO: 254 - DNA, 80 bp, Rat
ctctcaccctaccgagaaaatatatatggctagcaagacatatatgttttctcttaggtggtttatgtattcactctccc

SEQ ID NO: 255 - DNA, 80 bp, Rat
ctctcacctatacttgcagagaatactaaggttgactctctctcagggagacacatggtgccacttatatagactctccc

SEQ ID NO: 256 - DNA, 80 bp, Rat
ctctcaccggaaggtgatgatcatgcctcccttgaggatgtagggtacatagtgtggccttttggacttgagactctccc

SEQ ID NO: 257 - DNA, 80 bp, Rat
ctctcacccccaggatcagcacaaccactgtgacccacactgggtctccagaatgaagcggagttgtccaccactctccc

SEQ ID NO: 258 - DNA, 80 bp, Rat
ctctcaccggtaagagggatacccagcaagaagccagccacagccacacccaaggtcacgtaggtcttcttcactctccc

SEQ ID NO: 259 - DNA, 80 bp, Rat
ctctcaccgtctccagagctgttctgcagtggcgctggcgactgtgtttctttcattgtcttcttgggcttcactctccc

SEQ ID NO: 260 - DNA, 80 bp, Rat
ctctcaccaggcgggtagatgcacgttgattgtatttcaaaagatatgaactctgtcttatgaggttgacagactctccc

SEQ ID NO: 261 - DNA, 80 bp, Rat
ctctcaccttgcagtgtccacctggaaccctgctggtcaaacaaggcccaaaacatggggaggggaatgtacactctccc

SEQ ID NO: 262 - DNA, 80 bp, Rat
ctctcaccggcctcattcatggtctcattcaacagggagatgactgccttggttgtattggcctcctcggatactctccc

SEQ ID NO: 263 - DNA, 80 bp, Rat
ctctcaccaaacgaagaccagcaggggtgtccagagacctgggaggtctcaacagggtggaagagaggttcaactctccc

SEQ ID NO: 264 - DNA, 80 bp, Rat
ctctcaccaccctagagtttctgctttagggcttgaaccacagtaacccagcaacttgatttctcaccccagactctccc

SEQ ID NO: 265 - DNA, 80 bp, Rat
ctctcaccataaatcctgtgcagacgagcaagcccctaacattaggtgaaatacaaggttcaggattcatttactctccc

SEQ ID NO: 266 - DNA, 80 bp, Rat
ctctcaccatacaacatgcctgttctcggggagaacacctaggacataattatgtgtacttcttgatttcatactctccc

SEQ ID NO: 267 - DNA, 80 bp, Rat
ctctcacctagtaggccatgacggagaagatcaggcagaccaccaggaggaggcaggaaaacaaaacgaactactctccc

SEQ ID NO: 268 - DNA, 80 bp, Rat
ctctcaccctggccagtgctgccaggatggctgtagtggtggtaccaatgttggagcccagggtgagagggtactctccc

SEQ ID NO: 269 - DNA, 80 bp, Rat
ctctcacctacagctcccactcggggaaagtcctgggttggtgaccagaaaaggcaattgtgcttgtggcttactctccc

SEQ ID NO: 270 - DNA, 80 bp, Rat
ctctcacccctctgatacaggagggcccagttaaggccaaggaaggttcagctccctcctttcccggtaggaactctccc

SEQ ID NO: 271 - DNA, 80 bp, Rat
ctctcacctgatagatgcctctcagaacaccccgggtacagtagccaagcacagcctgccttggaagtacccactctccc

SEQ ID NO: 272 - DNA, 80 bp, Rat
ctctcaccgcgaggagtatccagagggtgacttcataagggatttgaacatagtcataatccaattcaaaaaactctccc

SEQ ID NO: 273 - DNA, 80 bp, Rat
ctctcacccaaaggcctcgtccctagcagttagactccagttctgtgagcctgaggagggacgtcatcggtgactctccc

SEQ ID NO: 274 - DNA, 80 bp, Rat
ctctcacccattgccctgatgctgataaagtcaggatgagagtcagaagaaagggcaaacctctcagctaccactctccc

SEQ ID NO: 275 - DNA, 80 bp, Rat
ctctcaccgcaggatcaagaatatacccctctttgcccctgttggggtaaacaggaacattcaaagactccaactctccc

SEQ ID NO: 276 - DNA, 80 bp, Rat
ctctcaccactatcttgacctgaagagtcttcatggtctcgtcatcgccgaactccagctctccacaagcatactctccc

SEQ ID NO: 277 - DNA, 80 bp, Rat
ctctcaccgtgatttcctctaaccagtagcacaggagggaggttcttggtcaggatttctaaccacgccatgactctccc

SEQ ID NO: 278 - DNA, 80 bp, Rat
ctctcacccgcggatcttgcatttgccccatctcttcttgcggtgcaggagataagggatgaggagggtgagactctccc

SEQ ID NO: 279 - DNA, 80 bp, Rat
ctctcacctccttctcactgctgctcatatatgggtggttgttggggtcatcaaagagaaggatgaaccagaactctccc

SEQ ID NO: 280 - DNA, 80 bp, Rat
ctctcacctgatgtcctctagccgcctggtggttgcagccacctcctcggtagtgggtggaggactccctgaactctccc

SEQ ID NO: 281 - DNA, 80 bp, Rat
ctctcaccggaattagacggattgagaaggcactggcttgtgttggagtgggcccaggaggggaatgtgtccactctccc

SEQ ID NO: 282 - DNA, 80 bp, Rat
ctctcaccacctcagacacaaacattggagaggtgttccctgtgtttcccctgctgcccttggtcagcccagactctccc

SEQ ID NO: 283 - DNA, 80 bp, Rat
ctctcaccctcactgcttctcatagtagaaccaatgctcattgataaaaatgaaccaagctcaaccctttgcactctccc

SEQ ID NO: 284 - DNA, 80 bp, Rat
ctctcacccatgagggttgctctggagtgaaagggcgtagccccttcacgctccacagcccagcggttgggcactctccc

SEQ ID NO: 285 - DNA, 80 bp, Rat
ctctcacccgccaatgatcacagcgctgactgtgagcaagatgaaggcatttcggcgcaggaagcgccgcacactctccc

SEQ ID NO: 286 - DNA, 80 bp, Rat
ctctcaccggataaaagatcccaccagcatgatcgcagtctgcaaggtgtctgtgtaaattactgctgccagactctccc

SEQ ID NO: 287 - DNA, 80 bp, Rat
ctctcaccttataaggaaagaaacttctaaactactaactaaaaaaaaaaaaaaaatcaacacaatagcatcactctccc

SEQ ID NO: 288 - DNA, 80 bp, Rat
ctctcaccgaccaggttgagtgcgtccacaaagcaggggttttggagaacaccatggtagatgttgtaggagactctccc

SEQ ID NO: 289 - DNA, 80 bp, Rat
ctctcacctgctggggtacaagattcttgaagctggcgatgatatttgtgctcttgaactcctggtagtagcactctccc

SEQ ID NO: 290 - DNA, 80 bp, Rat
ctctcaccgccagcaggggctgcaggtacttgggctgcaggagtttgccgtagtagggatagtactgcagagactctccc

SEQ ID NO: 291 - DNA, 80 bp, Rat
ctctcaccaagtaaggccggtgacgacatcgttctcgttatcccactctggttctggctctcctgcggggaaactctccc

SEQ ID NO: 292 - DNA, 80 bp, Rat
ctctcaccccgtgaggaaaccatagaagacgaggtagaagaggaggatgaaggcccagctggtcccggtgcgactctccc

SEQ ID NO: 293 - DNA, 80 bp, Rat
ctctcaccgacaggcagagacatctgaagcaccaccccccactgtcgaccactcagtggggtcacctggaaaactctccc

SEQ ID NO: 294 - DNA, 80 bp, Rat
ctctcaccaacttggtatggttcggagcagaaaaggtctcctggaagaagtgcttttcagacgaacagttgaactctccc

SEQ ID NO: 295 - DNA, 80 bp, Rat
ctctcacctgttcttatcccaccagcttccagggtagagcctgatgaacaactttcgcatctcgtcatacacactctccc

SEQ ID NO: 296 - DNA, 80 bp, Rat
ctctcacccagcaccatcagccactgggtcagattcagcggtgtgatctggaaaatgagtggcaaaggttccactctccc

SEQ ID NO: 297 - DNA, 80 bp, Rat
ctctcaccagagagcacgaggagaagtggcagctaatggggctctcagaacagcacgaggccccagagaactactctccc

SEQ ID NO: 298 - DNA, 80 bp, Rat
ctctcaccccgtggtgcctggctggggaagtggcatcactgtgcccatgcaccggtggcactgccggctcttactctccc

SEQ ID NO: 299 - DNA, 80 bp, Rat
ctctcaccgcaatcccacccagcctctgcagatgcaccacacactgctggcgaggctagcagggtgcttgccactctccc

SEQ ID NO: 300 - DNA, 80 bp, Rat
ctctcaccgcaactgctccagctttcgcataagcctggagttccttattagtaaatgaagtcagtacctttgactctccc

SEQ ID NO: 301 - DNA, 80 bp, Rat
ctctcaccgcttacacagggcattcaggaagccgaaggccgtcgtcctcttgtcggaagggtagagttcaacactctccc

SEQ ID NO: 302 - DNA, 80 bp, Rat
ctctcacccacattctcattcagaaggtctctgtcttcactgggacagtccgaagggacagtggtgttagccactctccc

SEQ ID NO: 303 - DNA, 80 bp, Rat
ctctcaccctctctcccttccccctggggttcccttgagctgctagtttgttttgaaaacttggtcccttgcactctccc

SEQ ID NO: 304 - DNA, 80 bp, Rat
ctctcaccgtcctctgtgaacggccctccacgcgacatcttacatgaatcaaacagactgtgtttagaagatactctccc

SEQ ID NO: 305 - DNA, 80 bp, Rat
ctctcacccatcagttaacattttggtaatattcacatagtttgtgttagccaaggtacagttggctgttttactctccc

SEQ ID NO: 306 - DNA, 80 bp, Rat
ctctcaccgggttccaaacccaacacagtaaaaaagaaagagggctggaaaaatgaacagtgtgcattagcaactctccc

SEQ ID NO: 307 - DNA, 80 bp, Rat
ctctcaccagcccacctcagatgatcttgcagtcttccttgagcattaaggcagaaaacagagctggcctagactctccc

SEQ ID NO: 308 - DNA, 80 bp, Rat
ctctcaccagcagcggcacgtcgtagctcatgcgatcaatgaaaggcccgcttaaggggttcaccagaagctactctccc

SEQ ID NO: 309 - DNA, 80 bp, Rat
ctctcaccctcatccacgaagccccgccacagcgtcatgtgttcaggaagataggtggcaaaaatgtttgcaactctccc

SEQ ID NO: 310 - DNA, 80 bp, Rat
ctctcaccctcttctcctcctctggatccagcttcctggaggatctcctcgaaccgctccacacctaaggtaactctccc

SEQ ID NO: 311 - DNA, 80 bp, Rat
ctctcacctcagccaaatattggactaaaactaaatcaaaacatgaaataatgcctaatggggtgttttgctactctccc

SEQ ID NO: 312 - DNA, 80 bp, Rat
ctctcaccggaactgggcgtatggttgcgcctacatttgcacttgccactcagggccatggcaattccagcgactctccc

SEQ ID NO: 313 - DNA, 80 bp, Rat
ctctcaccggcgacgtttgagctccacgtcaggacagaagacatactcgtaaagtgcacctgccagcacagcactctccc

SEQ ID NO: 314 - DNA, 80 bp, Rat
ctctcaccagccacgcgatcatggaggctcagagaggaggggaagagcaggtagaaatagaggatggccgccactctccc

SEQ ID NO: 315 - DNA, 80 bp, Rat
ctctcaccggctggcacgggatgcagaggccttgggagtccctcagtggctccatcgtgtcctcgttgacctactctccc

SEQ ID NO: 316 - DNA, 80 bp, Rat
ctctcacctgccccagacaaggctagaccatgtgggacagccaggtcttggggagtgactttggtcagcttgactctccc

SEQ ID NO: 317 - DNA, 80 bp, Rat
ctctcaccagcaaccggtgaaatagatcccaaggaggtggcccagggtaacagagaaaccaatggagagggcactctccc

SEQ ID NO: 318 - DNA, 80 bp, Rat
ctctcaccgcttaatgagaatgtatcttttcagctctaaatacttccaactgtgaaccataaattcaatgaaactctccc

SEQ ID NO: 319 - DNA, 80 bp, Rat
ctctcacctgcctggtgggcaggctgaaagccctgagtctggaagttgatgaaggacaggtgcaagcatcccactctccc

SEQ ID NO: 320 - DNA, 80 bp, Rat
ctctcaccacaggagcggatcagaactggagaaatgagcagtgccttcgtggtctgcatttttcaatctgcaactctccc

SEQ ID NO: 321 - DNA, 80 bp, Rat
ctctcaccgagaggtgctcctgatcagggagcgggtagagacgaacttggagcaggcatacatttttcagggactctccc

SEQ ID NO: 322 - DNA, 80 bp, Rat
ctctcaccattccatgggagaacaccatacattctggcatcagcgcagagactgcctatactggcagaggtcactctccc

SEQ ID NO: 323 - DNA, 80 bp, Rat
ctctcacctagttcttggggtagggactgttgaaataccagacgccatgtaccacagccggtctgagctcatactctccc

SEQ ID NO: 324 - DNA, 80 bp, Rat
ctctcaccctatcctggtgagctggctaatcttatcattgccaagccaatattcgcctggcaagccacagtaactctccc

SEQ ID NO: 325 - DNA, 80 bp, Rat
ctctcacctcctaaaacttagagccaacaatacttctaaggtaagaatatatttcccacaggtagcactgacactctccc

SEQ ID NO: 326 - DNA, 80 bp, Rat
ctctcaccgatgatgacagcattgtctccatcgtcgcttttcctacacgccgtgaggtccggcagccggtccactctccc

SEQ ID NO: 327 - DNA, 80 bp, Rat
ctctcaccatggggtaaagggaagtggccatgagagaaggcagcacggacagtggtcagtcactgggcaggaactctccc

SEQ ID NO: 328 - DNA, 80 bp, Rat
ctctcacccagtgaatactgcagtcggcctaactggtgcttgtccaatacctgctgtcctggcatggatggtactctccc

SEQ ID NO: 329 - DNA, 80 bp, Rat
ctctcaccacccttcagttccagatccagggctttgttcatgtcatgttgactgtactttctataattctgaactctccc

SEQ ID NO: 330 - DNA, 80 bp, Rat
ctctcaccgccacatggacatcttatttcatcccctggtcccctgacctgcaagcagctttcaggacgcgctactctccc

SEQ ID NO: 331 - DNA, 80 bp, Rat
ctctcacctgataacctcaacctccaactgacccttcttatcctccatcccgatttggatatcgcccatggcactctccc

SEQ ID NO: 332 - DNA, 80 bp, Rat
ctctcaccagtagtacagggatttgccatatttcctcttgaattcagatctgattttcaacatgtccacttcactctccc

SEQ ID NO: 333 - DNA, 80 bp, Rat
ctctcaccagtgcaggccctgcccagggcagctgccagaagaggacccaagccctgtccggtggcgcggccaactctccc

SEQ ID NO: 334 - DNA, 80 bp, Rat
ctctcacccctttcaaggaagggagaatctgggcaatggacatggggttctggaatttcccctgcttaatctactctccc

SEQ ID NO: 335 - DNA, 80 bp, Rat
ctctcaccaggggtgaagcctcctctgggggtgatggtgtctttgcaatgcgaagaggacccgaatcactggactctccc

SEQ ID NO: 336 - DNA, 80 bp, Rat
ctctcaccgctgaaggatgcggaagagcgggtagagatgtatgggtagtgctttgtatccagtttgtcttcaactctccc

SEQ ID NO: 337 - DNA, 80 bp, Rat
ctctcaccaggcaaagctgctttacgctttgcttcactcttgtcatctcctccaaacttctttttgctacttactctccc

SEQ ID NO: 338 - DNA, 80 bp, Rat
ctctcacccaatgagtggcatggaggagcccaccacctcaataatgtgatcccttccgtccttgccatgcagactctccc

SEQ ID NO: 339 - DNA, 80 bp, Rat
ctctcacctcctgagagcctgttatcctcaaagcccctcattccctgaaattatttggtgccttcatgaacaactctccc

SEQ ID NO: 340 - DNA, 80 bp, Rat
ctctcaccttctgttttgtcctactcctccccaaggcagtgatgacccccacacacactagagccacccctcactctccc

SEQ ID NO: 341 - DNA, 80 bp, Rat
ctctcaccaaataaaaccaaagaagtgaagcagtgtggttctgtacgacctgctcattgaattgagctattcactctccc

SEQ ID NO: 342 - DNA, 80 bp, Rat
ctctcaccagttcgcactcctcccccaaggtgaactcattgtggatcaccttggacccataggtgatggtgaactctccc

SEQ ID NO: 343 - DNA, 80 bp, Rat
ctctcaccacacaatgggacctgggtccaggctggtgtcccatatctccaggaaaagcccaaccaaagagtaactctccc

SEQ ID NO: 344 - DNA, 80 bp, Rat
ctctcaccctccttcatatgtgtaggtttggattagttcgttaccagaaatctctcggacagcaatcagctcactctccc

SEQ ID NO: 345 - DNA, 80 bp, Rat
ctctcacctgtcctgaggttgccaaagggactcttcacagcttagctttgtccggctgaagctgcaggctttactctccc

SEQ ID NO: 346 - DNA, 80 bp, Rat
ctctcaccatcccacttctgcacatgtaccaggaccccaccatccagggttatgatgctcttcactttcctgactctccc

SEQ ID NO: 347 - DNA, 80 bp, Rat
ctctcaccggtcctagtgctgacaacattgttcatggtgcactccactaccattcttccatccacaattcttactctccc

SEQ ID NO: 348 - DNA, 80 bp, Rat
ctctcacccaatatcactacggagtaagttgagtgctatccactgaggaaactacgtgtctaagcacacgggactctccc

SEQ ID NO: 349 - DNA, 80 bp, Rat
ctctcaccctctaataggtctctggctctgtgcaaagcacttatcgtcagatggtatttttccacttacacaactctccc

SEQ ID NO: 350 - DNA, 80 bp, Rat
ctctcaccgggcagtgcaggtggtcgctcctcactgcttgggtctgcttgggtggtcagggtctgctgggtgactctccc

SEQ ID NO: 351 - DNA, 80 bp, Rat
ctctcacctccacatgctctgagatcttgtcgatgttgagcctcgtttcctcaatctcagaaaagaactcatactctccc

SEQ ID NO: 352 - DNA, 80 bp, Rat
ctctcacctacgattgatcatctctccctgcatctccacctcggtagctagaaaagtgaagatctcatggagactctccc

SEQ ID NO: 353 - DNA, 80 bp, Rat
ctctcaccaagaagacaatgaggatgaggaagattttgaccatgagccaccgattggaggtgactgactggaactctccc

SEQ ID NO: 354 - DNA, 80 bp, Rat
ctctcaccgggagttggccagctgcagctcccgatcaaggcgcccatatcgatccgtcactccagcatcccaactctccc

SEQ ID NO: 355 - DNA, 80 bp, Rat
ctctcaccctgacatctccaaagaagccaaggtgggggcaggaggtgctgggccctgcagcagaatattctgactctccc

SEQ ID NO: 356 - DNA, 80 bp, Rat
ctctcacctctgcaggaagcagcagagaggagcataaatgatgttgatggtaccaatgatgaccatgagccaactctccc

SEQ ID NO: 357 - DNA, 80 bp, Rat
ctctcaccagtagctggataactctggaacttcaggaaaccaagttttcaaaagacccggcatttcttctatactctccc

SEQ ID NO: 358 - DNA, 80 bp, Rat
ctctcaccgatcttgtcatagggtggcacggaggcatccaacagcaccgggacaatcagtttcatggtgtccactctccc

SEQ ID NO: 359 - DNA, 80 bp, Rat
ctctcaccatgacggaggtttccgatgatgccgctcacctgctccaggttttcatccatttcattttcccggactctccc

SEQ ID NO: 360 - DNA, 80 bp, Rat
ctctcaccaaccaggaagatcttggtagtgccccctttgacgcaggagggtgcatcaaagtacacttggtgcactctccc

SEQ ID NO: 361 - DNA, 80 bp, Rat
ctctcaccctgcaggtgggaagtggcggcggcacacagggcacgttccggacttctgtagccaaatggagacactctccc

SEQ ID NO: 362 - DNA, 80 bp, Rat
ctctcaccctccacctgactgcttgactggagtgctctggattttaacattggacttctccgcacctccagcactctccc

SEQ ID NO: 363 - DNA, 80 bp, Rat
ctctcaccgcccaaggagcacttgttgctgttcttcttgtcagatactttcaggtcagtgcttgagggcttgactctccc

SEQ ID NO: 364 - DNA, 80 bp, Rat
ctctcaccatatcttgccaaagactcgctgtaaaagtttaattctgttaatcgttcacaacatggctggtctactctccc

SEQ ID NO: 365 - DNA, 80 bp, Rat
ctctcacctcaatctggcaaagccctgaggcagctcacccaggctatagagtgggtacaaatatgggctcttactctccc

SEQ ID NO: 366 - DNA, 80 bp, Rat
ctctcaccgcgatgtctcctgcaactaccaaagcgtatcgtccatcccaagagctggattcgatccagttcaactctccc

SEQ ID NO: 367 - DNA, 80 bp, Rat
ctctcaccgggtccaggcgagagatcagatcaacagcacacgcgctctgggtggtattctacgtggtgctccactctccc

SEQ ID NO: 368 - DNA, 80 bp, Rat
ctctcaccggcatcacctccaaatagtctccacgacgcacgtgaacacccacataggtacgaggacggatgcactctccc

SEQ ID NO: 369 - DNA, 80 bp, Rat
ctctcaccgtgggaccgaggacaggctggtgttaatgccttaaggagtggggacagatcggcagggatgcccactctccc

SEQ ID NO: 370 - DNA, 80 bp, Rat
ctctcaccagccaatgcactggcctggagggctctgccgtatgaaaagcttagtttccagggcctaggtagaactctccc

SEQ ID NO: 371 - DNA, 80 bp, Rat
ctctcaccgagagattcactggctgcggctccagactggccacttggagtgtactttccttgacaagcgaggactctccc

SEQ ID NO: 372 - DNA, 80 bp, Rat
ctctcaccaggattcttcctcatgttaaatcttgatggtggagtacagttcatctatctgtgacctgaaataactctccc

SEQ ID NO: 373 - DNA, 80 bp, Rat
ctctcaccccgtcactggttgtcaccaaggaggcaggtgggttaggctgagggttctgagtgttaccagccaactctccc

SEQ ID NO: 374 - DNA, 80 bp, Rat
ctctcaccaaatccataggaattaagtttatatccaggaggttgatctcttgtgaacattccccaagtgtgcactctccc

SEQ ID NO: 375 - DNA, 80 bp, Rat
ctctcaccaaagttcgtcacagatagaatggaccggtgactctcgagctctgctctggggcaggggcagggaactctccc

SEQ ID NO: 376 - DNA, 80 bp, Rat
ctctcaccaagaacagccagagcaggtgagtcgaggtgttctgggtgacttggggctcaaggtatcaaggtaactctccc

SEQ ID NO: 377 - DNA, 80 bp, Rat
ctctcaccctagattggggtcttccttggtgcccgaagctgcctttggaggtagcatattgtatggatccagactctccc

SEQ ID NO: 378 - DNA, 80 bp, Rat
ctctcacctaggacttcttccccaccaggatgctcttcgagaaacttggtcagatcgtacaccttatgatgtactctccc

SEQ ID NO: 379 - DNA, 80 bp, Rat
ctctcaccatacttcttcttctgttgatcatttccagcaataatcacaggcatttgccccaaagaatttgctactctccc

SEQ ID NO: 380 - DNA, 80 bp, Rat
ctctcaccgttgtgatgtggctggcgcttgattcggtaggcggaggagtcagaaaccgggaaggcattcatgactctccc

SEQ ID NO: 381 - DNA, 80 bp, Rat
ctctcaccaactcatgaggatgttgcctcttttaaatattgaataaggtcttctctctcactcttctttttgactctccc

SEQ ID NO: 382 - DNA, 80 bp, Rat
ctctcacccacaggggggctctggtccacagcagaacctgtggtgctcactgcttcagggagggtcacccagactctccc

SEQ ID NO: 383 - DNA, 80 bp, Rat
ctctcaccctcagtgaagaggaagggaggggctggaaaccaaggcttgaaacttaaatagagggtggcaaacactctccc

SEQ ID NO: 384 - DNA, 80 bp, Rat
ctctcaccgggagggaggagcccacagcagcaggctctcacttcttccattcattcttgttgtagtcccactactctccc

SEQ ID NO: 385 - DNA, 80 bp, Rat
ctctcaccggagtggagattcccaattcatttaaagttggtctaagttcctggatgacataggggtagatttactctccc

SEQ ID NO: 386 - DNA, 80 bp, Rat
ctctcaccgaatgcagccatagctaacatatagatggcatacgctgttccaccaactgtcagaagcattgtgactctccc

SEQ ID NO: 387 - DNA, 80 bp, Rat
ctctcacccttctcttatagctctccaagtgggataagacccatcctgctggaaccatgaagccagcgactaactctccc

SEQ ID NO: 388 - DNA, 80 bp, Rat
ctctcaccgaggttctggaccatctctttgcccaggtagtggtcaatgcggtagatctggtcctcacgaaacactctccc

SEQ ID NO: 389 - DNA, 80 bp, Rat
ctctcaccgggacatccgagggcacaggagacataccctctcacagagatgctggcagccctcgcggcctgcactctccc

SEQ ID NO: 390 - DNA, 80 bp, Rat
ctctcacctctcatattcccggaccctagcctgagatccagtcacgaactgggcacagttcttcacatcctcactctccc

SEQ ID NO: 391 - DNA, 80 bp, Rat
ctctcaccagtgctgttgtgtttggctgaagggcccctcacgacccatgctcctctgccagcttgtcagcctactctccc

SEQ ID NO: 392 - DNA, 80 bp, Rat
ctctcaccatcacatgcttctccacccagtttatcatgtgctctccctccttgcgacgcatcatgtcctgtaactctccc

SEQ ID NO: 393 - DNA, 80 bp, Rat
ctctcacccagtcaaagagccaccaccaaaggaatcgttcatcttggctgctctctccagcaggcgagagtgactctccc

SEQ ID NO: 394 - DNA, 80 bp, Rat
ctctcaccgacccaggacgggacaggatgggagggaggaagacatctagggctgaggctctgtatggccctgactctccc

SEQ ID NO: 395 - DNA, 80 bp, Rat
ctctcacctagcatggcttggggtacttgacagggcagttccacctcctgttccaacatcgactacatcttcactctccc

SEQ ID NO: 396 - DNA, 80 bp, Rat
ctctcaccacggagcagaatcccggcggcgagagacccagctccagccaaagccaggagcttgagtccggggactctccc

SEQ ID NO: 397 - DNA, 80 bp, Rat
ctctcaccctgctcagtagagattgtgccagacgtctggtgtctatggaaagggcagagaggtcccctgttgactctccc

SEQ ID NO: 398 - DNA, 80 bp, Rat
ctctcacccgagagttagcaaagctggtgtcatccagctgagggaatctggcctggaggtaagctcgcttctactctccc

SEQ ID NO: 399 - DNA, 80 bp, Rat
ctctcacccgcacagtgtcgctgctacagaagttgtactctttcccgtctacaccactttgcactgtgatctactctccc

SEQ ID NO: 400 - DNA, 80 bp, Rat
ctctcaccccatgtcagactctgtaacacaatcaagaaatgtcttccactcttgggagggctgctgtatggcactctccc

SEQ ID NO: 401 - DNA, 80 bp, Rat
ctctcacccatagaccctgactgaaggtgtccatgttgctgcaatggaagcctgtgttctgcaggttgctgtactctccc

SEQ ID NO: 402 - DNA, 80 bp, Rat
ctctcaccactttgggtagagttgggttgatcacattgttgtaccaaataaatttaactttctgcaaatctcactctccc

SEQ ID NO: 403 - DNA, 80 bp, Rat
ctctcaccgatatcatatatatacgttgttcccaaggactgtgcctgaaacctctttgattgaagaaggtctactctccc

SEQ ID NO: 404 - DNA, 80 bp, Rat
ctctcaccgcaagccacactctcccgctgttgtccagctatgcagcctttgactaccaagcccctccctctgactctccc

SEQ ID NO: 405 - DNA, 80 bp, Rat
ctctcacctttccagcagcctccgggcactgaggaggtggagctgatcctgctagaaccagcctctctttctactctccc

SEQ ID NO: 406 - DNA, 80 bp, Rat
ctctcaccaagcacacatttcaaagcaggtaaaaagccattatgaacattcacccggcccttcagtcgccacactctccc

SEQ ID NO: 407 - DNA, 80 bp, Rat
ctctcacctatcagctcttgttttcaaagcaaaagagtcgctaccagctccagcttcagagaggcaaaaactactctccc

SEQ ID NO: 408 - DNA, 80 bp, Rat
ctctcacctcctgggcccaggactcagtgtagatgtcaaaggtggcttgggtgcctttaagccccttggcgaactctccc

SEQ ID NO: 409 - DNA, 80 bp, Rat
ctctcaccgagccgggatcttttcctccaggtggtccaggcctctgcaggccaactcattagcagctgtgaaactctccc

SEQ ID NO: 410 - DNA, 80 bp, Rat
ctctcaccgttgattcccatccccagaataaacaggaagacaccaaagctaaaccgcacatctgtgtaccacactctccc

SEQ ID NO: 411 - DNA, 80 bp, Rat
ctctcaccgattggagtcagtgtgttgtattattggcctctgagagtcgagcatcacacaaaggagcttcgaactctccc

SEQ ID NO: 412 - DNA, 80 bp, Rat
ctctcacccacattatttacagctaataaataaaatttaaagtcaaagcatcattgagatatgtacgtatatactctccc

SEQ ID NO: 413 - DNA, 80 bp, Rat
ctctcacccgagacaccaccttctggagcccttcctccgaccttgcagtcaataccacatgggctcccatttactctccc

SEQ ID NO: 414 - DNA, 80 bp, Rat
ctctcaccgactcagggtccacgatctcctccaacatccccaggcgctccccaaatacaacactggtgatggactctccc

SEQ ID NO: 415 - DNA, 80 bp, Rat
ctctcaccattttgacgaagattttctcaaagtcagcaaagtcatgccaggaagactggaacatgtgcatgaactctccc

SEQ ID NO: 416 - DNA, 80 bp, Rat
ctctcaccgggcgggagccccccagatgatgccgagaacaggaatacacccaggcggccatgtgatgccatcactctccc

SEQ ID NO: 417 - DNA, 80 bp, Rat
ctctcacctccgcatgggcttgatggaaccgccccatggggccccaagagagatgaagccatcaatgaagtgactctccc

SEQ ID NO: 418 - DNA, 80 bp, Rat
ctctcaccgacagaactagggaggctttggagcaggagagtgatgtggttacagccagctctggagggtctgactctccc

SEQ ID NO: 419 - DNA, 80 bp, Rat
ctctcacccaggcagttttattccttctacatgatgtcccctgcaccttgttcaggtgactgggccagtgacactctccc

SEQ ID NO: 420 - DNA, 80 bp, Rat
ctctcaccagcaggcgctccaacttcagcaggataccaaaggccagtagaaaacacagcatacttaacgcctactctccc

SEQ ID NO: 421 - DNA, 80 bp, Rat
ctctcaccagtcctcttttctttctttgttagactctgtggtcttgaagaaatcagttacatacaaaaccacactctccc

SEQ ID NO: 422 - DNA, 80 bp, Rat
ctctcaccgtacatcctcctcaagtcccaatgacttccatgtggtttccctttcagtgaaccagatacttcaactctccc

SEQ ID NO: 423 - DNA, 80 bp, Rat
ctctcaccacgtttcagagctaatgtcatctctgagattcttcttatatgctgtataataggcctgcgagatactctccc

SEQ ID NO: 424 - DNA, 80 bp, Rat
ctctcacctcaggcatgaactgatctggctggtcccattcattctcatcgtgatgcagtgcccagagattgaactctccc

SEQ ID NO: 425 - DNA, 80 bp, Rat
ctctcaccaccgggacagtcatgacagactgtcacaatgtcacaaacaagcatcttcgggtgacacactatgactctccc

SEQ ID NO: 426 - DNA, 80 bp, Rat
ctctcaccatgtactggtatatgatagctttgacagctggcatgttggtggcatccatcatgagggtaactgactctccc

SEQ ID NO: 427 - DNA, 80 bp, Rat
ctctcaccaggcaggttgcccagtcgcgcggctgcaggacgaatgtactcgtacagcggccaggccatcgagactctccc

SEQ ID NO: 428 - DNA, 80 bp, Rat
ctctcaccactctcatgaactctccatacatcttattgtagtagttgcaggcacttccaatccccatccacaactctccc

SEQ ID NO: 429 - DNA, 80 bp, Rat
ctctcaccgtgtgtctctggacaggggattagggaagaaagcttgtggactagcaaggcttccagtgaagtcactctccc

SEQ ID NO: 430 - DNA, 80 bp, Rat
ctctcaccagtaccaagaatgaagtagacaggaccctcagaagagacattacagatgagagcttagagagtcactctccc

SEQ ID NO: 431 - DNA, 80 bp, Rat
ctctcacctggggatttgaagcagaagttctgcatgatgtttgtgaggaagagaaagagttccatccttgccactctccc

SEQ ID NO: 432 - DNA, 80 bp, Rat
ctctcaccaggggattgcagaaggcaacacagtagaagccaaaatgcctgatctattcaggaaggaatgaagactctccc

SEQ ID NO: 433 - DNA, 80 bp, Rat
ctctcaccacaaactgggatggagtctggaggaaaagctacagaaatgagggcaaaaaaatgagatgacgggactctccc

SEQ ID NO: 434 - DNA, 80 bp, Rat
ctctcaccgcagatggagtggggacaagagtgactgacagaaagacaaatggcagacagcatgctgttggagactctccc

SEQ ID NO: 435 - DNA, 80 bp, Rat
ctctcaccacacgaggacaggaggacctgtttgctcacaagtagaagcagggtctcagtcatctgtgtgaaaactctccc

SEQ ID NO: 436 - DNA, 80 bp, Rat
ctctcacccctccgtaagattctggtatgggtgacagttctgttcattctgggatcctcagaggtaggatggactctccc

SEQ ID NO: 437 - DNA, 80 bp, Rat
ctctcaccttctgtagaagatataggtctgagtcaagacacggaccaagagccaggaagctccgatcagcagactctccc

SEQ ID NO: 438 - DNA, 80 bp, Rat
ctctcaccgaaataaacaacttcagttttctccagttgctgaaaagtgtttggtgtgatccaggaagatactactctccc

SEQ ID NO: 439 - DNA, 80 bp, Rat
ctctcaccagtgaacaaaattactgcaatcttcatctctggattcattcacaatatgaactggtttctggtcactctccc

SEQ ID NO: 440 - DNA, 80 bp, Rat
ctctcaccacaactgtaagataataacgtcctgttattcccgaccatcaaggttgtgaccgttgattattggactctccc

SEQ ID NO: 441 - DNA, 80 bp, Rat
ctctcacccctctacagtgtgctctcccacagaagggtccctttgggaagaaggaacagaaggggttctggaactctccc

SEQ ID NO: 442 - DNA, 80 bp, Rat
ctctcacctggtcaggccagagatttgctgcaagttaaaggccagcctgctgtatccactaaaatttgagacactctccc

SEQ ID NO: 443 - DNA, 80 bp, Rat
ctctcacctcctccacaacctcctatcttctttgaaaactttttgtccctggcttccgacttgccggaagctactctccc

SEQ ID NO: 444 - DNA, 80 bp, Rat
ctctcaccaaaaacctatgaagggtccttcaatgccccttcttagggagctggtggcagagttcacgctgacactctccc

SEQ ID NO: 445 - DNA, 80 bp, Rat
ctctcacctgctgccagagggacaccaagagcatgacgctcagggaggccaatatgaccaccagaagcagtcactctccc

SEQ ID NO: 446 - DNA, 80 bp, Rat
ctctcaccgcagtggcccaaggtattctgacacatccactttgaattctgcttccgagtcccgacagggtcgactctccc

SEQ ID NO: 447 - DNA, 80 bp, Rat
ctctcaccttcattggaaggcagagggaagggggtggggttggaaggaaggagagtcaggctttcgtcacagactctccc

SEQ ID NO: 448 - DNA, 80 bp, Rat
ctctcaccatttaccaatccttttagtctttcaacagtaagagaaccaaacattaggggaactaaagggtcaactctccc

SEQ ID NO: 449 - DNA, 80 bp, Rat
ctctcaccaatgccgcgtttgtcatagaaggagatgacaggttctgtggctttgtaatacgtctcaagcctcactctccc

SEQ ID NO: 450 - DNA, 80 bp, Rat
ctctcaccgagcccttaataaaccacaacaactcctccaaaacacccttccagaatactcgtttggttgtgaactctccc

SEQ ID NO: 451 - DNA, 80 bp, Rat
ctctcaccgcatacttgatgaccaggcacttgtactgggcgatctggaagcgccttactctcctcatcagctactctccc

SEQ ID NO: 452 - DNA, 80 bp, Rat
ctctcacccaaagggcacgttgagatttatcactgtgtctatctgataaactctatccagggcttctgcctgactctccc

SEQ ID NO: 453 - DNA, 80 bp, Rat
ctctcaccggaggagaggcacaggaggcggatctcagacggacagtgacacaaagctgttgtactgctggatactctccc

SEQ ID NO: 454 - DNA, 80 bp, Rat
ctctcacctccgtacattctaaaaacaaaccacattttcagggagcgaaatcttcgccccagtgggatttgcactctccc

SEQ ID NO: 455 - DNA, 80 bp, Rat
ctctcacccatattgggaaaaaacattcttttttctaaaagaacaagcccttgggaggagcagcgttggcctactctccc

SEQ ID NO: 456 - DNA, 80 bp, Rat
ctctcaccccttcctctgggttgggtctgtgctctggcagtgtgaatccagggcagcctagcctgggaaatgactctccc

SEQ ID NO: 457 - DNA, 80 bp, Rat
ctctcaccttttggaagggttcaagtggaacacttctggttctaaaaggccagcctgaagtgtcttcagaaaactctccc

SEQ ID NO: 458 - DNA, 80 bp, Rat
ctctcaccctgtgcccactggtacagctgctccaactgtgttttgtctagatccgaggtgcctatagcaacaactctccc

SEQ ID NO: 459 - DNA, 80 bp, Rat
ctctcacctccatgaggatgtaggaggctctctcctcgctgtctttcagctgctccagagcgtgtaccatttactctccc

SEQ ID NO: 460 - DNA, 80 bp, Rat
ctctcaccaagatccagagccgacagcaaccctgtgctatgtatttcttcagtaatgtacagtccttctctgactctccc

SEQ ID NO: 461 - DNA, 80 bp, Rat
ctctcacccttcccaggtgccaggaagaggctgaatattattcattagatcaaaatgataataggacacagcactctccc

SEQ ID NO: 462 - DNA, 80 bp, Rat
ctctcaccagcagggtggtaggctcccagcaacggcctttccctgggtccctaggacactgcagtgggggtaactctccc

SEQ ID NO: 463 - DNA, 80 bp, Rat
ctctcacctaatggaaatcaatcactttagttgatctatcaaaacttttcaccacgtactgaagcaaaatgtactctccc

SEQ ID NO: 464 - DNA, 80 bp, Rat
ctctcaccgcagtaaccacatggagttccaagcccgatgaggtcttctttacttccttaacctgtgagaactactctccc

SEQ ID NO: 465 - DNA, 80 bp, Rat
ctctcacccaccgtagatcggctgaactcgagcatccacgtaggcttttgcaatcgggtattcccacatgtaactctccc

SEQ ID NO: 466 - DNA, 80 bp, Rat
ctctcaccgtcattcaggtctctgtggccctgcctccagcaggtcgctcaaagagagaggcactgttggtggactctccc

SEQ ID NO: 467 - DNA, 80 bp, Rat
ctctcacccaatggctgaaagcctggaggtaagacaagtaacttaactgtgtcaaaaagtggggtcaaaggcactctccc

SEQ ID NO: 468 - DNA, 80 bp, Rat
ctctcaccttgaagagaggagagcgcaagaagagatcaggagaacgggcatggccagctgcccgggccttcaactctccc

SEQ ID NO: 469 - DNA, 80 bp, Rat
ctctcacccggggagagtagtgaactggtccttgatgaatccttcaaatccagactgggttgttttcaagacactctccc

SEQ ID NO: 470 - DNA, 80 bp, Rat
ctctcaccctcacatgtctgtcttggatatcgtcgtccggtgagggcttgttgcagtcacgaacctggaaaaactctccc

SEQ ID NO: 471 - DNA, 80 bp, Rat
ctctcacctcgaaggtatcatcaaacacaactctgcaggtcttcccattgttcaggatggtcttagcagagcactctccc

SEQ ID NO: 472 - DNA, 80 bp, Rat
ctctcacctgggtgggcagatctcctccaaaaatatagatgtcctcccccagcgacatttccactgagtgttactctccc

SEQ ID NO: 473 - DNA, 80 bp, Rat
ctctcacccagcctctgcagggcctcgtgatgggccccgagcttcagaaacactccaatcaccgccagtccaactctccc

SEQ ID NO: 474 - DNA, 80 bp, Rat
ctctcaccgttgaggggtctataatggccaggggagagggccaagggctcaagaagcctctcctgctgaaacactctccc

SEQ ID NO: 475 - DNA, 80 bp, Rat
ctctcacctgcgtggttgtctccaccttcggactggatgggctccacgatgatcccagccactgttctcttcactctccc

SEQ ID NO: 476 - DNA, 80 bp, Rat
ctctcaccgtgaaggggttttctcttggcaggcgcagtgtgcttggtggtggcttttctcaggatcttgttgactctccc

SEQ ID NO: 477 - DNA, 80 bp, Rat
ctctcaccgaaaaataaggtgctttgggaatctgcgcagtctgtgtcctgccggcccaagagagaaacccacactctccc

SEQ ID NO: 478 - DNA, 80 bp, Rat
ctctcaccagacctcaggcttcagaagctgcacctgctgtagtcccgtatgtaggcctgaggtgttgatggcactctccc

SEQ ID NO: 479 - DNA, 80 bp, Rat
ctctcaccgtaggtgcaacgacattcttaggcttcaggctcatagtcttggtagccttcctctgaaggcattactctccc

SEQ ID NO: 480 - DNA, 80 bp, Rat
ctctcacctcagcttgggtactcagccggtacctctggcgtttccagctgcgcttgggactgaagatatctcactctccc

SEQ ID NO: 481 - DNA, 80 bp, Rat
ctctcacccatttggatgaaacattttggataaaaacctaacggttggtggtttatttggatattcttcagaactctccc

SEQ ID NO: 482 - DNA, 80 bp, Rat
ctctcacccatggcaaacttcttcccactctcatctaggatggctgcattgacgtcctctccactctcactcactctccc

SEQ ID NO: 483 - DNA, 80 bp, Rat
ctctcacctcccttaccactgcgcttaattaagatggccctcaccctcttgagaaggctgagaatgcttcagactctccc

SEQ ID NO: 484 - DNA, 80 bp, Rat
ctctcaccctttgtgaggctggtgcttgccacatttcttacagaaagtccttcgggttttaggtacgttgacactctccc

SEQ ID NO: 485 - DNA, 80 bp, Rat
ctctcacccgcagagatgggcttggcgcttgtctaaggctttggcagcttcgcgtatgccacgtgctaggccactctccc

SEQ ID NO: 486 - DNA, 80 bp, Rat
ctctcaccttatccagcatccagtgttttggagccgctacgcgcttcagatgtttcttgggaccacgagccaactctccc

SEQ ID NO: 487 - DNA, 80 bp, Rat
ctctcacctcaatgtgttcctgaatcccaaagccaaagttgccggtatctgagaagttattcttccgtaattactctccc

SEQ ID NO: 488 - DNA, 80 bp, Rat
ctctcaccgatggtgcgtgccattttcttgtggataccagccaccctgagctcctccaggctgaagcccctgactctccc

SEQ ID NO: 489 - DNA, 80 bp, Rat
ctctcacccttcaaattcatctgcattgaacttggtaaaaccccatttctttgagatgtggatcttctgccgactctccc

SEQ ID NO: 490 - DNA, 80 bp, Rat
ctctcaccaggcagagggcaccacctcaatctgggcctgtctgttctggatggtcagtttcactgtaatcctactctccc

SEQ ID NO: 491 - DNA, 80 bp, Rat
ctctcaccgaggtacaggtgccgtgctgtggaagcagctcgtgtgtagaaccagttctcatcatatggggcaactctccc

SEQ ID NO: 492 - DNA, 80 bp, Rat
ctctcacccttatggggcattcctttttgaacagtgcccattcccttgatgtctacaatatcacccttcttgactctccc

SEQ ID NO: 493 - DNA, 80 bp, Rat
ctctcaccctctccaacagtgacaatgtcgccaatctggacgtccctgaaacagggggacaggtgcacagacactctccc

SEQ ID NO: 494 - DNA, 80 bp, Rat
ctctcaccaagcttcagcatttccttggtgtcaggatctacctcaatgatctcctgatctagggctgagaccactctccc

SEQ ID NO: 495 - DNA, 80 bp, Rat
ctctcaccccagaactaccgccttcaccatggagctccatgagttttcccaattcaaacttgggtttcttcaactctccc

SEQ ID NO: 496 - DNA, 80 bp, Rat
ctctcacctaagacctgaccatttccctttaaagcaacagctctaagacaaaatcttgccaacctgaggccgactctccc

SEQ ID NO: 497 - DNA, 80 bp, Rat
ctctcaccaggggtccccaggcaggatctgccagtggtcaaacacacactgggggaaggcctggccaccagtactctccc

SEQ ID NO: 498 - DNA, 80 bp, Rat
ctctcaccaagaacacaggagtaaaggatctactcagagtagctctccggatcgctaactttaaatcagacaactctccc

SEQ ID NO: 499 - DNA, 80 bp, Rat
ctctcaccactgtggcgactccgggtctccatagtcccagcgcctccatgatgactgttattggctgcctgtactctccc

SEQ ID NO: 500 - DNA, 80 bp, Rat
ctctcacctcttcatgatctgttcagagctggtgggcccgccgcctccgagctgctccccggacccgtccatactctccc

SEQ ID NO: 501 - DNA, 80 bp, Rat
ctctcaccatccttccggggaaagaagctgcaggtaccaatagcacttcgcgtcccggctcccgccccacggactctccc

SEQ ID NO: 502 - DNA, 80 bp, Rat
ctctcaccagcctccaaggagctgatctgaggaaaaaccttgtgagttttgaggccaagggaactgagatgtactctccc

SEQ ID NO: 503 - DNA, 80 bp, Rat
ctctcacccgcgtgcgtagaacttcatgcaggtgtgcttcaggcagggcttacactcttcccagagggccatactctccc

SEQ ID NO: 504 - DNA, 80 bp, Rat
ctctcacctccttgctgggaaagggactcaacacaagcagagggtgtcttccagatcattctgtgtaattctactctccc

SEQ ID NO: 505 - DNA, 80 bp, Rat
ctctcaccacttcgtgcaggacagactgtgtctcttgggcgtctgctgcacctcggcccaccatcggtggtcactctccc

SEQ ID NO: 506 - DNA, 80 bp, Rat
ctctcaccggggcccagccatatgaacccaaattggtgaaaactggaggaggcatccctcaggaagggacatactctccc

SEQ ID NO: 507 - DNA, 80 bp, Rat
ctctcaccgcagtcgaagtcactccctttataatgctggtaatttaagcattccacagcatcagattcgagcactctccc

SEQ ID NO: 508 - DNA, 80 bp, Rat
ctctcaccttgttagctacttcacaggtcggggaaagcagatcagataggttttgctcctctttgtttccatactctccc

SEQ ID NO: 509 - DNA, 80 bp, Rat
ctctcaccaagacatacaaggagtcatttcatccatccccaactcccagaagtcatttcaaccctgtggcccactctccc

SEQ ID NO: 510 - DNA, 80 bp, Rat
ctctcaccgaaatttaaaaaataaaaagaaaatgaaaaagaaggttggatccatcggtcttctgtgctagtcactctccc

SEQ ID NO: 511 - DNA, 80 bp, Rat
ctctcaccccagactgtaccaggcaaggttagtggctattgaaaataccaccaggacagggctagctaaagaactctccc

SEQ ID NO: 512 - DNA, 80 bp, Rat
ctctcacctctgccacagagaagacccctggggagggcaccagaaagaggtctgtgttatacagctccatgtactctccc

SEQ ID NO: 513 - DNA, 80 bp, Rat
ctctcaccggatagtaaagcctttctcgctgtcccaggaaattctgtttccatccggaacaaatctcttttcactctccc

SEQ ID NO: 514 - DNA, 80 bp, Rat
ctctcaccttgggtaggggcctggaagtggttgagggcggggcagacatggcttccagatatttccctgaaaactctccc

SEQ ID NO: 515 - DNA, 80 bp, Rat
ctctcaccgctcagaccaggcgctccagaatccgctgaagctcggctcggccatgcgtgctcgaacagcgaaactctccc

SEQ ID NO: 516 - DNA, 80 bp, Rat
ctctcaccgccacgggcctacgagcccatgcaacatcgttcccaggctcatgtggtctgcgcctcaggaaatactctccc

SEQ ID NO: 517 - DNA, 80 bp, Rat
ctctcacctgcaccagggctccctgctctgtgggagggtgagaagccagtcatactttcagggctcaatggaactctccc

SEQ ID NO: 518 - DNA, 80 bp, Rat
ctctcaccgaggggcctgggtagctgggagtgaggggcctttgggagacatgttcacatctgtgccattaccactctccc

SEQ ID NO: 519 - DNA, 80 bp, Rat
ctctcaccaatttctcagcagggtcctattgaaacagtgctcgtggttgggtccataaaaatgctggagacaactctccc

SEQ ID NO: 520 - DNA, 80 bp, Rat
ctctcaccactggcagtcagcagtctcgtggctgaactcggggaccccgtccttcagcagagggtcacagtgactctccc

SEQ ID NO: 521 - DNA, 80 bp, Rat
ctctcaccatccacttcacaggcaggaaagtgctgccctttgacacgtagttagaatcgtgcatgatgtctcactctccc

SEQ ID NO: 522 - DNA, 80 bp, Rat
ctctcaccccatcatgcgctgagtgatgccctcgattctgtttcctatgttgctgctggaatccaggatgaaactctccc

SEQ ID NO: 523 - DNA, 80 bp, Rat
ctctcaccgggaagtgcgcactgcctcaccccggagagccgctccttgtcttgttaacactgtcagagcgaaactctccc

SEQ ID NO: 524 - DNA, 80 bp, Rat
ctctcaccaacagaagcagctggagaagacgatcatggcgacaatacagacagccatagagatgagcacagcactctccc

SEQ ID NO: 525 - DNA, 80 bp, Rat
ctctcacctatttatggaatcatctagaacttcaggggccatataccttttagtgcctactctgtggtttggactctccc

SEQ ID NO: 526 - DNA, 80 bp, Rat
ctctcacccttccagaccagggtgttgttctcccccatgatgcccgaagggcaggtcttgacacagtggggaactctccc

SEQ ID NO: 527 - DNA, 80 bp, Rat
ctctcaccagacagagtagggtccaggcgcaaggacagcccgaagtcacacaggcaacaggtcaaatcgttcactctccc

SEQ ID NO: 528 - DNA, 80 bp, Rat
ctctcaccgtcaatttgatttaaaaaaaaaataaataaaggcttccccgctgggctttccactgggaagcggactctccc

SEQ ID NO: 529 - DNA, 80 bp, Rat
ctctcacccatccaaagccttgtagaagcctggccgacaagcttggcatatgaaccctcgttcttcatacccactctccc

SEQ ID NO: 530 - DNA, 80 bp, Rat
ctctcaccaaagggtccggagcgccgcacacaggagaaggcacgtccagaggggcccctttagaggtgcggaactctccc

SEQ ID NO: 531 - DNA, 80 bp, Rat
ctctcacctcaatgtaggttttggtgcctggaaatttaaaatgaaagtagagttcttcatccccttcttggtactctccc

SEQ ID NO: 532 - DNA, 80 bp, Rat
ctctcaccggaccagggcctccagaacgctcacgacgtgagagaagcgaggccgctgggcacggtccttgtgactctccc

SEQ ID NO: 533 - DNA, 80 bp, Rat
ctctcacccgtttctagctggctctcctccttggaactattccccaagggtgtgggtgacagttttcgcggaactctccc

SEQ ID NO: 534 - DNA, 80 bp, Rat
ctctcaccgtagcaccagcagagacatgacagacatacaggccaccaccaccggagtgaagagggactcatcactctccc

SEQ ID NO: 535 - DNA, 80 bp, Rat
ctctcaccacgtcttggtaaaactccatgaacttggtcgtggcgttgagcatgcaggtcctgtgctgctcgcactctccc

SEQ ID NO: 536 - DNA, 80 bp, Rat
ctctcacctcttgagggccttgcgcttctggtggcccttggagtgtgacagcttggagatgatgatgcagtaactctccc

SEQ ID NO: 537 - DNA, 80 bp, Rat
ctctcaccacgtaaacaggataggcagtgccaagcacgagaggaggtcggccaccgccagattcagaaaccaactctccc

SEQ ID NO: 538 - DNA, 80 bp, Rat
ctctcacctatcaggtgtggaaagttagaacggttaaaaaacaatccctctcccatgcgtgcacacctttccactctccc

SEQ ID NO: 539 - DNA, 80 bp, Rat
ctctcaccactcaggcagctgttggagaaggctgccaggttgactatgtggcctgtcaaggggtaggcatgaactctccc

SEQ ID NO: 540 - DNA, 80 bp, Rat
ctctcaccctgagccatggcagggggaagatctccttaggatctgctggccttgtccgaaaggccatgggctactctccc

SEQ ID NO: 541 - DNA, 80 bp, Rat
ctctcaccgtccccgagagattccccaaggatggtgatttttcatcatagcaacagccgcagccagggcatgactctccc

SEQ ID NO: 542 - DNA, 80 bp, Rat
ctctcacctccttccatctgaagcttttcggggaagaaaatcagagcaggagtccctgtaccaaagcactatactctccc

SEQ ID NO: 543 - DNA, 80 bp, Rat
ctctcaccacgaagatccgatttctcttttgagtaggaattctttcatttgtatttgaataataccattccaactctccc

SEQ ID NO: 544 - DNA, 80 bp, Rat
ctctcacctctacgatgctgggagtggagtggctcccaagtctctgtgactcaaggcttgggagctgtcaggactctccc

SEQ ID NO: 545 - DNA, 80 bp, Rat
ctctcacccccagaaggtagccagcgctgttcagggtccagcctctcttctcctttgttggcatcccgagccactctccc

SEQ ID NO: 546 - DNA, 80 bp, Rat
ctctcaccgttgcgaggaccagcagcgccatgaggctggagtagagcacagagaagcctattaccgagaatgactctccc

SEQ ID NO: 547 - DNA, 80 bp, Rat
ctctcaccaggcgttcgtgcccagtcatttcttttgccattaaagataggaaaggctactgcatgattttgtactctccc

SEQ ID NO: 548 - DNA, 80 bp, Rat
ctctcaccggttcataagaaggaagaggacaatatgaggtcacccactccaagcagcgggtgcttcatgagcactctccc

SEQ ID NO: 549 - DNA, 80 bp, Rat
ctctcaccggaagcacgctgtctaccagcagagatgtatccgagacgatttcagcagcctctcttacgattgactctccc

SEQ ID NO: 550 - DNA, 80 bp, Rat
ctctcaccccaccctgactcctgggaagtgcagtgtgggtaaggggtgcgtaaccaaggcagccctaagaggactctccc

SEQ ID NO: 551 - DNA, 80 bp, Rat
ctctcacctcagttccgaagggccagttgaaacggttcccgatgttcccttgccagaagggcaggcccaggaactctccc

SEQ ID NO: 552 - DNA, 80 bp, Rat
ctctcaccgtgtccggggcaggacatgtgccaggtctatatccaggggctcttaagtgcctatatctacctgactctccc

SEQ ID NO: 553 - DNA, 80 bp, Rat
ctctcacccgggcggcagcagcttcctgccggtgccattgccctccacgctacgcagcgcgtactcgatggcactctccc

SEQ ID NO: 554 - DNA, 80 bp, Rat
ctctcacccatggtgccttcgccatcgctggaggcacgcaccctgaaggaacctggtttcccacccaccataactctccc

SEQ ID NO: 555 - DNA, 80 bp, Rat
ctctcacctgtacttattttaacaagacaaaactacagtaaaaatatatttttaaaatctccaaacttcactactctccc

SEQ ID NO: 556 - DNA, 80 bp, Rat
ctctcacccagcaaatgtaattctggcatgttctctgggagcacttcagacctgagtgggctcccacaagtgactctccc

SEQ ID NO: 557 - DNA, 80 bp, Rat
ctctcaccgcttggaggatcatgcagcgagtgctttatgggcaaaaatcagagtctatgaatagtatcattaactctccc

SEQ ID NO: 558 - DNA, 80 bp, Rat
ctctcaccaatttgtgttctgacacagccattagctcctgggaagtaccattatagttcaggacaaccctgaactctccc

SEQ ID NO: 559 - DNA, 80 bp, Rat
ctctcaccgctgcaatggcttcaaccctagtaatggggaaggtggaggtggtccagggtcctgagtaggaacactctccc

SEQ ID NO: 560 - DNA, 80 bp, Rat
ctctcaccacgtctctggggttcaggcgctcttgattgcgccttttaaacttcctgagagcaacgccaaggaactctccc

SEQ ID NO: 561 - DNA, 80 bp, Rat
ctctcaccgttaacaacccccccagccccaaagagccccaagttaccacatccagcttcaactccaacactgactctccc

SEQ ID NO: 562 - DNA, 80 bp, Rat
ctctcaccgggtgagggaggcggacaatctgagaagaggggaccctgagtagggtccgacaccggcgggcccactctccc

SEQ ID NO: 563 - DNA, 80 bp, Rat
ctctcaccatgcattttgaaacaattccactttctgtggaaagccagagcataacatttgaaggtgtacatgactctccc

SEQ ID NO: 564 - DNA, 80 bp, Rat
ctctcacctagcgagggtctggatggtgtgagacccagttagggcaatggagcagtttctgcctccagatttactctccc

SEQ ID NO: 565 - DNA, 80 bp, Rat
ctctcaccaggtgagatccaaggatgagggtgtggccttggggccttcctcgggctcctcttcatgggctccactctccc

SEQ ID NO: 566 - DNA, 80 bp, Rat
ctctcacccaataatgaagtcaaacgggaacagccacaatgtcctaagtcacttctgcactaggctatgggcactctccc

SEQ ID NO: 567 - DNA, 80 bp, Rat
ctctcaccggagctcggtgtggccactgcatcacttcacttgggaggggcgtttaagcagagggccgggtagactctccc

SEQ ID NO: 568 - DNA, 80 bp, Rat
ctctcacctcctccatcactggattgcctgtgacaggtgtctgtgctcagagttctttgttgacacctgtatactctccc

SEQ ID NO: 569 - DNA, 80 bp, Rat
ctctcaccaactgcaggaatgacatagatgattcccgggtgaaaccagttgtccatcttggggggactcagaactctccc

SEQ ID NO: 570 - DNA, 80 bp, Rat
ctctcaccctccagctcatcgtagctggagacttggatgaaaggacaccagcgaaatgctctcttgaagcctactctccc

SEQ ID NO: 571 - DNA, 80 bp, Rat
ctctcaccccaaaacttgccggatggcaggtcctatggtaggtccattgacttcctccaggtggcagtaaggactctccc

SEQ ID NO: 572 - DNA, 80 bp, Rat
ctctcaccggctgctggccatcctttcctcctgaagagctttgccttcttgccatcgcctccaacgccgcagactctccc

SEQ ID NO: 573 - DNA, 80 bp, Rat
ctctcaccgtcgtaaacaaatctatccctcactgctaaatagcacccaagggcaagaggtaatctgctttatactctccc

SEQ ID NO: 574 - DNA, 80 bp, Rat
ctctcacccaccacgttggtagagtctgtggccaggaagtaggatgcagtagccgcgatggatatccaaatgactctccc

SEQ ID NO: 575 - DNA, 80 bp, Rat
ctctcacctagactggctgggctgccggaggcttcgaagagggtgacagacagccaggtaccggtccagtgtactctccc

SEQ ID NO: 576 - DNA, 80 bp, Rat
ctctcaccgccaggagctgaatgaccatctccaggtggtgagacctgccttgcctgtgctgctgacttctaaactctccc

SEQ ID NO: 577 - DNA, 80 bp, Rat
ctctcaccctcggcagggcctgcaatttggtggaagaaatatccaggatgctgggccccgtggccccctggaactctccc

SEQ ID NO: 578 - DNA, 80 bp, Rat
ctctcacctgtccagttcgatgcttccgaagtgttagcatctctggagttgaagtcattatggtagagcctcactctccc

SEQ ID NO: 579 - DNA, 80 bp, Rat
ctctcaccctccaccatcaatgctggaacctcaccagggctcttcttggatatcctgttgttgtcgtgcatcactctccc

SEQ ID NO: 580 - DNA, 80 bp, Rat
ctctcaccgtagtcatttaaagtatctatacattattatgtaaggaggaagtaaagaatcccaaatgggatgactctccc

SEQ ID NO: 581 - DNA, 80 bp, Rat
ctctcaccgggagggtgaaatggttttcagaaagtagtggtcttggtgaaagggactcgcttgcaggcaggtactctccc

SEQ ID NO: 582 - DNA, 80 bp, Rat
ctctcaccgggcatggaagagaaaaacttccattcacagactccatctgtcttggagatcttatagaaagtcactctccc

SEQ ID NO: 583 - DNA, 80 bp, Rat
ctctcaccctagggctccaggcgcgctctggggccagcaggtcccgatctggtccaaggtcgtgttgcagtaactctccc

SEQ ID NO: 584 - DNA, 80 bp, Rat
  misc_feature 67: n = A,T,C or G
ctctcaccccagtttcctgtactgctttctcatgtcatgcccaacagcattggtctcccaaattttngtagcactctccc

SEQ ID NO: 585 - DNA, 80 bp, Rat
ctctcaccttggtgagcacaaacctcagttcaatggcgttccgggggaggtcggtcggaatctctgtcacctactctccc

SEQ ID NO: 586 - DNA, 80 bp, Rat
ctctcaccaggatcaattatggagttcactgataaaaatctaagagctcggaggtcccacttttcctttcggactctccc

SEQ ID NO: 587 - DNA, 80 bp, Rat
ctctcaccgtgagggcattagaagcttgtcactgcttatcagtttctcatttaatatggatgcttaggcataactctccc

SEQ ID NO: 588 - DNA, 80 bp, Rat
ctctcaccgctgtagtcacacaatgcctggtctccgggccactggcccttcatgtatgtggcgatggtcactactctccc

SEQ ID NO: 589 - DNA, 80 bp, Rat
ctctcacctctcatccttcagagtggaaagctttctctgggagaaggattcaaatcctagcagtttgaacacactctccc

SEQ ID NO: 590 - DNA, 80 bp, Rat
ctctcacctggggagcggctcaggggcccaggcgggctctcttcctctagggatgcagagcccgtgtccagcactctccc

SEQ ID NO: 591 - DNA, 80 bp, Rat
ctctcaccgatgggtcgagttgctcccagcgtcgctgcgacaggtacacgaggatgaccaccggactggtcaactctccc

SEQ ID NO: 592 - DNA, 80 bp, Rat
ctctcaccaatgatgaatgccaacacaacagcagctgccatcttcaagacttggtcacgggtaattctgttcactctccc

SEQ ID NO: 593 - DNA, 80 bp, Rat
ctctcaccggtgactgggacgttgggctgatgcaggctctggcgcacctgggcacagtgctgtaacctcacaactctccc

SEQ ID NO: 594 - DNA, 80 bp, Rat
ctctcaccgagccgccgcgggtccacggccggtaggtgtaagcgcacgcgcctagacggcgacggcggcggcactctccc

SEQ ID NO: 595 - DNA, 80 bp, Rat
ctctcaccatatcagtggccaagagggctgcgtagataaaaagagggcacagtgaggtagtgtcccaactttactctccc

SEQ ID NO: 596 - DNA, 80 bp, Rat
ctctcacctgaatgcatctcagcaaactctcagatcccagaagacaacaccgtggaggcctggaggacccccactctccc

SEQ ID NO: 597 - DNA, 80 bp, Rat
ctctcacctcatgtttgatgatacactgatcatcacggctatttccttggtgcctccagaagagaggaggcaactctccc

SEQ ID NO: 598 - DNA, 80 bp, Rat
ctctcacccccttggcagatggatcttgttcctctggacagctgggcagactacaggggtttgtgaggcaggactctccc

SEQ ID NO: 599 - DNA, 80 bp, Rat
ctctcacctgtccagcccttgtctgactccattatgttggctgctgtgtgcagaacttccttgagcagcttgactctccc

SEQ ID NO: 600 - DNA, 80 bp, Rat
ctctcacctcaccatgattgcttcaggcttaagcaaccagagtttcaaactgagaattaggaagttaacagaactctccc

SEQ ID NO: 601 - DNA, 80 bp, Rat
ctctcacctccctacagtctgtcccagagaagttttccaagttgtcccactgctgtcccgggactagagggaactctccc

SEQ ID NO: 602 - DNA, 80 bp, Rat
ctctcacccattctcccccgccgccttgcccaacccattctctgtggtcggggacgcgccgtcggggccggcactctccc

SEQ ID NO: 603 - DNA, 80 bp, Rat
ctctcaccgctgccaccgctgccgctagacagctgctgaagctccagcttcctcgggggccgggacttctgcactctccc

SEQ ID NO: 604 - DNA, 80 bp, Rat
ctctcaccgcacgatcatgatggccgcccttttgggagttcttttagcagaatagtccaccgcatcagtgatactctccc

SEQ ID NO: 605 - DNA, 80 bp, Rat
ctctcacccgcctagaaccaagcagcccagagatcttcctctcacctcagtggactctggggccaagggtctactctccc

SEQ ID NO: 606 - DNA, 80 bp, Rat
ctctcaccatggagcaggaaagagggactccatgctgtcaccctgcaaagccactcagtacagttcctccaaactctccc

SEQ ID NO: 607 - DNA, 80 bp, Rat
ctctcacccagggcaaaaagtacaaaaactacaaacatggtgacaaagttcctgaagtcctgcggcttcagtactctccc

SEQ ID NO: 608 - DNA, 80 bp, Rat
ctctcaccgaagacacagaatgtagagttagagttgtggttgaatttccttttctctatcacatcaactatgactctccc

SEQ ID NO: 609 - DNA, 80 bp, Rat
ctctcaccaaccgatgttagcaggagatatatgtgaattacgtgtgagttagatgtgtggtggtgtaaggtaactctccc

SEQ ID NO: 610 - DNA, 80 bp, Rat
ctctcaccagatgatcaacaccaccaggatcagaaccagggttttggccagcctaatgtccatgcgggcttgactctccc

SEQ ID NO: 611 - DNA, 80 bp, Rat
ctctcaccaccttagagtggttgtagtggaagaggtcgttcaggatgctattggccaggaagggcaaggagaactctccc

SEQ ID NO: 612 - DNA, 80 bp, Rat
ctctcaccagggccagcagctcatgttgtcccaaagcccctcacagcctgatgctgtctccgggcccagtgcactctccc

SEQ ID NO: 613 - DNA, 80 bp, Rat
ctctcaccccagcagtagcagcagggctgagagcgccaggctgagcacgtggtagcgcaggcgcccggggcgactctccc

SEQ ID NO: 614 - DNA, 80 bp, Rat
ctctcaccggcagccagcagaccgcgaaggcgaccacgatggagagcagcatgacgttgattcgtttggtctactctccc

SEQ ID NO: 615 - DNA, 80 bp, Rat
ctctcaccctagcgtgcgcaatccggtgcgtgcagagtggttttaaatgggatattcaaacatgtccctactactctccc

SEQ ID NO: 616 - DNA, 80 bp, Rat
ctctcaccaggctgtgtgtggtgtgcctctgagcacagcaaagcaggctacgcaactccgaggagacactgcactctccc

SEQ ID NO: 617 - DNA, 80 bp, Rat
ctctcaccatgctaaggctaggcacagtaccttgacagttcacaaagccttccatgccaggggcttcctcacactctccc

SEQ ID NO: 618 - DNA, 80 bp, Rat
ctctcacccttgtggaggtcctgtgtgaggctaagaaaggtaaagtgctctgatggataaggggtagaggagactctccc

SEQ ID NO: 619 - DNA, 80 bp, Rat
ctctcacccggtcggaggcagcatctgtctccttccagatgggtttaggggagagagggtcacaacggaaccactctccc

SEQ ID NO: 620 - DNA, 80 bp, Rat
ctctcaccatggcggatgaacgggcagatggaggaatggcggcactccagatgcacagagaaaagcctcttgactctccc

SEQ ID NO: 621 - DNA, 80 bp, Rat
ctctcaccccaaaggtctccttaagttccacaagactgtgaaacactgggaggggagaacagatggcaaagcactctccc

SEQ ID NO: 622 - DNA, 80 bp, Rat
ctctcaccgtaaggtcaataaacataacacgttgagactttttccccacgtcaaaagtcataaacttacattactctccc

SEQ ID NO: 623 - DNA, 80 bp, Rat
ctctcaccaggaccatctctgggaggggaagaatgaagtcctgacccagctgggacagtggctactccccatactctccc

SEQ ID NO: 624 - DNA, 80 bp, Rat
ctctcaccagtagtctggttgcaggaatcacacagagctaaagttacgtttgtaatgaaaaaggggcaccacactctccc

SEQ ID NO: 625 - DNA, 80 bp, Rat
ctctcaccagggatcaggtaaaccagattacagtccttggagatggagcctcggggctcatggtggctggaaactctccc

SEQ ID NO: 626 - DNA, 80 bp, Rat
ctctcaccgcagcgaccactttgtgatcagccgatctctttcccaggacgaggacccagctatagttagcatactctccc

SEQ ID NO: 627 - DNA, 80 bp, Rat
ctctcaccggcagggctctcctgtgtgtgtgtaggggtgagtaagggcacctgcccctattcctcattcctcactctccc

SEQ ID NO: 628 - DNA, 80 bp, Rat
ctctcaccggagggggcagaccaggagaccccatggaagaactgatgactgggaagggggagcccagtggcgactctccc

SEQ ID NO: 629 - DNA, 80 bp, Rat
ctctcaccagttcctgaagctggaacggctcctcccctcacgcgccaaaagccacgcccaacctgaggtggcactctccc

SEQ ID NO: 630 - DNA, 80 bp, Rat
ctctcaccttttgtgtatgtgtttgtgtgtttttaaataacaaaaggatagtaatccccatattgactagacactctccc

SEQ ID NO: 631 - DNA, 80 bp, Rat
ctctcaccctaacactgaaaccggcacataagaaacacattcaatctcagcttaccgttggggtgaccaaccactctccc

SEQ ID NO: 632 - DNA, 80 bp, Rat
ctctcaccacttaagcaggacaatctggtcatcggaggtgagatccctgaatcctgggatcatcttggcaaaactctccc

SEQ ID NO: 633 - DNA, 80 bp, Rat
ctctcacctccaaacagaggtgccacgcgggcatgtcgagaaccagcgacagcaaccagcaaccagaacaagactctccc

SEQ ID NO: 634 - DNA, 80 bp, Rat
ctctcaccactacaggtataaggctgattcagagtccaggcctgttccagatatatgctgctaccgggcacaactctccc

SEQ ID NO: 635 - DNA, 80 bp, Rat
ctctcaccatgtagtatctgcagctagctgcccacctgcgggaagctccagggaagaaaacttgcagggaggactctccc

SEQ ID NO: 636 - DNA, 80 bp, Rat
ctctcacctcaatgaagcccgggatcttttccgcccacttccggatgacgtccagggagccagagagcaagtactctccc

SEQ ID NO: 637 - DNA, 80 bp, Rat
ctctcaccaagtgacatttattgtacaaatggttaataaaaagacacattataaatatatatgtaacctgctactctccc

SEQ ID NO: 638 - DNA, 80 bp, Rat
ctctcaccgcccacgcaaccccgcggtgggtcagtgatattctccttctctaggagttcgtgaactgcctgtactctccc

SEQ ID NO: 639 - DNA, 80 bp, Rat
ctctcacctccatctacaggaagtgcaggctggtagagcaaaattgaggtaaagcccagggagcggacgctcactctccc

SEQ ID NO: 640 - DNA, 80 bp, Rat
ctctcaccaggtaggaatggctttaaactgtcattctcatttttacagcaacttctgtggtgcctgccaagaactctccc

SEQ ID NO: 641 - DNA, 80 bp, Rat
ctctcacctcctccaggcgcacgccggaacgaccacctcctccagacgtcttgttcaaggccaaagccaaggactctccc

SEQ ID NO: 642 - DNA, 80 bp, Rat
ctctcaccctgcatgattctggctgggatcgtgtctgtgtagttcaccagctggaaacctgtcacattgcgtactctccc

SEQ ID NO: 643 - DNA, 80 bp, Rat
ctctcaccgaaggcctggacttgaggggagggagggaggtgtaagagctatctctggtgttccgaggcctctactctccc

SEQ ID NO: 644 - DNA, 80 bp, Rat
ctctcaccgaggagctgcaaatgaatgagacctcacttggagctgggagctgagtggtcagggtggaggaagactctccc

SEQ ID NO: 645 - DNA, 80 bp, Rat
ctctcacccagtacttcctgtaattgacagccttgccttgctcctcatgacatcactcaaggtcatcccctaactctccc

SEQ ID NO: 646 - DNA, 80 bp, Rat
ctctcaccggcacaaacctttcatattcgttccagtaaccagcttttcgagaacccgagactttcatttcatactctccc

SEQ ID NO: 647 - DNA, 80 bp, Rat
ctctcaccagattgccagtctaaaagcagtgtattcctggtctgtgtttcggatgaagagaccacctatttgactctccc

SEQ ID NO: 648 - DNA, 80 bp, Rat
ctctcaccgcatcttctcgttgttggcattagcagctttctcagctgctttcttttggcgctggggtcctctactctccc

SEQ ID NO: 649 - DNA, 80 bp, Rat
ctctcaccaggccggccacagaagccagtccagcaggaaccaaagctgccagtaacctcacacttctgattcactctccc

SEQ ID NO: 650 - DNA, 80 bp, Rat
ctctcaccgttcgatatgaaatatggaaataaggaaaccttcgaattcttttaagacagaggcagtaaggcaactctccc

SEQ ID NO: 651 - DNA, 80 bp, Rat
ctctcacctgttgttgttgaagtaaacatggtgtgataataaaaactgacacacaagatcgaaaaccaactgactctccc

SEQ ID NO: 652 - DNA, 80 bp, Rat
ctctcacctccatgtttatgggatcaacaggtccaatgctgtttacataaacatcagtttcaattactgtggactctccc

SEQ ID NO: 653 - DNA, 80 bp, Rat
ctctcaccccattatatacatactatacatattgggtcataaattagcagttattagtcagactgtacaagcactctccc

SEQ ID NO: 654 - DNA, 80 bp, Rat
ctctcaccatattgctgaattcctggtgtcaccaacgctgcctgcaacctcattcatttcattgtgaacatcactctccc

SEQ ID NO: 655 - DNA, 80 bp, Rat
ctctcaccacgtcaatgtggatgcggcctcttctccaggagccagacttgcactcctcatagcagcagaagaactctccc

SEQ ID NO: 656 - DNA, 80 bp, Rat
ctctcaccacgccgaccgcaaaggagctggctgcagtgtcccgtgaggagagggcgatgcggcagacgaaagactctccc

SEQ ID NO: 657 - DNA, 80 bp, Rat
ctctcaccatttagaagaaaaaccgtcctttcctccaacttgatcttgaagctttctgaaatatttgccacaactctccc

SEQ ID NO: 658 - DNA, 80 bp, Rat
ctctcacctggaaaggtaaactatccagtacacaaggttgaatcctgcaaatgctactgggaagagaattcgactctccc

SEQ ID NO: 659 - DNA, 80 bp, Rat
ctctcaccttgagcgaggagattcagacacacaaaggccaaatagagactgtagctcatattgaaggagctgactctccc

SEQ ID NO: 660 - DNA, 80 bp, Rat
ctctcaccacaggggaagatgccctgtgacttaaccatggatcctgtctccagtctccggtcagcgcttctcactctccc

SEQ ID NO: 661 - DNA, 80 bp, Rat
ctctcacctgaaagctaattcttcaacattaacaggttcattttccacagtttcaaaaatccctccgatcctactctccc

SEQ ID NO: 662 - DNA, 80 bp, Rat
ctctcaccgccgtcataggtccaagtgcccagcttcatgctgcagttctgctcatcgaagggaaagtgggtgactctccc

SEQ ID NO: 663 - DNA, 80 bp, Rat
ctctcaccggctgcagcacccaggttctggcagagggcggctgcggtccaagttccatgccgatgcctctgtactctccc

SEQ ID NO: 664 - DNA, 80 bp, Rat
ctctcaccgggtggaggacaggagccaggcgatggacagggactggccttctcaacctctgatgtcttcaagactctccc

SEQ ID NO: 665 - DNA, 80 bp, Rat
ctctcaccgcaccacgtccgggagccaaacagattcagcagtgacacggagagactcgatgccatcgtgttcactctccc

SEQ ID NO: 666 - DNA, 80 bp, Rat
ctctcacctgccgatgctgtgtggccggtggtgatggtgatagtggtcttggtacacattgctgtccttcagactctccc

SEQ ID NO: 667 - DNA, 80 bp, Rat
ctctcacctacacatggctgctggcacatgtcaccacattaacaactctaggaacacaacacacagctagttactctccc

SEQ ID NO: 668 - DNA, 80 bp, Rat
ctctcacccagttattcaatggatcagtaaacaggtagcttctctcccgtgaggtcgataaaaacccattttactctccc

SEQ ID NO: 669 - DNA, 80 bp, Rat
ctctcaccttccaaacatgccgatgtacttcccttttttctgattcttttgagaaaaatttctgtggtatgtactctccc

SEQ ID NO: 670 - DNA, 80 bp, Rat
ctctcacccagaacatttacaaaaatgtcttccagggccccttgggtgttatctctcccagggacccacaggactctccc

SEQ ID NO: 671 - DNA, 80 bp, Rat
ctctcaccatcaaagggtaaaatttgtctttatcggataacagccattgatctgaccacagagctcagaactactctccc

SEQ ID NO: 672 - DNA, 80 bp, Rat
ctctcacctgagcaggtgggggttgtctgctttgctgggcttgatgctgacaggctggaagcccgaggtgagactctccc

SEQ ID NO: 673 - DNA, 80 bp, Rat
ctctcaccaacttgccctggggaatggctccatttggtaccaacaggctgacccctgtgccgggaatggtcaactctccc

SEQ ID NO: 674 - DNA, 80 bp, Rat
ctctcacctgaaaatcacagtacagtttgaatttttttcagttaatcaaggatatttaaaacgatattcactactctccc

SEQ ID NO: 675 - DNA, 80 bp, Rat
ctctcaccatagatgagagaccccttccccagcctcgagccccacaccccacctctgctccttctgaactctactctccc

SEQ ID NO: 676 - DNA, 80 bp, Rat
ctctcaccctcagtccgagcccactatgtgtggctctctcggggtagatcctgatgaatcttgtggagagagactctccc

SEQ ID NO: 677 - DNA, 80 bp, Rat
ctctcaccattgtttaaagctcctcccggagggtcttcacttctacatgacagccatccaccgggaatccacactctccc

SEQ ID NO: 678 - DNA, 80 bp, Rat
ctctcaccgggcggagggcgcaagtagcaccccgggggcgcccgtggccccatctgtcctgaggttaggtcgactctccc

SEQ ID NO: 679 - DNA, 80 bp, Rat
ctctcacctgccagggccactgagaaatgccagtcactgcttcgaaggacagcagtctggagaacttgcatcactctccc

SEQ ID NO: 680 - DNA, 80 bp, Rat
ctctcacctgttgcacagccctgtacagagctctggacacagccaccctgatcccagttatgttttgcagtgactctccc

SEQ ID NO: 681 - DNA, 80 bp, Rat
ctctcaccttcaaagtcgttgtaatcggctcatacgtaattttgaaagggttcgctaacataaacccagattactctccc

SEQ ID NO: 682 - DNA, 80 bp, Rat
ctctcaccagctaatgacaacttctatgtgcgatggtttgcagcgcatacgtcacagctgaccgcgtgccagactctccc

SEQ ID NO: 683 - DNA, 80 bp, Rat
ctctcaccatcttgcttctctttttttctcccccgtaacttctaatggcgctctttttggtgatagctttgcactctccc

SEQ ID NO: 684 - DNA, 80 bp, Rat
ctctcacctgcatgttatgcagtgcataagccatggaatacactgcatcaataacaaattgaacctttccttactctccc

SEQ ID NO: 685 - DNA, 80 bp, Rat
ctctcacctggcacccacactcaaccttgctacttggcgtcctctgcgttctcgttctggggtggggctgccactctccc

SEQ ID NO: 686 - DNA, 80 bp, Rat
ctctcaccggtgggtaagtggctctgggggcaggcatggggtgggtgttattgcagtgctcccagccaacttactctccc

SEQ ID NO: 687 - DNA, 80 bp, Rat
ctctcaccctgcaccaggcccccggttctctgtgctcttgggaaagggtttgatgaccgccgtttggttgggactctccc

SEQ ID NO: 688 - DNA, 80 bp, Rat
ctctcacccagggagacacagaacatgattaaccaaaacatcagaatcagaagggcacacagctgaaggttcactctccc

SEQ ID NO: 689 - DNA, 80 bp, Rat
ctctcaccgggggtgtcccagggcgtcagccagtctttggcacctggcactgtggctccgctggccggcgcaactctccc

SEQ ID NO: 690 - DNA, 80 bp, Rat
ctctcaccagtggtcagagctccaggaggtctcagatccttttcccaaagtactgaaggccagatttctgtgactctccc

SEQ ID NO: 691 - DNA, 80 bp, Rat
ctctcaccccatgtcctggggaaggggctgttaattagggcactaaaatcagcctagaaagttgtagcacatactctccc

SEQ ID NO: 692 - DNA, 80 bp, Rat
ctctcaccagagtagaagagaatgggggagatggccaccaccacaatgagccataccagcacactgacataaactctccc

SEQ ID NO: 693 - DNA, 80 bp, Rat
ctctcaccacacagcggaacgggtcagggtccggcgggatgcgcatatctgggcgatgacacagacgttcagactctccc

SEQ ID NO: 694 - DNA, 80 bp, Rat
ctctcaccgagccgttgatatcatatcagaggatatcagcccctttgaacaagctttggtccctctacagctactctccc

SEQ ID NO: 695 - DNA, 80 bp, Rat
ctctcacccccagcatgttggagcagggtgcatctgtgacccggcaaccaaagagctccagtcgaagggctaactctccc

SEQ ID NO: 696 - DNA, 80 bp, Rat
ctctcaccggtctcgttggtctccagaggaaagatattttcagaagggatgtgggaccatttcttctgtcgaactctccc

SEQ ID NO: 697 - DNA, 80 bp, Rat
ctctcaccgagattgttcactagagtggccagctctgacccagtgaatttattggcaccatacactgcttcaactctccc

SEQ ID NO: 698 - DNA, 80 bp, Rat
ctctcaccttgcagctgcatgctttgggatgaaaccacacagatgaccaccagcaggagaatgttgaaggccactctccc

SEQ ID NO: 699 - DNA, 80 bp, Rat
ctctcaccctgaagttacaactaatttctacttctgtctgaggaggtcaccacattagaattacaatgcttaactctccc

SEQ ID NO: 700 - DNA, 80 bp, Rat
ctctcacctctcacctggccctacgagagggcagaggatgagcaagaagatggggccattaaaatgttagctactctccc

SEQ ID NO: 701 - DNA, 80 bp, Rat
ctctcaccaggaaaagtcaggagagcaagtttcatttcccatcaccacatggaagggtcttcaagccttgttactctccc

SEQ ID NO: 702 - DNA, 80 bp, Rat
ctctcaccgagcgcaggcactgggaccgccacaggaaccctgtctttatgatgcagtccagggtctgagagtactctccc

SEQ ID NO: 703 - DNA, 80 bp, Rat
ctctcaccgaataatgaaataagaacctcttagcattatctttctcagtttgctcctgaggcttctggaaaaactctccc

SEQ ID NO: 704 - DNA, 80 bp, Rat
ctctcaccggccgggaaaccatccatctacagcgagtgatgaaaggctgagtgtaccttgacccagcgccctactctccc

SEQ ID NO: 705 - DNA, 80 bp, Rat
ctctcacccctcatatctgaagtgccttccagtctggccttcacctgaccactctgcccttcatcttccctaactctccc

SEQ ID NO: 706 - DNA, 80 bp, Rat
ctctcaccaaagagggcattggcactgccgttgagttgagccagttgactcttgatctggttcatgaggttgactctccc

SEQ ID NO: 707 - DNA, 80 bp, Rat
ctctcaccacctgggagtagataaggtacagcccatctgctggtaccaccagttggttgtctttgagatccaactctccc

SEQ ID NO: 708 - DNA, 80 bp, Rat
ctctcaccgacgcacccccgatttttgcccctctggcctctgcgacctttccctctggaattctctgggctgactctccc

SEQ ID NO: 709 - DNA, 80 bp, Rat
ctctcaccggggaggacactccttccccgaactgatgggtttcgggaaggggaacgttttcggacattcgagactctccc

SEQ ID NO: 710 - DNA, 80 bp, Rat
ctctcacctgtgggtttcttttcgaaaacgaaactttctcagcttttccactaaatcttcagcaacatcacaactctccc

SEQ ID NO: 711 - DNA, 80 bp, Rat
ctctcaccttctgaccagtcttaactactcttccctcctcaagtgaggcaatcaaaacccatggaggcatgcactctccc

SEQ ID NO: 712 - DNA, 80 bp, Rat
ctctcaccgtttaaatattaaacagggatgtcttgtcccccagaagggagaccagatgctgcctgggcccctactctccc

SEQ ID NO: 713 - DNA, 80 bp, Rat
ctctcacctgaggttgccccgtagaccctgcttgtatagcttcaggcgggtctgcacacatgtcggcctctgactctccc

SEQ ID NO: 714 - DNA, 80 bp, Rat
ctctcaccacaggcacagtcctgccttgaaagccatctgactgcgtctgtttgcccccccacctccctcatcactctccc

SEQ ID NO: 715 - DNA, 80 bp, Rat
ctctcaccctaggatactgtacggacagttttccttcttggtcccgctgaccttgccgttcttttcaatcttactctccc

SEQ ID NO: 716 - DNA, 80 bp, Rat
ctctcacctgacatactccactttggccaccttgacactgcggtggtggacccttgagggctggcacttgacactctccc

SEQ ID NO: 717 - DNA, 80 bp, Rat
ctctcaccgcgagagcaggatgtctagtgagctgcctcagagccttcctgaggaagcttgcccctgagagatactctccc

SEQ ID NO: 718 - DNA, 80 bp, Rat
ctctcaccgaagcgtgtcaacaagctcccctccgcacagagtctcgctggggcggtaagcagcgatgcagcaactctccc

SEQ ID NO: 719 - DNA, 80 bp, Rat
ctctcaccgacagcaggctgcctttgttgcaaagtgcagggcgaggtttctgcaacggactccaggtatcgcactctccc

SEQ ID NO: 720 - DNA, 80 bp, Rat
ctctcaccaggatcttgcggtgacgtggcattttctgttcctcgggaggctcctcctacattctgtaggtctactctccc

SEQ ID NO: 721 - DNA, 80 bp, Rat
ctctcacctggtgttatcccacagcatgtcaacaggacagagtttcttggaatgaggacattgatcttcttcactctccc

SEQ ID NO: 722 - DNA, 80 bp, Rat
ctctcacctcaagcgaaggtggaaccgttccagcccacgttggctgcgttcatgtcgtaatagttgatgtagactctccc

SEQ ID NO: 723 - DNA, 80 bp, Rat
ctctcaccgcagggtgaatgacgagcaggaccggctagggatgcgggcggagctgcggagctggaagctcatactctccc

SEQ ID NO: 724 - DNA, 80 bp, Rat
ctctcaccgtccgtaggacaggccggtaagaaggtaacaaacaaccccgtttgatgagggttgggagtggtgactctccc

SEQ ID NO: 725 - DNA, 80 bp, Rat
ctctcaccccgaacggtaccgcgtcctgcacaccgaaatagctcccaacttcacctaagggagccatcagagactctccc

SEQ ID NO: 726 - DNA, 80 bp, Rat
ctctcaccgtgaatcgctgacgcataggtattatagctgttttcttggaatctctccctgaacttacagtcaactctccc

SEQ ID NO: 727 - DNA, 80 bp, Rat
ctctcaccaatatacagatgagctactcgtaataaaccatctgcgttgatcaatccagtgtagccccagccgactctccc

SEQ ID NO: 728 - DNA, 80 bp, Rat
ctctcaccaaggaaccgccatctcagaagtagccatcctctgaaggctccttgagctcagatcctcctcagtactctccc

SEQ ID NO: 729 - DNA, 80 bp, Rat
ctctcaccagccagcagccgtccatcttccttcatagccaggtaccggttcgcacacactcccttgatggacactctccc

SEQ ID NO: 730 - DNA, 80 bp, Rat
ctctcacctcacacactgagtactctcctcggtgactcttatgctctgcatagcgtttcctccgtggtgatgactctccc

SEQ ID NO: 731 - DNA, 80 bp, Rat
ctctcacctccaacccagaacagtaaatattgcacacatctacaaataaatttggatttttactgaggggggactctccc

SEQ ID NO: 732 - DNA, 80 bp, Rat
ctctcaccttttttcctcctcaacctgagcggttcccaagcctggacgtcaggcacggcctgttcggctgagactctccc

SEQ ID NO: 733 - DNA, 80 bp, Rat
ctctcaccccagcctccttgtactgatactcggccccagggagctcgttgttctgcaatggcaacaagatccactctccc

SEQ ID NO: 734 - DNA, 80 bp, Rat
ctctcaccagccccgagggcggcatgggggaggcggcgccccacggaacttcggagagcgcgaaccccgggcactctccc

SEQ ID NO: 735 - DNA, 80 bp, Rat
ctctcacctccttctttcttctgcttcttcatattccctgaagaatcgtttccgaagcaggacggcggtaatactctccc

SEQ ID NO: 736 - DNA, 80 bp, Rat
ctctcacccaggtaccactgggtgcgacagaacagtctcctcacccttatatcccctccttccatgtagtcaactctccc

SEQ ID NO: 737 - DNA, 80 bp, Rat
ctctcaccgctaggaatgttgtcgataagagtcctcacgcacgtagaattcggatgcaaacaaattgcattcactctccc

SEQ ID NO: 738 - DNA, 80 bp, Rat
ctctcaccgagaaacaagatggctttctggccgtagtgagtccgaggaccgcgcttacaactcccgttcttcactctccc

SEQ ID NO: 739 - DNA, 80 bp, Rat
ctctcacctccgtgtctggtggaggtgagtcacctcaatggccagcccatagttgggttgcttttcccgggtactctccc

SEQ ID NO: 740 - DNA, 80 bp, Rat
ctctcaccatgtgagaaactcatcagtagggacagaacttaaattgaagaagaagcgtcgggaagttttcccactctccc

SEQ ID NO: 741 - DNA, 80 bp, Rat
ctctcaccttgcagcagcccgcacaccgcattaggggcacacaggacggcttgaagatatactctatctcatactctccc

SEQ ID NO: 742 - DNA, 80 bp, Rat
ctctcaccaatgtgacaataaaataaattaaaaaattaaatagtattaaatcaataggattaaattaaataaactctccc

SEQ ID NO: 743 - DNA, 80 bp, Rat
ctctcacccaggtgaaacctccaaccccagctgtgatcatttcctccttgggctgtgtccaattccatcccaactctccc

SEQ ID NO: 744 - DNA, 80 bp, Rat
ctctcaccagctgacctgttggaccttgtggtctttgtggctcaattggtcttaagaagactctcattgaagactctccc

SEQ ID NO: 745 - DNA, 80 bp, Rat
ctctcaccagcgactgactgcgccgatccggtctatcttctgcccaaagcagcttgaactatgtgccatcttactctccc

SEQ ID NO: 746 - DNA, 80 bp, Rat
ctctcacccaagccctccagcagctgcctgatcctcacaggtggcacctgtggctgggaaaaggatggcctgactctccc

SEQ ID NO: 747 - DNA, 80 bp, Rat
ctctcaccaagggtgggtgaatggcaatgtccattgttccttattctttcatttcaccgaaggagcagtagcactctccc

SEQ ID NO: 748 - DNA, 80 bp, Rat
ctctcaccttccacgttatttgagtccttcttgaggcagtgaaacaggttataaaaggcaaagtatctagtgactctccc

SEQ ID NO: 749 - DNA, 80 bp, Rat
ctctcacctcgagttggcagacctctgcagctccagcctcatctcgtcctgctcagctgcctggggcaaatcactctccc

SEQ ID NO: 750 - DNA, 80 bp, Rat
ctctcaccgtgaagatggcgtctgcatgccgccgcaccctgaagggaggtgaggggggcagtgagcagtgggactctccc

SEQ ID NO: 751 - DNA, 80 bp, Rat
ctctcaccacctaggagaggcactcaccacccctcccccaacacctggccacagtgatcctggttgcaggtaactctccc

SEQ ID NO: 752 - DNA, 80 bp, Rat
ctctcacctattagtcttcttcaactttgatttgtcaaatgtttcaacttctgataagtctggtttatcactactctccc

SEQ ID NO: 753 - DNA, 80 bp, Rat
ctctcacctgcaggtgctgggctcggagcacagcattggcaaacagactggacaagggcatggcaggtaaagactctccc

SEQ ID NO: 754 - DNA, 80 bp, Rat
ctctcaccggtgctcgtgcagaaggcgagcccacgccgcccgggacttggtgtccacacgcaggtcccgaagactctccc

SEQ ID NO: 755 - DNA, 80 bp, Rat
ctctcaccagcctctggtttagcggggtaggcgtccaccagcgctcccaggcaggcgagcaggacaagcagcactctccc

SEQ ID NO: 756 - DNA, 80 bp, Rat
ctctcacctcctatgggaaatttagctgagttttcttcatccccaatttccctcttttcctgtgtggactcaactctccc

SEQ ID NO: 757 - DNA, 80 bp, Rat
ctctcaccgacaccgcactgtatacgggatttgctccaatatggcctgggagccaaaaggccaggaagaggaactctccc

SEQ ID NO: 758 - DNA, 80 bp, Rat
ctctcaccctgtagtcagtgttacacttgccacacttgcagctcagggcaacggggtaggagaaataaggagactctccc

SEQ ID NO: 759 - DNA, 80 bp, Rat
ctctcaccaagaagtcctaagagaaccactgctttctcatctgcagacattgactttataaaccttcgacacactctccc

SEQ ID NO: 760 - DNA, 80 bp, Rat
ctctcacccagcgtcaatgtcacactcggatgttgtggatccttctgggccagtgcattacatcttcttctgactctccc

SEQ ID NO: 761 - DNA, 80 bp, Rat
ctctcacctgagcttgctgagctaactgctctgccctggccatttccaagacttccctcagaaggtggaaggactctccc

SEQ ID NO: 762 - DNA, 80 bp, Rat
ctctcaccgcaggcgctggatgctgctagacactgggaagatgtgccaagtgctcttccgagcatctaccacactctccc

SEQ ID NO: 763 - DNA, 80 bp, Rat
ctctcacctagagccagcactaagactagcatgcacacacacagtcgaggcatcttgtctgcagagctggggactctccc

SEQ ID NO: 764 - DNA, 80 bp, Rat
ctctcaccaagcaagatggaaactggaaaggagaggagggacctagttgccaaaataggggtggtggtttgtactctccc

SEQ ID NO: 765 - DNA, 80 bp, Rat
ctctcaccgaatgcatccttttttgctttactgttgctgaagaagttagtgatcaggtgcgattcgatgacaactctccc

SEQ ID NO: 766 - DNA, 80 bp, Rat
ctctcacccaatattttcaggtggattcatttcctcaaaggaaatgattttgtttttacaggagagggtagaactctccc

SEQ ID NO: 767 - DNA, 80 bp, Rat
ctctcaccgaaacagtgtacgtctgtctatagcatggtgtttcagaggacactgcatctaactgaccacctaactctccc

SEQ ID NO: 768 - DNA, 80 bp, Rat
ctctcaccgcaatccttaatcttttggggtctgtcagcctcaaagaacaggtcattctcctcactgtcgaaaactctccc

SEQ ID NO: 769 - DNA, 80 bp, Rat
ctctcaccctttagttttacaacagttactctgatattgctgatgaaatttccagcgtcttccaagtgaaagactctccc

SEQ ID NO: 770 - DNA, 80 bp, Rat
ctctcaccgaagtctcttgcggagagaaacttcatagctgttcctgaagggcagatggagttgactttcgttactctccc

SEQ ID NO: 771 - DNA, 80 bp, Rat
ctctcaccagacgatgtgaccggcagcagcacaaggatcaggggaggaattccaaagatatatctaaaagaaactctccc

SEQ ID NO: 772 - DNA, 80 bp, Rat
ctctcacctaaaacttgatcatttctgacaaggcttggcaacccaagtaacccttaaagtcctgcagtaaggactctccc

SEQ ID NO: 773 - DNA, 80 bp, Rat
ctctcaccatagcggaaaagttgcttggagtaattgagcagttttgatataaactgggctacttcgattttgactctccc

SEQ ID NO: 774 - DNA, 80 bp, Rat
ctctcaccttgttagaagacagagtgctgtttgcaaggtagagcacgtttcttactgtttcattaagagtcaactctccc

SEQ ID NO: 775 - DNA, 80 bp, Rat
ctctcaccctggctgtggtttactgcaccctttgggcgtgaaccccatcattctcctgctgctcgttgtagaactctccc

SEQ ID NO: 776 - DNA, 80 bp, Rat
ctctcaccctgcagcctgctcagagccaccacctctgtggagtagagcgaggcttccaggacgccatccaggactctccc

SEQ ID NO: 777 - DNA, 80 bp, Rat
ctctcaccttgcagtttggcagataaaatttgtagatctcatctcctgctttctgttgagcggcacgtaatcactctccc

SEQ ID NO: 778 - DNA, 80 bp, Rat
ctctcaccgggccaagtcatcaggaagctgcctgacaaaggaaggtccttgtccttgtctttatgggctctcactctccc

SEQ ID NO: 779 - DNA, 80 bp, Rat
ctctcaccacactggcatctgttcccttggtctgtggtctttgtgggaagtaagtctttcaaggatcgcttaactctccc

SEQ ID NO: 780 - DNA, 80 bp, Rat
ctctcacctcacatagagctcaaatgacagccccatgctcctgccagggtcctggaggctcccaccaggtttactctccc

SEQ ID NO: 781 - DNA, 80 bp, Rat
ctctcaccccggcacggcacaggcggccagcagcaggaggaccacgctgatcaccattttttccgagtctggactctccc

SEQ ID NO: 782 - DNA, 80 bp, Rat
ctctcaccagcttcttcaggacttcctgcagcgcttcaatctgcaacacagcgccgggagcccgaagttgccactctccc

SEQ ID NO: 783 - DNA, 80 bp, Rat
ctctcaccttttccactggagaccatcctcaccacagcattgtcgcagctaattttcatctgggcagggccaactctccc

SEQ ID NO: 784 - DNA, 80 bp, Rat
ctctcaccggatggagaggaccctctcatctccttggagggtctccagacactcagggctgacaggcatgggactctccc

SEQ ID NO: 785 - DNA, 80 bp, Rat
ctctcaccgtcctttggatagaggtgaagaacttggacgtggctgtttcaggcagctgaagtcaaaaggaccactctccc

SEQ ID NO: 786 - DNA, 80 bp, Rat
ctctcacccagaagccagttcacaaagtcttgttggcggatcttgtccatggcgatgctgtaatcactgatgactctccc

SEQ ID NO: 787 - DNA, 80 bp, Rat
ctctcaccggagtatgtggaatggactgtctgggtcttctggacgggcagcccttggtaagagacttgggacactctccc

SEQ ID NO: 788 - DNA, 80 bp, Rat
ctctcaccaaaaaggaggcggacaaacaggtttctgtaagagtgagcaggccaaaggtgagagaggaggatcactctccc

SEQ ID NO: 789 - DNA, 80 bp, Rat
ctctcaccgtagatgcgtttgatggcgttggcttcttccttatccaggatgcctttctccacataagcatcaactctccc

SEQ ID NO: 790 - DNA, 80 bp, Rat
ctctcaccttcgtactcgtggacaggtggggggaaatgtgcaactgagaacgaggacttaattgtaagagcaactctccc

SEQ ID NO: 791 - DNA, 80 bp, Rat
ctctcaccacttcccgatgtgggggctcctctttaggcttggggtgcattaaggtaaagggcagttccacagactctccc

SEQ ID NO: 792 - DNA, 80 bp, Rat
ctctcaccacagcttgccaccctagggagcagggctggggtggcagagctgaagtcaaaagattaatgcccaactctccc

SEQ ID NO: 793 - DNA, 80 bp, Rat
ctctcacctaaactcttcgacaactatgtcctccgagctggtagccactgctctctcaccgtgaggtggcttactctccc

SEQ ID NO: 794 - DNA, 80 bp, Rat
ctctcaccaccgttgggtccgaacacattcagctccttgaaacattctgtttctatcatctcgttttgccatactctccc

SEQ ID NO: 795 - DNA, 80 bp, Rat
ctctcaccccttcaggggaattcttgggtaaggcactcgcccgaaggaatagatttcccagagaaggattccactctccc

SEQ ID NO: 796 - DNA, 80 bp, Rat
ctctcacccgagctacataattgcttgggatatagccttctttcttggtagccagggaacgggccttccaccactctccc

SEQ ID NO: 797 - DNA, 80 bp, Rat
ctctcaccgacctctttgggccggatctcttcagggtcagcgtaaggactctcatatacctcggtgtccatgactctccc

SEQ ID NO: 798 - DNA, 80 bp, Rat
ctctcaccggtaatgcttaatcatgtcactgatacagggaaaagtgatcctcggagagatgtaataaccaccactctccc

SEQ ID NO: 799 - DNA, 80 bp, Rat
ctctcaccaaccccactggcaagcagccctctgtataggtcataaaatctccctttccaggtagggctgcatactctccc

SEQ ID NO: 800 - DNA, 80 bp, Rat
ctctcacccaggcttaaaatcgctaagatcaccaaggctagaacacagaaggcaattccactgaaagcatagactctccc

SEQ ID NO: 801 - DNA, 80 bp, Rat
ctctcaccgatgaaatcaggagcatagaagtctcaaatatctcggctccataatgcttggttaaggtttccaactctccc

SEQ ID NO: 802 - DNA, 80 bp, Rat
ctctcaccctgtctgcttcacagagttagcatttccaggaatatcaccattctgatgtgcaggagatgtggaactctccc

SEQ ID NO: 803 - DNA, 80 bp, Rat
ctctcaccggttgcggcccctacgcaccacgatcaaattggacttttccttgggtctggggacgcagcaagaactctccc

SEQ ID NO: 804 - DNA, 80 bp, Rat
ctctcaccttacacatgatctgtcaccaaggacaccacacgacttgaaaagaactcggctattctaattgctactctccc

SEQ ID NO: 805 - DNA, 80 bp, Rat
ctctcacctaaataccactgctcgtgtgatttctcatctacgggaaagggggtctacatcaacagcctcttgactctccc

SEQ ID NO: 806 - DNA, 80 bp, Rat
ctctcaccgcggtcatagcccttccattccagaaccgtctaccagagctggggctagcagggatagataaagactctccc

SEQ ID NO: 807 - DNA, 80 bp, Rat
ctctcaccgagcctgttcaacttcaatcctcttgtgagggttaaatgtcaacattttatccagtaaatccagactctccc

SEQ ID NO: 808 - DNA, 80 bp, Rat
ctctcacctagatacaagtgcatatgccgcggacgactatgggtgggttaggtttcgcccgcatacttttgtactctccc

SEQ ID NO: 809 - DNA, 80 bp, Rat
ctctcaccatgggactggacctctctctctctctgctttgggatttagatccctgtcaatacctgatgctacactctccc

SEQ ID NO: 810 - DNA, 80 bp, Rat
ctctcacctcgctcacagcgaggacagggcgcagagggggcaatgctggctgtggaatgtccccctgcagggactctccc

SEQ ID NO: 811 - DNA, 80 bp, Rat
ctctcaccaggtgcgtggataggactgctgggaagtcttcacacgcccggcccagggcttccagaagacaggactctccc

SEQ ID NO: 812 - DNA, 80 bp, Rat
ctctcacccacatctgaacatagacaagcatgggagaggcctgaaagcaacatggcatgccactgggcagagactctccc

SEQ ID NO: 813 - DNA, 80 bp, Rat
ctctcaccccacagcattacaggttaaagggtctcacatcctgcggcaaggagtaccgactggcccctacttactctccc

SEQ ID NO: 814 - DNA, 80 bp, Rat
ctctcaccgaagcagaaaatagaagcaacgaggtcttgaatatatgagactctgcctctctttgttttctgtactctccc

SEQ ID NO: 815 - DNA, 80 bp, Rat
ctctcaccaaaaactttgaccaggaacatcagcatctgactttaaaaattacaaatactgaagcattttggtactctccc

SEQ ID NO: 816 - DNA, 80 bp, Rat
ctctcaccaatactgcacacacatcagcaccttggcctgaggctgcaggtccagccagaactcagccttgccactctccc

SEQ ID NO: 817 - DNA, 80 bp, Rat
ctctcaccttggattttgctggacatctcaatgacagccttcactaggcccgtcacattttcatataccttgactctccc

SEQ ID NO: 818 - DNA, 80 bp, Rat
ctctcacctttgaattcttcctggttgatctgcttcacgattgcttcatccacaagtgtaagtattggctctactctccc

SEQ ID NO: 819 - DNA, 80 bp, Rat
ctctcacctctagaaccccacacggagtggggtttctgggaagagttggaacagcatccagatgttgggggtactctccc

SEQ ID NO: 820 - DNA, 80 bp, Rat
ctctcaccatgtttccgagatgaagaaatataagcaattccatcagtttcttctgaaggaaggtctacaccaactctccc

SEQ ID NO: 821 - DNA, 80 bp, Rat
ctctcaccaggaaaggagtgcggctgtcgggggtcccagatgccccgactgaggggggagggctccaagggaactctccc

SEQ ID NO: 822 - DNA, 80 bp, Rat
ctctcaccaactggcagaactctatgatgagcactttgggtattatatggtgtactaaaaaattagacattcactctccc

SEQ ID NO: 823 - DNA, 80 bp, Rat
ctctcacctgcatttgacaagcttgaagttcttccctgggttgaagctgttgctgtagaagcagaccttgagactctccc

SEQ ID NO: 824 - DNA, 80 bp, Rat
ctctcacccggtaagcatcgcgcagctcttttttccattgcaccagctcagggtcactatcacactgcaggaactctccc

SEQ ID NO: 825 - DNA, 80 bp, Rat
ctctcaccttcgagtgtggataggccagccccaaggatgtcctagttcctgaagggccacccggtgttgtggactctccc

SEQ ID NO: 826 - DNA, 80 bp, Rat
ctctcaccaatacagggtggcttgatggggactgttggggaccccgaacgatgaaagtgaatattctgccacactctccc

SEQ ID NO: 827 - DNA, 80 bp, Rat
ctctcacctgcggctggggcgccagggtcctggggattgggggtggggtaggggaaggatcaggaacgggaaactctccc

SEQ ID NO: 828 - DNA, 80 bp, Rat
ctctcacctttggggcacaggccctcacacttatggcagaaaatgctgctgccattacgggtgaagcctggaactctccc

SEQ ID NO: 829 - DNA, 80 bp, Rat
ctctcaccgtgacatcaaatctatacttgagggatgaccaccactagtctgactctcgggtgagacagacagactctccc

SEQ ID NO: 830 - DNA, 80 bp, Rat
ctctcacccactgccagcacatgactcctgcttatgtgggtgctggagggtgggaaaggggagccagcacaaactctccc

SEQ ID NO: 831 - DNA, 80 bp, Rat
ctctcacctgatgtcgatgatttttactgcgtattcttggcacgtaggtttgtgaatgcatctcctgaccacactctccc

SEQ ID NO: 832 - DNA, 80 bp, Rat
ctctcacccctgagccttctgtggccgtaggtcgtcatcggcagtgtcatagtcctcgatgtccttccagagactctccc

SEQ ID NO: 833 - DNA, 80 bp, Rat
ctctcacccctcccaaccaggcgcaagctgcattaaggagagggtggacgtcccacctctgcccaggcatcaactctccc

SEQ ID NO: 834 - DNA, 80 bp, Rat
ctctcaccgctgagcttggggtctgtggaaagggcctccctctgtcgcagagcagcagccgccagcagccctactctccc

SEQ ID NO: 835 - DNA, 80 bp, Rat
ctctcaccgagtcttggccacacatgggactgggtgttcctcctgggactgcaatggccaaaaagccaaatgactctccc

SEQ ID NO: 836 - DNA, 80 bp, Rat
ctctcaccgaagagggggggaagcgggcggttgttggggagctgggttccgagactccgcagttcatcaaagactctccc

SEQ ID NO: 837 - DNA, 80 bp, Rat
ctctcacccgcagctctcactgcagtggggccactggagcgcccgagcagtggaaatggacattctgccactactctccc

SEQ ID NO: 838 - DNA, 80 bp, Rat
ctctcaccggggtggtgcagggggagttctgagcctggctccacacagcagtctcgacagcagcagccagctactctccc

SEQ ID NO: 839 - DNA, 80 bp, Rat
ctctcacctcactgagaggaggatgtgctcttcggaaagcttccggaaagcttccgcgtcaacaagccgcgtactctccc

SEQ ID NO: 840 - DNA, 80 bp, Rat
ctctcaccggccttgagggaagagtagccgggggaaggggctgcgagaggcgccagctggaccctctgcatgactctccc

SEQ ID NO: 841 - DNA, 80 bp, Rat
ctctcacccaacttcttctcctgcagcaatgtccgtaccgagatagatgtctccgaaggagccgctgccgatactctccc

SEQ ID NO: 842 - DNA, 80 bp, Rat
ctctcaccaactatattacagatgcatgtgccagtatgagacatgcaagtgtgctgtatacagcagatgggcactctccc

SEQ ID NO: 843 - DNA, 80 bp, Rat
ctctcacccttgaaggcatccatgctcggcttcttggagtgggaggctcccaagtcgcagtcctcgatgttgactctccc

SEQ ID NO: 844 - DNA, 80 bp, Rat
ctctcaccgagcactagatcaggaaatgggagaagcaaggaaaggagaagaggacatgaggggtcggagttcactctccc

SEQ ID NO: 845 - DNA, 80 bp, Rat
ctctcaccgtgactctaggaggctaacattcccaccagcaaacgctactattccacggtaagagtggtcggcactctccc

SEQ ID NO: 846 - DNA, 80 bp, Rat
ctctcaccgagactggaagtaggggtgacttagggccgcctctgctgacatgcgactcttggattcatacagactctccc

SEQ ID NO: 847 - DNA, 80 bp, Rat
ctctcaccgatggggtcctgagaagattatcgggaaggagacttatttctttcattatatttgccaatctaaactctccc

SEQ ID NO: 848 - DNA, 80 bp, Rat
ctctcaccggccaaccaaggcctttattctcttctgagcgggtcatccagcaccccccggtgtatggggaggactctccc

SEQ ID NO: 849 - DNA, 80 bp, Rat
ctctcacctgaatcagcaatgttctcaggaagttctagctccttcctgctcagcagcctcttccggtcaaagactctccc

SEQ ID NO: 850 - DNA, 80 bp, Rat
ctctcaccagtggccactgtggggatctcgggctttgtacaccgtcccataggcaccgacaccaatttcagcactctccc

SEQ ID NO: 851 - DNA, 80 bp, Rat
ctctcaccgtggcattgagtttgggcacgacattcaccaaggatgttgtagctgggtacattgggtagggctactctccc

SEQ ID NO: 852 - DNA, 80 bp, Rat
ctctcaccggtacaagtaactcttaacgagcgaagaatccatgaactggccaggagggatagagtccaggtaactctccc

SEQ ID NO: 853 - DNA, 80 bp, Rat
ctctcaccaaagtgctcccagttggatatacaagtataattcacaatagaaaaaaagaaacgttaagattgaactctccc

SEQ ID NO: 854 - DNA, 80 bp, Rat
ctctcaccccctacctctcaaagggtgggagtgggacttcattgcacaaaatactgtgggagtgccacactcactctccc

SEQ ID NO: 855 - DNA, 80 bp, Rat
ctctcaccgaattgtctgaaaagaagtcttccgattggctgttggtcacaaagactgctaaaatccttttcgactctccc

SEQ ID NO: 856 - DNA, 80 bp, Rat
ctctcaccgtcagtcaaccccggcgagcttctgaacacgggtcctggaaactttctcgtctattcttgacatactctccc

SEQ ID NO: 857 - DNA, 80 bp, Rat
ctctcaccaacatccaacagtaattcagttcaggaaacttgaaaggaaatcttagtgttatgacaacatactactctccc

SEQ ID NO: 858 - DNA, 80 bp, Rat
ctctcaccctcgctgaaatattacaaccctaccccccttgtcccccgtcgctagtaactctccagtgtggttactctccc

SEQ ID NO: 859 - DNA, 80 bp, Rat
ctctcaccggccgtatcgcaacccgttcccctggccctgggcattatgcttttccatctttggcttgtctaaactctccc

SEQ ID NO: 860 - DNA, 80 bp, Rat
ctctcaccgcactatggttgccagtcccgtggttctcagtggtatgtgcggtgttcagggaattgaaaccatactctccc

SEQ ID NO: 861 - DNA, 80 bp, Rat
ctctcaccccagaatgattaaactctactgtagaaattatatctgcttctgctacgtcatcatcaactgctcactctccc

SEQ ID NO: 862 - DNA, 80 bp, Rat
ctctcaccaaacacgtggctccaagataacacatgttgccaaagagtcatgcatgccctgctcatgggctctactctccc

SEQ ID NO: 863 - DNA, 80 bp, Rat
ctctcaccgtgacgctcaccacaaagtcaaatcagagtgatggcaggacagtattggaagctagtcctaggtactctccc

SEQ ID NO: 864 - DNA, 80 bp, Rat
ctctcacctagacgccgcagaccaaatcctaacctgggcagtggcagcgcccaccacgctcatgcctcacagactctccc

SEQ ID NO: 865 - DNA, 80 bp, Rat
ctctcaccatcacgcccactgcggtagagccgctcctgtccatcccgttcctcaggtctgaaaagttacgcaactctccc

SEQ ID NO: 866 - DNA, 80 bp, Rat
ctctcacctgtaaacagacaacttgtgaggcaatctacagggttagcaagcctcactttagtttccggagtgactctccc

SEQ ID NO: 867 - DNA, 80 bp, Rat
ctctcaccccggttaatctcctcttccagcttgtccacaggcagtggtgggtactttctgttggtgctcgggactctccc

SEQ ID NO: 868 - DNA, 80 bp, Rat
ctctcaccaggctgctcctggcatcccaacttccaagtgtgaactagcctggtccctcccggaaggcagctgactctccc

SEQ ID NO: 869 - DNA, 80 bp, Rat
ctctcaccgtggaggcggtgtttcgaatctcaggagtgcattggccttgagggtccacaacaacattcagcaactctccc

SEQ ID NO: 870 - DNA, 80 bp, Rat
ctctcaccaactggttcattctaaagtggtcacggctgttgatgacaagaggctttgtattttatatggcacactctccc

SEQ ID NO: 871 - DNA, 80 bp, Rat
ctctcaccactgaagcacgattcatgagggccacgggttctttctgtcatatgccagcagaggtgccttctaactctccc

SEQ ID NO: 872 - DNA, 80 bp, Rat
ctctcaccgaattacagctgctggagacggggagccagggggaggccacctgggctcggcgcgctgcaatgtactctccc

SEQ ID NO: 873 - DNA, 80 bp, Rat
ctctcaccaggataaaactatcttgggggctcagagcagggtgcaaaagagtcgtttgtcgcggaagggattactctccc

SEQ ID NO: 874 - DNA, 80 bp, Rat
ctctcaccgtttgtgatctgagaaagagtcgcatctgcacatgctttacgggcatctctaatttggttcttcactctccc

SEQ ID NO: 875 - DNA, 80 bp, Rat
ctctcacctggcgtcccggatctggttccggagctgctcagcctcctgtctcagttgctccagctcactcatactctccc

SEQ ID NO: 876 - DNA, 80 bp, Rat
ctctcaccttatgaagggagaagctatgaggactgtggagaggagaggccctggaggaaatggggcagttctactctccc

SEQ ID NO: 877 - DNA, 80 bp, Rat
ctctcacccacagacctctttaataaaataatcattactcaaataatttaaaaatctacaaaagacacaagaactctccc

SEQ ID NO: 878 - DNA, 80 bp, Rat
ctctcacccacaatgacatatgcatcccccatggccatgcagtggccaggcagccagcagcctccatcctgtactctccc

SEQ ID NO: 879 - DNA, 80 bp, Rat
ctctcacctagcagtctgcgtctctgactgacagctatcctgcgtgtgtctgtacccaagagcagtttatcaactctccc

SEQ ID NO: 880 - DNA, 80 bp, Rat
ctctcaccggcctccaagcggcagagagtggggtaggcaggaggctccctgtaaacatttggacttgggtcaactctccc

SEQ ID NO: 881 - DNA, 80 bp, Rat
ctctcacctgtcaggatctgcatgtctgagttggtgaagctgcaggcagacaggtagttggtgtgcatagcgactctccc

SEQ ID NO: 882 - DNA, 80 bp, Rat
ctctcaccctcactgtctggagtttactggctgctaggatgcagtccccaagcctcggggatccttccatctactctccc

SEQ ID NO: 883 - DNA, 80 bp, Rat
ctctcaccgggtgtcgaggtgagggtggccggggtctggggctccggagccgagagaggaggcgcccgcgggactctccc

SEQ ID NO: 884 - DNA, 80 bp, Rat
ctctcacccgaaaggtccttctattaaagacacgatacagaacaactttggtcagaactctggtcaggtccaactctccc

SEQ ID NO: 885 - DNA, 80 bp, Rat
ctctcaccgagcacagcatgcttggatatgccattggtggtcttttatagaagtgagcttcaaaaactgggaactctccc

SEQ ID NO: 886 - DNA, 80 bp, Rat
ctctcacccaggaaaccttgaccgccggagagcgttgccgtctccagacgtgcgtgcgcgtgtgcgtgtgcgactctccc

SEQ ID NO: 887 - DNA, 80 bp, Rat
ctctcaccacagtccaagactgtgaggatgaaggtgcttatgtgatgtccctctgccgtggtggccggtgaaactctccc

SEQ ID NO: 888 - DNA, 80 bp, Rat
ctctcaccggcgcggccgggctgcggggcggcggggacggggcgggcggcctcgggcgggccgggaaagcggactctccc

SEQ ID NO: 889 - DNA, 80 bp, Rat
ctctcacctctctccttgtaatatcatacactagtaaagcccccgctgcacctctgtaatatgaccttgtgaactctccc

SEQ ID NO: 890 - DNA, 80 bp, Rat
ctctcaccctctaaactgagacaagctggcgacacggaccgtggcgactgcgctggcgaccgggccccagaaactctccc

SEQ ID NO: 891 - DNA, 80 bp, Rat
ctctcaccccaagaacttaaacaagaaatcgtaagtctcggacatggcggtctcgccatcctgcgccacagcactctccc

SEQ ID NO: 892 - DNA, 80 bp, Rat
ctctcaccttaagacatgggaagattcttaagtgtaaggaagtaataaaacacagaaagtgaccacaagaatactctccc

SEQ ID NO: 893 - DNA, 80 bp, Rat
ctctcaccctgtgcaaacttttctccttgctgcctggagatttctctgtctgtttcacagtccagcttatttactctccc

SEQ ID NO: 894 - DNA, 80 bp, Rat
ctctcaccgctgctaatgtcgtagactaaaaatattccctgggctcgccggtagtactgttttgtgatagtcactctccc

SEQ ID NO: 895 - DNA, 80 bp, Rat
ctctcaccactggttagccgtcctccctgcggggctgagggtttgtgttgtacaccagactcggcagcattcactctccc

SEQ ID NO: 896 - DNA, 80 bp, Rat
ctctcacccttggcagcggccagttcataggcagatttcaaggccaaactggtggacctgagatggagaggcactctccc

SEQ ID NO: 897 - DNA, 80 bp, Rat
ctctcacccaacaaaccatctccccgaagctgtggccacgggcatgtacagccacgcccagctttttacatgactctccc

SEQ ID NO: 898 - DNA, 80 bp, Rat
ctctcacctaacacagacttgcaacagcggggccaggcaggacgttggggccaattggccctggtctcaagcactctccc

SEQ ID NO: 899 - DNA, 80 bp, Rat
ctctcaccagctggccaatccggtctaagttatccaggaagtacttcactgactcacccagctggaactcgcactctccc

SEQ ID NO: 900 - DNA, 80 bp, Rat
ctctcaccgatccacagaggtgaataagagagcacaggctcccctgggcaagttagtcctccaggtcctcagactctccc

SEQ ID NO: 901 - DNA, 80 bp, Rat
ctctcaccggtctagcacctgctgagtggttgtgaaggctctgtaggtgcacaggaatacggtgacgtaggaactctccc

SEQ ID NO: 902 - DNA, 80 bp, Rat
ctctcaccaaaacaagttgggtcttaccccaatgaaaacccccacaccacagctaagtctagttttacatggactctccc

SEQ ID NO: 903 - DNA, 80 bp, Rat
ctctcaccgtggtagcagtttctcagggcccagctctgcactcagcatggcccgtgcccgactcagggcctgactctccc

SEQ ID NO: 904 - DNA, 80 bp, Rat
ctctcaccacagcccacagctccccctggtactcaagaaggaactgctggaactcagaaagggacacctggcactctccc

SEQ ID NO: 905 - DNA, 80 bp, Rat
ctctcaccgcaggtagacggctgaggcatcaggccggtctgtgttccctaggatgaacacggaggagtctttactctccc

SEQ ID NO: 906 - DNA, 80 bp, Rat
ctctcacctggatggagatcttcacaaaaagtgtggctgatggatgctggtctccattcttagacaagaggtactctccc

SEQ ID NO: 907 - DNA, 80 bp, Rat
ctctcaccgccccgaggagccctaacactcggatggcctctctccgagtgccctggttctgctccgtcttcaactctccc

SEQ ID NO: 908 - DNA, 80 bp, Rat
ctctcacctcttcttgcatgcaggtgtacacaaaatgggaggctggatggaggctagtgccgtctttggagaactctccc

SEQ ID NO: 909 - DNA, 80 bp, Rat
ctctcacccagtctttcaaatcgataggtgggggcgaaggtgatctcctcctcctcaaagtgcaggaagactactctccc

SEQ ID NO: 910 - DNA, 80 bp, Rat
ctctcaccaccacgtagtgttcgcgccttcctgtgagaacagggaggacatcattggatgtggcttccgttaactctccc

SEQ ID NO: 911 - DNA, 80 bp, Rat
ctctcacccctctctaggattctggagggtagggtgtaaccctgggctctgtccagcccagtggtacacagaactctccc

SEQ ID NO: 912 - DNA, 80 bp, Rat
ctctcaccaaaacaaagcttccatatctgagagtgagtgctcccggcttccgttctgaacccaacagctgtgactctccc

SEQ ID NO: 913 - DNA, 80 bp, Rat
ctctcaccgtctcagcactgtggcgagtggcttctctctagttacggagctctttttgaccttctttggctcactctccc

SEQ ID NO: 914 - DNA, 80 bp, Rat
ctctcacccatagtaacggttgaccaaaatttgccggagtgcctcaccccttttatggtcttccagttgcctactctccc

SEQ ID NO: 915 - DNA, 80 bp, Rat
ctctcaccgagccctggccaccaacagaatgaactctttccacccttccagggggttctctaatgtgggtccactctccc

SEQ ID NO: 916 - DNA, 80 bp, Rat
ctctcaccgttttcaggaatccgggcaggaacattagttgtgtcgaagttttcacattcaagaacttccacaactctccc

SEQ ID NO: 917 - DNA, 80 bp, Rat
ctctcacctgacagcagcagcgtcctacgcttcatgtagtacttgtgcagctgggaacctcggttggttttcactctccc

SEQ ID NO: 918 - DNA, 80 bp, Rat
ctctcaccgagcctgtttcagatgctccagcctccgttctgtctctgcatctgcctgggcctccctcagctcactctccc

SEQ ID NO: 919 - DNA, 80 bp, Rat
ctctcaccaataaatttgcctccttcctcatatgctgctatccttaaacaggccagagaaggcagaactaccactctccc

SEQ ID NO: 920 - DNA, 80 bp, Rat
ctctcaccctcctttgcccggaatgggtttgagaacaagttggtgacactactgagggtgttaacgaggcgtactctccc

SEQ ID NO: 921 - DNA, 80 bp, Rat
ctctcaccccgcgttgctggatgcggatcggggtcctcggctgccgccacaggtgctgggggggcggcttcaactctccc

SEQ ID NO: 922 - DNA, 80 bp, Rat
ctctcaccccaccgctgcgatcccaccgtgagtggtgctcactggtcagcaatcctgacgctcctgagctccactctccc

SEQ ID NO: 923 - DNA, 80 bp, Rat
ctctcaccgtcatgggatccaagggactccgtcccgctgatgtgccattgtccacatcgaaacatatccaggactctccc

SEQ ID NO: 924 - DNA, 80 bp, Rat
ctctcaccctcagaggacacgacaggctggcactgtggtgcagggagccagggtgtagacaaccccctcccaactctccc

SEQ ID NO: 925 - DNA, 80 bp, Rat
ctctcacccccaggaagtattaaaagccactgacgacacattgggagccaggaaagggcagtctgacgacacactctccc

SEQ ID NO: 926 - DNA, 80 bp, Rat
ctctcaccgaaggccgcttggcagcgatcccctctttcctaggctgctcgattgcaggagccctgagcagggactctccc

SEQ ID NO: 927 - DNA, 80 bp, Rat
ctctcacccgttggtgtgctccaggaaggctggtacgctccttgttgacccctggatcactgactgtggactactctccc

SEQ ID NO: 928 - DNA, 80 bp, Rat
ctctcaccggtgtcttcaagctccttagccagctgctcctcctggtctgtctggaccccaaagcgcgggatgactctccc

SEQ ID NO: 929 - DNA, 80 bp, Rat
ctctcacctcgccaagctcgccggcaaggcaaggcagcacggaactgctatttgcttcacggccttgcattcactctccc

SEQ ID NO: 930 - DNA, 80 bp, Rat
ctctcaccggcgatgatgcctgatgatttcaaaagccacatgagagaggtggaggcctttttgctgcattgcactctccc

SEQ ID NO: 931 - DNA, 80 bp, Rat
ctctcacctgaacctcatcgttaggaggaatgtgtttctcttctttaccacctgggcggactcccgctccagactctccc

SEQ ID NO: 932 - DNA, 80 bp, Rat
ctctcaccccaggcgctgctgctgttgtggggtcaccgctcccccagatcgcttggtggagccgcgggtcgaactctccc

SEQ ID NO: 933 - DNA, 80 bp, Rat
ctctcaccccggccatcaccatccttatccaggaccaagatgcttttgtccaccagctgctgcagctgccagactctccc

SEQ ID NO: 934 - DNA, 80 bp, Rat
ctctcaccaggctgtgatcagagatgactgcaggtgggattctgccatggtgcagcggctagtttgagagagactctccc

SEQ ID NO: 935 - DNA, 80 bp, Rat
ctctcaccgggtgtactcctggagcttaggttcatcatagggacggttggccttcttcaggaggtcggacagactctccc

SEQ ID NO: 936 - DNA, 80 bp, Rat
ctctcaccccgtccttatctgcgtttattatggttttgtctacaatctgctgtaactgcgtatctttcaggtactctccc

SEQ ID NO: 937 - DNA, 80 bp, Rat
ctctcacctgttccccaccatctggtaaatggcatccactatgtccagcatctcgtttctggtgatgtagccactctccc

SEQ ID NO: 938 - DNA, 80 bp, Rat
ctctcaccaaaaaaatatatatatttaattcagactaacggctaaaccgtctctgtgagtggttctctctcgactctccc

SEQ ID NO: 939 - DNA, 80 bp, Rat
ctctcacctgagatgggggtaaggaggcggccgtacctccttcttgaactggggactcggcttgaagtgaaaactctccc

SEQ ID NO: 940 - DNA, 80 bp, Rat
ctctcaccctggacgcgatccgctggcttctcgagctcaagttcggcggtggctgaggctggggagataaccactctccc

SEQ ID NO: 941 - DNA, 80 bp, Rat
ctctcaccgcagctcaaaaaaaaaaagtgaacaggaagtggtcactgaatattgctgctattactgccgttgactctccc

SEQ ID NO: 942 - DNA, 80 bp, Rat
ctctcacccggtgatgatggagaaaggataatgaaagacgcagttatgagctttcatcctaaaaggctctctactctccc

SEQ ID NO: 943 - DNA, 80 bp, Rat
ctctcaccctaactatgccaaacatgtgtaactcatgtaaaaattccacatcccatattggccacctcaagaactctccc

SEQ ID NO: 944 - DNA, 80 bp, Rat
ctctcaccatgagtggtataaaaattcacagtcaatgtacttcattttgttttacataagcaatcaaagggcactctccc

SEQ ID NO: 945 - DNA, 80 bp, Rat
ctctcacctccaggagacaaactgcacactctgcatgcaacagggcacgtttatgggaagatttatccacagactctccc

SEQ ID NO: 946 - DNA, 80 bp, Rat
ctctcacccaagttgtcgtgtgtcagtttccattagctgaaagaaggggtgagagggtcaaattcatttttgactctccc

SEQ ID NO: 947 - DNA, 80 bp, Rat
ctctcaccttttcccaaagctgcagtgtgaaaagactataaacagttgaccccatacacatgaatgggcttcactctccc

SEQ ID NO: 948 - DNA, 80 bp, Rat
ctctcacctacatccagaatgtcacaacagattaacttgagctcagtttcaaccatttgccggtactcccgaactctccc

SEQ ID NO: 949 - DNA, 80 bp, Rat
ctctcacccttgcgatgtcgtttgcggagcttgagtagtaggactgtcaaaaagatgataatgagcaggaagactctccc

SEQ ID NO: 950 - DNA, 80 bp, Rat
ctctcacctggggtgcaaagccaacagagatttcagtttatttacatagattctctagcttcttggtcttcgactctccc

SEQ ID NO: 951 - DNA, 80 bp, Rat
ctctcacccacagtaggtgactggctgtaggtccaagttattgtgtgctggggacatcggattcggggagagactctccc

SEQ ID NO: 952 - DNA, 80 bp, Rat
ctctcaccgtatgcagtggggagcatgggactgtactgggaagctggaggcactggagtatggacgccctggactctccc

SEQ ID NO: 953 - DNA, 80 bp, Rat
ctctcacctcgaacacatccaagtccactgaggggggatcacaatcaagaagctgtcgtaactctgcagggtactctccc

SEQ ID NO: 954 - DNA, 80 bp, Rat
ctctcaccctttttttttttcggggtctcgctgctgggagacggcgcggccccgtcctccgccttgggggacactctccc

SEQ ID NO: 955 - DNA, 80 bp, Rat
ctctcacctaggaagatagtcatgaccacaatgacgctgatcatgatcagagtgttcagcacggagttgaggactctccc

SEQ ID NO: 956 - DNA, 80 bp, Rat
ctctcaccaaagttactctggggtcggccattagatatcgactcagggttgtcaagtctctgtctgtcatgaactctccc

SEQ ID NO: 957 - DNA, 80 bp, Rat
ctctcacccagagtggatctgagaaagggcaggagagacagggttcaggtgggggcaacgtggaaagccgttactctccc

SEQ ID NO: 958 - DNA, 80 bp, Rat
ctctcaccgagggcagccatcagcaggactactagcaaagcacacgccacacccacaccgatgaagacgttcactctccc

SEQ ID NO: 959 - DNA, 80 bp, Rat
ctctcacccctatagttatgagcagaattcactgtaagtcacacgagtcatcctcagtggtcaacgtctgcaactctccc

SEQ ID NO: 960 - DNA, 80 bp, Rat
ctctcacctgcccagtttgtgtttgtttctctcacccatcagtcagtccacctcctctgtcttcggaagtccactctccc

SEQ ID NO: 961 - DNA, 80 bp, Rat
ctctcaccatgagaggaaagctgagggcggagaagaggaagtgagggggaaggaggggtctggttcacttctactctccc

SEQ ID NO: 962 - DNA, 80 bp, Rat
ctctcaccgcctcaggggactgagacgtgatgccagacacaagagctacctcacttcactccccacaaccttactctccc

SEQ ID NO: 963 - DNA, 80 bp, Rat
ctctcaccagggcccaggtaggggcagtggtaatctggttttagcctggtccctggtatggagggcttgatcactctccc

SEQ ID NO: 964 - DNA, 80 bp, Rat
ctctcacccataaaaaagataatataaaggaagacaatttgctgagtattaaaagttatctcaatatgaactactctccc

SEQ ID NO: 965 - DNA, 80 bp, Rat
ctctcaccctttgatagctcatgggacagccagtccagaccatcatacaggcctgtgccttgggtggcacagactctccc

SEQ ID NO: 966 - DNA, 80 bp, Rat
ctctcaccaagaaaacccccaaccccactaaacggtagaacagtaactcgagttcagtttcaaaaaagagagactctccc

SEQ ID NO: 967 - DNA, 80 bp, Rat
ctctcaccaaacagagcgaagagctaccagatacagacaggcttggtccacactcgctgctgcttgtcaagcactctccc

SEQ ID NO: 968 - DNA, 80 bp, Rat
ctctcacctgtggactccccaagttctaactctgataagacgtcaatctgtagggaggggtccacaccaagtactctccc

SEQ ID NO: 969 - DNA, 80 bp, Rat
ctctcaccttcccggcaggtcttggttaaaatccgctggccctcagcaagaacttttcctccatagatacatactctccc

SEQ ID NO: 970 - DNA, 80 bp, Rat
ctctcaccctctgctgcttgagagcttcaaagtaagggatgattttcgtcttccctcgactcttatcaatgaactctccc

SEQ ID NO: 971 - DNA, 80 bp, Rat
ctctcaccgggccgtcggcttttctgaagcattaccacaaatcagggacagtatcttttccagaagtaccaaactctccc

SEQ ID NO: 972 - DNA, 80 bp, Rat
ctctcaccctgggtcacccggatggtcttgggggttccatcccctgtgaaggtggtctccagatgtgtagtgactctccc

SEQ ID NO: 973 - DNA, 80 bp, Rat
ctctcacccctgtttttgaatcaagttttcaatgatggctttggtctcaggttgggaagcattacttgtgaaactctccc

SEQ ID NO: 974 - DNA, 80 bp, Rat
ctctcacctgtggtccaggtggtcagccacttgggacaaggtggcgctgtctgagcaggacacgaactggttactctccc

SEQ ID NO: 975 - DNA, 80 bp, Rat
ctctcaccgccccatcagcacttgggcctctagaagaccatgtatattgatttgcaggtagtttctctctcaactctccc

SEQ ID NO: 976 - DNA, 80 bp, Rat
ctctcacccacagtctcagagaactgggcaggagtggccagcttcagcagagtgatgtcattgcgcacagtgactctccc

SEQ ID NO: 977 - DNA, 80 bp, Rat
ctctcaccgtggcatagtccacaaccagcagacggccctgctgcaggacgtcaggagtggctccgttggtctactctccc

SEQ ID NO: 978 - DNA, 80 bp, Rat
ctctcacccacaacattcaccatcttttccactcctctcctgtcattctggacagtattgtaggacgtgtatactctccc

SEQ ID NO: 979 - DNA, 80 bp, Rat
ctctcaccaacggagcatcaccaaacctcgggggcaggcagatggtctgtatggtcctggatggctgtgcgcactctccc

SEQ ID NO: 980 - DNA, 80 bp, Rat
ctctcacctggcatcacagagggtcaggatggacacattctgggcccggttcagccaggtcacagcaaccttactctccc

SEQ ID NO: 981 - DNA, 80 bp, Rat
ctctcacccaggggagacagagtacaaagaaggccagggttccatctggccaggaagcagtgagttaaagacactctccc

SEQ ID NO: 982 - DNA, 80 bp, Rat
ctctcaccgtcagaccttcacccttaaaacgtacaagtttgtgaagtacctgtcacaaagtaaactctgtctactctccc

SEQ ID NO: 983 - DNA, 80 bp, Rat
ctctcacccccccttaaataagggaacaagatttggaccagcctagtaagggggaaaaaaattgaaattctgactctccc

SEQ ID NO: 984 - DNA, 80 bp, Rat
ctctcacccacattcaagtctagcccaggagtcccgtcagcttgcccagtgttgaacaggtaccattgctttactctccc

SEQ ID NO: 985 - DNA, 80 bp, Rat
ctctcacccccagcctgtcacccgccctttataaccagcctggagcaaactggttactgtctgcttgtctggactctccc

SEQ ID NO: 986 - DNA, 80 bp, Rat
ctctcaccggatagtgaaccggtgatttacgaaaggtaagagaactcagttgttcgccatagtctcctgaatactctccc

SEQ ID NO: 987 - DNA, 80 bp, Rat
ctctcacctccatccagcttaagcagggccaggtcgttctcgtatttgaggttgtagccagggtgtttaatgactctccc

SEQ ID NO: 988 - DNA, 80 bp, Rat
ctctcaccccaggagcttgaggtctctagtggcttggccactctgcttcagcgcaacactcagatgtcggagactctccc

SEQ ID NO: 989 - DNA, 80 bp, Rat
ctctcaccagaaaacctgcacgattccagctccagatttcctcacacactggcccgagatccagagttaattactctccc

SEQ ID NO: 990 - DNA, 80 bp, Rat
ctctcaccgtggggcagtagagcttggtgccatccccaggctgcctgagggtctggtcagagcaggtttctgactctccc

SEQ ID NO: 991 - DNA, 80 bp, Rat
ctctcaccacagttcacaacgtaattatttgctctcttctctttgactcccagggcttgcatgatcaactgcactctccc

SEQ ID NO: 992 - DNA, 80 bp, Rat
ctctcacctgtagttctccttggtgatgcttccgtcaaagaccctccagcgccactggtcaatgaggtagctactctccc

SEQ ID NO: 993 - DNA, 80 bp, Rat
ctctcaccatgtctgcagtcccccaactaaccctcttgaagttcagtgggatttgcatactccacattctgaactctccc

SEQ ID NO: 994 - DNA, 80 bp, Rat
ctctcacccaggaactgtattcgtgagcagtaccactccgcgtctcatcatacgggtaattagccacgaggtactctccc

SEQ ID NO: 995 - DNA, 80 bp, Rat
ctctcaccatgggctggctcatctggaagaagaacttatttggtcctctcctctcccatgtgcttgcactcaactctccc

SEQ ID NO: 996 - DNA, 80 bp, Rat
ctctcacctggctgtgttgtccagtttgatgacaatgtctacatcatctggtgggctgagaccttctttatcactctccc

SEQ ID NO: 997 - DNA, 80 bp, Rat
ctctcaccagtcccaaactagagtttgtccaaccacgttgttggcgatgctgacgatggtggaggtagcatcactctccc

SEQ ID NO: 998 - DNA, 80 bp, Rat
ctctcaccagcatcgctgtgatccccccattcccagtgctagtgttgaagtgcataaagaagccagagtcttactctccc

SEQ ID NO: 999 - DNA, 80 bp, Rat
ctctcaccataccggaggcgttcttgtccggtactggatgcctagggctgccaggcaggccaccagccctgcactctccc

SEQ ID NO: 1000 - DNA, 80 bp, Rat
ctctcacctatgtagctcccattttgctctgccatagcacatgctagagtaaggaaaccacttcagtgcgccactctccc

SEQ ID NO: 1001 - DNA, 80 bp, Rat
ctctcaccgcagtgcaatctcattggtatctgtcccagcctgggagctcaaggttggggttggggaggtgggactctccc

SEQ ID NO: 1002 - DNA, 80 bp, Rat
ctctcacccaggtcctagtaatccctttgtatatctaaagttctggatgcacacagagcctccagccacccgactctccc

SEQ ID NO: 1003 - DNA, 80 bp, Rat
ctctcacctgtgattgtgcaggtaaagaatgatggcatgctgaaacacgtcttcactaaggtaacttttcaaactctccc

SEQ ID NO: 1004 - DNA, 80 bp, Rat
ctctcaccggatccggaagtagccactctcaccccattgagagccccagctgttcttgacaatccagtagtcactctccc

SEQ ID NO: 1005 - DNA, 80 bp, Rat
ctctcacccatcctgccccacatacgggtaagcgtcttcagagtcaatgcctccattctgctgcacatattgactctccc

SEQ ID NO: 1006 - DNA, 80 bp, Rat
ctctcaccccattgcagcctcatcattgagtgtgatgttgacaacattcttgacgaacgcgacggccttttcactctccc

SEQ ID NO: 1007 - DNA, 80 bp, Rat
ctctcacccccatagaatgttccagattgtgaagcattataaacttgagattcttttcccagatgagacgtcactctccc

SEQ ID NO: 1008 - DNA, 80 bp, Rat
ctctcaccacgggtttggtcagttttaagatgaactccttcggatgtagtgtccgtcagtccttattatcgcactctccc

SEQ ID NO: 1009 - DNA, 80 bp, Rat
ctctcaccgtcagacatggcttccactgccccaaatgcccaacaagagccacaggacccctggtctctgatcactctccc

SEQ ID NO: 1010 - DNA, 80 bp, Rat
ctctcaccagtttcagcatggcgcaaagtgactggatgaaccatgacccgtcccttgaatttctccaggaatactctccc

SEQ ID NO: 1011 - DNA, 80 bp, Rat
ctctcaccttgtgtcaacctttaaaatatctgcaacttcattagagtatgtgatcccacatattccctcctgactctccc

SEQ ID NO: 1012 - DNA, 80 bp, Rat
ctctcaccttacagcttccattgcatattcgatttgatgaatcctgccctgagggctccaaacagtgacatcactctccc

SEQ ID NO: 1013 - DNA, 80 bp, Rat
ctctcaccaggatggatttctgctttttctcagtggctaataccacgccatttgcagctttaattcccactgactctccc

SEQ ID NO: 1014 - DNA, 80 bp, Rat
ctctcaccatggtgaaatgttacatattgtcgtcatctgattcatcttcttccttcaaagattccttggcatactctccc

SEQ ID NO: 1015 - DNA, 80 bp, Rat
ctctcaccggctctggcactctaccctggctctgttgatgactatccttgcatcggcagtgagacctgagacactctccc

SEQ ID NO: 1016 - DNA, 80 bp, Rat
ctctcacccaactgtcattgtctcattataggtgaaccagtggttctgtgtctccactctggctttatcaatactctccc

SEQ ID NO: 1017 - DNA, 80 bp, Rat
ctctcacccgtccagctcatgcaccagctgccgataatcacccacatgaggctgcttggctgctttggccacactctccc

SEQ ID NO: 1018 - DNA, 80 bp, Rat
ctctcaccgttgggctcccagagagactcacacatcccatacatttgttcagagcaggtgccactgactacgactctccc

SEQ ID NO: 1019 - DNA, 80 bp, Rat
ctctcaccgaaggtaatgttctccagattgtggatgccatctttgtcaatgacccgaacgctgaaggtgggcactctccc

SEQ ID NO: 1020 - DNA, 80 bp, Rat
ctctcaccctcttgtcattcacaatcaactggagttcttcaatcttggacagataatattgacggagtccacactctccc

SEQ ID NO: 1021 - DNA, 80 bp, Rat
ctctcacccgtcactgtatgtgacatcaggtttttcctccacctgcatcatggtaactgttgggtcaatcttactctccc

SEQ ID NO: 1022 - DNA, 80 bp, Rat
ctctcaccgaggaactccttcttcaggttcttctgctcatccttgatatactcctcctgcacctccaggaacactctccc

SEQ ID NO: 1023 - DNA, 80 bp, Rat
ctctcacctctacaaatgacaggatgctgacgtagtgttctgagcccaccgatgtggacacaatggcgtgatactctccc

SEQ ID NO: 1024 - DNA, 80 bp, Rat
ctctcaccggggcttcataagctacaccaagcatgtccacataaccaagaaagctttctccaccagcatagcactctccc

SEQ ID NO: 1025 - DNA, 80 bp, Rat
ctctcacccaatacagtacctccgttgaaggcataaggcgagaaacgcatctgcacagggccggcggagcccactctccc

SEQ ID NO: 1026 - DNA, 80 bp, Rat
ctctcaccaagacttcatcaagaagcttgtggatgttgcgcctctctgctgcaagcagaacgccatcattggactctccc

SEQ ID NO: 1027 - DNA, 80 bp, Rat
ctctcaccgatctgtccacaagcaatgactgtcactctccagtttgcaagggatggctgaacagggaaacacactctccc

SEQ ID NO: 1028 - DNA, 80 bp, Rat
ctctcacctagcatgggatcatagggcagcgtgtgatcttgcactcgcagcccatctggtacctgtggtttaactctccc

SEQ ID NO: 1029 - DNA, 80 bp, Rat
ctctcacccctctgtgattccagagagatcagcatcattgttgaagacccgggtgatgcccagtgagctcagactctccc

SEQ ID NO: 1030 - DNA, 80 bp, Rat
ctctcaccctgcagtagccacccttctgccggatgcaggcgtagtgtttggactgatagccagggtacccgaactctccc

SEQ ID NO: 1031 - DNA, 80 bp, Rat
ctctcaccaggacagtcatactcatggttaaggggcatgttgacggctgtctccgagttggggtcttgtttaactctccc

SEQ ID NO: 1032 - DNA, 80 bp, Rat
ctctcaccgaagctcaggtgcactttgttgcgaagacatttttcaacttgaaactttacagaatctgcaatcactctccc

SEQ ID NO: 1033 - DNA, 80 bp, Rat
ctctcacccatgcgggctgagactagaatggctgtggaggaagacgccactgtgccgctctcgttcacctcgactctccc

SEQ ID NO: 1034 - DNA, 80 bp, Rat
ctctcaccttttcttgatatgtaggttggacaaaatctgctgaaagctccccaatcagctctgactcactcaactctccc

SEQ ID NO: 1035 - DNA, 80 bp, Rat
ctctcaccaatgtagaccaaaacatcatcttcatccagtgtctctttgctgatccacttgttgaagttatcgactctccc

SEQ ID NO: 1036 - DNA, 80 bp, Rat
ctctcaccgggcactttctcttgcttaggttactcaggatctggtcctggttgggcctgtagagcaccacgaactctccc

SEQ ID NO: 1037 - DNA, 80 bp, Rat
ctctcacccgcccacacggtcgtacacacacttggtctgctcgatgagctgtgtggttagagctctgatctgactctccc

SEQ ID NO: 1038 - DNA, 80 bp, Rat
ctctcaccgctcctggctctgcctgaaatcatccagccccaccacgccgtctgccagttggcccagaccaccactctccc

SEQ ID NO: 1039 - DNA, 80 bp, Rat
ctctcacctcccaatgaaacatcttcatgattgagaacccatttcttatcctttggaataggattcagcagaactctccc

SEQ ID NO: 1040 - DNA, 80 bp, Rat
ctctcacccagggctctggccagagggctgggcagaaacaagaggtaggcctccacgcttgcacggtggctgactctccc

SEQ ID NO: 1041 - DNA, 80 bp, Rat
ctctcaccgagttcatgctggctgttgatgtcacggtagaccacggtatagttggtgatgcgaccattcctcactctccc

SEQ ID NO: 1042 - DNA, 80 bp, Rat
ctctcacctgttgtcaactgcagctttcgtgacatctgccgttgtcatgttttggatgttggcactcactttactctccc

SEQ ID NO: 1043 - DNA, 80 bp, Rat
ctctcaccaactggccaccacgagacttccgaagtgggaccatcacaatgaagtagttctgcactgtcacggactctccc

SEQ ID NO: 1044 - DNA, 80 bp, Rat
ctctcaccaaggtcaccaaaggagtgtccagagtgagtccccaaaatggccttggagaggcttctgctgaagactctccc

SEQ ID NO: 1045 - DNA, 80 bp, Rat
ctctcacccatggtgacgtaggtgatttgcaaccacaaggcactcgtcgtggtattgttggctttcatgctgactctccc

SEQ ID NO: 1046 - DNA, 80 bp, Rat
ctctcaccacggccacatcagctaccgccagtgacacgatgaagcagaaggtggcatcgcgaagtgcctggtactctccc

SEQ ID NO: 1047 - DNA, 80 bp, Rat
ctctcacccagacccagcaggcaggggaggggggaagtcaataaaatagaatgggtttcctcacatttttatactctccc

SEQ ID NO: 1048 - DNA, 80 bp, Rat
ctctcaccgaagtcccggttcctgtaggcatagacaatgggattgacaactgaattggcgtgtgacaggaggactctccc

SEQ ID NO: 1049 - DNA, 80 bp, Rat
ctctcacctcttggaggtggcaattaaaagtttcttcccaaaactctttggcaaaaccattgtggacagactactctccc

SEQ ID NO: 1050 - DNA, 80 bp, Rat
ctctcaccccacttcctctgcacggtacagagctttctccgtccttcaatggacatggtttctcatcagggtactctccc

SEQ ID NO: 1051 - DNA, 80 bp, Rat
ctctcacctctccagggagctggagctgcggaggggcaggagtcggtgaccagggttcccacctctgcgcccactctccc

SEQ ID NO: 1052 - DNA, 80 bp, Rat
ctctcaccgtacatggacagcaccgtgatgatgaggcggatagccctccgccttttcttctccgagtgctcgactctccc

SEQ ID NO: 1053 - DNA, 80 bp, Rat
ctctcaccgctgcggtggttctgggaccagacggggaggagcacggagatgcagcggtcgaagctgatgacaactctccc

SEQ ID NO: 1054 - DNA, 80 bp, Rat
ctctcaccacttcacccaggtcagaacgtggtgtctcatcagggtgagctcatactcctggaagttgttataactctccc

SEQ ID NO: 1055 - DNA, 80 bp, Rat
ctctcaccctgctgactgaggcggcgaaggacgccaccaggaagcccaccatgagcaggctcacagtctccgactctccc

SEQ ID NO: 1056 - DNA, 80 bp, Rat
ctctcaccgatgttctccggagtaccagcttctatacagtcccgaggcagcccgcttaaattgaccttcgggactctccc

SEQ ID NO: 1057 - DNA, 80 bp, Rat
ctctcacctcccaggagggtgggaggaagtagttacagtggctatcccagttagagccgtagaaaaagaagaactctccc

SEQ ID NO: 1058 - DNA, 80 bp, Rat
ctctcaccctaatgactctgaagctccccagctcagacctggcagagtgtcatcagatgaccacactgacggactctccc

SEQ ID NO: 1059 - DNA, 80 bp, Rat
ctctcaccgggagctcaccagtgtgtgtccctgtctcgcaaagtctgtggccgtcctgatcctccgagttccactctccc

SEQ ID NO: 1060 - DNA, 80 bp, Rat
ctctcaccagaccctcagccccaccctgccgaaggctgccaggacaaagctggagcctgggagatccgaatcactctccc

SEQ ID NO: 1061 - DNA, 80 bp, Rat
ctctcaccagggggtgcaggggcaggtgggaaacacccaaggtctaggagcactggatggacatgtaaggccactctccc

SEQ ID NO: 1062 - DNA, 80 bp, Rat
ctctcaccaaaggactcatgacaggcaggttgaccatggtacggatcttctgccaatggcttttctgccccaactctccc

SEQ ID NO: 1063 - DNA, 80 bp, Rat
ctctcacctgacactgaatcgcgtcattcgggccagccctccgaatccctgctccgaacgggaagtgattcgactctccc

SEQ ID NO: 1064 - DNA, 80 bp, Rat
ctctcaccgtccagcaggaagcccgaggctgcctgggcctcaagtaagataagggctgttccagggctgagcactctccc

SEQ ID NO: 1065 - DNA, 80 bp, Rat
ctctcaccgcgctccaggttggcgatggtctcctggcagcggcagagacaggccttgatggacttcttccagactctccc

SEQ ID NO: 1066 - DNA, 80 bp, Rat
ctctcacctcacgaatcacactgatcacgactttatttaaaacttagcagacaacactgtgcagaagcatccactctccc

SEQ ID NO: 1067 - DNA, 80 bp, Rat
ctctcacctaggggtcgtccacagtctgccccacatctcctaccagaatctccttgccctcctccaggatgaactctccc

SEQ ID NO: 1068 - DNA, 80 bp, Rat
ctctcaccaactgcgtgtgtgtccactgcatttccaggcatgcattccgctcccgttgcaggaagggggcagactctccc

SEQ ID NO: 1069 - DNA, 80 bp, Rat
ctctcaccaaaacaaaggaagccctgggggtccaggcagctggagtcagcagagagggctgctgtacagggaactctccc

SEQ ID NO: 1070 - DNA, 80 bp, Rat
ctctcacccgtactcctcgttcatagtgatcactccgtcgcacgtctcgtccagtaccagccggcgcgtcatactctccc

SEQ ID NO: 1071 - DNA, 80 bp, Rat
ctctcaccagtttctcccgcttgagatgctcatacatcaccgggatagccattgccgcccgaagctgccgctactctccc

SEQ ID NO: 1072 - DNA, 80 bp, Rat
ctctcaccagtgctaggtaaagggtgatgggacagtggtcactgccaagagccttggaccggatcttgctgtactctccc

SEQ ID NO: 1073 - DNA, 80 bp, Rat
ctctcacccccagggacagggtcctggggctggctgggcagacgcctgcttacaggctgcataaaggcatcgactctccc

SEQ ID NO: 1074 - DNA, 80 bp, Rat
ctctcaccccttcagcagcttccataacacctgtctggtgaatgaatcttgctggaacacagggtgatggagactctccc

SEQ ID NO: 1075 - DNA, 80 bp, Rat
ctctcaccgctgcggtcatgtcgtctattttgtagacaatgttggttggcgctttctctgcatgtctgattaactctccc

SEQ ID NO: 1076 - DNA, 80 bp, Rat
ctctcacctcgcacgggcaaagttgacgaaagagatctcatcgaagcggatgtgcacagggggcttgtgcacactctccc

SEQ ID NO: 1077 - DNA, 80 bp, Rat
ctctcaccgatgacaggaggttgagcgccagcatagaaggccgtggctggtaggcccaccgggagaggctctactctccc

SEQ ID NO: 1078 - DNA, 80 bp, Rat
ctctcaccgaatgcaggtgaggagcatgtaactgagggaggccgcgataaacacaatgaaagcattttcatgactctccc

SEQ ID NO: 1079 - DNA, 80 bp, Rat
ctctcacctggcagcgttcacgaaggcttgagaagtgtggttcatggtagcgatccaccgcgatcgcacacaactctccc

SEQ ID NO: 1080 - DNA, 80 bp, Rat
ctctcacctaacccaaataaacaaaaagtggactacattaccacaaaaaaagtactgaatatgccagaatctactctccc

SEQ ID NO: 1081 - DNA, 80 bp, Rat
ctctcaccggtaagtaataaacttaaaaatgagaaaagtggattggaaactgcataaagctacaagtgactgactctccc

SEQ ID NO: 1082 - DNA, 80 bp, Rat
ctctcaccgtcatggatgaccttggccaggggggctaagcagttggtggtgcaggatgcattgctgacaatcactctccc

SEQ ID NO: 1083 - DNA, 80 bp, Rat
ctctcaccaatggagaactccagcttggacttctttccgtagtcgacagagagcctctccatcagcagggagactctccc

SEQ ID NO: 1084 - DNA, 80 bp, Rat
ctctcaccactaggcagatgacactcttaagagactttgggcaccaagttcacagtaaacacttgagatgggactctccc

SEQ ID NO: 1085 - DNA, 80 bp, Rat
ctctcacctgtgaattgatccctgtattttttacacctccacaagtggaagatcctttttagctccttgtgaactctccc

SEQ ID NO: 1086 - DNA, 80 bp, Rat
ctctcaccgccaggatagagccaccaatccacacagagtacttgcgctcaggaggagcaatgatcttgatctactctccc

SEQ ID NO: 1087 - DNA, 80 bp, Rat
ctctcaccactccagtagagctgctggtgacccatcttgccttcagacgcccaaggaggaaaaaggaacgagactctccc

SEQ ID NO: 1088 - DNA, 80 bp, Rat
ctctcaccatgccatcagtgcccgccgggaatgcagactgaagaattaatagccacccctcaggcggaggacactctccc

SEQ ID NO: 1089 - DNA, 80 bp, Rat
ctctcaccgacaggaagtggggtgacagcctaacagtgtttcttggtgtcaaggtctttgtattccttgttgactctccc

SEQ ID NO: 1090 - DNA, 80 bp, Rat
ctctcaccggactcttgtagattcaacttgccgctgtcttttaggctttgtacttggcttttccactttcgcactctccc